(19) United States
(12) Patent Application Publication
(10) Pub. No.: US 2024/0317865 A1
(43) Pub. Date: Sep. 26, 2024

(54) ANTIBODIES AGAINST INTEGRIN ALPHA 11 BETA 1

(71) Applicant: MOMENTA PHARMACEUTICALS, INC., Cambridge, MA (US)

(72) Inventors: Elma KURTAGIC, Cambridge, MA (US); James W. MEADOR, III, Cambridge, MA (US); Christopher BENEDUCE, Cambridge, MA (US)

(21) Appl. No.: 18/258,189

(22) PCT Filed: Dec. 17, 2021
(86) PCT No.: PCT/IB2021/061926

§ 371 Date: Jun. 16, 2023

Related U.S. Application Data
(60) Provisional application No. 63/213,973, filed on Jun. 23, 2021.
(60) Provisional application No. 63/127,849, filed on Dec. 18, 2020.

(51) Int. Cl. C07K 16/28; A61P 13/12; C12N 15/10

(52) U.S. Cl. CPC C07K 16/2842; A61P 13/12; C12N 15/1037; C07K 2317/24

(57) ABSTRACT

The present disclosure includes antibodies that specifically bind integrin alpha 11 beta 1 (α11β1), as well as methods of making and using such antibodies.

### CROSS-REFERENCE TO RELATED APPLICATIONS

This application claims the benefit of priority of U.S. Provisional Application No. 63/127,849, filed Dec. 18, 2020, and U.S. Provisional Application No. 63/213,973, filed Jun. 23, 2021, which are incorporated by reference herein, in their entireties and for all purposes.

### SEQUENCE LISTING

The instant application contains a Sequence Listing which has been submitted electronically in ASCII format and is hereby incorporated by reference in its entirety. Said ASCII copy, created on Nov. 19, 2021, is named 2010403_0592_SL.txt and is 440,570 bytes in size.

### BACKGROUND

Fibrosis is a process of scarring that manifests itself in many tissues in the body, typically as a result of inflammation or tissue damage. Increased production of extracellular matrix results in organ failure and, often, death. Diseases associated with fibrosis account for approximately 45% of all deaths in industrialized nations (Wynn, T. A., 2008, J Pathol. 214:199-210). One such disease is Systemic Sclerosis (SSc). SSc is a complex autoimmune disease with a chronic progressive course and high interpatient variability. It is characterized by inflammation, vascular dysfunction and fibrosis. Fibrosis of the skin and visceral organs results in irreversible scarring and ultimately organ failure, accounting for high mortality. There is currently no approved targeted therapy with disease-modifying potential.

### SUMMARY

The present disclosure provides novel, function-blocking antibodies against type I collagen receptor integrin alpha 11 beta 1 (α11β1). The present disclosure also provides use of such antibodies to treat fibrotic disorders and/or cancers.

In one aspect, the present disclosure provides an anti-α11β1 antibody, or antigen-binding fragment thereof, comprising an amino acid sequence selected from a group consisting of SEQ ID NO: 103-443. In another aspect, the present disclosure provides an anti-α11β1 antibody, or antigen-binding fragment thereof, comprising a CDR sequence encompassed within any one of SEQ ID NO: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443. In another aspect, the present disclosure provides an anti-α11β1 antibody, or antigen-binding fragment thereof, comprising CDR1, CDR2, and CDR3 encompassed within any one of SEQ ID NO: 103-206, or 413-435. In some embodiments, an anti-α11β1 antibody, or antigen-binding fragment thereof, comprises an amino acid sequence selected from a group consisting of SEQ ID NO: 103-114, 207-311, and 312-443. In some embodiments, an anti-α11β1 antibody, or antigen-binding fragment thereof, comprises a CDR sequence encompassed within any one of SEQ ID NO: 103-114, 207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443. In some embodiments, an anti-α11β1 antibody, or antigen-binding fragment thereof, comprises one or more CDR sequences encompassed within any one of SEQ ID NO: 103-114, or 413-434. In some embodiments, an anti-α11β1 antibody, or antigen-binding fragment thereof, comprises CDR1, CDR2, and CDR3 encompassed within any one of SEQ ID NO: 103-114 or 413-434.

In some embodiments, an anti-α11β1 antibody, or antigen-binding fragment thereof, is a monoclonal antibody, or antigen-binding fragment thereof. In some embodiments, an anti-α11β1 antibody, or antigen-binding fragment thereof, is a humanized antibody, or antigen-binding fragment thereof. In some embodiments, an anti-α11β1 antibody, or antigen-binding fragment thereof, reduces interaction of α11β1 with collagen in human α11β1-expressing cells. In some embodiments, an anti-α11β1 antibody, or antigen-binding fragment thereof, competes with an antibody, or antigen-binding fragment thereof, described herein.

In another aspect, the present disclosure provides a nucleic acid, comprising a nucleic acid sequence encoding an antibody, or antigen-binding fragment thereof, described herein. In some embodiments, a nucleic acid sequence comprises a sequence selected from a group consisting of SEQ ID NO: 1-102.

In another aspect, the present disclosure provides a vector comprising a nucleic acid described herein.

In another aspect, the present disclosure provides a host cell comprising a nucleic acid described herein or a vector described herein.

In another aspect, the present disclosure provides a method of producing an antibody, or antigen-binding fragment thereof, comprising culturing a host cell described herein under conditions suitable for expression of the antibody or antigen-binding fragment thereof.

In another aspect, the present disclosure provides a method of treating a subject having or at risk of chronic kidney disease, the method comprising administering to the subject a therapeutically effective amount of the antibody, or antigen-binding fragment thereof, described herein. In some embodiments, a chronic kidney disease is or comprises Primary Glomerular Disease (including, but not limited to, IgA Nephropathy and focal segmental glomerular sclerosis), Secondary Glomerular Disease (including, but not limited to, lupus nephritis), Thrombotic Microangiopathy, Tubulointerstitial Diseases (including, but not limited to, Obstructive Uropathy), Diabetic Nephropathy, Hypertensive Nephropathy, Ischemic Nephropathy, Cardiorenal Syndromes in CKD, Inherited Disorders of the Glomerulus (including, but not limited to, Alport syndrome), Cystic Diseases of the Kidney (including, but not limited to, Polycystic Kidney Disease), or Inherited Disorders of the Renal Tubule. In some embodiments, administering a therapeutically effective amount of an antibody, or antigen-binding fragment thereof results in a reduction in a measured marker, sign and/or symptom by at least about 10%, 15%, 20%, 25%, 30%, 35%, 40%, 50%, 55%, 60%, 65%, 70%, 75%, 80%, 85% or 90% relative to a control. In some embodiments, a control comprises a level of measured marker, sign and/or symptom in a subject prior to administration of an antibody. In some embodiments, a control comprises a level of measured marker, sign and/or symptom in a subject suffering from a kidney-related disorder. In some embodiments, a control comprises an average level of measured marker, sign and/or symptom in a population of subjects suffering from a kidney-related disorder. In some embodiments, a measured marker, sign and/or symptom is or comprises: COL1A1, Fibronectin, PAI-1, IL-11, CXCL1, MCP-1, IL-6, TIMP-1, Hyaluronic acid, TGFβ, CTGF, PDGF, MMP9, or a combination thereof.

### BRIEF DESCRIPTION OF THE DRAWING

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows representations of an integrin structure. The panels illustrate the structure of collagen-binding integrins and three different conformations integrins can exist in on the surface of a cell.

FIG. 2A shows a chart illustrating an ELISA analysis of binding of exemplary mouse monoclonal antibodies to human α11β1.

FIG. 2B shows a chart illustrating an exemplary ELISA analysis of binding of mouse monoclonal antibodies to mouse α11β1.

FIG. 3A shows a graph illustrating an ELISA analysis of binding of exemplary rat monoclonal antibodies to a human α11β1 I domain.

FIG. 3B shows a graph illustrating an ELISA analysis of binding of exemplary mouse monoclonal antibodies to a human α11β1 I domain.

FIG. 4A shows a graph illustrating an FACS analysis of binding of exemplary rat monoclonal antibodies to CHO-K1 cells expressing human α11β1.

FIG. 4B shows a graph illustrating an FACS analysis of binding of exemplary mouse monoclonal antibodies to CHO-K1 cells expressing human α11β.

FIG. 5 shows graphs illustrating a FACS analysis of binding of exemplary mouse monoclonal antibodies to human pulmonary fibroblasts (HPFs) and myofibroblasts (MF).

FIG. 6A shows graphs illustrating the ability of exemplary rat monoclonal antibodies to inhibit adhesion of CHO-K1 cells expressing human α11 to rat tail type I collagen.

FIG. 6B shows graphs illustrating the ability of exemplary rabbit monoclonal antibodies to inhibit adhesion of CHO-K1 cells expressing human α11 to rat tail type I collagen.

FIG. 6C shows graphs illustrating the ability of exemplary mouse monoclonal antibodies to inhibit adhesion of CHO-K1 cells expressing human α11 to rat tail type I collagen.

FIG. 7A shows a graph illustrating the ability of exemplary rat monoclonal antibodies to inhibit Fibroblast-to-Myofibroblasts Transition (FMT) as measured by percent inhibition of αSMA upregulation.

FIG. 7B shows a graph illustrating the ability of exemplary rabbit monoclonal antibodies to inhibit Fibroblast-to-Myofibroblasts Transition (FMT) as measured by percent inhibition of αSMA upregulation.

FIG. 7C shows a graph illustrating the ability of exemplary mouse monoclonal antibodies to inhibit Fibroblast-to-Myofibroblasts Transition (FMT) as measured by percent inhibition of αSMA upregulation.

FIG. 8 shows graphs illustrating the ability of exemplary monoclonal antibodies to inhibit CHO-K1 human α11-mediated rat tail type I collagen gel contraction.

FIG. 9 shows graphs illustrating the affinity of exemplary monoclonal antibodies for human α11β1 via surface plasmon resonance (SPR).

FIG. 10A and FIG. 10B show graphs illustrating the affinity of exemplary monoclonal antibodies for human α11β1 via surface plasmon resonance (SPR).

FIG. 11A and FIG. 11B show graphs illustrating the binding ability of selected rabbit, rat, mouse and human monoclonal antibodies to α11β1 expressed on the surface of CHO cells.

FIG. 12 shows graphs illustrating a FACS analysis of binding of exemplary monoclonal antibodies to human pulmonary fibroblasts (HPFs) and myofibroblasts (MF).

FIG. 13 shows a graph and table illustrating a FACS analysis of binding of exemplary monoclonal antibodies to human myofibroblasts (MF).

FIG. 14 shows a graph illustrating the binding ability of selected monoclonal antibodies to α11β1 expressed on the surface of CHO cells.

FIG. 15A and FIG. 15B show graphs illustrating the ability of exemplary monoclonal antibodies to inhibit adhesion of CHO cells expressing human α11 to rat tail type I collagen.

FIG. 16 shows a graph illustrating the effect of exemplary monoclonal antibodies on xenograft growth in SCID mice.

FIG. 17A, FIG. 17B and FIG. 17 C illustrate the effect of exemplary monoclonal antibodies on soluble pro-fibrogenic markers from Precision-Cut Liver Slices (PCLS).

FIG. 18A, FIG. 18B and FIG. 18 C illustrate the effect of exemplary monoclonal antibodies on the soluble pro-fibrogenic marker Col1a1 from Precision-Cut Kidney Slices (PCKS).

### DETAILED DESCRIPTION

The present disclosure is based, in part, on the discovery of novel antibodies that selectively bind to α11β1. The disclosure also relates to nucleic acids encoding said antibodies and methods of use in the treatment of fibrosis and diseases comprising a fibrotic component.

### Fibrosis and Diseases

Fibrosis is a process of scarring that manifests itself in many tissues in the body, typically as a result of inflammation or tissue damage. Increased production of extracellular matrix results in organ failure anfd, often, death. Diseases associated with fibrosis account for approximately 45% of all deaths in industrialized nations (Wynn, T. A., 2008, J Pathol. 214:199-210). One such disease is Systemic Sclerosis (SSc). SSc is a complex autoimmune disease with a chronic progressive course and high interpatient variability. It is characterized by inflammation, vascular dysfunction and fibrosis. Fibrosis of the skin and visceral organs results in irreversible scarring and ultimately organ failure, accounting for high mortality. There is currently no approved targeted therapy with disease-modifying potential.

The cells responsible for producing extracellular matrix (ECM) for tissue repair (and in fibrosis) are a specialized type of fibroblasts called myofibroblasts (MF). Although mechanisms of fibrosis have been extensively studied, this complex process is far from well understood. In order to focus on the most important drivers of fibrosis, published patient-derived datasets (SSc patient data and normal controls) were interrogated using an in-house derived novel data analysis methodology. This analysis lead to the identification of the type I collagen-binding integrin alpha 11 beta 1 (α11β1) as one of the top targets for modulating fibrosis.

To this date, there are no truly disease-modifying therapeutics for fibrosis. Two of the approved therapies for idiopathic pulmonary fibrosis (IPF), nintedanib and pirfenidone, work poorly and do not modify the disease, and there is no approved therapy for systemic sclerosis (SSc) to date. In some embodiments, a fibrotic disorder is or comprises idiopathic pulmonary fibrosis (IPF), chronic kidney disease, diabetic cardiomyopathy, primary sclerosing cholangitis (PSC), primary biliary cirrhosis (PBC), non-alcoholic fatty liver disease (NAFLD/NASH), Crohn's disease, ulcerative colitis, or systemic sclerosis (SSc). In some embodiments, a fibrotic disorder is or comprises atrial fibrosis, endomyocardial fibrosis, arthrofibrosis, mediastinal fibrosis, myelofibrosis, progressive massive fibrosis, retroperitoneal fibrosis or skeletal muscle fibrosis.

One clinical feature of the tumor microenvironment is the interaction between tumor and stroma, which mainly relies on various integrins that interact with ECM components as well as growth factors. Such interaction can influence tumor survival, progression and eventually metastasis. α11β1 has been reported to be overexpressed in cancer-associated fibroblasts (CAFs) of metastatic tumors, and its expression has been correlated with aggressive tumors in patients. For example, integrin α11 was overexpressed in the stroma of most head and neck squamous cell carcinomas (HNSCC) and correlated positively with alpha smooth muscle actin expression (Parajuli et al., J. Oral Pathol. Med. 46:267-275 (2017)). Integrin α11 was also overexpressed by CAFs in Pancreatic Ductal Adenocarcinoma (PDAC) stroma (Schnittert et al., FASEB J. 33:6609-6621 (2019)). In addition, integrin α11β1 overexpression in the tumor stroma has been associated with tumor growth and metastatic potential of non-small cell lung cancer (NSCLC), and high expression of ITGA11 (gene encoding integrin alpha-11 in humans) was associated with lower recurrence-free survival in all NSCLC patients; the same study showed that α11 overexpression in lung cancer cell lines resulted in increased migration and invasion (Ando et al., Cancer Sci. 111:200-208 (2020)).

### Integrins

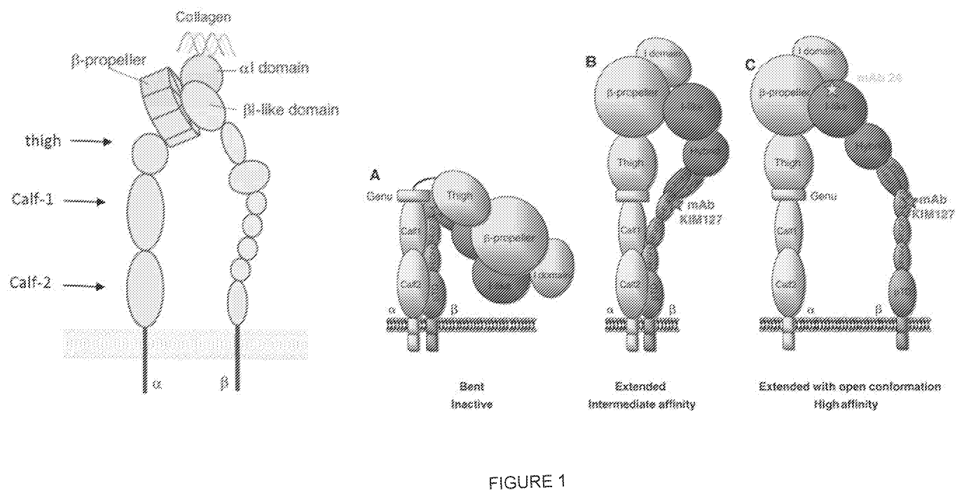

Integrins are a large family of type I transmembrane heterodimeric glycoprotein receptors and act as major receptors for cell adhesion. The integrin family of receptors plays key roles in modulating signal transduction pathways that control cell adhesion, migration, proliferation, differentiation and apoptosis. There are 18 α and 8 β subunits, which combine to form 24 integrin heterodimers. Each integrin receptor comprises two non-covalently bound subunits, α and β. Integrins α1β1, α2β1, α10β1, and α11β1 are the primary collagen receptors. α and β subunits are transmembrane proteins with large, modular, extracellular domains, single transmembrane helices, and short cytoplasmic regions, which mediate cytoskeletal interactions. Extracellular domain of integrins are generally large, approximately 80-150 kDa structures. The extracellular domains can be seen as comprising a headpiece connected to two legs (see FIG. 1 for structure of collagen-binding integrins). Collagen binding integrins contain an I domain, which serves as the ligand-binding site. The αI-domain contains a conserved “metal-ion-dependent adhesion site” (MIDAS) that binds divalent metal cations (Mg2+) and plays important role in ligand binding.

Integrins can exist in three different conformations: 1) a resting, low affinity state (bent conformation, FIG. 1, panel A) where the head piece containing ligand binding site is turned towards the membrane; 2) an extended, intermediate affinity state, where the integrin is extended but the head piece remains ‘closed’ (FIG. 1, panel B) and 3) an extended, high affinity state where the integrin is fully activated and readily binds the ligand. The complexity of the different integrin states allows for both allosteric and ligand-blocking ways of inhibiting integrin function. As marked with a star in FIG. 1, one of the allosteric ways to block the function of an integrin is to generate a monoclonal antibody that prevents the integrin from reaching the fully extended conformation from the extended intermediate conformation. Another allosteric option is to bind an integrin in its bent/inactive conformation and to keep it from extending to either of the two other states. A non-allosteric way of inhibiting integrin function is to bind to the I domain a prevent the integrin from attaching to collagen. Binding to the ligand binding site directly runs the risk of generating a recombinant activator of integrin function.

As cell surface receptors, integrins sense the stiffness of the surrounding matrix, triggering the cells to further produce and remodel connective tissue, which can perpetuate a fibrotic phenotype. Many integrins are overexpressed in fibrosis, but it is not clear which alpha subunit is sufficient for fibrosis to occur. α11β1 integrin is specifically expressed on a subset of fibroblasts and myofibroblasts (i.e., terminal scar producing cells). Recent literature has provided strong evidence that α11β1 is one of the main drivers of a fibrotic phenotype in cardiac tissue, liver, lungs and kidney (Romaine, A. et. al. Overexpression of integrin alpha 11 induces cardiac fibrosis in mice. Acta Physiol February 2018, 222(2); Bansal, R. et. al. Integrin alpha 11 in the regulation of the myofibroblast phenotype: implications for fibrotic diseases. Exp Mol Med. 2017 Nov. 17:49(11)). Blocking α11β1 function may inhibit myofibroblast differentiation and extracellular matrix deposition (i.e., the major event in scar formation) and blocking α11β1 function may provide a mechanism for local, injury-specific attenuation of fibrosis which could fundamentally change fibrotic microenvironment and modify disease progression in all diseases that have a fibrotic component.

In some embodiments, an anti-α11β1 antibody, or antigen-binding fragment thereof, of the present disclosure reduces interaction of α11β1 with collagen in human α11β1-expressing cells. In some embodiments, reducing interaction of α11β1 with collagen in human α11β1-expressing cells comprises an anti-α11β1 antibody, or antigen-binding fragment thereof, interacting with α11β1 that is in a resting, low affinity state (bent conformation). In some embodiments, reducing interaction of α11β1 with collagen in human α11β1-expressing cells comprises an anti-α11β1 antibody, or antigen-binding fragment thereof, interacting with α11β1 that is in an extended, intermediate affinity state. In some embodiments, reducing interaction of α11β1 with collagen in human α11β1-expressing cells comprises an anti-α11β1 antibody, or antigen-binding fragment thereof, interacting with α11β1 that is in an extended, high affinity state.

### Antibodies

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, antibody, diabody, triabody, tetrabody, minibody, maxibody, tandab, DVD, BiTe, TandAb, or the like, or any combination thereof. The subunit structures and three-dimensional configurations of different classes of antibodies are known in the art.

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

A “multispecific antibody” refers to an antibody comprising at least two different antigen binding domains that recognize and specifically bind to at least two different antigens. A “bispecific antibody” is a type of multispecific antibody and refers to an antibody comprising two different antigen binding domains that recognize and specifically bind to at least two different antigens.

A “different antigen” may refer to different and/or distinct proteins, polypeptides, or molecules; as well as different and/or distinct epitopes, which epitopes may be contained within one protein, polypeptide, or other molecule.

The term “epitope” refers to an antigenic determinant that interacts with a specific antigen binding site in the variable region of an antibody molecule known as a paratope. A single antigen may have more than one epitope. Thus, different antibodies may bind to different areas of an antigen and may have different biological effects. The term “epitope” also refers to a site of an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

As used herein, “selective binding”, “selectively binds” “specific binding”, or “specifically binds” refers, with respect to an antigen binding moiety and an antigen target, preferential association of an antigen binding moiety to an antigen target and not to an entity that is not the antigen target. A certain degree of non-specific binding may occur between an antigen binding moiety and a non-target. In some embodiments, an antigen binding moiety selectively binds an antigen target if binding between the antigen binding moiety and the antigen target is greater than 2-fold, greater than 5-fold, greater than 10-fold, or greater than 100-fold as compared with binding of the antigen binding moiety and a non-target. In some embodiments, an antigen binding moiety selectively binds an antigen target if the binding affinity is less than about 10⁻⁵ M, less than about 10⁻⁶ M, less than about 10⁻⁷ M, less than about 10⁻⁸ M, or less than about 10⁻⁹ M.

In some embodiments, antibodies or fragments thereof that selectively bind to an identical epitope or overlapping epitope that will often cross-compete for binding to an antigen. Thus, in some embodiments, the disclosure provides an antibody or fragment thereof that cross-competes with an exemplary antibody or fragment thereof as disclosed herein. In some embodiments, to “cross-compete”, “compete”, “cross-competition”, or “competition” means antibodies or fragments thereof compete for the same epitope or binding site on a target. Such competition can be determined by an assay in which the reference antibody or fragment thereof prevents or inhibits specific binding of a test antibody or fragment thereof, and vice versa. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al. (1983) Methods in Enzymology 9:242-253), solid phase direct biotin-avidin EIA (see, e.g., Kirkland et al., (1986) J. Immunol. 137:3614-9), solid phase direct labeled assay, solid phase direct labeled sandwich assay, Luminex (Jia et al. “A novel method of Multiplexed Competitive Antibody Binning for the characterization of monoclonal antibodies” J. Immunological Methods (2004) 288, 91-98) and surface plasmon resonance (Song et al. “Epitope Mapping of Ibalizumab, a Humanized Anti-CD4 Monoclonal Antibody with Anti-HIV-1 Activity in Infected Patients” J. Virol. (2010) 84, 6935-42). Usually, when a competing antibody or fragment thereof is present in excess, it will inhibit binding of a reference antibody or fragment thereof to a common antigen by at least 50%, 55%, 60%, 65%, 70%, or 75%. In some instances, binding is inhibited by at least 80%, 85%, 90%, 95%, 96%, 97%, 98%, 99%, or more.

An antibody can be an immunoglobulin molecule of four polypeptide chains, e.g., two heavy (H) chains and two light (L) chains. In some embodiments, a light chain is a lambda light chain. In some embodiments, a light chain is a kappa light chain. A heavy chain can include a heavy chain variable domain and a heavy chain constant domain. A heavy chain constant domain can include CH1, hinge, CH2, CH3, and in some instances CH4 regions. A light chain can include a light chain variable domain and a light chain constant domain. A light chain constant domain can include a CL.

A heavy chain variable domain of a heavy chain and a light chain variable domain of a light chain can typically be further subdivided into regions of variability, termed complementarity determining regions (CDRs), interspersed with regions that are more conserved, termed framework regions (FR). Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein. The CDRs in a heavy chain are designated “CDRH1”, “CDRH2”, and “CDRH3”, respectively, and the CDRs in a light chain are designated “CDRL1”, “CDRL2”, and “CDRL3”.

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

### Exemplary Antibodies

The present disclosure provides antibodies that can include various heavy chains and light chains described herein. In some embodiments, an antibody comprises two heavy chains and light chains. In some embodiments, the present disclosure encompasses an antibody including at least one heavy chain and/or light chain as disclosed herein, at least one heavy chain and/or light chain framework domain as disclosed herein, at least one heavy chain and/or light chain CDR domain as disclosed herein, and/or any heavy chain and/or light chain constant domain as disclosed herein.

In some embodiments, an antibody disclosed herein is a homodimeric monoclonal antibody. In some embodiments, an antibody disclosed herein is a heterodimeric antibody. In some embodiments, an antibody is, e.g., a typical antibody or a diabody, triabody, tetrabody, minibody, maxibody, tandab, DVD, BiTe, scFv, TandAb scFv, Fab, Fab2, Fab3, F(ab′)2, or the like, or any combination thereof.

The present disclosure provides, among other things, an anti-integrin alpha 11 beta 1 (α11β1) antibody, or antigen-binding fragment thereof. In some embodiments, an α11β1 antibody, or antigen-binding fragment thereof, comprises an amino acid sequence selected from a group consisting of SEQ ID NO: 103-443. In some embodiments, an anti-α11β1 antibody, or antigen-binding fragment thereof, comprises a CDR sequence encompassed within any one of SEQ ID NO: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-435. In some embodiments, an anti-11β1 antibody, or antigen-binding fragment thereof, comprises CDR1, CDR2, and CDR3 encompassed within any one of SEQ ID NO: 103-206, or 413-443. In some embodiments, an anti-α11β1 antibody, or antigen-binding fragment thereof, comprises an amino acid sequence selected from a group consisting of SEQ ID NO: 103-114, 207-311, and 312-443. In some embodiments, an anti-α11β1 antibody, or antigen-binding fragment thereof, comprises a CDR sequence encompassed within any one of SEQ ID NO: 103-114, 207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443. In some embodiments, an anti-α11β1 antibody, or antigen-binding fragment thereof, comprises one or more CDR sequences encompassed within any one of SEQ ID NO: 103-114, or 413-434. In some embodiments, an anti-α11β1 antibody, or antigen-binding fragment thereof, comprises CDR1, CDR2, and CDR3 encompassed within any one of SEQ ID NO: 103-114, or 413-434. In some embodiments, an anti-α11β1 antibody, or antigen-binding fragment thereof, is a monoclonal antibody, or antigen-binding fragment thereof. In some embodiments, an anti-α11β1 antibody, or antigen-binding fragment thereof, is a humanized antibody, or antigen-binding fragment thereof. In some embodiments, an anti-α11β1 antibody, or antigen-binding fragment thereof, reduces interaction of α11β1 with collagen in human α11β1-expressing cells. In some embodiments, the present disclosure provides an anti-α11β1 antibody, or antigen-binding fragment thereof, that competes with an antibody, or antigen-binding fragment thereof, comprising an amino acid sequence selected from a group consisting of SEQ ID NO: 103-443. In some embodiments, the present disclosure provides an anti-α11β1 antibody, or antigen-binding fragment thereof, that competes with an antibody, or antigen-binding fragment thereof, comprising an amino acid sequence selected from a group consisting of SEQ ID NO: 103-443.

In some embodiments, the present disclosure provides an anti-α11β1 antibody, or antigen-binding fragment thereof, comprising a heavy chain provided herein and a light chain provided herein. In some embodiments, the present disclosure provides an anti-α11β1 antibody, or antigen-binding fragment thereof, comprising a heavy chain variable domain provided herein and a light chain variable region provided herein. In some embodiments, the present disclosure provides an anti-α11β1 antibody, or antigen-binding fragment thereof, comprising a specific combination of heavy chain variable domain and light chain variable domain. For example, in some embodiments, an anti-α11β1 antibody, or antigen-binding fragment thereof, comprises a combination of heavy chain variable domain and light chain variable domain selected from Table 1.

TABLE 1
Combinations of 16E10 variant heavy chain variable
regions and light chain variable regions

| Light Chain | Heavy Chain | |
|---|---|---|
| Variable Region | Variable Region | Description |
| 16E10_VL | 16E10_VH | Parental light chain variable region; Parental heavy |
| (SEQ ID NO: 428) | (SEQ ID NO: 421) | chain variable region |
| 16E10_VL_1 | 16E10_VH_1 | Conservatively humanized light chain variable |
| (SEQ ID NO: 429) | (SEQ ID NO: 422) | region; Conservatively humanized heavy chain |
| | | variable region |
| 16E10_VL_2 | 16E10_VH_2 | Humanized light chain variable region; humanized |
| (SEQ ID NO: 430) | (SEQ ID NO: 423) | heavy chain variable region |
| 16E10_VL_3 | 16E10_VH_1 | Deimmunized conservatively humanized light chai |
| (SEQ ID NO: 431) | (SEQ ID NO: 422) | variable region; Conservatively humanized heavy |
| | | chain variable region |
| 16E10_VL_4 | 16E10_VH_2 | Deimmunized humanized light chain variable |
| (SEQ ID NO: 432) | (SEQ ID NO: 423) | region; Humanized heavy chain variable region |
| 16E10_VL_1 | 16E10_VH_3 | Conservatively humanized light chain variable |
| (SEQ ID NO: 429) | (SEQ ID NO: 424) | region; Deimmunized conservatively humanized |
| | | heavy chain variable region |
| 16E10_VL_2 | 16E10_VH_4 | Humanized light chain variable region; |
| (SEQ ID NO: 430) | (SEQ ID NO: 425) | Deimmunized humanized heavy chain variable |
| | | region |
| 16E10_VL_3 | 16E10_VH_3 | Deimmunized conservatively humanized light chain |
| (SEQ ID NO: 431) | (SEQ ID NO: 424) | variable region; Deimmunized conservatively |
| | | humanized heavy chain variable region |
| 16E10_VL_4 | 16E10_VH_4 | Deimmunized humanized light chain variable |
| (SEQ ID NO: 432) | (SEQ ID NO: 425) | region; Deimmunized humanized heavy chain |
| | | variable region |
| 16E10_VL_5 | 16E10_VH_3 | De-risked deimmunized conservatively humanized |
| (SEQ ID NO: 433) | (SEQ ID NO: 424) | light chain variable region; Deimmunized |
| | | conservatively humanized heavy chain variable |
| | | region |
| 16E10_VL_6 | 16E10_VH_4 | De-risked deimmunized humanized light chain |
| (SEQ ID NO: 434) | (SEQ ID NO: 425) | variable region; Deimmunised humanised heavy |
| | | chain variable region |
| 16E10_VL_3 | 16E10_VH_5 | Deimmunised conservatively humanised light chain |
| (SEQ ID NO: 431) | (SEQ ID NO: 426) | variable region; De-risked deimmunised |
| | | conservatively humanised heavy chain variable |
| | | region |
| 16E10_VL_4 | 16E10_VH_6 | Deimmunised humanised light chain variable |
| (SEQ ID NO: 432) | (SEQ ID NO: 427) | region; De-risked deimmunised humanised heavy |
| | | chain variable region |
| 16E10_VL_5 | 16E10_VH_5 | De-risked deimmunised conservatively humanised |
| (SEQ ID NO: 433) | (SEQ ID NO: 426) | light chain variable region; De-risked deimmunised |
| | | conservatively humanised heavy chain variable |
| | | region |
| 16E10_VL_6 | 16E10_VH_6 | De-risked deimmunised humanised light chain |
| (SEQ ID NO: 434) | (SEQ ID NO: 427) | variable region; De-risked deimmunised humanised |
| | | heavy chain variable region |

In some embodiments, the present disclosure provides an anti-α11β1 antibody, or antigen-binding fragment thereof, comprising between 1 and 30 (e.g., 1, 2, 3, 4, 5, 10, or more) additions, deletions, or substitutions relative to an anti-α11β1 antibody, or antigen-binding fragment thereof, wherein the anti-α11β1 antibody comprises an amino acid sequence selected from a group consisting of SEQ ID NO: 103-158, 413, 414 and 421-434 and, e.g., the antibody or fragment selectively binds α11β1. In some embodiments, the present disclosure provides an anti-α11β1 antibody, or antigen-binding fragment thereof, comprising between 1 and 30 additions, deletions, or substitutions relative to an anti-α11β1 antibody, or antigen-binding fragment thereof, wherein the anti-α11β1 antibody comprises an amino acid sequence selected from a group consisting of SEQ ID NO: 103-114, 413, 414 and 421-434 and, e.g., the antibody or fragment selectively binds α11β1. In some embodiments, the present disclosure provides an anti-α11β1 antibody, or antigen-binding fragment thereof, comprising an amino acid sequence having at least 80%, 85%, 90%, 91%, 92%, 93%, 94%, 95%, 96%, 97%, 98%, or 99% sequence identity to an amino acid sequence selected from a group consisting of SEQ ID NO: 103-158, 413, 414 and 421-434 and, e.g., the antibody or fragment selectively binds α11β1. In some embodiments, the present disclosure provides an anti-α11β1 antibody, or antigen-binding fragment thereof, comprising an amino acid sequence having at least 80%, 85%, 90%, 91%, 92%, 93%, 94%, 95%, 96%, 97%, 98%, or 99% identity to an amino acid sequence selected from a group consisting of SEQ ID NO: 103-114, 413, 414 and 421-434 and, e.g., the antibody or fragment selectively binds α11β1.

In some embodiments, the present disclosure provides an anti-α11β1 antibody, or antigen-binding fragment thereof, comprising between 1 and 90 (e.g., between 1 and 50, e.g., at least 1, 2, 3, 4, 5, 6, 7, 8, 9, 10 or more) additions, deletions, or substitutions relative to an anti-α11β1 antibody, or antigen-binding fragment thereof, wherein the anti-α11β1 antibody, or antigen-binding fragment thereof comprises an amino acid sequence selected from a group consisting of SEQ ID NO: 159-206 and 415-420 and, e.g., the antibody or fragment selectively binds α11β1. In some embodiments, the present disclosure provides an anti-α11β1 antibody, or antigen-binding fragment thereof, comprising an amino acid sequence having at least 80%, 85%, 90%, 91%, 92%, 93%, 94%, 95%, 96%, 97%, 98%, or 99% identity to an amino acid sequence selected from the group consisting of SEQ ID NO: 159-206 and 415-420 and, e.g., the antibody or fragment selectively binds α11β1.

In some embodiments, the disclosure provides an antibody or fragment thereof that selectively binds α11β1, wherein the antibody or fragment comprises one or more CDR sequences depicted in the list of exemplary sequences provided herein. For example, in some embodiments, an antibody or fragment thereof comprises one or more CDRs from SEQ ID NOs: 103-114. In some embodiments, the disclosure provides an antibody or fragment thereof that selectively binds α11β1, wherein the antibody or fragment comprises an amino acid sequence that is at least 95%, 96%, 97%, 98%, or 99% identical to one or more CDRs from SEQ ID NOs: 103-114. In some embodiments, an antibody or fragment comprises an amino acid sequence having at least 90%, 91%, 92%, 93%, 94%, 95%, 96%, 97%, 98%, or 99% identity to one of SEQ ID NOs: 103-114, wherein the antibody comprises one or more CDRs depicted in one of SEQ ID NOs: 103-114. For example, the antibody or fragment comprises an amino acid sequence having at least 90%, 91%, 92%, 93%, 94%, 95%, 96%, 97%, 98%, or 99% identity to SEQ ID NO: 103, wherein the antibody comprises one or more CDRs (e.g., 1, 2, or 3 CDRs) depicted in SEQ ID NO:103.

In some embodiments, the disclosure provides an antibody or fragment thereof that selectively binds α11β1, wherein the antibody or fragment comprises one or more CDR sequences depicted in the list of exemplary sequences provided herein. For example, in some embodiments, an antibody or fragment thereof comprises one or more CDRs from SEQ ID NOS: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443. In some embodiments, the disclosure provides an antibody or fragment thereof that selectively binds α11β1, wherein the antibody or fragment comprises an amino acid sequence that is at least 95%, 96%, 97%, 98%, or 99% identical to one or more CDRs from SEQ ID NOs: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443. In some embodiments, an antibody or fragment comprises an amino acid sequence having at least 90%, 91%, 92%, 93%, 94%, 95%, 96%, 97%, 98%, or 99% identity to one of SEQ ID NOs: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443, wherein the antibody comprises one or more CDRs depicted in one of SEQ ID NOs: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443. For example, the antibody or fragment comprises an amino acid sequence having at least 90%, 91%, 92%, 93%, 94%, 95%, 96%, 97%, 98%, or 99% identity to SEQ ID NO: 103, wherein the antibody comprises one or more CDRs (e.g., 1, 2, or 3 CDRs) depicted in SEQ ID NO:103.

The present disclosure provides, among other things, methods of making an anti-α11β1 antibody, or antigen-binding fragment thereof. Methods of making antibodies are known in the art. In some embodiments, the present disclosure provides methods of producing an antibody, or antigen-binding fragment thereof, comprising culturing a host cell comprising a nucleic acid comprising a nucleic acid sequence selected from a group consisting of SEQ ID NO: 1-102 under conditions suitable for expression of the antibody or antigen-binding fragment thereof.

### Exemplary Nucleotide Sequences

The present disclosure includes nucleotide sequences encoding one or more heavy chains, heavy chain variable domains, heavy chain framework regions, heavy chain CDRs, heavy chain constant domains, light chains, light chain variable domains, light chain framework regions, light chain CDRs, light chain constant domains, or other immunoglobulin-like sequences, antibodies, or binding molecules disclosed herein. In some embodiments, such nucleotide sequences may be present in a vector. In some embodiments such nucleotides may be present in the genome of a cell, e.g., a cell of a subject in need of treatment or a cell for production of an antibody, e.g. a mammalian cell for production of a an antibody.

In some embodiments, the present disclosure provides a nucleic acid comprising a nucleic acid sequence encoding an antibody, or antigen-binding fragment thereof, comprising an amino acid sequence selected from a group consisting of SEQ ID NO: 103-206. In some embodiments, the present disclosure provides a nucleic acid comprising a nucleic acid sequence encoding an antibody, or antigen-binding fragment thereof, comprising an amino acid sequence selected from a group consisting of SEQ ID NO: 103-114. In some embodiments, the present disclosure provides a nucleic acid comprising a nucleic acid sequence selected from a group consisting of SEQ ID NO: 1-102. In some embodiments, the present disclosure provides a vector comprising a nucleic acid comprising a nucleic acid sequence selected from a group consisting of SEQ ID NO: 1-102. In some embodiments, the present disclosure provides a host cell comprising a nucleic acid comprising a nucleic acid sequence selected from a group consisting of SEQ ID NO: 1-102. In some embodiments, the present disclosure provides a vector comprising a nucleic acid comprising a nucleic acid sequence selected from a group consisting of SEQ ID NO: 1-102.

In some embodiments, the present disclosure provides a nucleic acid comprising a nucleic acid sequence having at least 80%, 85%, 90%, 91%, 92%, 93%, 94%, 95%, 96%, 97%, 98%, or 99% sequence identity a nucleic acid sequence selected from a group consisting of SEQ ID NO: 1-102.

### Measuring Interactions of Antibodies and α11β1

The binding properties of an antibody described herein to α11β1 can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and α11β1 can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of an antibody to α11β1. Such data can be used to compare different molecules. Information from SPR can also be used to develop structure-activity relationships (SAR). Variant amino acids at given positions can be identified that correlate with particular binding parameters, e.g., high affinity.

In certain embodiments, an antibody described herein exhibits high affinity for binding α11β1. In various embodiments, K_{D} of an antibody as described herein for α11β1 is less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for α11β1 is between 0.001 and 1 nM, e.g., 0.001 nM, 0.005 nM, 0.01 nM, 0.05 nM, 0.1 nM, 0.5 nM, or 1 nM.

### Methods of Treatment

In some embodiments, one or more anti-α11β1 antibodies described herein are used in a method of treating one or more disorders described herein, e.g., one or more fibrotic disorders and/or one or more cancers. In some embodiments, the method comprises administering to a subject in need thereof a therapeutically effective amount of an antibody, or antigen-binding fragment thereof, described herein. In some embodiments, a fibrotic disorder is or comprises idiopathic pulmonary fibrosis (IPF), chronic kidney disease, diabetic cardiomyopathy, primary sclerosing cholangitis (PSC), primary biliary cirrhosis (PBC), non-alcoholic fatty liver disease (NAFLD/NASH), Crohn's disease, ulcerative colitis, or systemic sclerosis.

In some embodiments, a fibrotic disorder is or comprises atrial fibrosis, endomyocardial fibrosis, arthrofibrosis, mediastinal fibrosis, myelofibrosis, progressive massive fibrosis, retroperitoneal fibrosis or skeletal muscle fibrosis.

In some embodiments, one or more anti-α11β1 antibodies described herein are used in a method of treating cancer, such as one or more of the following: head and neck squamous cell carcinomas, pancreatic ductal adenocarcinoma, non-small cell lung cancer, adrenocortical carcinoma, acute myeloid leukemia, bladder urothelial carcinoma, invasive breast carcinoma, cervical squamous cell carcinoma, cholangiocarcinoma, colorectal adenocarcinoma, diffuse large B-cell lymphoma, esophageal adenocarcinoma, glioblastoma multiforme, liver hepatocellular carcinoma, lung adenocarcinoma, lung squamous cell carcinoma, skin cutaneous melanoma, mesothelioma, ovarian serous cystadenocarcinoma, pheochromocytoma and paraganglioma, prostate adenocarcinoma, sarcoma, stomach adenocarcinoma, testicular germ cell tumors, thymoma, thyroid carcinoma, uterine corpus endometrial carcinoma, uterine carcinosarcoma, uveal melanoma, kidney renal clear cell carcinoma, kidney chromophobe, and kidney renal papillary cell carcinoma.

In some embodiments, one or more anti-α11β1 antibodies described herein are used to treat a subject having or at risk of a chronic kidney disease (CKD), e.g., CKD associated with fibrosis. CKDs are known in the art (see, e.g., Brenner, Barry M. (ed) Brenner & Rector's The Kidney, 11^{th} edition 2019). CKDs include, e.g., Primary Glomerular Disease (including, but not limited to, IgA Nephropathy and focal segmental glomerular sclerosis), Secondary Glomerular Disease (including, but not limited to, lupus nephritis), Thrombotic Microangiopathy, Tubulointerstitial Diseases (including, but not limited to, Obstructive Uropathy), Diabetic Nephropathy, Hypertensive Nephropathy, Ischemic Nephropathy, Cardiorenal Syndromes in CKD, Inherited Disorders of the Glomerulus (including, but not limited to, Alport syndrome), Cystic Diseases of the Kidney (including, but not limited to, Polycystic Kidney Disease), and Inherited Disorders of the Renal Tubule (Brenner, Barry M. (ed) Brenner & Rector's The Kidney, 11^{th} edition 2019).

In some embodiments, an anti-α11β1 antibody described herein, upon administration to a subject, reduces one or more markers, signs and/or symptoms of a kidney-related disorder described herein. Markers, signs and/or symptoms of kidney-related disorders include, e.g., COL1A1, IL-6, TIMP-1, Hyaluronic acid, TGFβ, CTGF, PDGF, and MMP9. In some embodiments, upon administration to a subject, an anti-α11β1 antibody can reduce a measured marker, sign and/or symptom by at least about 10%, 15%, 20%, 25%, 30%, 35%, 40%, 50%, 55%, 60%, 65%, 70%, 75%, 80%, 85% or 90%, relative to a control (e.g., a level of measured marker, sign and/or symptom in the subject prior to administration of the antibody, a level of measured marker, sign and/or symptom in a subject suffering from the kidney-related disorder, and/or an average level of measured marker, sign and/or symptom in a population of subjects suffering from the kidney-related disorder).

In some embodiments, an anti-α11β1 antibody described herein reduces levels of COL1A1, IL-6, TIMP-1, Hyaluronic acid, TGFβ, CTGF, PDGF, MMP9, or a combination thereof by at least about 10%, 15%, 20%, 25%, 30%, 35%, 40%, 50%, 55%, 60%, 65%, 70%, 75%, 80%, 85% or 90%, relative to a control, as measured in a model of kidney-related disorder (e.g., human Precision-Cut Kidney Slices (PCKS), a ReninAAV Unx db/db mouse model, or a ⅚ Nephrectomy model). In some embodiments, markers, signs and/or symptoms of kidney-related disorders can be determined by measuring protein levels, RNA levels, DNA levels, or a combination thereof. In some embodiments, markers, signs and/or symptoms of kidney-related disorders can be determined using ELISA, PCR, RNAseq, a biochemical assay (e.g., an analytical procedure to detect and quantify cellular processes (e.g. apoptosis, cell signaling) or metabolic reactions), cytology, immunohistochemistry, or a combination thereof.

In some embodiments, markers, signs and/or symptoms of kidney-related disorders can be determined by testing a biological sample from a subject. Examples of suitable biological samples include, but are not limited to, serum, plasma, cerebrospinal fluid, urine, circulating blood cells (e.g., peripheral blood mononuclear cells), and biopsy specimens. In some embodiments, a sample comprises cells or tissues. In some embodiments, provided methods further comprises a step of lysing cells or performing a tissue biopsy and one or more markers include one or more intracellular markers. Biological samples suitable for the present disclosure may be fresh or frozen samples collected from a subject, or archival samples with known diagnosis, treatment and/or outcome history. Biological samples may be collected by any invasive or non-invasive means, such as, for example, by drawing CSF or blood from a subject, or using fine needle aspiration or needle biopsy, or by surgical biopsy. In some embodiments, biological samples may be used without or with limited processing of the sample.

### Combination Therapy

In some embodiments, an anti-α11β1 antibody described herein is administered in combination with one or more additional therapeutic agents, such as a chemotherapeutic agent, or an oncolytic therapeutic agent. “Combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. Administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “chemotherapeutic agent” or “oncolytic therapeutic agent” (e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In some embodiments, a chemotherapeutic agent and/or oncolytic therapeutic agent may be or comprise platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

In certain embodiments, chemotherapeutic agents and/or oncolytic therapeutic agents for anti-cancer treatment comprise biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors or other immunologic agents. In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-OO1, Bicalutamide, Nilutamide, Flutamide, Enzalutamide, ARN-509).

Additional chemotherapeutic agents and/or oncolytic therapeutic agents include immune checkpoint therapeutics (e.g., pembrolizumab, nivolumab, ipilimumab, atezolizumab, avelumab, durvalumab, tremelimumab, or cemiplimab), other monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, nimotuzumab, lambrolizumab, pidilizumab, siltuximab, BMS-936559, RG7446/MPDL3280A, MEDI4736), antibody-drug conjugates (e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. 1045:1-27 (2013); Bouchard et al., Bioorganic Med. Chem. Lett. 24: 5357-5363 (2014)), or any combination thereof.

In some embodiments, combined administration of an anti-α11β1 antibody and an additional therapeutic agent results in an improvement in cancer to an extent that is greater than one produced by either the anti-α11β1 antibody or the additional therapeutic agent alone. The difference between the combined effect and the effect of each agent alone can be a statistically significant difference. In some embodiments, the combined effect can be a synergistic effect. In some embodiments, combined administration of an anti-α11β1 antibody and an additional therapeutic agent allows administration of the additional therapeutic agent at a reduced dose, at a reduced number of doses, and/or at a reduced frequency of dosage compared to a standard dosing regimen, e.g., an approved dosing regimen for the additional therapeutic agent.

In some embodiments, treatment methods described herein are performed on subjects for whom other treatments of the medical condition have failed or have had less success in treatment through other means. Additionally, the treatment methods described herein can be performed in conjunction with one or more additional treatments of the medical condition. For instance, the method can comprise administering a cancer regimen, e.g., non-myeloablative chemotherapy, surgery, hormone therapy, and/or radiation, prior to, substantially simultaneously with, or after the administration of an anti-α11β1 antibody described herein, or composition thereof.

### Formulations and Administration

In various embodiments, an antibody described herein can be incorporated into a pharmaceutical composition. Such a pharmaceutical composition can be useful, e.g., for the prevention and/or treatment of diseases, e.g., fibrotic disorders. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

In some embodiments, a pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In some embodiments, a composition including an antibody as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, such as, for example, an alcohol such as ethanol and/or a polyalcohol such as propylene glycol or polyethylene glycol, and/or a nonionic surfactant such as polysorbate 80™ or HCO-50.

As disclosed herein, a pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion. In some embodiments, compositions can be targeted to the kidneys. In some embodiments, compositions can be targeted to renal cells.

Route of administration can be parenteral, for example, administration by injection, transnasal administration, transpulmonary administration, or transcutaneous administration. Administration can be systemic or local by intravenous injection, intramuscular injection, intraperitoneal injection, or subcutaneous injection. In some embodiments, the route of administration is or comprises dialysis. In some embodiments, the route of administration is or comprises hemodialysis. In some embodiments, the route of administration is or comprises peritoneal dialysis.

In some embodiments, a pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in a pharmaceutical preparation is such that a suitable dose within the designated range is provided. Non-limiting examples of oily liquid include sesame oil and soybean oil, and may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. A formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with antibody drug for subcutaneous injection.

An injection system of the present disclosure may employ a delivery pen as described in U.S. Pat. No. 5,308,341. Pen devices, most commonly used for self-delivery of insulin to patients with diabetes, are well known in the art. Such devices can comprise at least one injection needle (e.g., a 31 gauge needle of about 5 to 8 mm in length), are typically prefilled with one or more therapeutic unit doses of a therapeutic solution, and are useful for rapidly delivering solution to a subject with as little pain as possible. One medication delivery pen includes a vial holder into which a vial of a therapeutic or other medication may be received. The pen may be an entirely mechanical device or it may be combined with electronic circuitry to accurately set and/or indicate the dosage of medication that is injected into the user. See, e.g., U.S. Pat. No. 6,192,891. In some embodiments, the needle of the pen device is disposable and the kits include one or more disposable replacement needles. Pen devices suitable for delivery of any one of the presently featured compositions are also described in, e.g., U.S. Pat. Nos. 6,277,099; 6,200,296; and 6,146,361, the disclosures of each of which are incorporated herein by reference in their entirety. A microneedle-based pen device is described in, e.g., U.S. Pat. No. 7,556,615, the disclosure of which is incorporated herein by reference in its entirety. See also the Precision Pen Injector (PPI) device, MOLLY™, manufactured by Scandinavian Health Ltd.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In some embodiments, a pharmaceutical composition can be formulated as a solution. In some embodiments, a composition can be formulated, for example, as a buffered solution at a concentration suitable for storage at 2-8° C. (e.g., 4° C.).

Compositions including one or more antibodies as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, administration of an antibody as described herein is achieved by administering to a subject a nucleic acid encoding the antibody. Nucleic acids encoding a therapeutic antibody described herein can be incorporated into a gene construct to be used as a part of a gene therapy protocol to deliver nucleic acids that can be used to express and produce antibody within cells. Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., WO04/060407). Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc Natl Acad Sci USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; and PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 dl324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J Virol 63:3822-3828; and McLaughlin et al. (1989) J Virol 62:1963-1973.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

A suitable dose of a composition described herein, which dose is capable of treating or preventing a disorder in a subject, can depend on a variety of factors including, e.g., the age, sex, and weight of a subject to be treated and the particular inhibitor compound used. For example, a different dose of one composition including an antibody as described herein may be required to treat a subject with a fibrotic disorder as compared to the dose of a different formulation of that antibody. Other factors affecting the dose administered to the subject include, e.g., the type or severity of the disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A composition described herein can be administered as a fixed dose, or in a milligram per kilogram (mg/kg) dose. In some embodiments, the dose can also be chosen to reduce or avoid production of antibodies or other host immune responses against one or more of the antigen-binding molecules in the composition. Exemplary dosages of an antibody, such as a composition described herein, include, e.g., 0.0001 to 100 mg/kg, 0.01 to 5 mg/kg, 1-1000 mg/kg, 1-100 mg/kg, 0.5-50 mg/kg, 0.1-100 mg/kg, 0.5-25 mg/kg, 1-20 mg/kg, and 1-10 mg/kg of the subject body weight. For example dosages can be 0.1 mg/kg, 0.3 mg/kg, 0.5 mg/kg, 1.0 mg/kg, 2.0 mg/kg, 3.0 mg/kg, 4.0 mg/kg, 5.0 mg/kg, 10 mg/kg or 20 mg/kg body weight or within the range of 1-20 mg/kg body weight. An exemplary treatment regime entails administration once per week, once every two weeks, once every three weeks, once every four weeks, once a month, once every 3 months or once every three to 6 months, or with a short administration interval at the beginning (such as once per week to once every three weeks), and then an extended interval later (such as once a month to once every three to 6 months).

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of a fibrotic disorder. For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the compositions described herein can further be evaluated in, e.g., Phase I dose escalation studies. See, e.g., van Gurp et al. (2008) Am J Transplantation 8(8): 1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of compositions can be determined by known pharmaceutical procedures in cell cultures or experimental animals (e.g., animal models of any of the fibrotic disorders described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A composition described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

Those of skill in the art will appreciate that data obtained from cell culture assays and animal studies can be used in formulating a range of dosage for use in humans. Appropriate dosages of compositions described herein lie generally within a range of circulating concentrations of the compositions that include the ED₅₀ with little or no toxicity. The dosage may vary within this range depending upon the dosage form employed and the route of administration utilized. For a composition described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Methods

### Generation of Novel Antibodies Against α11β1

### Rat Immunization

Wistar rats were immunized with recombinant human α11β1 protein. An enzyme-linked immunosorbent assay (ELISA) was used to test an immune response against target human and mouse proteins. Subsequently, cell fusion (by electro fusion) was performed with animals that produced a good immune response. All fused cells were plated in a 96-well plate and supernatants were screened by ELISA against soluble human and mouse α11β1. Positive clones were counter-screened against human α1β1, α2β1 and α10β1. Clones that specifically bound to human and mouse α11β1 and did not bind to α1β1, α2β1 and α10β1 were selected, subcloned, expanded and cryopreserved. Purified antibodies were then generated from the selected clones and heavy chain and light chain variable domain sequences were obtained from each purified antibody.

### Rabbit Immunization

Rabbits were immunized employing a cell-based monoclonal antibody platform. Two rabbits were immunized with recombinant human α11β1 protein. Splenocytes from the immunized rabbits were sorted and selected against human β1, in order to reduce the number of β1-specific B cell clones. Sorted splenocytes were then cultured for approximately 1 week and culture supernatants were screened for binding to human α11β1. Top results were sequenced and subsequently rabbit antibodies were recombinantly produced using a HEK cell system.

### Mouse Immunization

10 mice from 5 different strains were immunized with an appropriate mixture of human α11β1, mouse α11β1 and tolerance breaking protein. Plasma titers were evaluated by ELISA against a mixture of human and mouse α11β1. Popliteal, inguinal, and iliac lymph nodes were collected. ELISA-positive anti-human/mouse α11β1 hybridomas were expanded and subjected to a secondary screen against human and mouse α11β1, control HIS protein, and a counter-screen was performed against human α11β1, α2β1 and α10β1. Supernatant IgG concentration was sufficient for functional screening. Selected hybridomas satisfying all the criteria were cloned and clonal hybridomas were confirmed by ELISA against human and mouse α11β1 and subsequently scaled-up and IgGs were purified. Heavy and light chain variable regions of selected hybridomas were then sequenced.

### Phage Library Display

Phage library display was employed for generation of fully human anti-α11β1 antibodies. Fully human anti-α11β1 antibodies were discovered using single chain fragment variable (scFv) antigen-binding fragments displayed on phages (phage display library). Three rounds of selection were performed on purified human and mouse α11β1 antigen as well as deselection against α10β1, to enrich for α11 subunit-specific antibodies. Subsequently, optimal populations were subcloned into a bacterial soluble expression vector, recombinant antibody expression was induced and supernatant was screened for binding via ELISA assays. Antibodies with appropriate binding profiles were sequenced and subsequently converted from scFv to IgG.

### ELISA

0.25 μg/mL of target antigen (recombinant human or mouse α11β1) was plated in a 96-well plate over night at 4° C. Plates were washed (PBS with 0.1% Tween-20), blocked (PBS with 2% BSA and 0.05% Tween-20) for 1 hour at room temperature and incubated with a range of antibody concentrations for 1 hour at room temperature. Subsequently, plates were washed and incubated with biotinylated anti-rabbit/mouse/human IgG in a 1:1000 dilution buffer and incubated for 1 hour at room temperature. After washing the plates, Streptavidin HRP was added at 1:200 in dilution buffer and incubated for 1 hour at room temperature. Ultra TMB ELISA substrate solution was added and the plates incubated for 5 minutes on a plate shaker. The reaction was stopped by adding stop solution to each well and plates were read at 450 nm.

### FACS

### Antibody Binding to CHO-K1

200,000 cells (wild-type CHO-K1 cells or CHO-K1 cells expressing human α11) were incubated with each antibody at desired concentrations in FACS buffer for 30 min at 4° C. Then, the cells were washed with FACS buffer and incubated with secondary antibody at 1:100 dilution for 30 minutes at 4° C. Cells were then washed and fixed with 1% PFA in PBS for 20 minutes at room temperature; washed again and read on a cytometer in FACS buffer.

### Antibody Binding to HPF/MF

Human Pulmonary Fibroblasts (ScienCell) were cultured in complete Fibroblast Growth Medium (ScienCell) until 80% confluent in T-150 flasks. Cells were washed and harvested using Accutase. Cells were seeded into T-150 flasks at 7,500 cells/cm² in complete FGF and cultured for 72 hours. Cells were then washed and starved in serum reduced medium for 24 hours. After starvation, cells were treated with TGFβ-1 (R&D Systems) for 72 hours. Cells were harvested using Accutase and seeded in 96-well conical bottom plates. Cells were blocked with Heat Inactived Fetal Bovine Serum (Gibco) for 30 minutes at 4° C. Cells were then incubated with anti-α11 antibodies at the doses described in each figure for 30 minutes at 4° C. A human anti-α11 antibody (Creative BioLabs) was included as a positive control as well as the appropriate IgG isotype negative controls. Cells were washed twice and incubated with PE conjugated secondary antibodies specific to the IgG class of the anti-α11 antibodies being tested for 30 minutes at 4° C. Cells were washed twice and fixed in 1% PFA for 30 minutes. Cells were acquired on a FACS Verse (Benton Dickson) binding of each antibody. Data were analyzed by gating on single cells and determining the geometric Mean Fluorescence Intensity (gMFI) in the PE channel for each sample.

### Surface Plasmon Resonance (SPR)

The affinity of antibodies for human α11β1 was measured by surface plasmon resonance assay (SPR). Affinity was measured at pH 7.6 and 25° C. with a Biacore T200 instrument. Anti-HIS antibody was immobilized on the SPR sensor surface using EDC/NHS covalent attachment. HIS-tagged human α11β1 was captured on the sensor surface and a single-cycle kinetics assay was used. Increasing concentrations of test antibody were injected in series over the sensor-bound α11β1. Dissociation was monitored for 1000 seconds. A sensor surface with only anti-HIS antibody and a series of blank injections were used to double-reference subtract the data. A 1:1 Langmuir model was fit to the data to estimate the kinetic association and dissociation constants. The affinity (equilibrium dissociation constant) of the interaction was calculated by dividing the kinetic dissociation constant by the kinetic association constant. Between injection cycles α11β1 and bound antibody were removed with an injection of 10 mM glycine at pH 1.5.

### Cell Adhesion Inhibition

0.6×10⁶ cells/mL were incubated with each antibody at a range of concentrations for 20 minutes at 37° C. E-Plate VIEW 96 PET plate coated with 100 ng/ml type I collagen or PBS overnight at room temperature was blocked in 3% BSA for 1 house at room temperature. After washing the plate with PBS, cell and antibody mixture was added to the wells and the plate was places into an xCelligence machine. Cell adhesion was recorded over 6 hours. The time point of maximum cell adhesion was used for comparison relative to control.

### Fibroblast-to-Myofibroblasts Transition (FMT)

Human Pulmonary Fibroblasts (ScienCell) were cultured in complete Fibroblast Growth Medium (ScienCell) until 80% confluent. Cells were washed and harvested using Accutase. Cells were seeded onto tissue culture-treated 96 well plates at 20,000 cells/well in complete Fibroblast Growth Medium. After 24 hours, cells were washed and starved in serum reduced medium for an additional 24 hours. After starvation, cells were treated with TGFβ-1 (R&D Systems) with or without anti-α11 antibodies. A polyclonal rabbit anti-human α11 antibody was used as a positive control. Appropriate IgG isotype controls were also included. After 48 hours, cells were harvested, fixed, permeabilized and stained with AlexaFluor488 Labeled Anti-αSMA (α-smooth muscle actin) (Invitrogen). Cells were acquired on a FACS Verse (Benton Dickson) to determine expression levels of αSMA. Data were analyzed by gating on single cells and determining the geometric Mean Fluorescence Intensity (gMFI) in the FITC channel for each sample. gMFI for each sample was normalized to the untreated control and presented as % inhibition.

### Collagen Gel Contraction Assay

24-well plates were blocked with 2% BSA in PBS overnight at 37° C. The following day, the plates were washed 3 times with PBS before being used in the assay. Human CHO cell lines expressing α11 were harvested and resuspended at 1.25×10⁶ in ExpiCHO Expression Medium (Gibco™ Cat #A2910002). The collagen gel solution was prepared by diluting 3 mg/mL stock collagen type I (Gibco™ Collagen I Rat Protein, Tail Cat #A1048301) to 1 mg/mL in the media containing the CHO cells. Sodium hydroxide was added to the solution to neutralize the pH and 400 μL of the collagen solution was added to each well of the 24-well plates. For the wells where the antibodies were included in the collagen gel solution, CHO cells were prepared at 2.5×10⁶ and the antibodies were prepared at 2× final concentration in ExpiCHO media. The cells and antibodies were then combined 1:1 before the addition of the stock collagen type 1. The gels were allowed to polymerize for 60 minutes at 37° C. Antibodies were added to the ExpiCHO media, which was then layered on top of the polymerized gel (400 μL/well). The gels were incubated for 6 days at 37° C. before gel contraction was quantified. Images of each well were analyzed using Image J and gel contraction was determined as a percentage of the initial gel area.

### Tumor Xenograft Model

Fifty-six female C.B-17 SCID mice were inoculated with A549 cells (5×10⁶ cells/mouse) subcutaneously in the flank. Once tumor volume reached ˜100 mm³, animals were randomized amongst 7 groups of 8 mice each. Mice were then treated intraperitonealy every 3 days for a total of 7 doses with isotype controls or novel mAbs 79E3E3, 16E10 and 9G04 (2 and 20 mg/kg) or with docetaxel at 10 mg/kg every 4 days for a total of 6 doses. Tumor volumes and body weights were recorded twice a week with a gap of 2-3 days in between two measurements until any of the following conditions defined were observed: loss of 20% or more body weight; tumors that inhibit normal physiological function such as eating, drinking, and mobility; ulcerated tumors; tumor size greater than 2000 mm³ and clinical observations of prostration, paralysis, seizures and hemorrhages.

### Precision-Cut Liver Slices (PCLS)

Precision-Cut Liver Slices (PCLS) were prepared from resected liver tissue and rested for 24 hours to allow the post-slicing stress period to elapse before experiments began. PCLS were cultured without exogenous challenge (Group 1), with 100 μg/mL control antibodies (Groups 2 and 3-either mouse IgG2a or rabbit IgG), or with a combination of TGF-β1 (3 ng/mL) and PDGF-ββ (50 ng/ml) (Groups 2-10). PCLS were cultured in the presence or absence of 10 μM Alk5i (Group 4) as a positive control or novel inhibitors (16E10, 79E3E3, and 9G05) at 2 escalating doses (10 and 100 μg/mL) in Groups 5-10. Each of the 10 groups included n=6 human PCLS prepared from a single human liver. PCLS culture media, including all stimuli and compounds, was refreshed and harvested at 24 hour intervals. Cell culture supernatant (n=⅔ paired wells) was collected every 24 hours and snap frozen for quantification of soluble outputs. All PCLS were harvested at 96 hours.

Tissue culture levels of markers of liver damage (lactate dehydrogenase (LDH) and aspartate transaminase (AST)) and hepatocyte function/viability (albumin) were quantified on all PCLS at all time points. Albumin secretion was quantified by ELISA as a marker of PCLS integrity and function. Levels of collagen 1a1, IL-6, hyaluronic acid and Timp-1 in the cell culture supernatants were quantified using R&D Duoset ELISA kits.

Total RNA extraction from PCLS was performed on all samples. RNeasy Mini kits (Qiagen) were used for RNA extraction. RNA was reverse-transcribed to cDNA and used in qPCR to measure transcript levels of Col1a1, αSMA, TIMP-1, TGF-β1, IL-6 and β-actin/GAPDH.

### Precision-Cut Kidney Slices (PCKS)

PCKS were prepared from explanted fibrotic human kidney tissue and rested for 24 hours to allow the post-slicing stress period to elapse before experiments began. PCKS were cultured with TGF-β1 (3 ng/mL) and PDGFββ (50 ng/mL) in the presence or absence of Alk5i (10 μM, positive control), test anti-α11β1 antibodies over three doses (1, 10 and 50 μg/mL) or in the presence of IgG control antibodies at a single high dose. PCKS culture media was harvested every 24 hours for a total of 3 time points. Levels of Collagen type I α1 (col1a1) in the tissue culture supernatants were quantified using R&D Duoset ELISA kits. Statistics were performed using Two-way ANOVA followed by Dunnett's multiple comparisons test.

### Example 1. Generation of Novel Monoclonal Antibodies Against α11β1 and Determination of Binding Affinity

Antibody discovery was performed by immunizing rats and rabbits with recombinant human α11β1, and by immunizing mice with both human and mouse α11β1. 51 novel anti-human α11β1 monoclonal antibodies were generated (24 rabbit, 7 rat and 20 mouse). Heavy chain and light chain variable region sequences were determined for the mouse and rat antibodies, while full heavy chain and light chain sequences were determined for the rabbit antibodies.

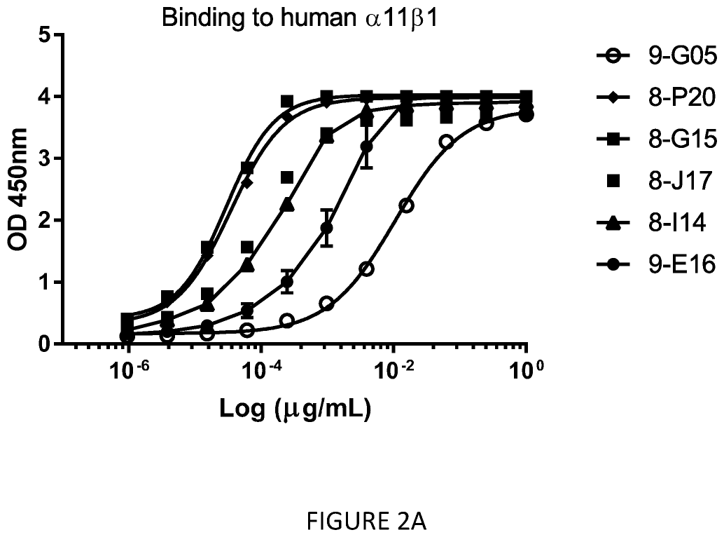

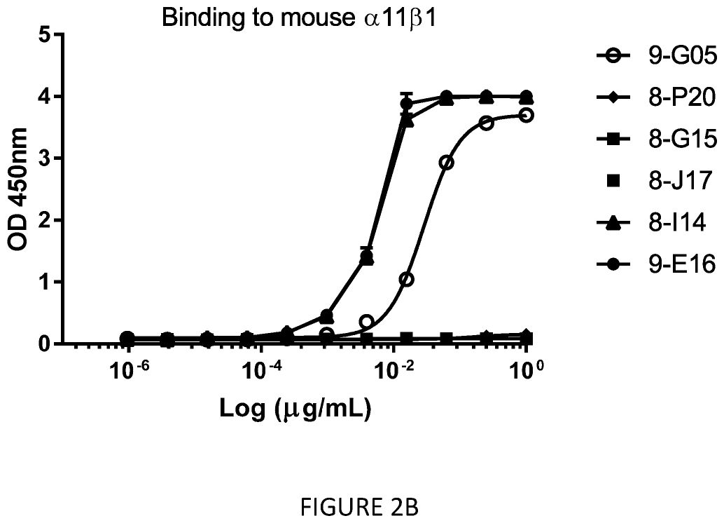

ELISA results illustrating exemplary binding of selected mouse monoclonal antibodies to recombinant human α11β1 are shown in FIG. 2A. Three of those mAbs also bound to mouse α11β1, as shown in FIG. 2B.

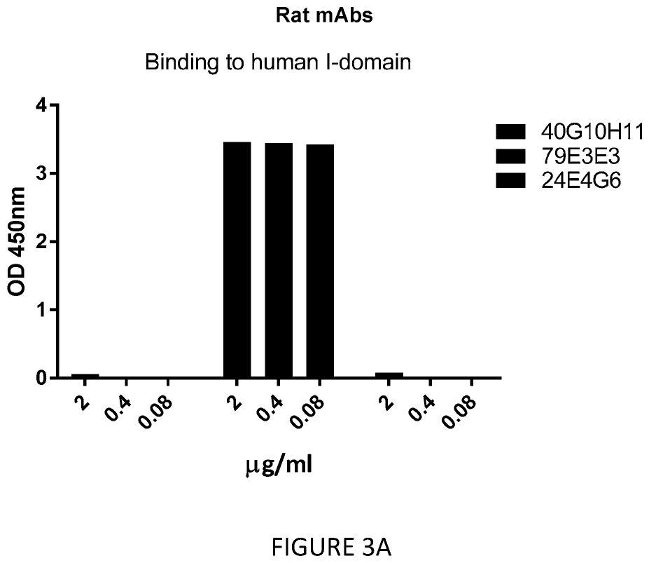

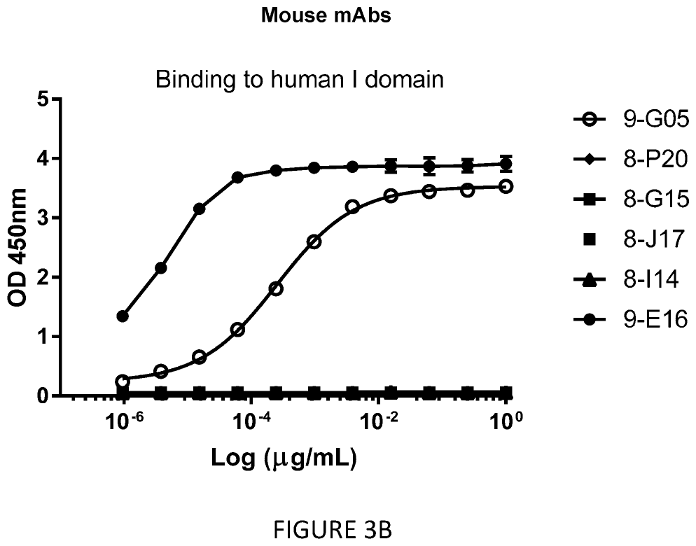

Data was also collected to determine whether antibodies of interest bind to the I domain of α11β1. An in-house generated I domain of α11β1 was used. The rat clones 79E3E3, 8H8E9 and 6E5C11 exhibited high, medium, and low binding, respectively, as determined by ELISA. Mouse antibodies 10-F23, 10-L15, 7-O8, 6-A12, 9-G05 and 9-E16 and rabbit antibodies 7-H12 and 2-D3 were also tested for binding against the in-house generated α11β1 I domain. FIGS. 3A and 3B show graphs illustrating binding data from exemplary mAbs.

α11β1 belongs to a family of collagen receptors and has a relatively high homology to them. Therefore, the novel antibodies of this invention were counter-screened against α1β1, α2β1 and/or α10β1. Table 2 includes results of cross-reactivity to the other receptors.

TABLE 2
Data summary of tested monoclonal antibodies

| | | | | | | | | | Inhibition of | | Inhibition of |
|---|---|---|---|---|---|---|---|---|---|---|---|
| | | Human | Murine | | | | | | CHO-K1 | | CHO-K1 |
| | | α11β1 | α11β1 | Off | I Domain | CHO-K1 | HPF | MF | adhesion to | Inhibition | gel |
| | Clone ID | ELISA | ELISA | Target | binding | FACS | FACS | FACS | collagen | of FMT | contraction |
| MOUSE | 10-L15 | Yes | Yes | No | Yes | No | No | No | No | No | |
| | 8-I14 | Yes | Yes | No | No | Yes | No | Yes | No | Yes | No |
| | 3-G5 | Yes | Yes/Low | No | No | No | No | No | No | No | |
| | 2-A3 | Yes | Yes/Low | No | No | No | No | No | No | No | |
| | 8-G15 | Yes | No | No | No | Yes | No | Yes | Yes | Yes | |
| | 8-P20 | Yes | No | No | No | Yes | No | Yes | No | No | |
| | 10-F23 | Yes | Yes/Low | No | Yes | Yes | No | Yes | Yes | No | |
| | 7-O8 | Yes | Yes/Low | Yes | Yes | Yes | No | Yes | No | No | |
| | | | | (α10β1) | | | | | | | |
| | 8-J17 | Yes | No | Yes | No | Yes | No | Yes | No | Yes | |
| | | | | (at high | | | | | | | |
| | | | | doses) | | | | | | | |
| | 9-E16 | Yes | Yes | No | Yes | Yes | Low | Yes | Yes | Yes | |
| | 9-G05 | Yes | Yes | No | Yes | Yes | No | Yes | Yes | Yes | Yes |
| | 10-K10 | Yes | Yes/Low | No | No | No | No | Yes | No | No | |
| | 6-O12 | Yes | No | No | No | No | No | No | No | No | |
| | 6-A15 | Yes | Yes/Low | No | No | No | No | No | No | No | |
| | 6-B21 | Yes | Yes/Low | No | No | No | No | No | No | No | |
| | 6-A12 | Yes | Yes/Low | No | Yes | Yes | No | Yes | Yes | No | |
| | 6-M8 | Yes | No | No | No | No | No | No | No | No | |
| | 6-P20 | Yes | No | No | No | Yes | No | No | No | No | |
| | 6-O17 | Yes | Yes/Low | No | No | Yes | No | Yes | No | No | |
| | 9-B11 | Yes | No | No | Yes | Yes | No | Yes | Yes | Yes | No |
| | 7-H14 | Yes | Yes/Low | No | No | Yes | No | Yes | No | No | |
| RAT | 24E4G6 | Yes | No/Low | No | No | Yes | No | Yes | No | Yes | Yes |
| | 40G10H11 | Yes | Yes | Yes (all) | No | Yes | N/A | N/A | Yes | Yes | |
| | 18E10F10 | Yes | No | No | No | Yes | N/A | N/A | Yes | Yes | |
| | 8H8E9 | Yes | No | No | No | Yes | N/A | N/A | Yes | Yes | |
| | 6E5C11 | Yes | No | No | No | Yes | N/A | N/A | No | Yes | |
| | 7D8B10 | Yes | No | No | No | Yes | No | Yes | No | Yes | |
| | 79E3E3 | Yes | Yes | No | Yes | Yes | No | Yes | Yes | No | Yes |
| RABBIT | 16E10 | Yes | No | No | No | Yes | No | Yes | Yes | Yes | Yes |
| | 6F9 | Yes | No | No | No | Yes | No | Yes | Yes | No | |
| | 6G4 | Yes | No | No | No | Yes | No | Yes | Yes | No | |
| | 4E1 | Yes | No | No | No | Yes | No | Yes | Yes | No | |
| | 6C7 | Yes | No | No | No | Yes | No | Yes | Yes | No | |
| | 5D7 | Yes | No | No | No | Yes | No | Yes | Yes | No | |
| | 5A7 | Yes | No | No | No | Yes | No | Yes | Yes | No | |
| | 3B1 | Yes | No | No | No | Yes | No | Yes | Yes | No | |
| | 16G7 | Yes | Yes | No | No | Yes | No | Yes | No | Yes | No |

N/A = not tested

Since integrins are large, transmembrane receptors that exist in different conformational shapes, experiments were performed to confirm that the novel antibodies also bound cell-expressed α11β1. A CHO-K1 cell line that endogenously expressed high levels of the β1 subunit was engineered to stably express human α11 (CHO-K1 hu α11).

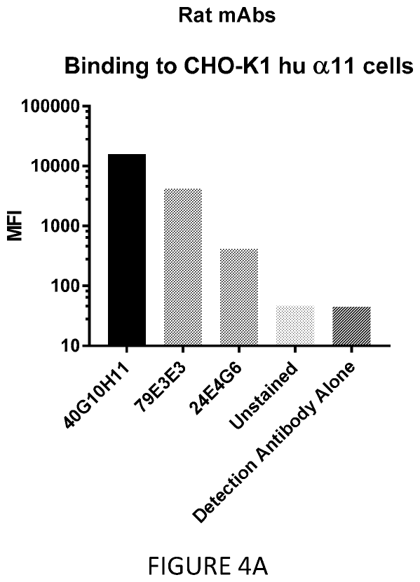

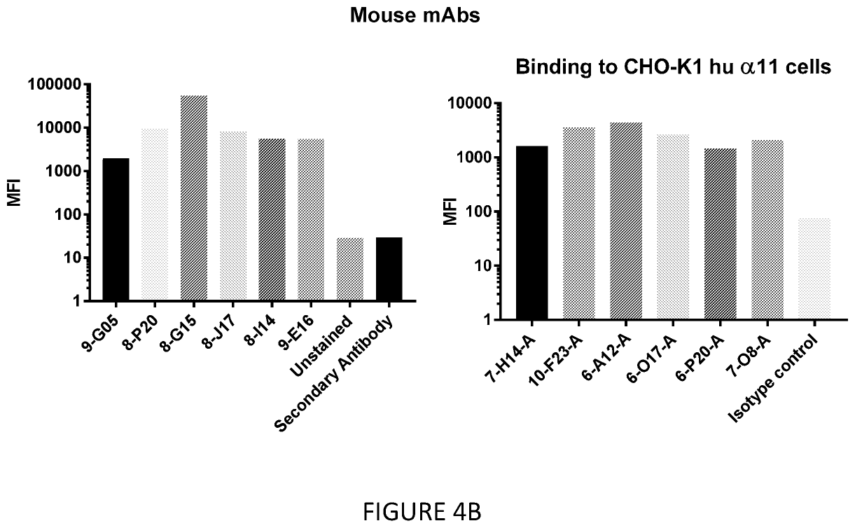

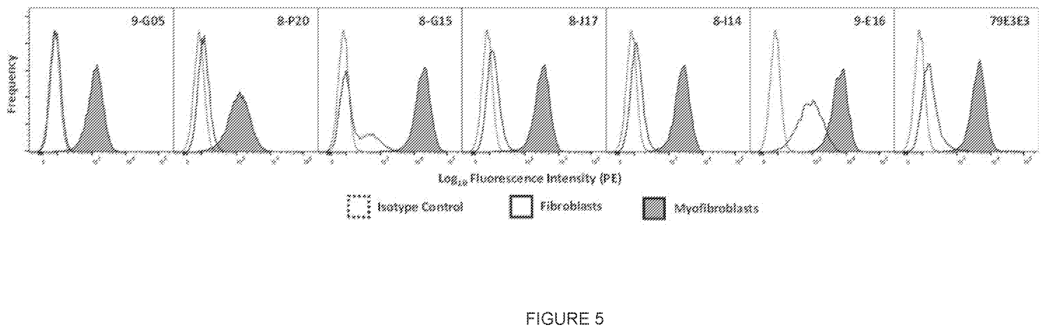

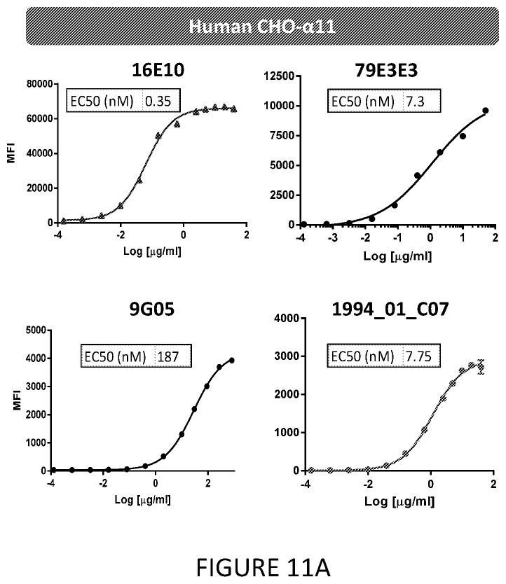

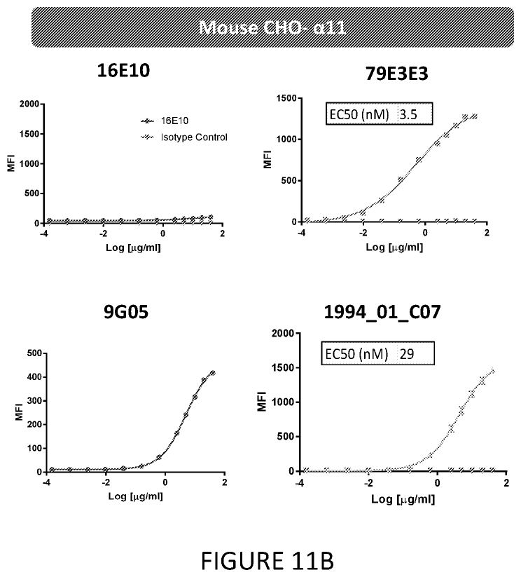

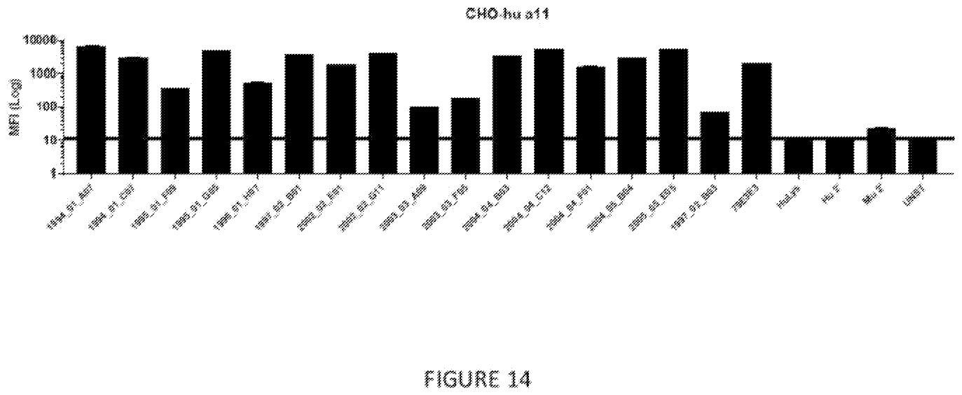

FIGS. 4A and 4B show selected rat and mouse mAbs that bound human α11β1 (as tested by ELISA) and also demonstrated binding ability to α11β1 expressed on the surface of CHO-K1 cells. FIGS. 11A and 11B show selected rabbit, rat, mouse and human mAbs that demonstrated binding ability to α11β1 expressed on the surface of CHO cells. Additionally, FIG. 14 shows selected fully human mAbs that demonstrated binding ability to α11β1 expressed on the surface of CHO cells. However, as shown in Table 1, there were several mAbs that were shown to bind α11β1 by ELISA, but did not bind cell-expressed α11β1.

Binding EC₅₀ was estimated using data from Fluorescence-activated cell sorting (FACS) performed with CHO-K1 hu α11β1 cells. The results are shown in Table 3. Four out of six mAbs tested had EC₅₀ results in the low nanomolar range (8-P20, 8-G15, 8-J17, 8-I14), while the remaining two mAbs were not as potent (9-G05 and 9-E16; both I domain binders).

TABLE 3
Estimated CHO-K1 binding EC50 for mouse mAbs

| mAb | Conc. (μg/mL) | Molarity (nM) |
|---|---|---|
| 9-G05 | 21.03 | 140.2 |
| 8-P20 | 0.12 | 0.8 |
| 8-G15 | 0.22 | 1.5 |
| 8-J17 | 0.33 | 2.2 |
| 8-I14 | 1.22 | 8.1 |
| 9-E16 | 42.80 | 285.3 |

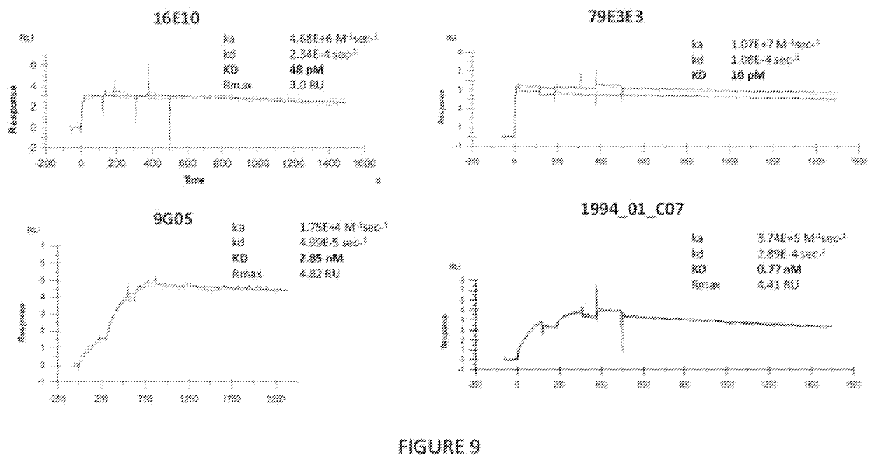

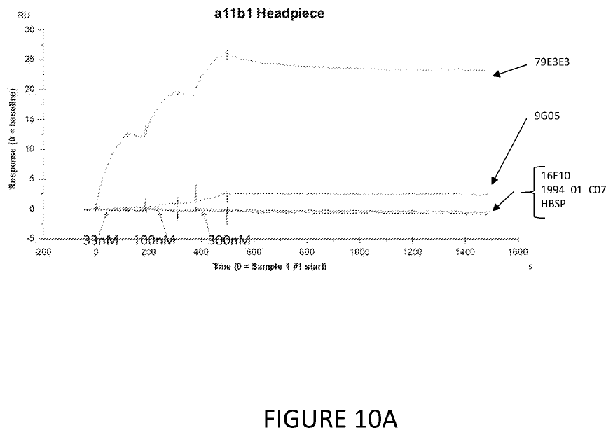

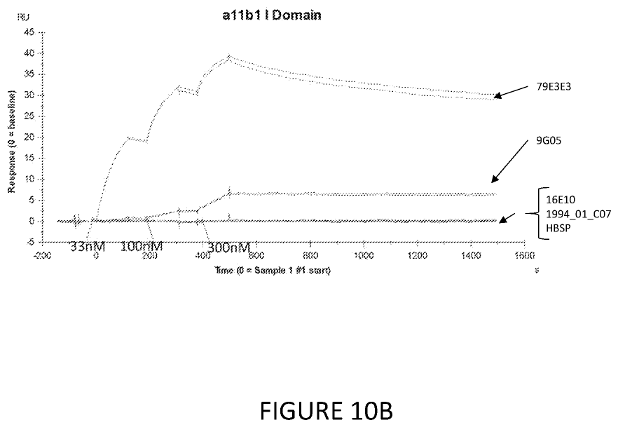

As shown in FIG. 9, when antibodies 16E10, 79E3E3, 9G05 and 1994_01_C07 were tested for their affinity to human α11β1 via surface plasmon resonance (SPR), they exhibited KDs of 48 pM, 10 pM, 2.85 nM and 0.77 nM, respectively. Interestingly, 16E10 and 1994_01_C07 do not bind either the I domain or the headpiece domain of α11β1, which indicates that both might act as allosteric inhibitors by not binding to the ligand binding domain but still inhibiting α11β1 function. 9G05 and 79E3E3 do bind to the I domain (the ligand binding domain) and therefore might directly inhibit the ligand binding site. The binding affinity of antibodies for the α11β1 Headpiece and α11β1 I Domain as measured by SPR is shown in FIG. 10A and FIG. 10B, respectively.

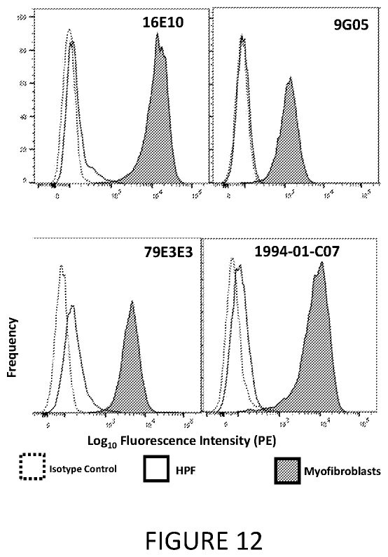

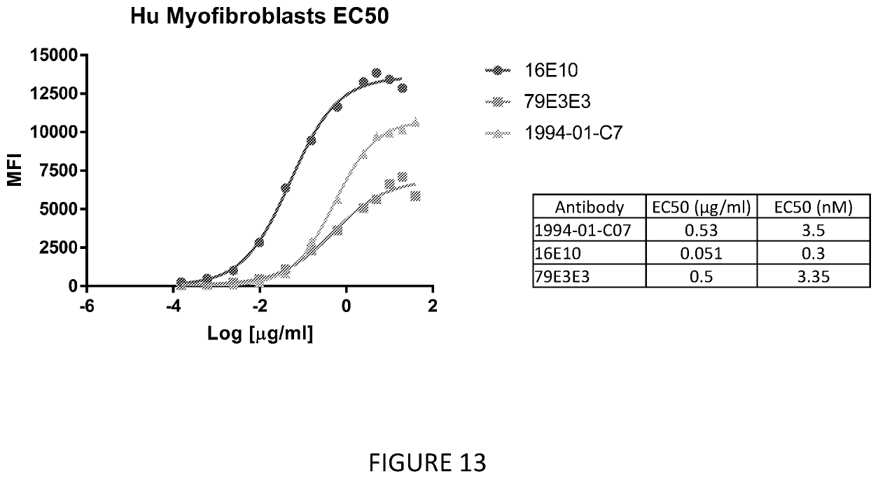

Binding to a physiologically relevant primary human cell type was also tested. Human pulmonary fibroblasts (HPF) and TGFβ treatment were used to induce a fibroblast-to-myofibroblast transition (FMT), giving rise to myofibroblasts (MF). While HPFs do not express α11β1, significant expression of α11β1 is exhibited by MFs. Additionally, HPFs express α1β1 and α2β1, other collagen binding receptors, which means that HPFs can be to test cross-reactivity of antibodies of interest. Selected mAbs were assessed for binding to HPFs and MFs, and as shown in FIG. 5, FIG. 12, and FIG. 13, it is apparent that the tested antibodies bound MFs strongly while not binding HPFs, with the exception of 9-E16, which showed some HPF binding, indicative of off-target binding.

### Example 2. Biological Activity of Novel Monoclonal Antibodies Against α11β1

Myofibroblasts are responsible for secreting fibrotic matrix, so blocking and/or reducing MF accumulation is an important step for treating and/or preventing fibrosis. This can be achieved by using anti-α11β1 antibodies to inhibit the fibroblast-to-myofibroblast transition. Typical functional inhibitors of a receptor block ligand binding, and while preventing the binding of α11β1 to type I collagen is a desired feature of anti-α11β1 antibodies, it may not be necessary for therapeutic efficacy. Unlike many other receptors, integrins are able to perform both “outside-in” (canonical, ligand-mediated) signaling and also “inside-out” signaling. Therefore, it might be possible for an antibody to bind α11β1 in a way that affects the structure of α11β1 in a way that prevents inside-out signaling and FMT, but does not affect the ability of α11β1 to bind type I collagen. For this reason, both mAbs that block ligand binding and those that do not were included these studies.

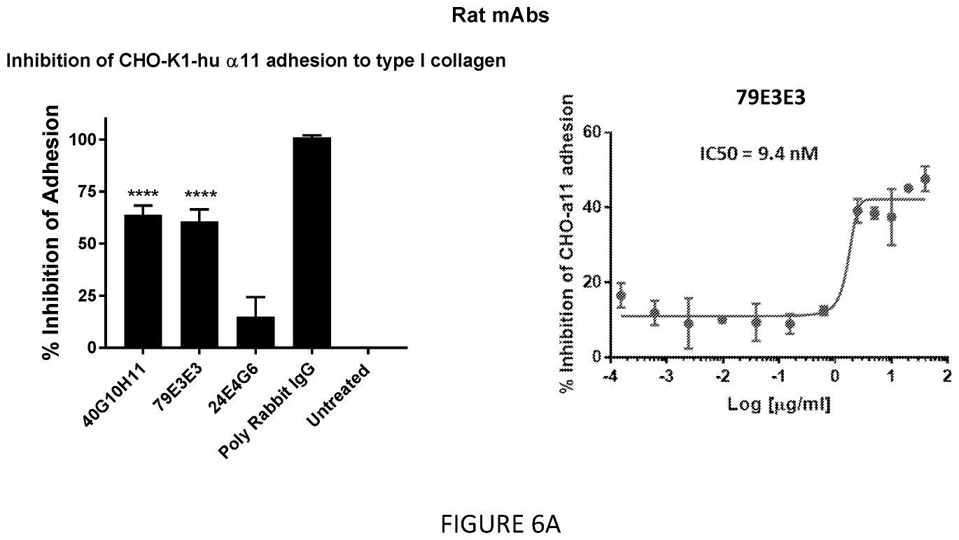

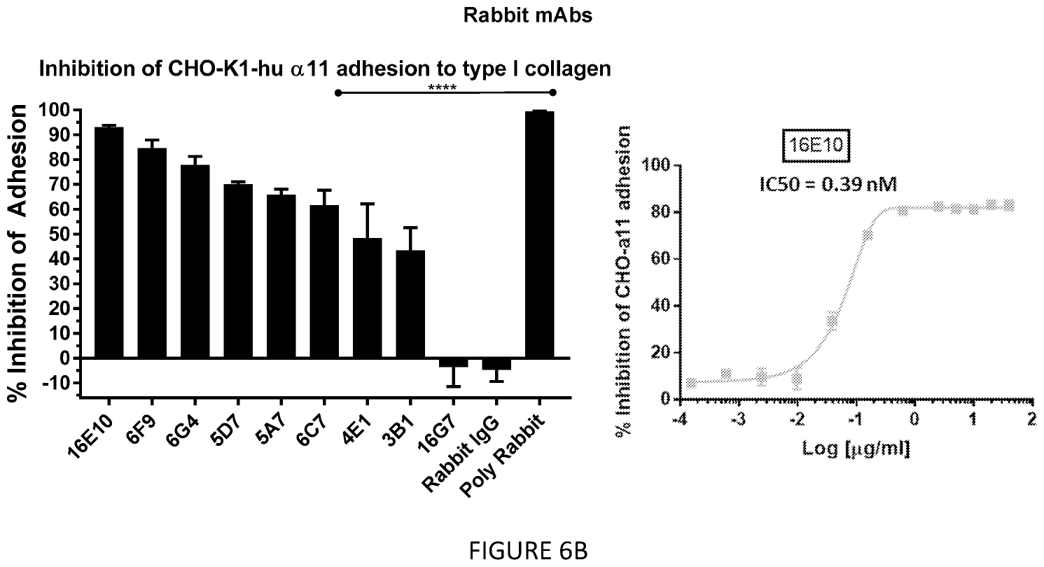

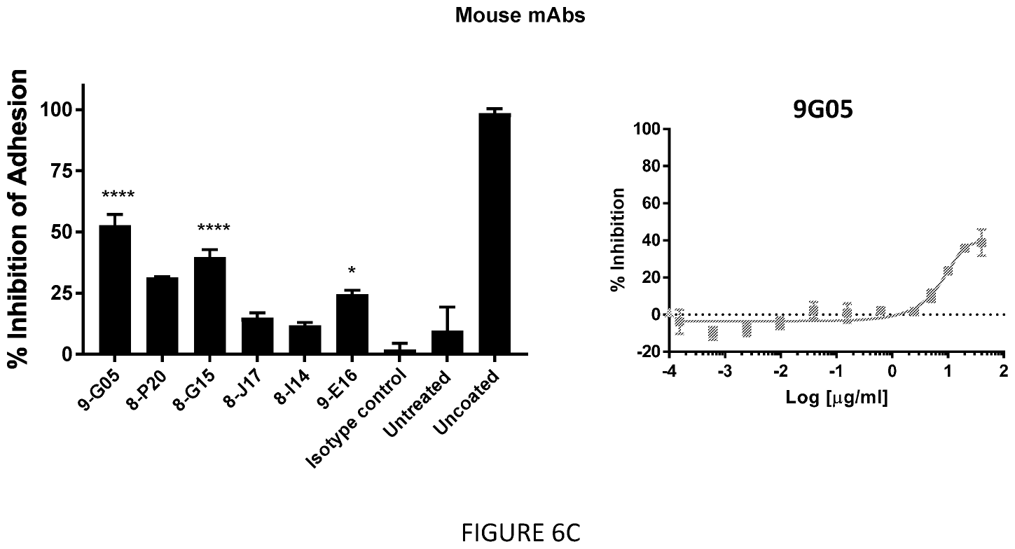

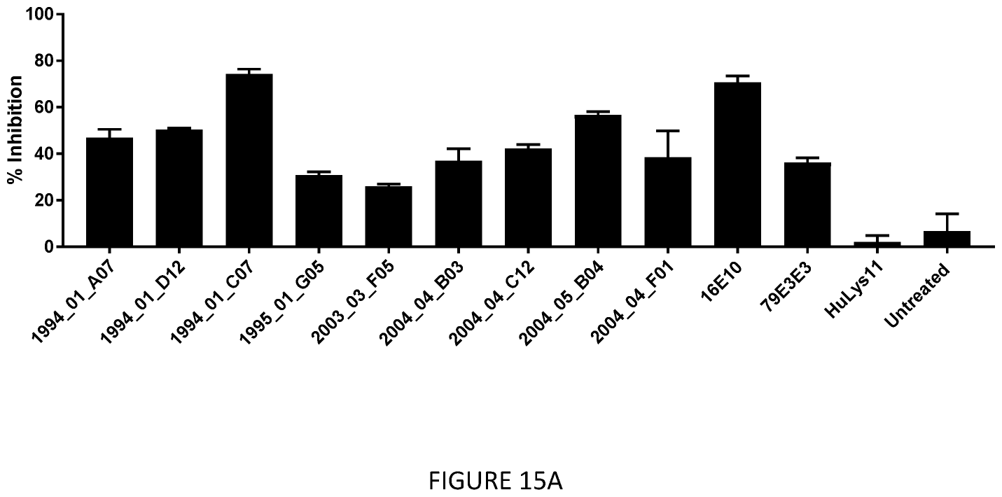

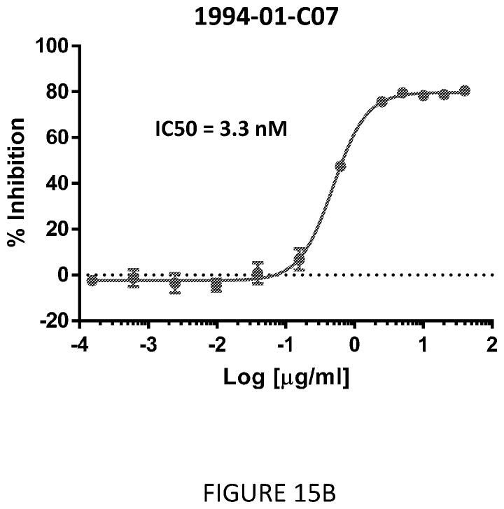

A CHO-K1 hu α11 cell line was used to assess the ability of mAbs to block α11β1-mediated binding to type I collagen. As shown in FIG. 6A, out of three rat mAbs tested, two significantly inhibited adhesion of CHO-K1 hu α11 cells to type I collagen-coated plates. In the “Untreated” condition, cells were plated on type I collagen but no antibody was added and in the “Uncoated” condition, cells were seeded onto BSA-coated wells containing no type I collagen. Statistics were performed using one-way ANOVA followed by Dunnett's multiple comparisons test. 79E3E3, which is an I-domain binder, was able to block cell adhesion with an IC50 of 9.4 nM. However, 40G10H11 strongly inhibited cell adhesion but was not found to be an I-domain binder, though it does cross-react with other collagen receptors (α1β1, α2β1 and α101). 24E4G6 did not bind to the I-domain nor did it inhibit cell adhesion to collagen. When rabbit mAbs were tested, eight of the nine mAbs strongly and significantly inhibited cell adhesion and none of those mAbs were found to be I-domain binders (FIG. 6B). Therefore, it is possible that these mAbs bind on an α11β1 domain that keeps the integrin in a low or intermediate affinity state. FIG. 6C shows the activity of selected mouse mAbs. Three of six mAbs significantly blocked cell adhesion, and two of those mAbs were found to be I-domain binders (9-G05 and 9-E16). 8-G15, however, is a strong blocker of cell adhesion but was not found to bind the I-domain. 8-P20, 8-J17 and 8-I14 did not block cell adhesion to type I collagen. Nine human mAbs were also tested for their ability to inhibit the binding of human CHO-α11 cells to type I collagen. As shown in FIGS. 15A and 15B, all of the human Abs inhibited cell adhesion relative to the control, with 1994-01-C07 having an IC50 of 3.3 nM. As illustrated by these data (and summarized in Table 1), some anti-α11β1 mAbs were found to strongly bind human α11β1 when tested by ELISA and FACS, but not all of those antibodies were able to block ligand interaction. Furthermore, binding to the I-domain (ligand binding domain on α11β1) was found to not be necessary for blocking the interaction of α11β1 with type I collagen.

In addition to the binding abilities described above, it is important for an anti-α11β1 antibody to inhibit the fibroblast-to-myofibroblast transition (FMT). It is now appreciated that myofibroblasts are a heterogeneous cell population, existing in different activation states, with the main function of producing and contracting collagen extracellular matrix (ECM). FMT is a multi-step event that is controlled by a changing mechanical environment in tissues undergoing repair. TGFβ is one of the potent factors that potentiates this process and alpha smooth muscle actin (αSMA) is one of the main markers that becomes overexpressed when fibroblasts are undergoing the transition to becoming myofibroblasts. The presence of αSMA enhances fibroblast contraction and guides myofibroblast activation through an intracellular feedback loop. Because αSMA is the main molecular marker of myofibroblasts, the ability of the novel anti-α11β1 mAbs to inhibit αSMA expression in TGFβ-induced FMT was tested.

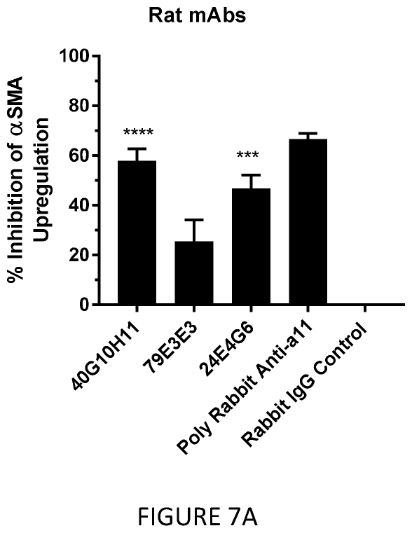

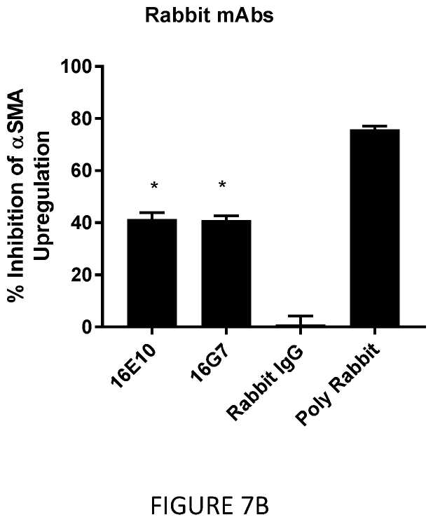

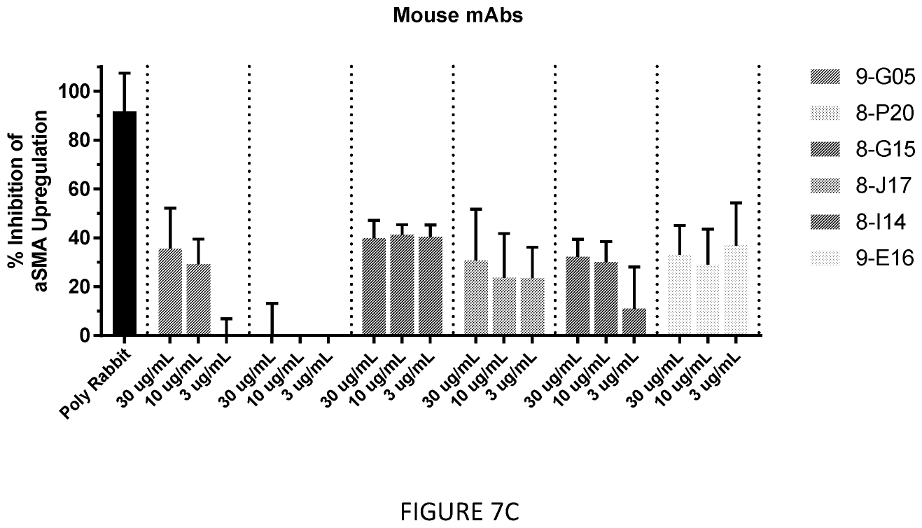

As shown in FIG. 7A, two rat mAbs (40G10H11 and 24E4G6) significantly inhibited αSMA expression compared to control, but neither of the antibodies was found to be an I-domain binder. Statistics were performed using one-way ANOVA followed by Dunnett's multiple comparisons test. Furthermore, only 40G10H11 inhibited cell adhesion to type I collagen. Interestingly, the 79E3E3 mAb was found to be an I-domain binder and strongly inhibited cell adhesion to collagen but failed to decrease αSMA expression (a surrogate for myofibroblast generation). As shown in FIG. 7B, two rabbit mAbs significantly inhibited αSMA expression compared to control, but neither of the antibodies was found to be an I-domain binder. 16E10 was found to inhibit both ligand binding and FMT (% inhibition of αSMA upregulation), but 16G7 was found to inhibit FMT but didn't have an effect on cell adhesion to collagen. Finally, as shown in FIG. 7C, five of the six tested mouse mAb significantly inhibited αSMA expression compared to control. Three of the FMT inhibitors (9-G05, 8-G15, 9-E16) were also found to decrease cell adhesion to collagen and two of those (9-G05, 9-E16) also bound the I domain. Mouse antibodies 8-J17 and 8-I14 only inhibited FMT and had effect on ligand binding.

### Example 3. Ability of Selected Antibodies to Inhibit Cell-Mediated Contraction of Collagen Gels

Cell-mediated contraction of CD collagen I gels is a process previously shown to be α11β1-mediated, and a more recent study showed that α11β1-mediated downstream signaling was indispensable for gel contraction to occur. Selected exemplary antibodies were tested for the ability to inhibit cell-mediated 3D gel contraction because this ability is directly linked to the functionality of the exemplary antibodies.

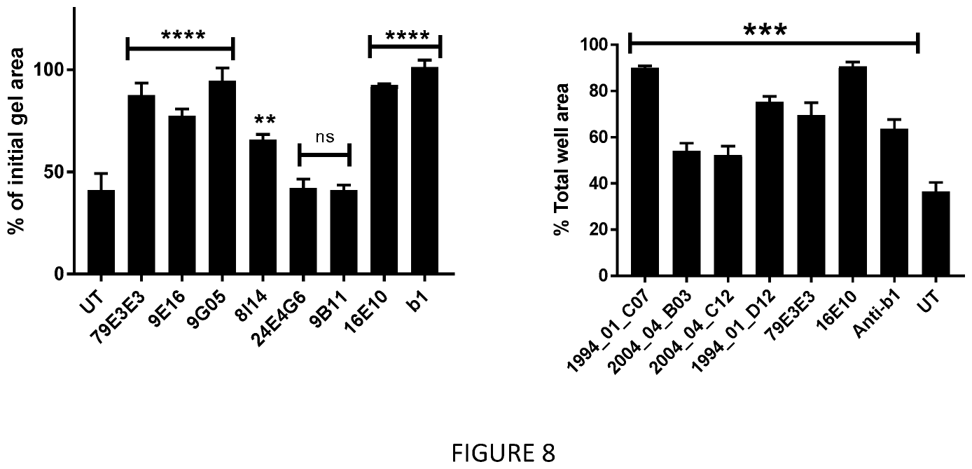

As can be seen in FIG. 8, rat 79E3E3, mouse 9E16, 9G05 and 8I14, rabbit 16E10, and human 1994_01_C07, 2004_04_β03, 2004_04_C12, and 1994_01_D12 antibodies all inhibited CHO-hu α11-mediated collagen gel contraction. Of note, CHO-hu α11 cells were able to contract collagen gel without the addition of TGFβ, as shown in the UT (untreated) condition. In the untreated condition, cells were embedded in the collagen gel but no antibody was added. Statistics were performed using one-way ANOVA followed by Dunnett's multiple comparisons test; each treatment conditions was compared to untreated condition. Asterisks indicate statistical significance and “ns” indicates that the difference was not statistically significant.

### Example 4. Assessing Effect of Selected Antibodies on Tumor Xenograft Growth

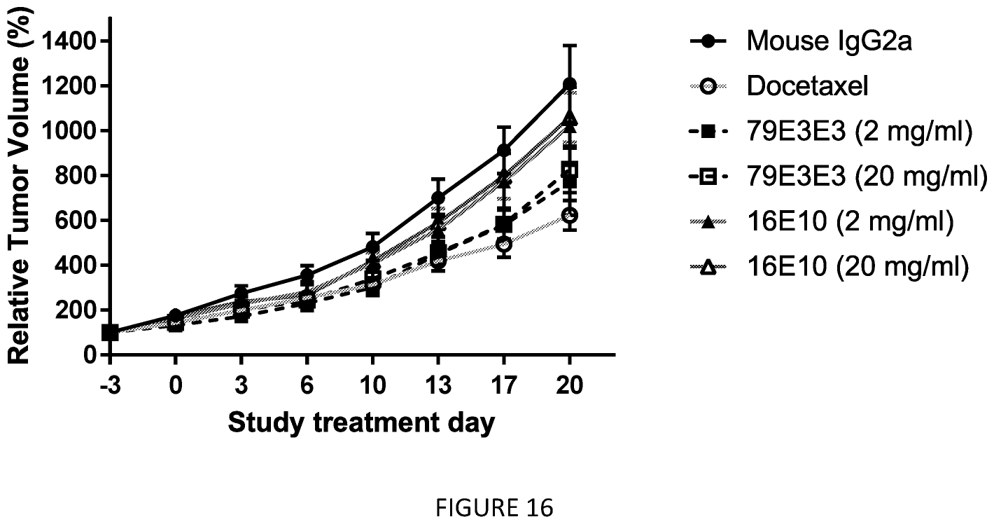

Previous studies have shown growth of A549 cell xenografts in α11 knockout SCID mice to be significantly impeded compared to wild-type mice. In this example, studies were performed to determine if inhibition of α11β1 function with mAb results in xenograft growth inhibition. As shown in FIG. 16 and Table 4, blocking α11β1 on mouse CAFs impeded xenograft growth in SCID mice. Specifically, 79E3E3, an effectorless mAb that cross-reacts with mouse α11β1, significantly inhibited tumor growth compared to isotype control, while 16E10, a mAb that does not bind mouse α11β1, showed no significant inhibition of tumor growth. Inhibition of tumor-expressed α11β1 did not affect tumor growth as 16E10 did not show any effect.

TABLE 4
Days to volume doubling after treatment with mAbs

| | Days to Volume Doubling |
|---|---|
| Treatment | (ave, 95% CI) |
| Mouse IgG2a | 7.6 (7.3, 8.0) |
| Docetaxel | 11.0 (10.0, 12.0) |
| 79E3E3 2 mpk | 8.5 (8.0, 9.0) |
| 79E3E3 20 mpk | 8.5 (8.0, 9.0) |
| 16E10 2 mpk | 7.9 (7.5, 8.3) |
| 16E10 20 mpk | 7.9 (7.5, 8.3) |

### Example 5. Effect of Anti-α11β1 Antibodies on Human Precision-Cut Liver Slices (PCLS)

Precision-Cut Liver Slices (PCLS) from human liver tissue are physiologically and structurally representative of the tissue architecture and testing therapeutic targets in human PCLS allow for assessment of their effectiveness and relevance to the clinical situation, overcoming the limitations of in vivo rodent models and in vitro 2D cell culture methods. Tissue bioreactor technology maintains viability and functionality of PCLS from human liver tissue for at least 6 days in vitro.

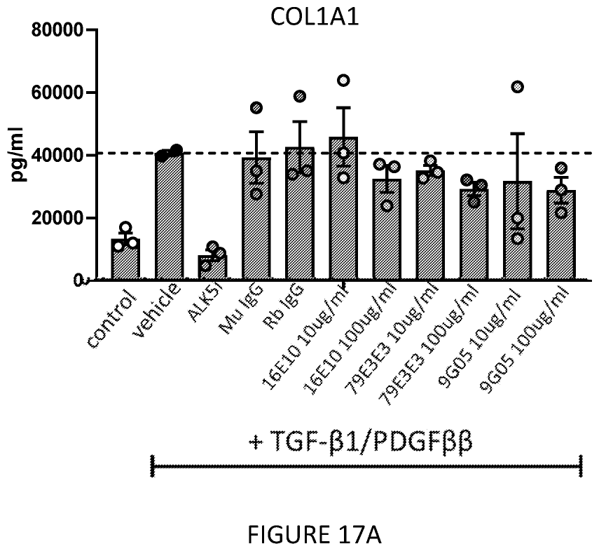

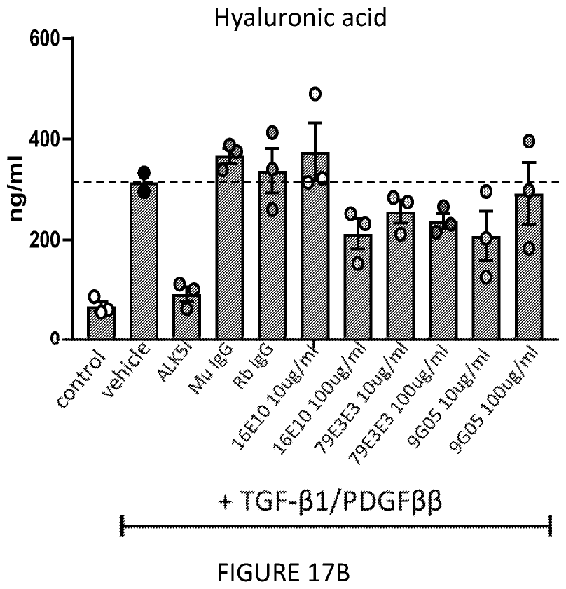

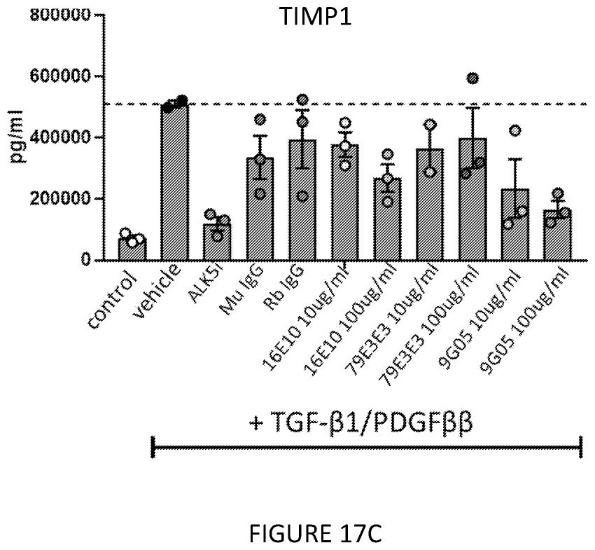

As shown in FIGS. 17A-17C, all anti-α11-β1 antibodies that were tested provided partial inhibition of soluble pro-fibrogenic markers (COL1A1, hyaluronic acid and TIMP1) either in a dose-dependent manner or at the highest dose tested. No toxicity was observed after treatment with any of the antibodies (i.e., no elevation in ALT, AST, or albumin; data not shown).

### Example 6. Effect of Anti-α11β1 Antibodies on Human Precision-Cut Kidney Slices (PCKS)

Human Precision-Cut Kidney Slices (PCKS) are prepared from human kidney tissue with moderate fibrosis. After resting for 24 hours, PCKS are cultured with novel anti-α11β1 monoclonal antibodies, at different doses and over time. At each time point, PCKS culture media is collected. At the harvest time point, culture media is collected and subsequently, RNA is extracted from each tissue slices. A range of profibrotic mediators are measured in the culture media as secreted proteins (including, but not limited to, COL1A1, Fibronectin, PAI-1, IL-11, CXCL1, MCP-1, IL-6, TIMP-1, Hyaluronic acid, TGFβ, CTGF, PDGF, and MMP9) and also at the transcriptional level (including, but not limited to, COL1A1, IL-6, TIMP-1, Hyaluronic acid, TGFβ, CTGF, αSMA, and ITGA11).

As described in the methods above, PCKS were prepared from explanted fibrotic human kidney tissue and rested for 24 hours to allow the post-slicing stress period to elapse before experiments began. PCKS were cultured with TGF-β1 (3 ng/ml) and PDGFββ (50 ng/mL) in the presence or absence of Alk5i (10 μM, positive control), test anti-α11β1 antibodies over three doses (1, 10 and 50 μg/mL) or in the presence of IgG control antibodies at a single high dose. PCKS culture media was harvested every 24 hours for a total of 3 time points. Levels of Collagen type I α1 (col1a1) in the tissue culture supernatants were quantified using R&D Duoset ELISA kits. Statistics were performed using Two-way ANOVA followed by Dunnett's multiple comparisons test.

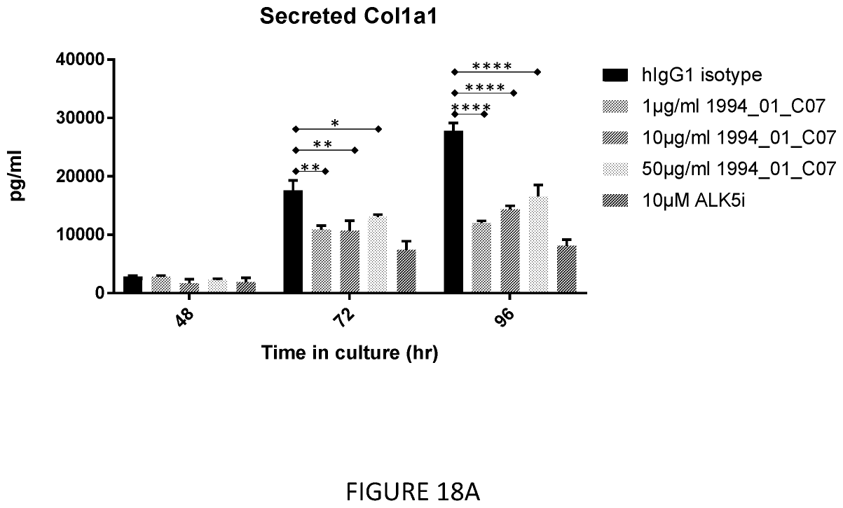

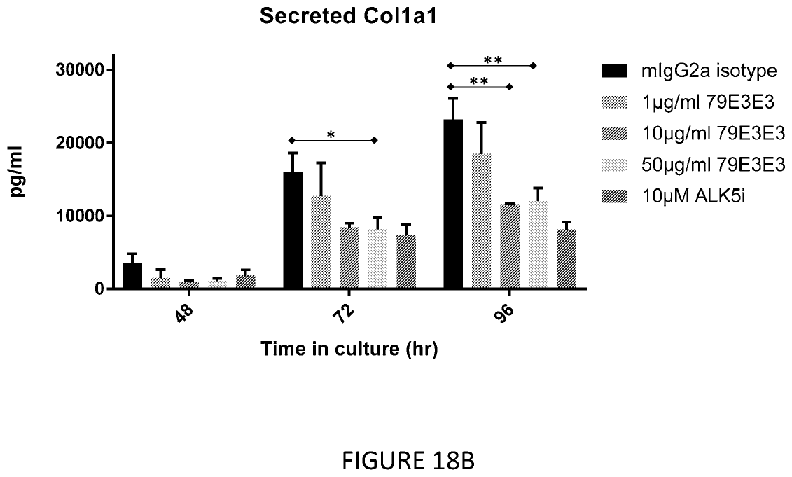

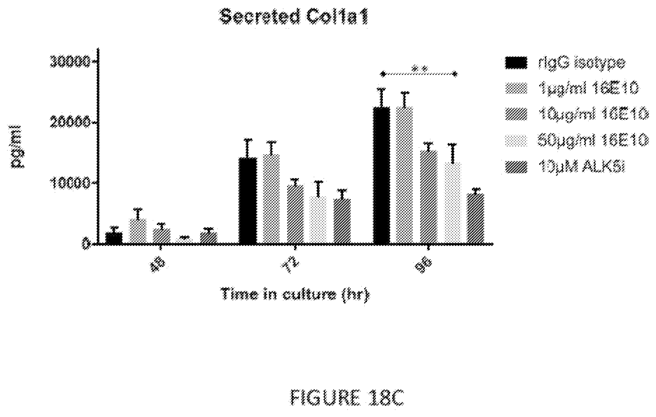

As shown in FIGS. 18A-18C, for each anti-α11-β1 antibody, at least one dose at at least one time point provided a significant inhibition of col1a1 secretion. There was a significant inhibition of col1a1 secretion by 1994_01_C07 novel mAb at 72- and 96-hour time points at each dose tested. This is an important finding as collagen type I deposition is the major culprit of fibrotic tissue.

### Example 7. Effect of Anti-α11β1 Antibodies on In Vivo Kidney Fibrosis Model

Two different kidney fibrosis models are employed, a ⅚ Nephrectomy model and a ReninAAV Unx db/db mouse model. These two models are characterized by significant renal fibrosis with measurable reduced renal function. Anti-α11β1 mAbs are tested in those models and the effect they have on renal morphology (fibrosis) as well as renal function (GFR and albuminuria) are measured.

EXEMPLARY SEQUENCES
DNA sequences of anti-α11β1 monoclonal antibodies
Rat mAb sequences
Signal sequence-FR1-CDR1-FR2-CDR2-FR3-CDR3-FR4
79E3E3 Heavy Chain Variable Region
Signal sequence-FR1-CDR1-FR2-CDR2-FR3-CDR3-FR4
ATGGATTGGTTGTGGAACTTGCTATTCCTGATGGTAGTTGCCCAAAGTGCTCAAGCACAG
ATCCAGTTGGTACAGTCTGGACCTGAAGTAAAGAAGCCTGGAGAGTCAGTGAAGAT
CTCCTGCAAGGCCTCTGGGTATACCTTCACAGACTATGCAATGAACTGGGTGAAAC
AGGCTCCAGGAAATGGCCTGAAGTGGATGGGCTGGATCAACACCCAAACTGGAAA
GCCAACATATGCGGATGATTTCAAACAACGGTTTGTCTTCTCTTTGGAAACTTCTG
CCAGAACTACATATTTGCAGATCAACAACCTCAATATTGAAGACACAGCTACATATT
TCTGTACGAGATTGGGTACAGGTAATACGAAGGGGTTTGCTTACTGGGGCCAAG
GCACTCTGGTCACTGTCTCTTCA (SEQ ID NO: 1)
79E3E3 Light Chain Variable Region
ATGGAATCACAGACGCATGTCCTCATTTCCCTTCTGCTCTGTGTATCAGGTACCTGTGGGG
ACATTTTGATAAACCAGTCTCCAGCCTCTCTGACTGTGTCAGCAGGAGAGAGGGTCA
CTATGAGCTGCAAGTCCAGTCAGAGTCTTCTATACAGTGAAAACAACCAGGACT
ATTTGGCTTGGTACCAGCAGAAACCAGGACAGTTTCCTAAATTGCTTATCTATGGG
GCATCCAACCGGCACACTGGGGTCCCTGATCGCTTCACAGGCAGTGGATCTGGGA
CAGACTTCACTCTGACCATCAGCAGTGTGCAGGCTGAAGACCTGGCTGATTATTATT
GTGAGCAGACCTACAGATATCCATTCACGTTCGGCTCAGGGACGAAGTTGGAAAT
AAAA (SEQ ID NO: 2)
24E4G6 Heavy Chain Variable Region
ATGGAGTTGGAATTGAGCTTAATTTTTATTTTTTCTCTTTTAAAAGATGTCCAGTGTGAAGT
ACAGCTGGTGGAGTCTGGAGGAAGCTTGGTTCAACCTGGGGGTTCTCTGAAACTCTC
CTGTGTAGCCTCAGGATACACTTTCAGTAACTACTGGATGGACTGGGTTCGGCAGT
CTCCTGGAAAGTCCCTGGAATGGATTGGAGAGATTAACACGGATGGCAGAAGGAC
CAACTATGCACCATCCATAAAGGATCGATTCACAATCTCCAGAGACAATGCCAAG
AGCACCCTGTATCTGCAGATGAGCAATGTGAAATCAGATGACACAGCCATTTATTAC
TGTACCATACTACGGGTATACCCCCACTACTTTGATTACTGGGGCCAAGGAGTCA
TGGTCACAGTCTCCTCA (SEQ ID NO: 3)
24E4G6 Light Chain Variable Region
ATGATGAGTCCTGCCCAGTTCCTGTTTCTGCTAATGCTCTGGATCCAGGAAGCCCGC
GGAGATGTTGTGATGACCCAGACACCACCGTCTTTGTCGGTTGCCATTGGACAATCA
GTCTCCATCTCTTGCAAGTCAAGTCAGAGCCTCGTATATAGTGATGGAGAGACAT
ATTTGCATTGGTTTTTACAGAGTCCTGGCAGGTCTCCGAAGCGCCTAATTTATCACG
TGTCTAATCTGGGCTCTGGAGTCCCTGACAGGTTCAGTGGCACTGGATCACTGACA
GATTTTACACTTAGAATCAGCAGAGTGGAGGCTGAGGATTTGGGAGTTTATTACTGC
GCGCAAACTACACATTTTCCTCCCACGTTTGGAGCTGGGACCAAGCTGGAACTGA
AA (SEQ ID NO: 4)
8H8E9 Heavy Chain Variable Region
ATGGCTGTCCTGGTGCTGTTGCTCTGCCTGGTGACATTTCCAAGCTGTGCCCTGTCCCAG
GTGCAGTTGAAGGAGTCAGGACCTGGTCTGGTGCAGCCCTCACAGACCCTGTCCCTC
ACCTGCACTGTCTCTGGGTTCTCATTAACCAGCAATAGTGTTAGCTGGGTTCGCCA
GGCTCCGGGAAAGGGTCTGGAGTGGATGGGAGCAATATGGAGTGGTGGAAGCAC
AGATTATAATTCAGCTCTCAAATCCCGACTGAGCATCAGCAGGGACACCTCCAAG
AGCCAAGTTTTCTTAAAAATGAACAGTCTGCAAACTGAAGACACAGCCATTTACTTC
TGTACCAGATCTCACTGGGAGCCCTTTGATTACTGGGGCCAAGGAGTCATGGTCA
CAGTCTCCTCA (SEQ ID NO: 5)
8H8E9 Light Chain Variable Region
ATGGAATCACAAACTCAGGCCCTCATATCCCTGCTGCTCTGGGTATATGGTACCTGTGGG
GACATTGTGATGACCCAGTCTCCATTCTCCCTGGCTGTGTCAGAAGGAGAGATGGTC
ACTATAAACTGCAAGTCCAGTCAGGGTCTTTTATCCAGTGGAAACCAAAAGAAC
TACTTGGCCTGGTACCAGCAGAGACCAGGGCAGTCTCCTAAACTACTGATCTACTA
TGCATCCACTAGGCAATCAGGGGTCCCTGATCGCTTCATAGGCGGTGGATCTGGGA
CAGACTTCACTCTGACCATCAGCGATGTGCAGGCTGAAGACCTGGCAGATTATTACT
GCCTGCAGCATTACAGCTATCCTCCCACGTTCGGTTCTGGGACCAAGCTGGAGAT
CAAA (SEQ ID NO: 6)
6E5C11 Heavy Chain Variable Region
ATGGCTGTCCTGGTGCTGTTGCTCTGCCTGGTGACATTTCCAAGCTGTGCCCTGTCCCAG
GTGCAGCTGAGGGAGTCAGGACCTGGTCTGGTGCAGCCCTCACAGACCCTGTCCCTC
ACCTGCACTGTCTCTGGGTTCTCATTGACCAGCAATAGTGTGACCTGGGTTCGCCA
GCCTCCGGGAAAGGGTCTGGAGTGGATGGGAGCGATATGGAGTGATGGAAGCAC
AGATTATAATTCAACTCTCAAATCCCGACTGAGCATCAGTAGGGACACCTCCAAG
AGCCAAGTTTTCTTAAAAATGAGCAGTCTGCAAACTGAAGACACAGCCATTTACTTC
TGTACCAGATCCCACTGGGAGCCCTTTGATTACTGGGGCCAAGGAGTCATGGTCA
CAGTCTCCTCA (SEQ ID NO: 7)
6E5C11 Light Chain Variable Region
ATGGAATCACAAACTCAGGCCCTCATATCCCTGCTGCTCTGGGTATATGGTACCTGTGGG
GACATTGTGATGACCCAGTCTCCACTCTCCCTGGCTGTGTCAGAAGGAGAGACGGTC
ACTATGAACTGCAAGTCCAGTCAGAGTCTTTTTTCCAGTGGAAATCAAAAGAAC
TACTTGGCCTGGTACCAGCAGAAACCAGGGCAGTCTCCTAAACTACTGATCTACTA
TGCATCCACTAGGCAATCAGGGGTCCCTGATCGCTTCATAGGCAGTGGATCTGGGA
CAGACTTCACTCTGACCATCAGCGATGTGCAGACTGAAGACCTGGCAGATTATTACT
GCCTGCAGCATTACAACTATCCTCCCACGTTCGGTTCTGGGACCAAGCTGGAGA
(SEQ ID NO: 8)
7D8B10 Heavy Chain Variable Region
ATGGACTTGCGACTGACTTATGTCTTTATTGTTGCTATTTTAAAAGGTGTCTTGTGTGAGGT
GAAACTGGAGGAATCTGGGGGAGGTTTGGTGCAACCTGGAATGTCCGTGAAACTCT
CTTGTGCAACCTCTGGATTCATTTTCAGTGACTACTGGATGGAATGGGTCCGCCAG
GCTCCAGGGAAGGGGCTAGAATGGGTAGCCGAAATTAGAAACAAAGCTAATAATT
ATGCAACATACTATGGGAAGTCTATGAAAGGCAGATTCACCATCTCAAGAGATGA
TTCCAAAAGTATAGTCTACCTACAAGTGAACAGCATAAGATCTGAAGATACTGCTAT
TTATTACTGTGCACCGAATTTTGATTACTGGGGCCAAGGAGTCATGGTCACGGTCTC
CTCA (SEQ ID NO: 9)
7D8B10 Light Chain Variable Region
ATGAGTCCTGTCCAGTCCCTGTTTTTGCTATTGCTTTGGATTCTGGGAACCCATGGTGATG
TTGTGCTGACCCAGACTCCACCCACTTTATCGGCTACCATTGGACAATCAGTCTCTAT
CTCTTGCAGGTCAAGTCAGAGTCTCTTACATAGTACTGGAAACACCTATTTAAAT
TGGTTGCTACAGAGGCCAGGCCAACCTCCGCAACTTCTAATTTATTTGGTTTCCAG
ACTGGAATCTGGGGTCCCCAACAGGTTCAGTGCCAGTGGGTCAGGAACTGATTTCA
CACTCAAAATCAGTGGAATAGAGGCTGAGGATTTGGGGGTTTATTACTGCGTGCAA
AGTTCCCATACTCCGTACACGTTTGGGACTGGGACCAAGCTGGAACTGAAA (SEQ
ID NO: 10)
18E10F10 Heavy Chain Variable Region
ATGGACATCAGGCTCAGCTTGGTTTTCCTTGTCCTTTTTATGAAAGGTGTCCAGTGTGAGG
TGCAGTTGGTGGAGTCTGGGGGAGGCTTAGTGCAGCCTGGAAGGTCCCTGAAACTCT
CCTGTGCAGCCTCACGATTCACTTTTAGTGACTATAACATGGCCTGGGTCCGCCAG
GCTCCAAAGAAGGGTCTGGAGTGGGTCGCAACCATTTATCATGATGATAGTGGTT
CTTACTATCGAGACTCCGTGAAGGGCCGATTCACCATCTCCAGAAATAATGCAAA
AAGCACTCTGTACCTGCAGATGGACAGTCTGAGGTCTGAGGACATGGCCACTTATTA
CTGTGCAAGACATAACAATGGCTTTGATTACTGGGGCCAAGGAGTCATGGTCACA
GTCGCCTCA (SEQ ID NO: 11)
18E10F10 Light Chain Variable Region
ATGAAGTGGCCTGTTAGGCTGTTGGTGCTGTTCTTCTGGATTCCTGCTTCCGGGGGTGAT
GTTGTGATGACACAAACTCCAGTCTCCCTGCCTGTCCGCCTTGGAGGTCAAGCCTCT
ATCTCTTGCCGGTCAAGTCAGAGCCTGGTACACAGTAATGGAAACACCTACTTG
CATTGGTACCTACAGAAGCCAGGCCAGTCTCCACAGCTCCTCATCAATCGGGTTTC
CAACAGATTTTCTGGGGTGCCAGACAGGTTCAGTGGCAGTGGGTCAGGGACAGATT
TCACCCTCAAGATCAACAGAGTAGAGCCTGAGGACTTGGGAGATTATTACTGCTTA
CAAAGTACACATTTTCCACTCACGTTCGGTTCTGGGACCAAGCTGGAGACCAAA
(SEQ ID NO: 12)
40G10H11 Heavy Chain Variable Region
ATGGACATCAGGCTCAGCTTGGGTTTCCTTGTCCTTTTCATAAAAGGTGACCAGTGTGCGG
TGCAACTGGTGGAGTCTGGGGGAGGCTTAGTGCAGCCTGGAAGGTCCCTGAAACTC
TCCTGTGCAGCCTCAAGAATCACTTTCACTGACTATTACATGGCCTGGGTCCGCCA
GGCTCCAACGAAGGGTCTGGAGTGGGTCGCAACCATTAGTTCTGATGGTGGTGAC
ACTTTCTATCGAGACTCCGTGAAGGGCCGATTTACTATCTCCAGAGACAATGCAA
AAAGCACCCTATATTTGCAAATGGTCAGTCTGAGGTCTGAGGACACGGCCACTTATT
ACTGTTCAACAGATCGGGGAGCTCAGTTTGGTTACTGGGGCCAAGGCACTCTGGT
CACTGTCTCTTCA (SEQ ID NO: 13)
40G10H11 Light Chain Variable Region
ATGGCTCCAGTCCAGCTCTTAGGGCTGCTGCTGATTTGGCTCCCAGCCATGAGATGTGAC
ATCCAGATGACCCAGTCTCCTTCATTCCTGTCTGCATCTGTGGGAGACAGAGTCTCT
ATCAACTGCAAAGCAAGTCAGAATGTTCACGAGAACCTAAACTGGTATCAGCAAA
AGCTTGGAGAAGCTCCCAAACGCCTGATATATAATACAAACAATTTGCAAACAGG
CATCCCATCAAGGTTCAGTGGCAGTGGATCTGGTGCAGATTACACACTCACCATCAG
CAGCCTGCAGCCTGAAGATTTTGCCACATATTTCTGTTTGCAGCATAATGCTTTTC
CGTACACGTTTGGACCTGGGACGAAGCTGGAACTGAAA (SEQ ID NO: 14)
Mouse mAb sequences
9-G05 Heavy Chain Variable Region
GAGGTCCAGCTGCAACAGTCTGGACCTGAGCTGGTGAAGCCTGGGGCTTCAGTGAA
GATACCCTGCAAGGCTTCTGGATACACATTCCCTGACTACAACATGGACTGGGTGAA
GCAGAGCCATGGAAAGAGCCTTGAGTGGATTGGATATATTAATCCTGACAATGGTG
GTACTATCTACAACCAGAAGTTCAAGGGCAAGGCCACATTGACTGTAGACAAGTCC
TCCAGCACAGCCTACATGGAGCTCCGCAGCCTGACATCTGAGGACACTGCAGTCTAT
TACTGTGCAAGATTAGACAGCTCAGGCTACGGTTACTATGCTATGGACTACTGGGGT
CAAGGAACCTCAGTCACCGTCTCCTCA (SEQ ID NO: 15)
9-G05 Light Chain Variable Region
GACATTGTGCTGACCCAATCTCCAGCTTCTTTGGCTGTGTCTCTAGGGCAGAGGGCC
ACCATCTCCTGCAGAGCCAGCGAAAGTGTTGATAATTATGGCATTAGTTTTATGCAC
TGGTACCAGCAGAAACCAGGACAGCCACCCAAACTCCTCATCTATCGTGCATCCAA
CCTAGACTCTGAGATCCCTGCCAGGTTCAGTGGCAGTGGGTCTAGGACAGACTTCAC
CCTCACCATTGATCCTGTGGAGACTGATGATGTTGCAACCTATTACTGTCAGCAAAG
TTATAAGGATCCTCGGACGTTCGGTGGAGGCACCAAGCTGGAAATCAAA (SEQ ID
NO: 16)
8-P20 Heavy Chain Variable Region
AAAGTGATGCTGGTGGAGTCTGGGGGAGCCTTAGTGAAGCCTGGAGGGTCCCTGAA
ACTCTCCTGTGTAGCCTCTGGATTCACTTTCAGTAACTATGCCATGTCTTGGGTTCGC
CAGACTCCAGAGAAGAGGCTGGAGTGGGTCGCAACCATTAGTAGTGGTGGTTATTA
CACTTACTATCCAGACAGTGTGAAGGGTCGATTCACCATCTCCAGAGACAATGCCAG
GAACACCCTGTTCCTGCAAATGAGCAGTCTGAGGTCTGAGGACACGGCCATGTTTTA
CTGTGCAAGAGAGGATGATTACGGAAGATATTCCTATACTATGGACTACTGGGGTCA
AGGAACCTCAGTCACCGTCTCCTCA (SEQ ID NO: 17)
8-P20 Light Chain Variable Region
GATGTTGTGATGACCCAAACTCCACTCTCCCTGCCTGTCAGTCTTGGAGATCAAGTC
TCCATCTCTTGCAGATGTAGTCAGAGCCTTGTACACAGTAATGGAAACACCTATTTA
CATTGGTACCTGCAGAAGCCAGGCCAGTCTCCACAGCTCCTGATCTACAAAATTTCC
AACCGATTTTCTGGGGTCCCAGACAGGTTCAGTGGCAGTGGATCAGGGACAGATTTC
ACACTCAAGATCAGCAGAGTGGAGGCTGAGGATCTGGGAGTTTATTTCTGCTCTCAA
AGTACACATGTTCCGTACACGTTCGGAGGGGGGACCGAGCTGGAAATAAAA (SEQ
ID NO: 18)
8-G15 Heavy Chain Variable Region
GAGGTCCAGCTGCAGCAGTCTGGACCTGAGCTGGTAAAGCCTGGGGCTTCAGTGAG
GATATCCTGCAAGGCTTCTGGATACACATTCACTGACTACTACATACACTGGGTGAA
GCAGAAGCCTGGGCAGGGCCTTGAATGCATTGGAGAGATTTATCCTGGAACTGATA
ATACTTACTACAGTAAAAAATTCAGGGGCAAGGCCACACTGACTGCAGACAAATCC
TCCGACACAGCCTACATGCAGCTCAGCAGCCTGACATCTGAGGACTCTGCAGTCTAT
TTCTGTGCAAGAGGAGACTACTATAGGGGGTACTTCGATGTCTGGGGCGCAGGGAC
CACGGTCACCGTCTCCTCA (SEQ ID NO: 19)
8-G15 Light Chain Variable Region
GATGTTGTGATGACTCAGACCTCACTCACTTTGTCGGTTACCATTGGACAACCAGCC
TCCATCTCTTGCAAGTCAAGTCAGAGCCTCTTACATAGTAATGGAAAGACATATTTG
AATTGGTTATTACAGAGGCCAGGCCAGTCTCCAAAGTTCCTAATCTATCTGGTGTCT
AAACTGGAATCTGGAGTCCCTGACAGGTTCAGTGGCAGTGGATCAGGGACAGATTT
CACACTGAAAATCAGCAGAGTGGAGGCTGAGGATTTGGGGGTTTATTACTGCTTGCA
ATCTACACATTTTCCTTGGACGTTCGGTGGAGGCACCAAGCTGGAAATCAAA (SEQ
ID NO: 20)
8-114 Heavy Chain Variable Region
GAGGTCCAGCTGCAGCAGTCTGGACCTGAGCTGGTAAAGCCTGGGGCTTCAGTGAA
GAAATCCTGCAAGGCTTCTGGATACACATTCACTGACTACTACATGCACTGGGTGAA
GCAGAAGCCTGGGCAGGGCCTTGAGTGGATTGGAAAGATTTATCCTGGAAGTGGTA
ATACTCACTACAATGAGAAGTTCAAGGGCAAGGCCACACTGACTGCAGACAAATCC
TCCAGCACAGCCTACATGCAGCTCAGCAGCCTGACATCTGAGGACTCTGCAGTCTAT
TTCTGTGCAACCAATTACTACGGCTACAGGGCAATGAACTATTGGGGTCAAGGATCC
TCAGTCACCGTCTCCTCA (SEQ ID NO: 21)
8-114 Light Chain Variable Region
GACATCCATTTGACCCAGTCTCCATCCTCCTTATCTGCCTCTCTGGGAGAAAGAATC
AGTCTCACTTGCCGGGCAAGTCAGGACATTTATATTAGCTTAAACTGGTTTCAGCAG
AAACCAGATGGAACTATTAAACTCCTGATCTACGGCACATCCAGTTTAGATTCTGGT
GTCCCCAAAAGGTTCAGTGGCAGTAGGTCTGGGTCAGATTATTCTCTCACCATCAGC
AGCCTTGAGTCTGAAGATTTTGCAGACTATTACTGTCTACAATATGCTAGTTCTCCGT
ACACGTTCGGAGGGGGGACCAAGCTGGAAATAAAA (SEQ ID NO: 22)
9-E16 Heavy Chain Variable Region
GAGGTCCAGCTGCAGCAGTCTGGACCTGAGCTGGTAAAGCCTGGGGCTTCAGTGAA
GATATCCTGCAAGGCTTCTGGATACACATTCACTGACTACTACATGCACTGGGTGAA
GCAGAAGCCTGGGCAGGGCCTTGAGTGGATTGGAGAGATTTATCCTGGAAGTGGTA
ATCCTTACTACAATGAGAAGTTCAAGGGCAAGGCCACACTGACTGCAGACAAATCA
TCCAGCTCAGCCTACATGCAGCTCAGCAGCCTGACATCTGAGGACTCTGCAGTCTAT
TTCTGTGCAAGAACCTCCTACGGTAGAGTAGGGACAGGGTTTGCTTACTGGGGCCAA
GGGACTCTGGTCACTGTCTCTGCA (SEQ ID NO: 23)
9-E16 Light Chain Variable Region
AATTTTGTGATGACCCAAACTCCACTCTCCCTGCCTGTCAGTCTTGGAGATCAAGCCT
CCATCTCTTGCAGATCTAGTCAGAGCCTTCTACACAGTAACGGAAACACCTATTTAC
ATTGGTACCTGCAGAAGCCAGGCCAGTCTCCAAAGCTCCTGATCTACAAAGTTTCCA
ACCGATTTTCTGGGGTCCCAGACAGGTTCAGTGGCAGTGGATCAGGGACAGATTTCA
CACTCAAGATCAACAGAGTGGAGACTGAGGATCTGGGAATTTATTTCTGCTCTCAAA
GTTCACATGTTCCCACGTTCGGTGCTGGGACCAAGCTGGAGCTGAAA (SEQ ID NO:
24)
8-J17 Heavy Chain Variable Region
CAGGTCCAGCTGCAGCAGTCTGGGGCTGAACTGGCAAAACCTGGGGCCTCAGTGAA
GATGTCCTGCAAGGCTTCTGGCTACACCTTTACTAACTACTGGATGCACTGGGTAAA
ACAGAGGCCTGGACAGGGTCTGGAATGGATTGGATACATTAATCCTAACAATGGTT
ATACTGAGTACAATCAGCGATTCAAGGACAAGGCCACATTGACTGCAGACAGATCC
TCCACCACAGCCTACATGCAACTAAGCAGCCTGACATCTGAGGACTCTGCAGTCTAT
TACTGTGCAAGATCCGATATCATTACGACAGACTACTGGGGCCAAGGCACCACTCTC
ACAGTCTCCTCA (SEQ ID NO: 25)
8-J17 Light Chain Variable Region
GATGTTGTGATGACCCAAACTCCACTCTCCCTGCCTGTCAGTCTTGGAGATCAAGCC
TCCATCTCTTGCAGATCTAGTCAGAGCCTTGTATATAGTAATGGAAATACCTATTTAC
ATTGGTACCTGCAGAAGCCAGGCCAGTCTCCAAAGCTCCTGATCTACAAAGTTTCCA
ACCGATTTTCTGGGGTCCCAGACAGGTTCAGTGGCAGTGGATCAGGGACAGATTTCA
CACTCAAGATAAGCAGAGTGGAGGCTGAGGATCTGGGAGTTTATTTCTGCTCTCAAA
GTACACATGTTCCGTGGACGTTCGGTGGAGGCACCAAGCTGGAAATCAAA (SEQ ID
NO: 26)
6-012 Heavy Chain Variable Region
GAAGTGAAGCTTGAGGAGTCTGGAGGAGGCTTGGTGCAACCTGGAGGATCCATGAA
ACTCTCTTGTGCTGCCTCTGGATTCACTTTTAGTGACGCCTGGATGGACTGGGTCCGC
CAGTCTCCAGAGGCGGGGCTTGAGTGGGTTGCTGAAATTAGAAACAAAGCTCATAA
TCCTGCAACATACTATGCTGAGTCTGTGAAAGGGAGATTCACCATCTCAAGAGATGA
TTCCAAAAGTAGTGTCTACCTGCAAATGAACAGCTTAAGAGCTGAAGACACTGGCA
TTTATTACTGTACCTTAGTAGCCCCTGATGCTATGGACTACTGGGGTCAAGGAACCT
CAGTCACCGTCTCCTCA (SEQ ID NO: 27)
6-012 Light Chain Variable Region
GACATTGTGATGTCACTGTCTCCATCCTCCCTAGCTGTGTCAGTTGGAGAGAAGGTT
ACTATGAGCTGCAAGTCCAGTCAGAGCCTTTTATATAGTCGCAATCAAAAGAACTAC
TTGGCCTGGTACCAGCAGAAACCAGGGCAGTCTCCTAAACTGCTGATTTACTGGGCA
TCCACTAGGGCATCTGGAGTCCCTGATCGCTTCACAGGCAGTGGATCTGGGACAGAT
TTCACTCTCACCATCAGCAGTGTGAAGGCTGAAGACCTGGCAGTTTATTACTGTCAG
CAATATTATAGCTATCCGTACACGTTCGGAGGGGGGACCAAGCTGGAAATAAAA
(SEQ ID NO: 28)
10-L15 Heavy Chain Variable Region
CAGGTCCAACTGCAGCAGTCTGGGCCTGAGCTGGTGAGGCCTGGGGCTTCAGTGAA
GATGTCCTGCAAGGCTTCAGGCTATACCTTCACCAGCTACTGGATGCACTGGGTGAA
ACAGAGGCCTGGACAAGGCCTTGAGTGGATTGGCATGATTGATCCTTCCAATAGTGA
AACTTGGTTAAATCAGAAGTTCAAGGACAAGGCCACATTGAATGTAGACAAATCCT
CCAACACAGCCTACATGCAGCTCAGCAGCCTGACATCTGAGGACTCTGCAGTCTATT
ACTGTGCAAGATATGATGGTTACTACGACTACTGGGGCCAAGGCACCACTCTCACAG
TCTCCTCA (SEQ ID NO: 29)
10-L15 Light Chain Variable Region
AACATTGTGCTGACCCAATCTCCAGCTTCTTTGGCTGTGTCTCTAGGGCAGAGGGCC
ACCATATCCTGCAGAGCCAGTGAAAGTGTTGATAGTTATGGCAATAGTTTTATGCAC
TGGTACCAGCAGAAACCAGGACAGCCACCCAAACTCCTCATCTATCTTGCATCCAAC
GTAGAATCTGGGGTCCCTGCCAGGTTCAGTGGCAGTGGGTCTAGGACAGACTTCACC
CTCACCATTGATCCTGTGGAGGCTGATGATGCTGCAACCTATTACTGTCAGCAAAAT
AATGAGGATCCGTGGACGTTCGGTGGAGGCACCAAGCTGGAAATCAAA (SEQ ID
NO: 30)
7-H14 Heavy Chain Variable Region
CAGGTCCAACTGCAGCAGCCTGGGGCTGAGCTGGTGAGGCCTGGGGCTTCAGTGAA
GCTGTCCTGCAAGCCTTCTGGCTACACCTTCACCAGCTACTGGATGAACTGGGTGAA
GCAGAGGCCTGGACAAGGCCTTGAATGGATTGGTATGATTGATCCTTCAGACAGTG
AAACTCACTACAATCAAATGTTCAAGGACAAGGCCACATTGACTGTTGACAAATCCT
CCAACACAGCCTACATGCAGCTCAGCAGCCTGACATCTGAGGACTCTGCGGTCTATT
ACTGTGCGCAGATCTACTATGCTTACGACAAGGCTTACTGGGGCCAAGGGACTCTGG
TCACTGTCTCTGCA (SEQ ID NO: 31)
7-H14 Light Chain Variable Region
GACATTGTGATGTCACAGTCTCCATCCTCCCTAGCTGTGTCAGTTGGAGAGAAGGTT
ACTATGAGCTGCAAGTCCAGTCAGAGCCTTTTATATAGTAGCCATCAAAAGAACTAC
TTGGCCTGGTACCAGCAGAAACCAGGGCAGTCTCCTAAACTGCTGATTTACTGGGCA
TCCACTAGGGAATCTGGGGTCCCTGATCGCTTCACAGGCAGTGGATCTGGGACAGAT
TTCAGTCTCACCATCAGCAGTGTGAAGGCTGAAGACCTGGCAGTTTATTACTGTCAG
GAATATTATAGCTGGACGTTCGGTGGAGGCACCAAGCTGGAAATCAAA (SEQ ID
NO: 32)
6-B21 Heavy Chain Variable Region
GAGGTCCAGCTGCAACAATCTGGACCTGAGCTGGTGAAGCCTGGGGCTTCAGTGAA
GATATCCTGTAAGGCTTCTGGATACACGTTCACTGACTACTACATGAACTGGGTGAA
GCAGAGCCATGGAAAGAGCCTTGAGTGGATTGGAGATATTAATCCTCACAATGGTG
GTACTAGCTTCATCCAGAAGTTCAAGGGCAAGGCCACATTGACTGTAGACAAGTCCT
CCAGCACAGCCTACATGGAGCTCCGCAGCCTGACATCTGAGGACTCTGCAGTCTATT
ATTGTGCCCCTCTGGGACGAAAGGAGGGGTTTGCTTACTGGGGCCAAGGGACTCTG
GTCACTGTCTCTGCA (SEQ ID NO: 33)
6-B21 Light Chain Variable Region
GACACTGTGCTGACACAGTCTCCTGCTTCCTTAGTTGTATCTCTGGGGCAGAGGGCC
ACCATCTCATGCAGGGCCAGCAAAAGTGTCAGTACATCTGGCTATAGTTATATGCAC
TGGTACCAACAGAAACCAGGACAGCCACCCAAACTCCTCATCTATCTTGCATCCAAC
CTAGAATCTGGGGTCCCTGCCAGGTTCAGTGGCAGTGGGTCTGGGACAGCCTTCACC
CTCAACATCCATCCTGTGGAGGAGGAGGATGCTGCAACCTATTACTGTCAGCACAGT
AGGGAGCTTCCGTACACGTTCGGAGGGGGGACCAAGCTGGAAATAAAA (SEQ ID
NO: 34)
10-F23 Heavy Chain Variable Region
CAGGTTACTCTGAAAGAGTCTGGCCCTGGGATATTGCAGCCCTCCCAGACCCTCAGT
CTGACTTGTTCTTTCTCTGGGTTTTCACTGAGCACTTTTGCTATGGGTGTAGGCTGGA
TTCGTCAGCCTTCAGGGAAGGGTCTGGAGTGGCTGGCACACATTTGGTGGGATGATG
ATAAGTACTATAACCCAGCCCTGAAGAGCCGGCTCACAATCTCCAAGGATACCTCC
AAAAACCATGTATTCCTCAAGATCGCCAATGTGGACACTGCAGATACTGCCACATAC
TACTGTGCTCGAATGCCGCTAACTTTCTACTTTGACTACTGGGGCCAAGGCACCACT
CTCACAGTCTCCTCA (SEQ ID NO: 35)
10-F23 Light Chain Variable Region
GATGTTTTGCTGACCCAAACTCCACTCTCCCTGCCTGTCAGTCTTGGAGATCAAGCCT
CCATCTCTTGCAGATCTAGTCAGAGCATTGTACATAGTAATGGACACACCTATTTAG
AATGGTACCTGCAGAAACCAGGCCAGTCTCCAAAGCTCCTGATCTACAAAGTTTCCA
ACCGATTTTCTGGGGTCCCAGACAGGTTCAGTGGCAGTGGATCAGGGACAGATTTCA
CACTCAAGATCAGCAGAGTGGAGGCTGAGGATCTGGGAGTTTATTACTGCTTTCAAG
GTTCACATGTTCCGTTCACGTTCGGAGGGGGGACCAAGCTGGAAATAAAA (SEQ ID
NO: 36)
6-A12 Heavy Chain Variable Region
CAGGTTACTCTGAAAGAGTCTGGCCCTGGGATATTGCAGCCCTCCCAGACCCTCAGT
CTGACTTGTTCTTTCTCTGGGTTTTCACTGAGAACTTTTGCTATGGGTGTAGGCTGGA
TTCGTCAGCCTTCAGGGAAGGGTCTGGAGTGGCTGGCACACATTTGGTGGGATGATG
ATAAGTACTATAACCCAGCCCTGAAGAGCCGGCTCACAATCTCCAAGGATACCTCC
AAAAACCAGGTATTCCTCAAGATCGCCAATGTGGACACTGCAGATACTGCCACATA
CTACTGTGCTCGAATGCCGCTAACTTTCTACTTTGACTACTGGGGCCAAGGCACCAC
TCTCACAGTCTCCTCA (SEQ ID NO: 37)
6-A12 Light Chain Variable Region
GATGTTTTGATGACCCAAACTCCACTCTCCCTGCCTGTCAGTCTTGGAGATCAAGCCT
CCATCTCTTGTAGATCTAGTCAGAGCATTGTACATAGTAATGGAAACACCTATTTAG
AATGGTACCTGCAGAAACCAGGCCAGTCTCCAAAGCTCCTGATCTACAAAGTTTCCA
CCCGATTTTCTGGGGTCCCAGACAGGTTCAGTGGCAGTGGATCAGGGACAGATTTCA
CACTCAAGATCAGCAGAGTGGAGGCTGAGGATCTGGGAGTTTATTACTGCTTTCAAG
GTTCACATGTTCCGTTCACGTTCGGAGGGGGGACCAAGCTGGAAATAAAA (SEQ ID
NO: 38)
6-M8 Heavy Chain Variable Region
CAGGTCCAACTGCAGCAGCCTGGGGCTGAACTTGTGATGCCTGGGGCTTCAGTGAA
GCTGTCCTGCAAGGCTTCTGGCTACACCTTCACCAACTACTGGATGCACTGGGTGAA
ACAGAGGCCTGGACAAGGCCTTGAGTGGATCGGAGAGATTGATCCTTCTGATAGTT
ATACTAACTACAATCAAAAGTTCAAGGGCAAGGCCACATTGACTGTAGACAAATCC
TCCAGCACAGCCTACATGCAGCTCAGCAGCCTGACATCTGAGGACTCTGCGGTCTAT
TACTGTACAAGACAGGGTAGTACCTACGCGTGGGGTCAAGGAACCTCAGTCACCGT
CTCCTCA (SEQ ID NO: 39)
6-M8 Light Chain Variable Region
GATATTGTGATGACGCAGGCTGCATTCTCCAATCCAGTCACTCTTGGAACATCAGCT
TCCATCTCCTGCAGGTCTAGTAAGAGTCTCCTACATAGTAATGGCATCACTTATTTGT
ATTGGTATCTGCAGAAGCCAGGCCAGTCTCCTCAGCTCCTGATTTATCAGATGTCCA
ACCTTGCCTCAGGAGTCCCAGACAGGTTCAGTAGCAGTGGGTCAGGAACTGATTTCA
CACTGAGAATCAGCAGAGTGGAGGCTGAGGATGTGGGTGTTTATTACTGTGCTCAA
AATCTAGAACTTCCTCCGACGTTCGGTGGAGGCACCAAGCTGGAAATCAAA (SEQ ID
NO: 40)
2-A3 Heavy Chain Variable Region
GAGGTCCAGCTGCAACAATCTGGACCTGAGCTGGTGAAGCCTGGGGCTTCAGTGAA
GATGTCCTGTAAGGCTTCTGGATACACATTCACTGACTACTACATGATGTGGGTGAA
GCAGAGTCATGGAAAGAGCCTTGAGTGGATTGGAGATATTAATCCTTACAATGGTG
GTTCTAGCTACAACCCGAAGTTCAAGGGCAGGGCCACATTGACTGTAGACAAATCCT
CCAGCACAGCCTACATGCAGCTCAACAGCCTGACATCTGAGGACTCTGCAGTCTATT
ACTGTGCAAGAGGGACTTACTGGGGCCAAGGGACTCTGGTCACTGTCTCTGCA (SEQ
ID NO: 41)
2-A3 Light Chain Variable Region
GATGTTGTGATGACCCAGACTCCACTCACTTTGTCGGTTACCATTGGACAACCAGCC
TCCATCTCTTGCAAGTCAAGTCAGAGCCTCTTAGATAGTGCTGGAAAGACATATTTG
AATTGGTTGTTACAGAGGCCAGGCCAGTCTCCAAAGCGCCTAATGTATCTGGTGTCT
AAACTGGACTCTGGAGTCCCTGACAGGTTCACTGGCAGCGGATCAGGGACAGATTT
CACACTGAAAATCAGCAGAGTGGAGGCTGAGGATTTGGGAGTTTATTATTGCTGGC
AAGGTACACATTTTCCGTACACGTTCGGAGGGGGGACCAAGCTGGAAATAAAA
(SEQ ID NO: 42)
6-017 Heavy Chain Variable Region
CAGGTCCAACTGCAGCAGCCTGGGGCTGAGCTTGTGAAGCCTGGGGCTTCAGTGAA
GTTGTCCTGCAAGGCTTCTGGCTACACCTTCACCAGCTACTGGATGCACTGGATAAA
GCAGAGACCTGGACAAGGCCTTGAGTGGATTGGAGAGATTAACCCTAGCAATGGTG
GTTCTAACTACAATGAGAAGTTCAAGAGCAAGGCCACACTGACTGTAGACAAATCC
TCCAGCACAGCCTACATGCAACTCAGCAGCCTGACATCTGAGGACTCTGCGGTCTAT
CACTGTAAAAGCAGAGGCTACTGGGGCCAAGGCACCACTCTCACAGTCTCCTCA
(SEQ ID NO: 43)
6-017 Light Chain Variable Region
GATGTTGTGATGACCCAGACTCCACTCACTTTGTCGGTTACCATTGGACAACCAGCC
TCCATCTCTTGCAAGTCAAGTCAGAGCCTCTTAGATAGTTATGGAAAGACATATTTG
AATTGGTTGTTACAGCGGCCAGGCCAGTCTCCAAAGCGCCTAATCTATCTGGTGTCT
AAATTGGACTCTGGAGTCCCTGACAGGTTCACTGGCAGTGGATCAGGGACAGATTTC
ACACTGAAAATCAGCAGAGTGGAGGCTGAGGATTTGGGAATTTATTATTGCTGGCA
AGGTACACATTTTCCTCACACGTTCGGCTCGGGGACAAAGTTGGAAATAAAA (SEQ
ID NO: 44)
3-G5 Heavy Chain Variable Region
CAGGTTCAGCTGCAGCAGTCTGGAGCTGAGCTGGCGAGGCCTGGGGCTTCAGTGAA
ACTGTCCTGCAAGGCTTCTGGCTACACCTTCACAAGCTATGGTATAAGCTGGGTGAA
ACAGAGAACTGGACAGGGCCTTGAGTGGATTGGAGAGATTTTTCCTAGAAGTAGTA
ATACTTACTATAATGAGAAGTTCAAGGGCAAGGCCACACTGACTGCAGACAAGTCC
TCCAGCACAGTGTACATGGAGTTCCGCAGCCTGACATCTGAGGACTCTGCGGTCTAT
TTCTGTGCAAGAGAGGGGGGCCTGGCCTGGTTTGCTTACTGGGGCCAAGGGACTCTG
GTCACTGTCTCTGCA (SEQ ID NO: 45)
3-G5 Light Chain Variable Region
GATGTTGTGATGACCCAGACTCCACTCACTTTGTCGGTTACCATTGGACAACCAGCT
TCCATCTCTTGCAAGTCAAGTCAGAGCCTCTTATATACTAATGGAAACACCTATTTG
AATTGGTTATTACAGAGGCCAGGCCAGTCTCCAAAACGCCTAATCTATCTGGTGTCT
AAATTGGACTCTGGAATCCCTGACAGGTTCAGTGGCAGTGGATCAGGGACAGATTTC
ACACTGAGAATCAGCAGAGTGGAGGCTGAGGATTTGGGAGTTTATTACTGCTTGCA
GAGTACACATTTTCCATTCACGTTCGGCTCGGGGACAAAGTTGGAAATAAAA (SEQ
ID NO: 46)
6-A15 Heavy Chain Variable Region
GAGGTCCAGCTGCAACAATCTGGACCTGAGCTGGTGAAGCCTGGGGCTTCAGTGAA
GATGTCCTGTAAGGCTTCTGGATACACAATCACTGACTACTACATGATGTGGTTGAA
GCAGAGTCATGGAAAGAGCCTTGAATGGATTGGAGATATTAATCCTTACACTGGTG
GTACTAGCTACAACCAGAAGTTCAAGGGCAAGGCCACATTGACTGTAGACAAATCC
TCCAGCACAGCCTACCTGCAGCTCCACAGCCTGACATCTGAGGACTCTGCAGTCTAT
TACTGTGCAAGAGGGGCCTACTGGGGCCAAGGCACCACTCTCACAGTCTCCTCA
(SEQ ID NO: 47)
6-A15 Light Chain Variable Region
GATGTTGTGATGACCCAGACTCCACTCACTTTGTCGGTTACCATTGGACAACCAGCC
TCCATCTCTTGCAAGTCAAGTCAGAGCCTCTTAGATAGTGATGGAAAGACATATTTG
AATTGGTTGTTACAGAGGCCAGGCCAGTCTCCAAAGCGCCTAATCTATCTGGTGTCT
AAACTGGACTCTGGAGTCCCTGACAGGTTCACTGGCAGTGGATCAGGGACAGATTTC
ACACTGAAAATCAGCAGAGTGGAGGCTGAGGATTTGGGAGTTTATTATTGCTGGCA
AGGTACACATTTTCCGTACACGTTCGGAGGGGGGACCAAGCTGGAAATAAAA (SEQ
ID NO: 48)
10-K10 Heavy Chain Variable Region
GAGGTCCAGCTGCAACAATCTGGACCTGAGCTGGTGAAGCCTGGGGCTTCAGTGAA
GATGTCCTGTAAGGCTTCTGGATACACAATCACTGACTACTACATGATGTGGTTGAA
GCAGAGTCATGGAAAGAGCCTTGAATGGATTGGAGATATTAATCCTTACACTGGTG
GTACTAGCTACAACCAGAAGTTCAAGGGCAAGGCCACATTGACTGTAGACAAATCC
TCCAGCACAGCCTACATGCAGCTCAACAGCCTGACATCTGAGGACTCTGCAGTCTAT
TACTGTGCAAGAGGGGCCTACTGGGGCCAAGGCACCACTCTCACAGTCTCCTCA
(SEQ ID NO: 49)
10-K10 Light Chain Variable Region
GATGTTGTGATGACCCAGACTCCACTCACTTTGTCGGTTACCATTGGACAACCAGCC
TCCATCTCTTGCAAGTCAAGTCAGAGCCTCTTAGATAGTGATGGAAAGACATATTTG
AATTGGTTGTTACAGAGGCCAGGCCAGTCTCCAAAGCGCCTAATCTATCTGGTGTCT
AAACTGGACTCTGGAGTCCCTGACAGGTTCACTGGCAGTGGATCAGGGACAGATTTC
ACACTGAAAATCAGCAGAGTGGAGGCTGAGGATTTGGGAGTTTATTATTGCTGGCA
AGGTACACATTTTCCGTACACGTTCGGAGGGGGGACCAAGCTGGAAATAAAA (SEQ
ID NO: 50)
6-P20 Heavy Chain Variable Region
GAGGTCCAGCTGCAACAATCTGGACCTGAGCTGGTGAAGCCTGGGGCTTCAGTGAA
GATATCCTGTAAGGCTTCTGGATACACGTTCACTGACTACTACATGAACTGGGTGAA
GCAGAGCCATGGCAGGAGCCTTGAGTTGATTGGAGATATTAATCCTAACAATGGTG
GTTCTAACTTCAACCAGAAGTTCAGGGGCAAGGCCACATTGACTGTAGACAAGTCCT
CCAGCACAGCCTATATGGAGCTCCGCAGCCTGACATCTGAGGACTCTGCAATCTATT
ACTGTGCAAGAATGGGTTACTGGGGCCAAGGGACTCTGGTCACTGTCTCTGCA (SEQ
ID NO: 51)
6-P20 Light Chain Variable Region
GATGTTGTGATGACCCAGACTCCACTCACTTTGTCGGTTACCATTGGACAACCAGCC
TCCATCTCTTGCAAGTCAAGTCAGAGCCTCTTACATAGTGATGGAAAGACATATTTG
AATTGGATGTTCCAGAGGCCAGGCCAGTCTCCAAAGCGCCTAATCTATCTGGTGTCT
AAACTGGACTCTGGAGTCCCTTACAGGTTCACTGGCGGTGGATCAGGGACAGATTTC
ACACTGCAAATCAGCAGAGTGGAGACTGAGGATTTGGGAGTTTATTATTGCTGGCA
AGGTACACATTTTCCTCGGACGTTCGGTGGAGGCACCAAGCTGGAAATCAAA (SEQ
ID NO: 52)
7-08 Heavy Chain Variable Region
GAGGTCCAGCTGCAGCAGTCTGGACCTGAACTGGTCAAGCCTGGGGCTTCAGTGAA
GATGTCCTGCAAGGCTTCTGGATACACATTCACTGACTACTACATACACTGGGTGAA
GCAGAAGCCTGGGCAGGGCCTTGAGTACATTGGAGAGATTTATCCTGGAAGTGGTA
ATACTTACTACAATGGGAAGTTCAGGGGCAAGGCCACACTGACTGCAGACAAGTCC
TCCAGCACAGCCTACATGCAGCTCAGCAGCCTGACATCTGAGGACTCTGCAGTCTAT
TTCTGTGGTAGTGGCTACTTTGACTACTGGGGCCAAGGCACCACTCTCACAGTCTCC
TCA (SEQ ID NO: 53)
7-08 Light Chain Variable Region
GATGTTGTGATGACCCAGACTCCACTCACTTTGTCTGTTACCATTGGACAGCCAGCTT
CCATTTCTTGCAAGTCAAGTCAGAGCCTCTTATATAGTAATGGAAAAACCTATTTGA
ATTGGTTATTACAGAGTCCAGGCCAGTCTCCAAAGCTCCTAATCTATCTGGTGTCTA
AACTGGAATCTGGAGTCCCTGACAGATTCAGTGGCAGTGGATCAGGGACAGATTTT
ACACTGAAACTCAGCAGAGTGGAGGCTGAGGATTTGGGAGTATATTACTGCGTGCA
AGGTACACATTTCCCATTCACGTTCGGCTCGGGGACAAAGTTGGAAATAAAA (SEQ
ID NO: 54)
Rabbit mAb sequences
A11B1_16G7 Heavy Chain
ATGGAGACTGGGCTGCGCTGGCTTCTCCTGGTCGCTGTGTTCAAAGGTGTCCAGTGT
CAGGAGCAACTGGTGGAGTCCGGGGGAGACCTGGTCAAGCCTGGGGCATCCCTGAC
ACTCACCTGCACAGCCTCTGGATTCTCCTTCAATAAGAATTATTGGATGTGCTGGGT
CCGCCAGGCTCCAGGGAAGGGGCTGGAGTGGATCGGATGCATTTATAATGGTGATG
GCAACACATACTACGCGAGCTGGGTGAATGGCCGATTCACCATCTCCAAAACCTCGT
CGACCACGGTGACTCTGCAAATGACCAGTCTGACAGTCGCGGACACGGCCATCTATT
TCTGTGCGAGACTACTTAATATGTGGGGCCCAGGCACCCTGGTCACCGTCTCCTCAG
GGCAACCTAAGGCTCCATCAGTCTTCCCACTGGCCCCCTGCTGCGGGGACACACCCA
GCTCCACGGTGACCCTGGGCTGCCTGGTCAAAGGCTACCTCCCGGAGCCAGTGACC
GTGACCTGGAACTCGGGCACCCTCACCAATGGGGTACGCACCTTCCCGTCCGTCCGG
CAGTCCTCAGGCCTCTACTCGCTGAGCAGCGTGGTGAGCGTGACCTCAAGCAGCCA
GCCCGTCACCTGCAACGTGGCCCACCCAGCCACCAACACCAAAGTGGACAAGACCG
TTGCGCCCTCGACATGCAGCAAGCCCATGTGCCCACCCCCTGAACTCCCGGGGGGAC
CGTCTGTCTTCATCTTCCCCCCAAAACCCAAGGACACCCTCATGATCTCACGCACCC
CCGAGGTCACATGCGTGGTGGTGGACGTGAGCCAGGATGACCCCGAGGTGCAGTTC
ACATGGTACATAAACAACGAGCAGGTGCGCACCGCCCGGCCGCCGCTACGGGAGCA
GCAGTTCAACAGCACGATCCGCGTGGTCAGCACCCTCCCCATCGCGCACCAGGACT
GGCTGAGGGGCAAGGAGTTCAAGTGCAAAGTCCACAACAAGGCACTCCCGGCCCCC
ATCGAGAAAACCATCTCCAAAGCCAGAGGGCAGCCCCTGGAGCCGAAGGTCTACAC
CATGGGCCCTCCCCGGGAGGAGCTGAGCAGCAGGTCGGTCAGCCTGACCTGCATGA
TCAACGGCTTCTACCCTTCCGACATCTCGGTGGAGTGGGAGAAGAACGGGAAGGCA
GAGGACAACTACAAGACCACGCCGACCGTGCTGGACAGCGACGGCTCCTACTTCCT
CTACAGCAAGCTCTCAGTGCCCACGAGTGAGTGGCAGCGGGGCGACGTCTTCACCT
GCTCCGTGATGCACGAGGCCTTGCACAACCACTACACGCAGAAGTCCATCTCCCGCT
CTCCGG GTAAATAG (SEQ ID NO: 55)
A11B1_16G7 Light Chain
ATGGACACGAGGGCCCCCACTCAGCTGCTGGGGCTCCTACTGCTCTGGCTCCCAGGT
GCCAGATGTGCTGACATTGTGATGACCCAGACTCCAGCCTCCGTGGAGGCAGCTGTG
GGAGGCACAGTCACCATCAAGTGCCAGGCCAGTGAGAGCATTGGCAATGCATTAGC
CTGGTATCAGCAGAAACCAGGGCAGCCTCCCAAGCTCCTGATCTATACTGCAGCCAC
TCTGGCATCTGGGGTCCCATCGCGGTTCAGCGGCAGTGGATCTGGGACAGAGTTCAC
TCTCACCATCAGTGGCGTGCAGTGTGACGATGCTGCCACTTACTACTGTCAAAGCTA
TTATTTTACTAGTGTTAGTAGTTATGGCAATGCTTTCGGCGGAGGGACCGAGGTGGT
GGTCAAAGGTGATCCAGTTGCACCTACTGTCCTCATCTTCCCACCAGCTGCTGATCA
GGTGGCAACTGGAACAGTCACCATCGTGTGTGTGGCGAATAAATACTTTCCCGATGT
CACCGTCACCTGGGAGGTGGATGGCACCACCCAAACAACTGGCATCGAGAACAGTA
AAACACCGCAGAATTCTGCAGATTGTACCTACAACCTCAGCAGCACTCTGACACTGA
CCAGCACACAGTACAACAGCCACAAAGAGTACACCTGCAAGGTGACCCAGGGCACG
ACCTCAGTCGTCCAGAGCTTCAATAGGGGTGACTGTTAG (SEQ ID NO: 56)
A11B1_16E10 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCAGAGTCTCTGGATTCTCCTTCAGTAGCAGTTATTATATGTGTTGGGTCCGC
CAGGCTCCAGGGAAGGGGCTGGAATGGATCGCATGTATTGGTACTACTCGTGGTAG
CACTTACTACGCGACCTGGGCGAAAGGCCGATTCACCATTTCTAAAATCTCGTCGAC
CACGGTGACTCTACAAATGACCAGTCTGACAGACGCGGACACGGCCACCTATTTCTG
TGCGAGAGATGCTACTGGTTATAGGATTAACACGATTGGCCTCTATTTTAATTTGTG
GGGCCCAGGCACCCTGGTCACCGTCTCCTCAGGGCAACCTAAGGCTCCATCAGTCTT
CCCACTGGCCCCCTGCTGCGGGGACACACCCAGCTCCACGGTGACCCTGGGCTGCCT
GGTCAAAGGCTACCTCCCGGAGCCAGTGACCGTGACCTGGAACTCGGGCACCCTCA
CCAATGGGGTACGCACCTTCCCGTCCGTCCGGCAGTCCTCAGGCCTCTACTCGCTGA
GCAGCGTGGTGAGCGTGACCTCAAGCAGCCAGCCCGTCACCTGCAACGTGGCCCAC
CCAGCCACCAACACCAAAGTGGACAAGACCGTTGCGCCCTCGACATGCAGCAAGCC
CATGTGCCCACCCCCTGAACTCCCGGGGGGACCGTCTGTCTTCATCTTCCCCCCAAA
ACCCAAGGACACCCTCATGATCTCACGCACCCCCGAGGTCACATGCGTGGTGGTGG
ACGTGAGCCAGGATGACCCCGAGGTGCAGTTCACATGGTACATAAACAACGAGCAG
GTGCGCACCGCCCGGCCGCCGCTACGGGAGCAGCAGTTCAACAGCACGATCCGCGT
GGTCAGCACCCTCCCCATCGCGCACCAGGACTGGCTGAGGGGCAAGGAGTTCAAGT
GCAAAGTCCACAACAAGGCACTCCCGGCCCCCATCGAGAAAACCATCTCCAAAGCC
AGAGGGCAGCCCCTGGAGCCGAAGGTCTACACCATGGGCCCTCCCCGGGAGGAGCT
GAGCAGCAGGTCGGTCAGCCTGACCTGCATGATCAACGGCTTCTACCCTTCCGACAT
CTCGGTGGAGTGGGAGAAGAACGGGAAGGCAGAGGACAACTACAAGACCACGCCG
ACCGTGCTGGACAGCGACGGCTCCTACTTCCTCTACAGCAAGCTCTCAGTGCCCACG
AGTGAGTGGCAGCGGGGCGACGTCTTCACCTGCTCCGTGATGCACGAGGCCTTGCA
CAACCACTACACGCAGAAGTCCATCTCCCGCTCTCCGGGTAAATAG (SEQ ID NO:
57)
A11B1_16E10 Light Chain
ATGGACACGAGGGCCCCCACTCAGCTGCTGGGGCTCCTGCTGCTCTGGCTCCCAGGT
GCCAGATGTGCGTTCGAATTGACCCAGACTCCATCCTCCGTGGAGGCTGCTGTGGGA
GGCACGCCCACCATCAAGTGCCAGGCCAGTCAGACCATTTACAGTTACTTATCCTGG
TATCAGCAGAAACCAGGGCAGCCTCCCAAGCTCCTGATCTATGAAGCGTCCAAACT
GGCCTCTGGGGTCCCATCGCGGTTCAGCGGCAGTGGATCTGGGACAGACTACACTCT
CACCATCAGCGACCTGGAGTGTGCCGATGCTGCCACTTACTACTGTCAAAGCTATCA
TGGTACTGCTAGTACTGAATATAATACTTTCGGCGGGGGGACCGAGGTGGTGGTCAG
AGGTGATCCAGTTGCACCTACTGTCCTCATCTTCCCACCAGCTGCTGATCAGGTGGC
AACTGGAACAGTCACCATCGTGTGTGTGGCGAATAAATACTTTCCCGATGTCACCGT
CACCTGGGAGGTGGATGGCACCACCCAAACAACTGGCATCGAGAACAGTAAAACAC
CGCAGAATTCTGCAGATTGTACCTACAACCTCAGCAGCACTCTGACACTGACCAGCA
CACAGTACAACAGCCACAAAGAGTACACCTGCAAGGTGACCCAGGGCACGACCTCA
GTCGTCCAGAGCTTCAATAGGGGTGACTGTTAG (SEQ ID NO: 58)
A11B1_15G10 Heavy Chain
ATGGAGACTGGGCTGCGCTGGCTTCTCCTGGTCGCTGTGCTCAAAGGTGTCCAGTGT
CAGCAGCAGCTGGTGGAGTCCGGGGGAGGCCTGGTCAAGCCTGGGGCAGCCCTGAC
ATTCACCTGCACAGCCTCTGGATTCTCCTTCAGTGGCAATTATTGGATATGCTGGGTC
CGCCAGGCTCCAGGGAAGGGGTTGGAGTGGATCGCGTGCATTGGTACTATTACTAGT
AGGACATACTACGCGAGCTGGGCGAAAGGCCGATTCACCATTTCCAAAACCTCGTC
GACCACGGTGACTCTGCAAATGACCAGTCTGACAGCCGCGGACACGGCCACGTATT
TCTGTGCGAGAGGTGCGGTTGTTAGTAGTGGTAATGCTCCCTACTACTTTACCTTGTG
GGGCCCAGGCACCCTGGTCACCGTCTCCTCAGGGCAACCTAAGGCTCCATCAGTCTT
CCCACTGGCCCCCTGCTGCGGGGACACACCCAGCTCCACGGTGACCCTGGGCTGCCT
GGTCAAAGGCTACCTCCCGGAGCCAGTGACCGTGACCTGGAACTCGGGCACCCTCA
CCAATGGGGTACGCACCTTCCCGTCCGTCCGGCAGTCCTCAGGCCTCTACTCGCTGA
GCAGCGTGGTGAGCGTGACCTCAAGCAGCCAGCCCGTCACCTGCAACGTGGCCCAC
CCAGCCACCAACACCAAAGTGGACAAGACCGTTGCGCCCTCGACATGCAGCAAGCC
CATGTGCCCACCCCCTGAACTCCCGGGGGGACCGTCTGTCTTCATCTTCCCCCCAAA
ACCCAAGGACACCCTCATGATCTCACGCACCCCCGAGGTCACATGCGTGGTGGTGG
ACGTGAGCCAGGATGACCCCGAGGTGCAGTTCACATGGTACATAAACAACGAGCAG
GTGCGCACCGCCCGGCCGCCGCTACGGGAGCAGCAGTTCAACAGCACGATCCGCGT
GGTCAGCACCCTCCCCATCGCGCACCAGGACTGGCTGAGGGGCAAGGAGTTCAAGT
GCAAAGTCCACAACAAGGCACTCCCGGCCCCCATCGAGAAAACCATCTCCAAAGCC
AGAGGGCAGCCCCTGGAGCCGAAGGTCTACACCATGGGCCCTCCCCGGGAGGAGCT
GAGCAGCAGGTCGGTCAGCCTGACCTGCATGATCAACGGCTTCTACCCTTCCGACAT
CTCGGTGGAGTGGGAGAAGAACGGGAAGGCAGAGGACAACTACAAGACCACGCCG
ACCGTGCTGGACAGCGACGGCTCCTACTTCCTCTACAGCAAGCTCTCAGTGCCCACG
AGTGAGTGGCAGCGGGGCGACGTCTTCACCTGCTCCGTGATGCACGAGGCCTTGCA
CAACCACTACACGCAGAAGTCCATCTCCCGCTCTCCGGGTAAATAG (SEQ ID NO:
59)
A11B1_15G10 Light Chain
ATGGACACGAGGGCCCCCACTCAGCTGCTGGGGCTCCTGCTGCTCTGGCTCCCAGGT
GCCAGATGTGCATTCGAATTGACGCAGACTCCATCCTCCGTGGAGGCAGCTGTGGGA
GGCACAGTCACCATCAAGTGCCAGGCCAGTCAGAGCATTAGTAGTTACTTATCCTGG
TATCAGCAGAAACCAGGGCAGCCTCCCAAGCTCCTGATCTACAGGGCATCCACTCTG
GAATCTGGGGTCCCATCGCGGTTTAAAGGCAGTGGATCTGGGACAGAGTTCACTCTC
ACCATCAGCGACCTGGAGTGTGCCGATGCTGCCACTTACTTCTGTCAAAGCTATTAT
GGTGTTACTTTTAGTGGTTTTGCTTTCGGCGGAGGGACCGAGGTGGTGGTCAAAGGT
GATCCAGTTGCACCTACTGTCCTCATCTTCCCACCAGCTGCTGATCAGGTGGCAACT
GGAACAGTCACCATCGTGTGTGTGGCGAATAAATACTTTCCCGATGTCACCGTCACC
TGGGAGGTGGATGGCACCACCCAAACAACTGGCATCGAGAACAGTAAAACACCGCA
GAATTCTGCAGATTGTACCTACAACCTCAGCAGCACTCTGACACTGACCAGCACACA
GTACAACAGCCACAAAGAGTACACCTGCAAGGTGACCCAGGGCACGACCTCAGTCG
TCCAGAGCTTCAATAGGGGTGACTGTTAG (SEQ ID NO: 60)
A11B1_14H1 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCAAAGCCTCTGGAATCGACTTCAATAACTATTGGATAACCTGGGTCCGCCA
GGCTCCAGGGAAGGGGCTGGAGTGGATCGCATGCATTTATGTTGGAATTACCGGCC
GCACATGGTACGCGAACTGGGCGAAAGGCCGATTCACCATCTCCAAGGCCTCGAGC
ACGGTGGATCTGAAAATGACCAGTCTGACAGCCGCGGACACGGCCACCTATTTCTGT
GCGAGGAATGGTGATGGTGGTATTTATGCTCTTAACTTGTGGGGCCCAGGCACCCTG
GTCACCGTCTCCTCAGGGCAACCTAAGGCTCCATCAGTCTTCCCACTGGCCCCCTGC
TGCGGGGACACACCCAGCTCCACGGTGACCCTGGGCTGCCTGGTCAAAGGCTACCT
CCCGGAGCCAGTGACCGTGACCTGGAACTCGGGCACCCTCACCAATGGGGTACGCA
CCTTCCCGTCCGTCCGGCAGTCCTCAGGCCTCTACTCGCTGAGCAGCGTGGTGAGCG
TGACCTCAAGCAGCCAGCCCGTCACCTGCAACGTGGCCCACCCAGCCACCAACACC
AAAGTGGACAAGACCGTTGCGCCCTCGACATGCAGCAAGCCCATGTGCCCACCCCC
TGAACTCCCGGGGGGACCGTCTGTCTTCATCTTCCCCCCAAAACCCAAGGACACCCT
CATGATCTCACGCACCCCCGAGGTCACATGCGTGGTGGTGGACGTGAGCCAGGATG
ACCCCGAGGTGCAGTTCACATGGTACATAAACAACGAGCAGGTGCGCACCGCCCGG
CCGCCGCTACGGGAGCAGCAGTTCAACAGCACGATCCGCGTGGTCAGCACCCTCCC
CATCGCGCACCAGGACTGGCTGAGGGGCAAGGAGTTCAAGTGCAAAGTCCACAACA
AGGCACTCCCGGCCCCCATCGAGAAAACCATCTCCAAAGCCAGAGGGCAGCCCCTG
GAGCCGAAGGTCTACACCATGGGCCCTCCCCGGGAGGAGCTGAGCAGCAGGTCGGT
CAGCCTGACCTGCATGATCAACGGCTTCTACCCTTCCGACATCTCGGTGGAGTGGGA
GAAGAACGGGAAGGCAGAGGACAACTACAAGACCACGCCGACCGTGCTGGACAGC
GACGGCTCCTACTTCCTCTACAGCAAGCTCTCAGTGCCCACGAGTGAGTGGCAGCGG
GGCGACGTCTTCACCTGCTCCGTGATGCACGAGGCCTTGCACAACCACTACACGCAG
AAGTCCATCTCCCGCTCTCCGGGTAAATAG (SEQ ID NO: 61)
A11B1_14H1 Light Chain
ATGGACACGAGGGCCCCCACTCAGCTGCTGGGGCTCCTGCTGCTCTGGCTCCCAGGT
GCCACATTTGCACAAGTGCTGACCCAGACTGCATCGTCCGTGTCTGCAGCTGTGGGA
GGCACAGTCACCATCAGTTGCCAGTCCAGTCAGAGTGTTTATAATAATAATTGGTTA
GCCTGGTATCAGCAGAAACCAGGGCAGCCTCCCAAGCTCCTGATCTACAGGGCATC
CACTCTGACATCTGGGGTCCCATCGCGGTTCAAAGGCAGTGGATCTGGGACACAGTT
CACTCTCACCATCAGCGACCTGGAGTGTGACGATGCTGCCACTTACTACTGTGCAGG
CGGTTATAGTGGTAATATTTACGTAAATGATTTCGGCGGAGGGACCGAGGTGGTGGT
CAAAGGTGATCCAGTTGCACCTACTGTCCTCATCTTCCCACCAGCTGCTGATCAGGT
GGCAACTGGAACAGTCACCATCGTGTGTGTGGCGAATAAATACTTTCCCGATGTCAC
CGTCACCTGGGAGGTGGATGGCACCACCCAAACAACTGGCATCGAGAACAGTAAAA
CACCGCAGAATTCTGCAGATTGTACCTACAACCTCAGCAGCACTCTGACACTGACCA
GCACACAGTACAACAGCCACAAAGAGTACACCTGCAAGGTGACCCAGGGCACGACC
TCAGTCGTCCAGAGCTTCAATAGGGGTGACTGTTAG (SEQ ID NO: 62)
A11B1_13G4 Heavy Chain
ATGGAGACTGGGCTGCGCTGGCTTCTCCTGGTCGCTGTGCTCAAAGGTGTCCAGTGT
CAGGAGCAGCTGGAGGAGTCCGGGGGAGACCTGGTCAAGCCTGGGGGATCCCTGAC
ACTCACCTGCAAAGCCTCTGGATTCTCCTTCAGTAATACCTACTGGGCATGCTGGGT
CCGCCAGGCTCCAGGGAAGGGGCTGGAGTGGATCGCATGCATGAATCCTGCTAGTA
GTGGTAGCTCTTACTACGCGAGCTGGGCGAAAGGCCGATTCACCATCTCCAAAACCT
CGTCGACCACGGTGACTCTGCACATGCCCAGTCTGACAGCCGCGGACACGGCCACC
TATTTCTGTGCGAAATGGGATACTGCTTTCGATGTGTGGGGCCCAGGCACCCTGGTC
ACCGTCTCCTCAGGGCAACCTAAGGCTCCATCAGTCTTCCCACTGGCCCCCTGCTGC
GGGGACACACCCAGCTCCACGGTGACCCTGGGCTGCCTGGTCAAAGGCTACCTCCC
GGAGCCAGTGACCGTGACCTGGAACTCGGGCACCCTCACCAATGGGGTACGCACCT
TCCCGTCCGTCCGGCAGTCCTCAGGCCTCTACTCGCTGAGCAGCGTGGTGAGCGTGA
CCTCAAGCAGCCAGCCCGTCACCTGCAACGTGGCCCACCCAGCCACCAACACCAAA
GTGGACAAGACCGTTGCGCCCTCGACATGCAGCAAGCCCATGTGCCCACCCCCTGA
ACTCCCGGGGGGACCGTCTGTCTTCATCTTCCCCCCAAAACCCAAGGACACCCTCAT
GATCTCACGCACCCCCGAGGTCACATGCGTGGTGGTGGACGTGAGCCAGGATGACC
CCGAGGTGCAGTTCACATGGTACATAAACAACGAGCAGGTGCGCACCGCCCGGCCG
CCGCTACGGGAGCAGCAGTTCAACAGCACGATCCGCGTGGTCAGCACCCTCCCCAT
CGCGCACCAGGACTGGCTGAGGGGCAAGGAGTTCAAGTGCAAAGTCCACAACAAG
GCACTCCCGGCCCCCATCGAGAAAACCATCTCCAAAGCCAGAGGGCAGCCCCTGGA
GCCGAAGGTCTACACCATGGGCCCTCCCCGGGAGGAGCTGAGCAGCAGGTCGGTCA
GCCTGACCTGCATGATCAACGGCTTCTACCCTTCCGACATCTCGGTGGAGTGGGAGA
AGAACGGGAAGGCAGAGGACAACTACAAGACCACGCCGACCGTGCTGGACAGCGA
CGGCTCCTACTTCCTCTACAGCAAGCTCTCAGTGCCCACGAGTGAGTGGCAGCGGGG
CGACGTCTTCACCTGCTCCGTGATGCACGAGGCCTTGCACAACCACTACACGCAGAA
GTCCATCTCCCGCTCTCCGGGTAAATAG (SEQ ID NO: 63)
A11B1_13G4 Light Chain
ATGGACACGAGGGCCCCCACTCAGCTGCTGGGGCTCCTGCTGCTCTGGCTCCCAGGT
GCCAGATGTGCCGATGTTGTGATGACCCAGACTCCATCCTCCGTGGAGGCAGCTGTG
GGAGGCACAGTCACCATCAAGTGCCAGGCCAGTCAGAGCATTAGTAGCTACTTAGC
CTGGTATCAGCAGAAACCAGGGCAGCCTCCCAAGCTCCTGATCTATGGTGCATCCAA
TCTGGAGTCTGGGGTCCCATCGCGGTTCAAAGGCAGTGGATCTGGGACAGAGTACA
CTCTCACCATCAGCGGCGTGCAGTGTGACGATGCTGCCACTTACTACTGTCAAAACT
ATTATGCTATTGATACTTATGGTCATGCTTTCGGCGGAGGGACCGAGGTGGTGGTCA
AAGGTGATCCAGTTGCACCTACTGTCCTCATCTTCCCACCAGCTGCTGATCAGGTGG
CAACTGGAACAGTCACCATCGTGTGTGTGGCGAATAAATACTTTCCCGATGTCACCG
TCACCTGGGAGGTGGATGGCACCACCCAAACAACTGGCATCGAGAACAGTAAAACA
CCGCAGAATTCTGCAGATTGTACCTACAACCTCAGCAGCACTCTGACACTGACCAGC
ACACAGTACAACAGCCACAAAGAGTACACCTGCAAGGTGACCCAGGGCACGACCTC
AGTCGTCCAGAGCTTCAATAGGGGTGACTGTTAG (SEQ ID NO: 64)
A11B1_13C3 Heavy Chain
ATGGAGACTGGGCTGCGCTGGCTTCTCCTGGTCGCTGTGCTCAAAGGTGTCCAGTGT
CAGGAGCAGCTGGAGGAGTCCGGGGGAGACCTGGTCAAGCCTGGGGCATCCCTGAC
ACTCACCTGCACAGCCTCTGGATTCTCCTTTAGTAGCAACTATCACATCTGCTGGGTC
CGCCAGGCTCCAGGAAAGGGGCTGGAGTTGATCGCATGCATTTATGTTGGTGATGGC
AGCACATACTACGCGAGCTGGGCGAAAGGCCGATTCACCATCTCCAAATCCTCGTC
GACCACGGTAGCTCTGCAAATGACCAGTCTGACAGCCGCGGACACGGCCACCTATT
TCTGTGGGAGAATGTTTAACTTGTGGGGCCCAGGCACCCTGGTCACCGTCTCCTCAG
GGCAACCTAAGGCTCCATCAGTCTTCCCACTGGCCCCCTGCTGCGGGGACACACCCA
GCTCCACGGTGACCCTGGGCTGCCTGGTCAAAGGCTACCTCCCGGAGCCAGTGACC
GTGACCTGGAACTCGGGCACCCTCACCAATGGGGTACGCACCTTCCCGTCCGTCCGG
CAGTCCTCAGGCCTCTACTCGCTGAGCAGCGTGGTGAGCGTGACCTCAAGCAGCCA
GCCCGTCACCTGCAACGTGGCCCACCCAGCCACCAACACCAAAGTGGACAAGACCG
TTGCGCCCTCGACATGCAGCAAGCCCATGTGCCCACCCCCTGAACTCCCGGGGGGAC
CGTCTGTCTTCATCTTCCCCCCAAAACCCAAGGACACCCTCATGATCTCACGCACCC
CCGAGGTCACATGCGTGGTGGTGGACGTGAGCCAGGATGACCCCGAGGTGCAGTTC
ACATGGTACATAAACAACGAGCAGGTGCGCACCGCCCGGCCGCCGCTACGGGAGCA
GCAGTTCAACAGCACGATCCGCGTGGTCAGCACCCTCCCCATCGCGCACCAGGACT
GGCTGAGGGGCAAGGAGTTCAAGTGCAAAGTCCACAACAAGGCACTCCCGGCCCCC
ATCGAGAAAACCATCTCCAAAGCCAGAGGGCAGCCCCTGGAGCCGAAGGTCTACAC
CATGGGCCCTCCCCGGGAGGAGCTGAGCAGCAGGTCGGTCAGCCTGACCTGCATGA
TCAACGGCTTCTACCCTTCCGACATCTCGGTGGAGTGGGAGAAGAACGGGAAGGCA
GAGGACAACTACAAGACCACGCCGACCGTGCTGGACAGCGACGGCTCCTACTTCCT
CTACAGCAAGCTCTCAGTGCCCACGAGTGAGTGGCAGCGGGGCGACGTCTTCACCT
GCTCCGTGATGCACGAGGCCTTGCACAACCACTACACGCAGAAGTCCATCTCCCGCT
CTCCGGGTAAATAG (SEQ ID NO: 65)
A11B1_13C3 Light Chain
ATGGACACGAGGGCCCCCACTCAGCTGCTGGGGCTCCTGCTGCTCTGGCTCCCAGGT
GCCATATGTGACCCTGTGCTGACCCAGACTCCATCCTCCGTGTCTGCGGCTGTGGGA
GTCACAGTCACCATCAACTGCCAGTCCAGTCCGAGTGTTTATAGTAACTACTTATCC
TGGTATCAGCAGAAACCAGGGCAGCCTCCCAAGCTCCTCATCTATCTGGCATCTACT
CTGGCATCTGGGGTCCCATCGCGGTTCAAAGGCAGTGGATCTGGGACACAGTTCACT
CTCACCATCAGCGACGTGCAGTGTGACGATGCTGCCACTTACTACTGTGCAGGCACT
TATAGTGGTAATATTTGGTCTTTCGGCGGAGGGACCGAGGTGGTGGTCAAAGGTGAT
CCAGTTGCACCTACTGTCCTCATCTTCCCACCAGCTGCTGATCAGGTGGCAACTGGA
ACAGTCACCATCGTGTGTGTGGCGAATAAATACTTTCCCGATGTCACCGTCACCTGG
GAGGTGGATGGCACCACCCAAACAACTGGCATCGAGAACAGTAAAACACCGCAGA
ATTCTGCAGATTGTACCTACAACCTCAGCAGCACTCTGACACTGACCAGCACACAGT
ACAACAGCCACAAAGAGTACACCTGCAAGGTGACCCAGGGCACGACCTCAGTCGTC
CAGAGCTTCAATAGGGGTGACTGTTAG (SEQ ID NO: 66)
A11B1_12F2 Heavy Chain
ATGGAGACTGGGCTGCGCTGGCTTCTCCTGGTCGCTGTGCTCAAAGGTGTCCAGTGT
CAGCAGCAGCTGGTGGAGTCCGGGGGAGGCCTGGTCAAGCCTGGGGCATCCCTGAC
ACTCACCTGCACAGCCTCTGGATTCTCCTTCAGTAGCGGCTATCACATGTGCTGGGT
CCGCCAGGCTCCAGGGAAGGGGCTGGAGTGGATCGCATGCTTTGGTGTTTATACTGG
TACCACTACCTACGCGAGCTGGGCGAAAGGTCGATTCACCATCTCCAAAACCTCGTC
GACCACGGTGACTCTACAAATGACCAGTCTAACAGTCGCGGACACGGCCACCTATTT
CTGTGCGAGAATCAGTGCTGAAAATGGTGGGGACTTGTGGGGCCCAGGCACCCTGG
TCACCGTCTCCTCAGGGCAACCTAAGGCTCCATCAGTCTTCCCACTGGCCCCCTGCT
GCGGGGACACACCCAGCTCCACGGTGACCCTGGGCTGCCTGGTCAAAGGCTACCTC
CCGGAGCCAGTGACCGTGACCTGGAACTCGGGCACCCTCACCAATGGGGTACGCAC
CTTCCCGTCCGTCCGGCAGTCCTCAGGCCTCTACTCGCTGAGCAGCGTGGTGAGCGT
GACCTCAAGCAGCCAGCCCGTCACCTGCAACGTGGCCCACCCAGCCACCAACACCA
AAGTGGACAAGACCGTTGCGCCCTCGACATGCAGCAAGCCCATGTGCCCACCCCCT
GAACTCCCGGGGGGACCGTCTGTCTTCATCTTCCCCCCAAAACCCAAGGACACCCTC
ATGATCTCACGCACCCCCGAGGTCACATGCGTGGTGGTGGACGTGAGCCAGGATGA
CCCCGAGGTGCAGTTCACATGGTACATAAACAACGAGCAGGTGCGCACCGCCCGGC
CGCCGCTACGGGAGCAGCAGTTCAACAGCACGATCCGCGTGGTCAGCACCCTCCCC
ATCGCGCACCAGGACTGGCTGAGGGGCAAGGAGTTCAAGTGCAAAGTCCACAACAA
GGCACTCCCGGCCCCCATCGAGAAAACCATCTCCAAAGCCAGAGGGCAGCCCCTGG
AGCCGAAGGTCTACACCATGGGCCCTCCCCGGGAGGAGCTGAGCAGCAGGTCGGTC
AGCCTGACCTGCATGATCAACGGCTTCTACCCTTCCGACATCTCGGTGGAGTGGGAG
AAGAACGGGAAGGCAGAGGACAACTACAAGACCACGCCGACCGTGCTGGACAGCG
ACGGCTCCTACTTCCTCTACAGCAAGCTCTCAGTGCCCACGAGTGAGTGGCAGCGGG
GCGACGTCTTCACCTGCTCCGTGATGCACGAGGCCTTGCACAACCACTACACGCAGA
AGTCCATCTCCCGCTCTCCGGGTAAATAG (SEQ ID NO: 67)
A11B1_12F2 Light Chain
ATGGACACGAGGGCCCCCACTCAGCTGCTGGGGCTCCTGCTGCTCTGGCTCCCAGGT
GCCAGATGTGATGTTGTGATGACCCAGACTCCAGCCTCCGTGGAGGCAGCTGTGGG
AGGCACAGTCACCATCAAGTGCCAGGCCAGTCAGAGCATTAGCAACTACTTTTCTTG
GTATCAGCAGAAACCAGGGCAGCCTCCCAAGCTCCTGATCTACAGGGCGTCCACTCT
GGCATCTGGGGTCCCATCGCGGTTCAGCGGCAGTGGATCTGGGACAGAGTTCACTCT
CACCATCAGCGACCTGGAGTGTGCCGATTCTGCCACTTACTACTGTCAGTGCACTTA
TGGTAGTAGTAGTACTGGTTTTGGTTTCGGCGGAGGGACCGAGGTGGTGGTCAAAG
GTGATCCAGTTGCACCTACTGTCCCCATCTTCCCACCAGCTGCTGATCAGGTGGCAA
CTGGAACAGTCACCATCGTGTGTGTGGCGAATAAATACTTTCCCGATGTCACCGTCA
CCTGGGAGGTGGATGGCACCACCCAAACAACTGGCATCGAGAACAGTAAAACACCG
CAGAATTCTGCAGATTGTACCTACAACCTCAGCAGCACTCTGACACTGACCAGCACA
CAGTACAACAGCCACAAAGAGTACACCTGCAAGGTGACCCAGGGCACGACCTCAGT
CGTCCAGAGCTTCAATAGGGGTGACTGTTAG (SEQ ID NO: 68)
A11B1_11D10 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCATGGCCTCTGGAATCGACTTCAGTAGCGGCTACGGCATGTGGTGGGTCCG
CCAGGCTCCAGGGAAGGGACTGGAGTATATCGGATACATTGATACTGGTGATGATA
ACACATACTACGCGAACTGGGCGAAAGGCCGATTCACCATCTCCAAAACCTCGTCG
ACCACGGTGACTCTGCAAATGACCAGTCTGACAGTCGCGGACACGGCCACCTATTTC
TGTGCGAAAGGGGGCGCCATAGACCTCTGGGGCCCAGGGACCCTCGTCACCGTCTC
TTCAGGGCAACCTAAGGCTCCATCAGTCTTCCCACTGGCCCCCTGCTGCGGGGACAC
ACCCAGCTCCACGGTGACCCTGGGCTGCCTGGTCAAAGGCTACCTCCCGGAGCCAGT
GACCGTGACCTGGAACTCGGGCACCCTCACCAATGGGGTACGCACCTTCCCGTCCGT
CCGGCAGTCCTCAGGCCTCTACTCGCTGAGCAGCGTGGTGAGCGTGACCTCAAGCA
GCCAGCCCGTCACCTGCAACGTGGCCCACCCAGCCACCAACACCAAAGTGGACAAG
ACCGTTGCGCCCTCGACATGCAGCAAGCCCATGTGCCCACCCCCTGAACTCCCGGGG
GGACCGTCTGTCTTCATCTTCCCCCCAAAACCCAAGGACACCCTCATGATCTCACGC
ACCCCCGAGGTCACATGCGTGGTGGTGGACGTGAGCCAGGATGACCCCGAGGTGCA
GTTCACATGGTACATAAACAACGAGCAGGTGCGCACCGCCCGGCCGCCGCTACGGG
AGCAGCAGTTCAACAGCACGATCCGCGTGGTCAGCACCCTCCCCATCGCGCACCAG
GACTGGCTGAGGGGCAAGGAGTTCAAGTGCAAAGTCCACAACAAGGCACTCCCGGC
CCCCATCGAGAAAACCATCTCCAAAGCCAGAGGGCAGCCCCTGGAGCCGAAGGTCT
ACACCATGGGCCCTCCCCGGGAGGAGCTGAGCAGCAGGTCGGTCAGCCTGACCTGC
ATGATCAACGGCTTCTACCCTTCCGACATCTCGGTGGAGTGGGAGAAGAACGGGAA
GGCAGAGGACAACTACAAGACCACGCCGACCGTGCTGGACAGCGACGGCTCCTACT
TCCTCTACAGCAAGCTCTCAGTGCCCACGAGTGAGTGGCAGCGGGGCGACGTCTTCA
CCTGCTCCGTGATGCACGAGGCCTTGCACAACCACTACACGCAGAAGTCCATCTCCC
GCTCTCCGGGTAAATAG (SEQ ID NO: 69)
A11B1_11D10 Light Chain
ATGGACACGAGGGCCCCCACTCAGCTGCTGGGACTCCTACTGCTCTGGCTCCCAGGT
GCCAGATGTGCTGACATTGTGATGACCCAGACTCCAGCCTCCGTGGAGGCAGCTGTG
GGAGGCACAGTCACCATCAAGTGCCAGGCCAGTCAGAGCATTAGTAGTTACTTAGC
CTGGTATCAGCAGAAACCAGGGCAGCGTCCCAAGCTCCTGATCTACAGGGCATCCA
CTCTAAAATCTGGGGTCCCATCGCGGTTCAAAGGCAGTGGATCTGGGACAGAGTAC
ACTCTCACCATCAGCGACCTGGAGTGTGCCGATGCTGCCACTTATTATTGTCAAGCG
TATTATCTTAGTAGTAGTATCAGTTATGGTAATACTTTCGGCGGAGGGACCGAGGTG
GTGGTCAAAGGTGATCCAGTTGCACCTACTGTCCTCATCTTCCCACCAGCTGCTGAT
CAGGTGGCAACTGGAACAGTCACCATCGTGTGTGTGGCGAATAAATACTTTCCCGAT
GTCACCGTCACCTGGGAGGTGGATGGCACCACCCAAACAACTGGCATCGAGAACAG
TAAAACACCGCAGAATTCTGCAGATTGTACCTACAACCTCAGCAGCACTCTGACACT
GACCAGCACACAGTACAACAGCCACAAAGAGTACACCTGCAAGGTGACCCAGGGC
ACGACCTCAGTCGTCCAGAGCTTCAATAGGGGTGACTGTTAG (SEQ ID NO: 70)
A11B1_10F9 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCGTCCCTGACACT
CACCTGCACAGCCTCTGGATTCTCCCTCAGTAGCGGGTATGGCATGTGCTGGGTCCG
CCAGGCTCCAGGGAAGGGACTGGAGTGGATCGGATACACTGATACTGCTACTGGTA
CCATTCACTACGCGAGCTGGGCGAAAGGCCGATTCACCATCTCCAAAACCTCGTCGA
CCACGGTGACTCTGCAAATGACCAGTCTGACAGCCGCGGACACGGCCACCTATTTCT
GTGCGAAAGGGGGCGCCATGGACCTCTGGGGCCCAGGGACCCTCGTCACCGTCTCT
TCAGGGCAACCTAAGGCTCCATCAGTCTTCCCACTGGCCCCCTGCTGCGGGGACACA
CCCAGCTCCACGGTGACCCTGGGCTGCCTGGTCAAAGGCTACCTCCCGGAGCCAGTG
ACCGTGACCTGGAACTCGGGCACCCTCACCAATGGGGTACGCACCTTCCCGTCCGTC
CGGCAGTCCTCAGGCCTCTACTCGCTGAGCAGCGTGGTGAGCGTGACCTCAAGCAG
CCAGCCCGTCACCTGCAACGTGGCCCACCCAGCCACCAACACCAAAGTGGACAAGA
CCGTTGCGCCCTCGACATGCAGCAAGCCCATGTGCCCACCCCCTGAACTCCCGGGGG
GACCGTCTGTCTTCATCTTCCCCCCAAAACCCAAGGACACCCTCATGATCTCACGCA
CCCCCGAGGTCACATGCGTGGTGGTGGACGTGAGCCAGGATGACCCCGAGGTGCAG
TTCACATGGTACATAAACAACGAGCAGGTGCGCACCGCCCGGCCGCCGCTACGGGA
GCAGCAGTTCAACAGCACGATCCGCGTGGTCAGCACCCTCCCCATCGCGCACCAGG
ACTGGCTGAGGGGCAAGGAGTTCAAGTGCAAAGTCCACAACAAGGCACTCCCGGCC
CCCATCGAGAAAACCATCTCCAAAGCCAGAGGGCAGCCCCTGGAGCCGAAGGTCTA
CACCATGGGCCCTCCCCGGGAGGAGCTGAGCAGCAGGTCGGTCAGCCTGACCTGCA
TGATCAACGGCTTCTACCCTTCCGACATCTCGGTGGAGTGGGAGAAGAACGGGAAG
GCAGAGGACAACTACAAGACCACGCCGACCGTGCTGGACAGCGACGGCTCCTACTT
CCTCTACAGCAAGCTCTCAGTGCCCACGAGTGAGTGGCAGCGGGGCGACGTCTTCA
CCTGCTCCGTGATGCACGAGGCCTTGCACAACCACTACACGCAGAAGTCCATCTCCC
GCTCTCCGGGTAAATAG (SEQ ID NO: 71)
A11B1_10F9 Light Chain
ATGGACACGAGGGCCCCCACTCAGCTGCTGGGGCTCCTACTGCTCTGGCTCCCAGGT
GCCAGATGTGCTGACATTGTGATGACCCAGACTCCAGCCTCCGTGGAGGCAGCTGTG
GGAGGCACAGTCACCATCAAGTGCCAGGCCAGTCAGAGCATTAGTAGCTACTTAGC
CTGGTATCAGCAGAAACCAGGGCAGCCTCCCAAGCTCCTGATCTACAGGACATCCA
CTCTGGCATCTGGGGTCCCATCGCGGTTCAAAGGCAGTGGATCTGGGACAGAGTAC
ACTCTCACCATCAGCGACCTGGAGTGTGCCGATGCTGCCACTTACTATTGTCAAAGC
TATGCTTATAGTAGTAGTAGCAGTTATGGTAATGCTTTCGGCGGAGGGACCGAGGTG
GTGGTCAAAGGTGATCCAGTTGCACCTACTGTCCTCATCTTCCCACCAGCTGCTGAT
CAGGTGGCAACTGGAACAGTCACCATCGTGTGTGTGGCGAATAAATACTTTCCCGAT
GTCACCGTCACCTGGGAGGTGGATGGCACCACCCAAACAACTGGCATCGAGAACAG
TAAAACACCGCAGAATTCTGCAGATTGTACCTACAACCTCAGCAGCACTCTGACACT
GACCAGCACACAGTACAACAGCCACAAAGAGTACACCTGCAAGGTGACCCAGGGC
ACGACCTCAGTCGTCCAGAGCTTCAATAGGGGTGACTGTTAG (SEQ ID NO: 72)
A11B1_7H12 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCACAGGCTCTGGAATCGACTTCAGTAGCAGCTACTGGATATGCTGGGTCCG
CCAGGCTCCAGGGAAGGGGCTGGAGTGGATCGCATGCATCGATGGTAGTGATGGTA
ACACTTACTACGCGAGCTGGGCGAGAGGCCGATTCACCATCTCCAAAACCTCGTCG
ACCACGGTGACTCTGCAAATGGCCAGTCTGACAGCCGCGGACACGGCCACCTATTTC
TGTACGAGAGATCTCAGGTTGTGGGGCCCAGGCACCCTGGTCACCGTCTCCTCAGGG
CAACCTAAGGCTCCATCAGTCTTCCCACTGGCCCCCTGCTGCGGGGACACACCCAGC
TCCACGGTGACCCTGGGCTGCCTGGTCAAAGGCTACCTCCCGGAGCCAGTGACCGTG
ACCTGGAACTCGGGCACCCTCACCAATGGGGTACGCACCTTCCCGTCCGTCCGGCAG
TCCTCAGGCCTCTACTCGCTGAGCAGCGTGGTGAGCGTGACCTCAAGCAGCCAGCCC
GTCACCTGCAACGTGGCCCACCCAGCCACCAACACCAAAGTGGACAAGACCGTTGC
GCCCTCGACATGCAGCAAGCCCATGTGCCCACCCCCTGAACTCCCGGGGGGACCGT
CTGTCTTCATCTTCCCCCCAAAACCCAAGGACACCCTCATGATCTCACGCACCCCCG
AGGTCACATGCGTGGTGGTGGACGTGAGCCAGGATGACCCCGAGGTGCAGTTCACA
TGGTACATAAACAACGAGCAGGTGCGCACCGCCCGGCCGCCGCTACGGGAGCAGCA
GTTCAACAGCACGATCCGCGTGGTCAGCACCCTCCCCATCGCGCACCAGGACTGGCT
GAGGGGCAAGGAGTTCAAGTGCAAAGTCCACAACAAGGCACTCCCGGCCCCCATCG
AGAAAACCATCTCCAAAGCCAGAGGGCAGCCCCTGGAGCCGAAGGTCTACACCATG
GGCCCTCCCCGGGAGGAGCTGAGCAGCAGGTCGGTCAGCCTGACCTGCATGATCAA
CGGCTTCTACCCTTCCGACATCTCGGTGGAGTGGGAGAAGAACGGGAAGGCAGAGG
ACAACTACAAGACCACGCCGACCGTGCTGGACAGCGACGGCTCCTACTTCCTCTACA
GCAAGCTCTCAGTGCCCACGAGTGAGTGGCAGCGGGGCGACGTCTTCACCTGCTCC
GTGATGCACGAGGCCTTGCACAACCACTACACGCAGAAGTCCATCTCCCGCTCTCCG
GGTAAATAG (SEQ ID NO: 73)
A11B1_7H12 Light Chain
ATGGACACGAGGGCCCCCACTCAGCTGCTGGGGCTCCTGCTGCTCTGGCTCCCAGGT
GCCAGATGTGCTGACATTGTGCTGACCCAGACTCCAGCCTCGGTGTCTGCAGCTGTG
GGAGGCACAGTCACCATCAACTGCCAGGCCAGTCAGAATGTTTATAGTAACAATGC
CTTAGCCTGGCATCAGCAGAAACCAGGGCAGCGTCCCAACCTCCTGATCTACAAGG
CTTCCACTCTGGCATCTGGGGTCCCATCGCGGTTCAAAGGCAGTGGATCTGGGACAC
AGTTTACTCTCACCATCAGCGACGTGCAGTGTGACGATGCTGCCACTTACTACTGTC
TAGGCGAATTTAGTTGTAGTAGTGGTGATTGTTTTGTTTTCGGCGGAGGGACCGAGG
TGGTGGTCAAAGGTGATCCAGTTGCACCTACTGTCCTCATCTTCCCACCAGCTGCTG
ATCAGGTGGCAACTGGAACAGTCACCATCGTGTGTGTGGCGAATAAATACTTTCCCG
ATGTCACCGTCACCTGGGAGGTGGATGGCACCACCCAAACAACTGGCATCGAGAAC
AGTAAAACACCGCAGAATTCTGCAGATTGTACCTACAACCTCAGCAGCACTCTGAC
ACTGACCAGCACACAGTACAACAGCCACAAAGAGTACACCTGCAAGGTGACCCAGG
GCACGACCTCAGTCGTCCAGAGCTTCAATAGGGGTGACTGTTAG (SEQ ID NO: 74)
A11B1_7G12 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCATGGCCTCTGGAATCGACTTCAGTAGCGGCTACGGCATGTGGTGGGTCCG
CCAGGCTCCAGGGAAGGGACTGGAGTATATCGGATACATTGATACTGGTGATGATA
ACACATACTACGCGAACTGGGCGAAAGGCCGATTCACCATCTCCAAAACCTCGTCG
ACCACGGTGACTCTGCAAATGACCAGTCTGACAGTCGCGGACACGGCCACCTATTTC
TGTGCGAAAGGGGGCGCCATAGACCTCTGGGGCCCAGGGACCCTCGTCACCGTCTC
TTCAGGGCAACCTAAGGCTCCATCAGTCTTCCCACTGGCCCCCTGCTGCGGGGACAC
ACCCAGCTCCACGGTGACCCTGGGCTGCCTGGTCAAAGGCTACCTCCCGGAGCCAGT
GACCGTGACCTGGAACTCGGGCACCCTCACCAATGGGGTACGCACCTTCCCGTCCGT
CCGGCAGTCCTCAGGCCTCTACTCGCTGAGCAGCGTGGTGAGCGTGACCTCAAGCA
GCCAGCCCGTCACCTGCAACGTGGCCCACCCAGCCACCAACACCAAAGTGGACAAG
ACCGTTGCGCCCTCGACATGCAGCAAGCCCATGTGCCCACCCCCTGAACTCCCGGGG
GGACCGTCTGTCTTCATCTTCCCCCCAAAACCCAAGGACACCCTCATGATCTCACGC
ACCCCCGAGGTCACATGCGTGGTGGTGGACGTGAGCCAGGATGACCCCGAGGTGCA
GTTCACATGGTACATAAACAACGAGCAGGTGCGCACCGCCCGGCCGCCGCTACGGG
AGCAGCAGTTCAACAGCACGATCCGCGTGGTCAGCACCCTCCCCATCGCGCACCAG
GACTGGCTGAGGGGCAAGGAGTTCAAGTGCAAAGTCCACAACAAGGCACTCCCGGC
CCCCATCGAGAAAACCATCTCCAAAGCCAGAGGGCAGCCCCTGGAGCCGAAGGTCT
ACACCATGGGCCCTCCCCGGGAGGAGCTGAGCAGCAGGTCGGTCAGCCTGACCTGC
ATGATCAACGGCTTCTACCCTTCCGACATCTCGGTGGAGTGGGAGAAGAACGGGAA
GGCAGAGGACAACTACAAGACCACGCCGACCGTGCTGGACAGCGACGGCTCCTACT
TCCTCTACAGCAAGCTCTCAGTGCCCACGAGTGAGTGGCAGCGGGGCGACGTCTTCA
CCTGCTCCGTGATGCACGAGGCCTTGCACAACCACTACACGCAGAAGTCCATCTCCC
GCTCTCCGGGTAAATAG (SEQ ID NO: 75)
A11B1_7G12 Light Chain
ATGGACACGAGGGCCCCCACTCAGCTGCTGGGACTCCTACTGCTCTGGCTCCCAGGT
GCCAGATGTGCTGACATTGTGATGACCCAGACTCCAGCCTCCGTGGAGGCAGCTGTG
GGAGGCACAGTCACCATCAAGTGCCAGGCCAGTCAGAGCATTAGTAGTTACTTAGC
CTGGTATCAGCAGAAACCAGGGCAGCGTCCCAAGCTCCTGATCTACAGGGCATCCA
CTCTAAAATCTGGGGTCCCATCGCGGTTCAAAGGCAGTGGATCTGGGACAGAGTAC
ACTCTCACCATCAGCGACCTGGAGTGTGCCGATGCTGCCACTTATTATTGTCAAGCG
TATTATCTTAGTAGTAGTATCAGTTATGGTAATACTTTCGGCGGAGGGACCGAGGTG
GTGGTCAAAGGTGATCCAGTTGCACCTACTGTCCTCATCTTCCCACCAGCTGCTGAT
CAGGTGGCAACTGGAACAGTCACCATCGTGTGTGTGGCGAATAAATACTTTCCCGAT
GTCACCGTCACCTGGGAGGTGGATGGCACCACCCAAACAACTGGCATCGAGAACAG
TAAAACACCGCAGAATTCTGCAGATTGTACCTACAACCTCAGCAGCACTCTGACACT
GACCAGCACACAGTACAACAGCCACAAAGAGTACACCTGCAAGGTGACCCAGGGC
ACGACCTCAGTCGTCCAGAGCTTCAATAGGGGTGACTGTTAG (SEQ ID NO: 76)
A11B1_6G4 Heavy Chain
ATGGAGACTGGGCTGCGCTGGCTTCTCCTGGTCGCTGTGCTCAAAGGTGTCCAGTGT
CAGCAGCAGCTGGAGGAGTCCGGGGGAGGCCTGGTCAAGCCTGGAGGAACCCTGAC
ACTCACCTGCAAAGCCTCTGGAGTCGCCCTCAATCCCTACTACTATATGTGCTGGGT
CCGCCAGGCTCCAGGGAAGGGGCTGGAGTGGATCGCATGCGTGGATGCTGATAGTA
GTGGTAGCACTTACTACGCGAGCTGGGCGAAAGGCCGATTCACCATCTCCAAAACC
TCGTCGACCACGGTGACTCTGAAAATGACCAGTCTGACAGCCGCGGACACGGCCAC
CTATTTCTGTGCGAGAGAATCGGTTGACTATAGTTCTGTTGGTATTGGCTATGTACAT
GGTACGGATGGCTTGTGGGGCCCAGGCACCCTGGTCACCGTCTCCTCAGGGCAACCT
AAGGCTCCATCAGTCTTCCCACTGGCCCCCTGCTGCGGGGACACACCCAGCTCCACG
GTGACCCTGGGCTGCCTGGTCAAAGGCTACCTCCCGGAGCCAGTGACCGTGACCTG
GAACTCGGGCACCCTCACCAATGGGGTACGCACCTTCCCGTCCGTCCGGCAGTCCTC
AGGCCTCTACTCGCTGAGCAGCGTGGTGAGCGTGACCTCAAGCAGCCAGCCCGTCA
CCTGCAACGTGGCCCACCCAGCCACCAACACCAAAGTGGACAAGACCGTTGCGCCC
TCGACATGCAGCAAGCCCATGTGCCCACCCCCTGAACTCCCGGGGGGACCGTCTGTC
TTCATCTTCCCCCCAAAACCCAAGGACACCCTCATGATCTCACGCACCCCCGAGGTC
ACATGCGTGGTGGTGGACGTGAGCCAGGATGACCCCGAGGTGCAGTTCACATGGTA
CATAAACAACGAGCAGGTGCGCACCGCCCGGCCGCCGCTACGGGAGCAGCAGTTCA
ACAGCACGATCCGCGTGGTCAGCACCCTCCCCATCGCGCACCAGGACTGGCTGAGG
GGCAAGGAGTTCAAGTGCAAAGTCCACAACAAGGCACTCCCGGCCCCCATCGAGAA
AACCATCTCCAAAGCCAGAGGGCAGCCCCTGGAGCCGAAGGTCTACACCATGGGCC
CTCCCCGGGAGGAGCTGAGCAGCAGGTCGGTCAGCCTGACCTGCATGATCAACGGC
TTCTACCCTTCCGACATCTCGGTGGAGTGGGAGAAGAACGGGAAGGCAGAGGACAA
CTACAAGACCACGCCGACCGTGCTGGACAGCGACGGCTCCTACTTCCTCTACAGCAA
GCTCTCAGTGCCCACGAGTGAGTGGCAGCGGGGCGACGTCTTCACCTGCTCCGTGAT
GCACGAGGCCTTGCACAACCACTACACGCAGAAGTCCATCTCCCGCTCTCCGGGTAA
ATAG (SEQ ID NO: 77)
A11B1_6G4 Light Chain
ATGGACACGAGGGCCCCCACTCAGCTGCTGGGGCTCCTGCTGCTCTGGCTCCCAGGT
GCCAGATGTGCCGACATCGTGGTGACCCAGACTCCATCCTCCGTGTCTGCAGCTGTG
GGAGGCACAGTCACCATCAAGTGCCAGGCCAGTCAGAGCATTAGCAACTACTTTTCT
TGGTATCAGCAGAAACCAGGGCAGCCTCCCAAGCTCCTGATCTACAGGGCGTCCAC
TCTGGCATCTGGGGTCCCATCGCGGTTCAAAGGCAGTGGATCTGGGACAGAGTTCAC
TCTCACCATCAGCGACCTGGAGTGTGCCGATGCTGCCACTTACTACTGTCAATGCAC
TTACGGTAGAAGTAATAGTAATTTTTTTTATGGTTTCGGCGGAGGGACCGAGGTGGT
GGTCAAAGGTGATCCAGTTGCACCTACTGTCCTCATCTTCCCACCAGCTGCTGATCA
GGTGGCAACTGGAACAGTCACCATCGTGTGTGTGGCGAATAAATACTTTCCCGATGT
CACCGTCACCTGGGAGGTGGATGGCACCACCCAAACAACTGGCATCGAGAACAGTA
AAACACCGCAGAATTCTGCAGATTGTACCTACAACCTCAGCAGCACTCTGACACTGA
CCAGCACACAGTACAACAGCCACAAAGAGTACACCTGCAAGGTGACCCAGGGCACG
ACCTCAGTCGTCCAGAGCTTCAATAGGGGTGACTGTTAG (SEQ ID NO: 78)
A11B1_6F9 Heavy Chain
ATGGAGACTGGGCTGCGCTGGCTTCTCCTGGTCGCCGTGCTCAAAGGTGTCCAGTGT
CAGTCGTTGGAGGAGTCCGGGGGAGACCTGGTCAAGCCTGGGGCCTCCCTGACACT
CACCTGCACAGCCTCTGGATCCTCCTTCAGTAGTACCTACTGGAACTGCTGGGTCCG
CCAGGCTCCAGGGAAGGGGCTGGAGTGGATCGCATGCATTAATGCTGGTAGTGGTA
CCACTTACTACGCGAGCTGGGCGAAAGGCCGATTCACCGTCTCCAAAACCTCGTCGA
CCACGGTGACTCTGCAAATGACCAGTCTGACAGCCGCGGACACGGCCACCTATTTCT
GTACGAGAGATAGTGATGGTCGTTTTAGTAGTGGCTACTATTTTAACTTGTGGGGCC
CAGGCACCCTGGTCACCGTCTCCTCAGGGCAACCTAAGGCTCCATCAGTCTTCCCAC
TGGCCCCCTGCTGCGGGGACACACCCAGCTCCACGGTGACCCTGGGCTGCCTGGTCA
AAGGCTACCTCCCGGAGCCAGTGACCGTGACCTGGAACTCGGGCACCCTCACCAAT
GGGGTACGCACCTTCCCGTCCGTCCGGCAGTCCTCAGGCCTCTACTCGCTGAGCAGC
GTGGTGAGCGTGACCTCAAGCAGCCAGCCCGTCACCTGCAACGTGGCCCACCCAGC
CACCAACACCAAAGTGGACAAGACCGTTGCGCCCTCGACATGCAGCAAGCCCATGT
GCCCACCCCCTGAACTCCCGGGGGGACCGTCTGTCTTCATCTTCCCCCCAAAACCCA
AGGACACCCTCATGATCTCACGCACCCCCGAGGTCACATGCGTGGTGGTGGACGTG
AGCCAGGATGACCCCGAGGTGCAGTTCACATGGTACATAAACAACGAGCAGGTGCG
CACCGCCCGGCCGCCGCTACGGGAGCAGCAGTTCAACAGCACGATCCGCGTGGTCA
GCACCCTCCCCATCGCGCACCAGGACTGGCTGAGGGGCAAGGAGTTCAAGTGCAAA
GTCCACAACAAGGCACTCCCGGCCCCCATCGAGAAAACCATCTCCAAAGCCAGAGG
GCAGCCCCTGGAGCCGAAGGTCTACACCATGGGCCCTCCCCGGGAGGAGCTGAGCA
GCAGGTCGGTCAGCCTGACCTGCATGATCAACGGCTTCTACCCTTCCGACATCTCGG
TGGAGTGGGAGAAGAACGGGAAGGCAGAGGACAACTACAAGACCACGCCGACCGT
GCTGGACAGCGACGGCTCCTACTTCCTCTACAGCAAGCTCTCAGTGCCCACGAGTGA
GTGGCAGCGGGGCGACGTCTTCACCTGCTCCGTGATGCACGAGGCCTTGCACAACC
ACTACACGCAGAAGTCCATCTCCCGCTCTCCGGGTAAATAG (SEQ ID NO: 79)
A11B1_6F9 Light Chain
ATGGACACGAGGGCCCCCACTCAGCTGCTGGGGCTCCTGCTGCTCTGGCTCCCAGGT
GCCACATTTGCCCAAGTGCTGACCCAGACTGCATCCCCTGTGTCTGCAGCTGTGGGA
GGCACAGTCACCATCAATTGTCAGTCCAGTCAGAGTGTTTATGATAACAACTGGTTA
GCCTGGTATCAGCAAAAACCAGGGCAGCCTCCCAAACTCTTGATCGACGATGCATC
CAAATTGACATCTGGGGTCTCATCGCGGTTCAAAGGCAGTGGATCTGGGACGCAGTT
CACTCTCACCATCAGCGGCGTGCAGTGTGACGATGCTGCCACTTACTACTGTCAAGG
CGCTTATTATAGTAGTGGTTGGTACTGGGCTTTCGGCGGAGGGACCGAGGTGGTGGT
CAAAGGTGATCCAGTTGCACCTACTGTCCTCATCTTCCCACCAGCTGCTGATCAGGT
GGCAACTGGAACAGTCACCATCGTGTGTGTGGCGAATAAATACTTTCCCGATGTCAC
CGTCACCTGGGAGGTGGATGGCACCACCCAAACAACTGGCATCGAGAACAGTAAAA
CACCGCAGAATTCTGCAGATTGTACCTACAACCTCAGCAGCACTCTGACACTGACCA
GCACACAGTACAACAGCCACAAAGAGTACACCTGCAAGGTGACCCAGGGCACGACC
TCAGTCGTCCAGAGCTTCAATAGGGGTGACTGTTAG (SEQ ID NO: 80)
A11B1_6C7 Heavy Chain
ATGGAGACTGGGCTGCGCTGGCTTCTCCTGGTCGCTGTGCTCAAAGGTGTCCAGTGT
CAGCAGCAGCTGGAGGAGTCCGGGGGAGGCCTGGTCAAGCCTGGAGGAACCCTGAC
ACTCACCTGCAAAGCCTCTGGAATCGACTTCAGTAGCTACTACTACATGTGTTGGGT
CCGCCAGGCTCCAGGGAAGGGGCTGGAGTTGATCGTATGTATTTATACTAGTAGTGG
TGGCACATGGTACGCGAGCTGGGTGAATGGCCGACTCACCATCTCCAGAAGCACCA
GCCTAAACACGGTGGATCTGAAAATGACCAGTCTGACAGCCGCGGACACGGCCACC
TATTTCTGTGCCAGAGGGGTTTATTCTGGTAGTAGTGATTATCCAACTCGGTTGGATC
TCTGGGGCCAGGGCACCCTGGTCACCGTCTCCTTAGGGCAACCTAAGGCTCCATCAG
TCTTCCCACTGGCCCCCTGCTGCGGGGACACACCCAGCTCCACGGTGACCCTGGGCT
GCCTGGTCAAAGGCTACCTCCCGGAGCCAGTGACCGTGACCTGGAACTCGGGCACC
CTCACCAATGGGGTACGCACCTTCCCGTCCGTCCGGCAGTCCTCAGGCCTCTACTCG
CTGAGCAGCGTGGTGAGCGTGACCTCAAGCAGCCAGCCCGTCACCTGCAACGTGGC
CCACCCAGCCACCAACACCAAAGTGGACAAGACCGTTGCGCCCTCGACATGCAGCA
AGCCCATGTGCCCACCCCCTGAACTCCCGGGGGGACCGTCTGTCTTCATCTTCCCCC
CAAAACCCAAGGACACCCTCATGATCTCACGCACCCCCGAGGTCACATGCGTGGTG
GTGGACGTGAGCCAGGATGACCCCGAGGTGCAGTTCACATGGTACATAAACAACGA
GCAGGTGCGCACCGCCCGGCCGCCGCTACGGGAGCAGCAGTTCAACAGCACGATCC
GCGTGGTCAGCACCCTCCCCATCGCGCACCAGGACTGGCTGAGGGGCAAGGAGTTC
AAGTGCAAAGTCCACAACAAGGCACTCCCGGCCCCCATCGAGAAAACCATCTCCAA
AGCCAGAGGGCAGCCCCTGGAGCCGAAGGTCTACACCATGGGCCCTCCCCGGGAGG
AGCTGAGCAGCAGGTCGGTCAGCCTGACCTGCATGATCAACGGCTTCTACCCTTCCG
ACATCTCGGTGGAGTGGGAGAAGAACGGGAAGGCAGAGGACAACTACAAGACCAC
GCCGACCGTGCTGGACAGCGACGGCTCCTACTTCCTCTACAGCAAGCTCTCAGTGCC
CACGAGTGAGTGGCAGCGGGGCGACGTCTTCACCTGCTCCGTGATGCACGAGGCCT
TGCACAACCACTACACGCAGAAGTCCATCTCCCGCTCTCCGGGTAAATAG (SEQ ID
NO: 81)
A11B1_6C7 Light Chain
ATGGACACGAGCACCTCCACTGCGCTCCTGGGGCTCCTGCTGCTCTGGCTCACAGGT
GCCAGATGTGCCATCGAGATGACCCAGTCTCCACCCTCCCTGTCTGCATCTGTGGGA
GAAACTGTCAGGATTAGGTGCCTGGCCAGTGAGGACATTTACAGTGGTATATCCTGG
TACCAACAGAAGCCAGAGAAACCTCCTACACTCCTGATCTCTGGTGCATCCAATTTA
GAATCTGGGGTCCCACCACGGTTCAGTGGCGGTGGATCCGGGACAGATTACACCCT
CACCATCGGCGGCGTGCAGGCTGAAGATGTTGCCACCTACTACTGTCTAGGCGGTTA
TAGTTTCAGTAGTACCGGTTTGACTTTTGGAGCTGGCACCAAGGTGGAAATCAAACG
TGATCCAGTTGCGCCTTCTGTCCTCCTCTTCCCACCATCTAAGGAGGAGCTGACAAC
TGGAACAGCCACCATCGTGTGTGTGGCGAATAAATACTTTCCCGATGTCACCGTCAC
CTGGGAGGTGGATGGCACCACCCAAACAACTGGCATCGAGAACAGTAAAACACCGC
AGAATTCTGCAGATTGTACCTACAACCTCAGCAGCACTCTGACACTGACCAGCACAC
AGTACAACAGCCACAAAGAGTACACCTGCAAGGTGACCCAGGGCACGACCTCAGTC
GTCCAGAGCTTCAATAGGGGTGACTGTTAG (SEQ ID NO: 82)
A11B1_6B6 Heavy Chain
ATGGAGACTGGGCTGCGCTGGCTTCTCCTGGTCGCTGTGCTCAAAGGTGTCCAGTGT
CAGCAACATCTGGTGGAGTCCGGGGGAGGCCTGGTCAAGCCTGGGGCATCCCTGAC
ACTCACCTGCACAGCCTCTGGATTCTCCTTCACTACCGGCTATCACATGTGCTGGGTC
CGCCAGGCTCCAGGGAAGGGGCTGGAGTGGATCGCATGTTTTGGTGTTTATACTAGT
ACCACTACCTACGCGAGCTGGGCGAAAGGTCGATTCACCATCTCCAAAACCTCGTCG
ACCACGGTGACTCTACAAATGACCAGTCTAACAGTCGCGGACACGGCCACCTATTTC
TGTGCGAGAATCAGTGCTGAAGATGGTGGGGACTTGTGGGGCCCAGGCACCCTGGT
CACCGTCTCCTCAGGGCAACCTAAGGCTCCATCAGTCTTCCCACTGGCCCCCTGCTG
CGGGGACACACCCAGCTCCACGGTGACCCTGGGCTGCCTGGTCAAAGGCTACCTCC
CGGAGCCAGTGACCGTGACCTGGAACTCGGGCACCCTCACCAATGGGGTACGCACC
TTCCCGTCCGTCCGGCAGTCCTCAGGCCTCTACTCGCTGAGCAGCGTGGTGAGCGTG
ACCTCAAGCAGCCAGCCCGTCACCTGCAACGTGGCCCACCCAGCCACCAACACCAA
AGTGGACAAGACCGTTGCGCCCTCGACATGCAGCAAGCCCATGTGCCCACCCCCTG
AACTCCCGGGGGGACCGTCTGTCTTCATCTTCCCCCCAAAACCCAAGGACACCCTCA
TGATCTCACGCACCCCCGAGGTCACATGCGTGGTGGTGGACGTGAGCCAGGATGAC
CCCGAGGTGCAGTTCACATGGTACATAAACAACGAGCAGGTGCGCACCGCCCGGCC
GCCGCTACGGGAGCAGCAGTTCAACAGCACGATCCGCGTGGTCAGCACCCTCCCCA
TCGCGCACCAGGACTGGCTGAGGGGCAAGGAGTTCAAGTGCAAAGTCCACAACAAG
GCACTCCCGGCCCCCATCGAGAAAACCATCTCCAAAGCCAGAGGGCAGCCCCTGGA
GCCGAAGGTCTACACCATGGGCCCTCCCCGGGAGGAGCTGAGCAGCAGGTCGGTCA
GCCTGACCTGCATGATCAACGGCTTCTACCCTTCCGACATCTCGGTGGAGTGGGAGA
AGAACGGGAAGGCAGAGGACAACTACAAGACCACGCCGACCGTGCTGGACAGCGA
CGGCTCCTACTTCCTCTACAGCAAGCTCTCAGTGCCCACGAGTGAGTGGCAGCGGGG
CGACGTCTTCACCTGCTCCGTGATGCACGAGGCCTTGCACAACCACTACACGCAGAA
GTCCATCTCCCGCTCTCCGGGTAAATAG (SEQ ID NO: 83)
A11B1_6B6 Light Chain
ATGGACACGAGGGCCCCCACTCAGCTGCTGGGGCTCCTGCTGCTCTGGCTCCCAGGT
GCCAGATGTGATGTTGTGATGACCCAGACTCCAGCCTCCGTGGAGGCAGCTGTGGG
AGGCACAGTCACCATCACGTGCCAGGCCAGTCAGAGCATTAGCAACTACTTTTCTTG
GTATCAGCAGAAACCAGGGCAGCCTCCCAAGCTCCTGATCTACAGGGCGTCCACTCT
GGCATCTGGGGTCCCATCGCGGTTCAGCGGCAGTGGATCTGGGACACAGTTCACTCT
CACCATCAGCGACCTGGAGTGTGCCGATTCTGCCACTTACGCCTGTCAGTGCACTTA
TGGTAGTAGTAGTACTGGTTTTGGTTTCGGCGGAGGGACCGAGGTGGTGGTCAAAG
GTGATCCAGTTGCACCTACTGTCCTCATCTTCCCACCAGCTGCTGATCAGGTGGCAA
CTGGAACAGTCACCATCGTGTGTGTGGCGAATAAATACTTTCCCGATGTCACCGTCA
CCTGGGAGGTGGATGGCACCACCCAAACAACTGGCATCGAGAACAGTAAAACACCG
CAGAATTCTGCAGATTGTACCTACAACCTCAGCAGCACTCTGACACTGACCAGCACA
CAGTACAACAGCCACAAAGAGTACACCTGCAAGGTGACCCAGGGCACGACCTCAGT
CGTCCAGAGCTTCAATAGGGGTGACTGTTAG (SEQ ID NO: 84)
A11B1_5F7 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCAAAGCCTCTGGATTCTCCTTCAGTAGTTACTTCTGGATATGCTGGGTCCGC
CAGGCTCCAGGGAAGGGGCTGGAGTGGAGCGCATGCATCTATGGTGATAGTAGTGG
TAGTAGTTACTACGCGAGCTGGGCGAAAGGCCGATTCACCATCTCCAAAACCTCGTC
GACCACGGTGACTCTGCAAATGACCAGTCTGACAGCCGCGGACACGGCCACCTATT
TCTGTGCGAGTTATGGTAGTAGTAGTTATTACTACTCTAATTTATGGGGCCCAGGCA
CCCTGGTCACCGTCTCCTCAGGGCAACCTAAGGCTCCATCAGTCTTCCCACTGGCCC
CCTGCTGCGGGGACACACCCAGCTCCACGGTGACCCTGGGCTGCCTGGTCAAAGGC
TACCTCCCGGAGCCAGTGACCGTGACCTGGAACTCGGGCACCCTCACCAATGGGGT
ACGCACCTTCCCGTCCGTCCGGCAGTCCTCAGGCCTCTACTCGCTGAGCAGCGTGGT
GAGCGTGACCTCAAGCAGCCAGCCCGTCACCTGCAACGTGGCCCACCCAGCCACCA
ACACCAAAGTGGACAAGACCGTTGCGCCCTCGACATGCAGCAAGCCCATGTGCCCA
CCCCCTGAACTCCCGGGGGGACCGTCTGTCTTCATCTTCCCCCCAAAACCCAAGGAC
ACCCTCATGATCTCACGCACCCCCGAGGTCACATGCGTGGTGGTGGACGTGAGCCA
GGATGACCCCGAGGTGCAGTTCACATGGTACATAAACAACGAGCAGGTGCGCACCG
CCCGGCCGCCGCTACGGGAGCAGCAGTTCAACAGCACGATCCGCGTGGTCAGCACC
CTCCCCATCGCGCACCAGGACTGGCTGAGGGGCAAGGAGTTCAAGTGCAAAGTCCA
CAACAAGGCACTCCCGGCCCCCATCGAGAAAACCATCTCCAAAGCCAGAGGGCAGC
CCCTGGAGCCGAAGGTCTACACCATGGGCCCTCCCCGGGAGGAGCTGAGCAGCAGG
TCGGTCAGCCTGACCTGCATGATCAACGGCTTCTACCCTTCCGACATCTCGGTGGAG
TGGGAGAAGAACGGGAAGGCAGAGGACAACTACAAGACCACGCCGACCGTGCTGG
ACAGCGACGGCTCCTACTTCCTCTACAGCAAGCTCTCAGTGCCCACGAGTGAGTGGC
AGCGGGGCGACGTCTTCACCTGCTCCGTGATGCACGAGGCCTTGCACAACCACTACA
CGCAGAAGTCCATCTCCCGCTCTCCGGGTAAATAG (SEQ ID NO: 85)
A11B1_5F7 Light Chain
ATGGACACGAGGGCCCCCACTCAGCTGCTGGGGCTCCTGCTGCTCTGGCTCCCAGGT
GCCATATGTGACCCTGTGATGACCCAGACTCCATCTTCCACGTCTGCGGCTGTGGGA
GGCACAGTCACCATCAGTTGCCAGTCCAGTCAGAGTGTTTATAATAACAACTACTTA
GCCTGGTATCAGCAGAAACCAGGGCAGCCTCCCAAACGCCTGATCTACGAATCATC
CAAACTGGCATCTGGGGTCCCATCGCGGTTCAGAGGCAGTGGATCTGGGGCACAGT
TCACTCTCACCATCAGCGACCTGGAGTGTGACGATGCTGCCACTTACTACTGTCTAG
GCGCATATTATACTACTCTTGATTTCGGCGGAGGGACCGAGGTGGTGGTCAGAGGTG
ATCCAGTTGCACCTACTGTCCTCATCTTCCCACCAGCTGCTGATCAGGTGGCAACTG
GAACAGTCACCATCGTGTGTGTGGCGAATAAATACTTTCCCGATGTCACCGTCACCT
GGGAGGTGGATGGCACCACCCAAACAACTGGCATCGAGAACAGTAAAACACCGCA
GAATTCTGCAGATTGTACCTACAACCTCAGCAGCACTCTGACACTGACCAGCACACA
GTACAACAGCCACAAAGAGTACACCTGCAAGGTGACCCAGGGCACGACCTCAGTCG
TCCAGAGCTTCAATAGGGGTGACTGTTAG (SEQ ID NO: 86)
A11B1_5D7 Heavy Chain
ATGGAGACTGGGCTGCGCTGGCTTCTCCTGGTCGCTGTGCTCAAAGGTGTCCAGTGT
CAGGAGCAGTTGGTGGAGTCCGGGGGAGGCCTGGTCCAGCCTGAGGGATCCCTGAC
ACTCACCTGCAAAGCCTCTGGATTCGACTTCAGTAGCAATGCAATGTGCTGGGTCCG
CCAGGCTCCAGGGAAGGGGCTGGAGTGGATCGCATGCATTTATAATGGTGATGGCA
GCACATACTACGCGAGCTGGGTGAATGGCCGATTCACCATCTCCAAGACCTCGTCGA
CCACGGTGACTCTGCAAATGACCAGTCTGACAGCCGCGGACACGGCCACCTATTTCT
GTGCGAGAGGTCTCTCTAATTGGAATAGGGATAACTTATGGGGCCCTGGCACCCTGG
TCACCGTCTCCTCAGGGCAACCTAAGGCTCCATCAGTCTTCCCACTGGCCCCCTGCT
GCGGGGACACACCCAGCTCCACGGTGACCCTGGGCTGCCTGGTCAAAGGCTACCTC
CCGGAGCCAGTGACCGTGACCTGGAACTCGGGCACCCTCACCAATGGGGTACGCAC
CTTCCCGTCCGTCCGGCAGTCCTCAGGCCTCTACTCGCTGAGCAGCGTGGTGAGCGT
GACCTCAAGCAGCCAGCCCGTCACCTGCAACGTGGCCCACCCAGCCACCAACACCA
AAGTGGACAAGACCGTTGCGCCCTCGACATGCAGCAAGCCCATGTGCCCACCCCCT
GAACTCCCGGGGGGACCGTCTGTCTTCATCTTCCCCCCAAAACCCAAGGACACCCTC
ATGATCTCACGCACCCCCGAGGTCACATGCGTGGTGGTGGACGTGAGCCAGGATGA
CCCCGAGGTGCAGTTCACATGGTACATAAACAACGAGCAGGTGCGCACCGCCCGGC
CGCCGCTACGGGAGCAGCAGTTCAACAGCACGATCCGCGTGGTCAGCACCCTCCCC
ATCGCGCACCAGGACTGGCTGAGGGGCAAGGAGTTCAAGTGCAAAGTCCACAACAA
GGCACTCCCGGCCCCCATCGAGAAAACCATCTCCAAAGCCAGAGGGCAGCCCCTGG
AGCCGAAGGTCTACACCATGGGCCCTCCCCGGGAGGAGCTGAGCAGCAGGTCGGTC
AGCCTGACCTGCATGATCAACGGCTTCTACCCTTCCGACATCTCGGTGGAGTGGGAG
AAGAACGGGAAGGCAGAGGACAACTACAAGACCACGCCGACCGTGCTGGACAGCG
ACGGCTCCTACTTCCTCTACAGCAAGCTCTCAGTGCCCACGAGTGAGTGGCAGCGGG
GCGACGTCTTCACCTGCTCCGTGATGCACGAGGCCTTGCACAACCACTACACGCAGA
AGTCCATCTCCCGCTCTCCGGGTAAATAG (SEQ ID NO: 87)
A11B1_5D7 Light Chain
ATGGACACGAGGGCCCCCACTCAGCTGCTGGGGCTCCTGCTGCTCTGGCTCCCAGGT
GCCACATTTGCCCAAGTGCTGACCCAGACTCCATCCTCCGTGTCTGCAGCTGTGGGA
GGCACAGCCACCATCAACTGCCAGGCCAGTCAGAGTCTTTATAGTCCCAAGAATTTA
GCCTGGTATCAGCAGACACCAGGGCAGCCTCCCAAGCTCCTGATCTATTCTGCATCG
AAACTGGCATCTGGGGTCCCATCGCGGTTCAAAGGCAGTGGATCTGGGACACAGTT
CACTCTCACCATCAGCGGCGTGCAGTGTGACGATGCTGCAATTTACTACTGTCAAGG
CGAATTTAGTTGTACTACTGCTGCTTGTTTTGCTTTTGGCGGAGGGACCGAGGTGGT
GGTCAAAGGTGATCCAGTTGCACCTACTGTCCTCATCTTCCCACCAGCTGCTGATCA
GGTGGCAACTGGAACAGTCACCATCGTGTGTGTGGCGAATAAATACTTTCCCGATGT
CACCGTCACCTGGGAGGTGGATGGCACCACCCAAACAACTGGCATCGAGAACAGTA
AAACACCGCAGAATTCTGCAGATTGTACCTACAACCTCAGCAGCACTCTGACACTGA
CCAGCACACAGTACAACAGCCACAAAGAGTACACCTGCAAGGTGACCCAGGGCACG
ACCTCAGTCGTCCAGAGCTTCAATAGGGGTGACTGTTAG (SEQ ID NO: 88)
A11B1_5A7 Heavy Chain
ATGGAGACTGGGCTGCGCTGGCTTCTCCTGGTCGCTGTGCTCAAAGGTGTCCAGTGT
CAGTCTTTGGAGGAGTCCGGGGGAGGCCTGGTCCAGCCTGAGGGATCCCTGACACT
CGCCTGCACAGCTTCGGGATTCTCCTTCAGTAGCTACTACTACATCTGCTGGGTCCG
CCAGGCTCCAGGGACGGGGCTGGAGTGGATCGGATGCATTAATACTGGTAGTGATG
ACACTCACTACGCGAGCTGGTTGAAAGGCCGATTCACCTTCTCCAAGGCCTCGTCGA
CCACGTTGACTCTGCAAATGACCAGTCTGACAGCCGCGGACACGGCCACCTATTTCT
GTGCGAGATCATCTGGTAGTAGTGATGATGCTTATGATCTCTGGGGCCCAGGCACCC
TGGTCACTGTCTCCTCAGGGCAACCTAAGGCTCCATCAGTCTTCCCACTGGCCCCCT
GCTGCGGGGACACACCCAGCTCCACGGTGACCCTGGGCTGCCTGGTCAAAGGCTAC
CTCCCGGAGCCAGTGACCGTGACCTGGAACTCGGGCACCCTCACCAATGGGGTACG
CACCTTCCCGTCCGTCCGGCAGTCCTCAGGCCTCTACTCGCTGAGCAGCGTGGTGAG
CGTGACCTCAAGCAGCCAGCCCGTCACCTGCAACGTGGCCCACCCAGCCACCAACA
CCAAAGTGGACAAGACCGTTGCGCCCTCGACATGCAGCAAGCCCATGTGCCCACCC
CCTGAACTCCCGGGGGGACCGTCTGTCTTCATCTTCCCCCCAAAACCCAAGGACACC
CTCATGATCTCACGCACCCCCGAGGTCACATGCGTGGTGGTGGACGTGAGCCAGGA
TGACCCCGAGGTGCAGTTCACATGGTACATAAACAACGAGCAGGTGCGCACCGCCC
GGCCGCCGCTACGGGAGCAGCAGTTCAACAGCACGATCCGCGTGGTCAGCACCCTC
CCCATCGCGCACCAGGACTGGCTGAGGGGCAAGGAGTTCAAGTGCAAAGTCCACAA
CAAGGCACTCCCGGCCCCCATCGAGAAAACCATCTCCAAAGCCAGAGGGCAGCCCC
TGGAGCCGAAGGTCTACACCATGGGCCCTCCCCGGGAGGAGCTGAGCAGCAGGTCG
GTCAGCCTGACCTGCATGATCAACGGCTTCTACCCTTCCGACATCTCGGTGGAGTGG
GAGAAGAACGGGAAGGCAGAGGACAACTACAAGACCACGCCGACCGTGCTGGACA
GCGACGGCTCCTACTTCCTCTACAGCAAGCTCTCAGTGCCCACGAGTGAGTGGCAGC
GGGGCGACGTCTTCACCTGCTCCGTGATGCACGAGGCCTTGCACAACCACTACACGC
AGAAGTCCATCTCCCGCTCTCCGGGTAAATAG (SEQ ID NO: 89)
A11B1_5A7 Light Chain
ATGGACACGAGGGCCCCCACTCAGCTGCTGGGGCTCCTGCTGCTCTGGCTCCCAGGT
GCCAGATGTGATGTTGTGATGACCCAGACTCCAGCCTCCGTGTCTGAACCTGTGGGA
GGCGCAGTCACCATCAAGTGCCAGGCCAGTCAGAGCATTGGTAGTAATTTAGCCTG
GTATCAGCACAAACCAGGGCAGCCTCCCAAGCTCCTGATCTATTTTGCATCCAGCCT
GGCATCTGGGGTCTCGTCGCGGTTCAAGGGCGGTAGATCTGGGACACAGTTCACTCT
CACCATCAGCGACCTGGAGTGTGCCGATGCTGCCACTTACTACTGTCACTGTACTTA
TTATCCTCTTAGTTATGTTACTTTCGGCGGAGGGACCGAGGTGGTGGTCAAAGGTGA
TCCAGTTGCACCTACTGTCCTCATCTTCCCACCAGCTGCTGATCAGGTGGCAACTGG
AACAGTCACCATCGTGTGTGTGGCGAATAAATACTTTCCCGATGTCACCGTCACCTG
GGAGGTGGATGGCACCACCCAAACAACTGGCATCGAGAACAGTAAAACACCGCAG
AATTCTGCAGATTGTACCTACAACCTCAGCAGCACTCTGACACTGACCAGCACACAG
TACAACAGCCACAAAGAGTACACCTGCAAGGTGACCCAGGGCACGACCTCAGTCGT
CCAGAGCTTCAATAGGGGTGACTGTTAG (SEQ ID NO: 90)
A11B1_4E1 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGACATCCCTGACACT
CTCCTGCACAGCCTCTGGATTCTCCTTCGGTAGCTATTATTATATGTGCTGGGTCCGC
CAGGCTCCAGGGAAGGGGCTGGAGTGGATCGCATGCATTGATGTTGGTAGTAGTGG
TGACACATACTACGCGAGCTGGGTGAATGGCCGATTCACCATCTCCAAAACCTCGTC
GACCACGGTGACTCTGCAAATGACCAGTCTGACAGCCGCGGACACGGCCACCTATT
TCTGTGCGAGAGATGATACTGCTGCTGGTGGTTTTGGTAATTTGGAATTGTGGGGCC
CAGGCACCCTGGTCACCGTCTCCTCAGGGCAACCTAAGGCTCCATCAGTCTTCCCAC
TGGCCCCCTGCTGCGGGGACACACCCAGCTCCACGGTGACCCTGGGCTGCCTGGTCA
AAGGCTACCTCCCGGAGCCAGTGACCGTGACCTGGAACTCGGGCACCCTCACCAAT
GGGGTACGCACCTTCCCGTCCGTCCGGCAGTCCTCAGGCCTCTACTCGCTGAGCAGC
GTGGTGAGCGTGACCTCAAGCAGCCAGCCCGTCACCTGCAACGTGGCCCACCCAGC
CACCAACACCAAAGTGGACAAGACCGTTGCGCCCTCGACATGCAGCAAGCCCATGT
GCCCACCCCCTGAACTCCCGGGGGGACCGTCTGTCTTCATCTTCCCCCCAAAACCCA
AGGACACCCTCATGATCTCACGCACCCCCGAGGTCACATGCGTGGTGGTGGACGTG
AGCCAGGATGACCCCGAGGTGCAGTTCACATGGTACATAAACAACGAGCAGGTGCG
CACCGCCCGGCCGCCGCTACGGGAGCAGCAGTTCAACAGCACGATCCGCGTGGTCA
GCACCCTCCCCATCGCGCACCAGGACTGGCTGAGGGGCAAGGAGTTCAAGTGCAAA
GTCCACAACAAGGCACTCCCGGCCCCCATCGAGAAAACCATCTCCAAAGCCAGAGG
GCAGCCCCTGGAGCCGAAGGTCTACACCATGGGCCCTCCCCGGGAGGAGCTGAGCA
GCAGGTCGGTCAGCCTGACCTGCATGATCAACGGCTTCTACCCTTCCGACATCTCGG
TGGAGTGGGAGAAGAACGGGAAGGCAGAGGACAACTACAAGACCACGCCGACCGT
GCTGGACAGCGACGGCTCCTACTTCCTCTACAGCAAGCTCTCAGTGCCCACGAGTGA
GTGGCAGCGGGGCGACGTCTTCACCTGCTCCGTGATGCACGAGGCCTTGCACAACC
ACTACACGCAGAAGTCCATCTCCCGCTCTCCGGGTAAATAG (SEQ ID NO: 91)
A11B1_4E1 Light Chain
ATGGACACGAGGGCCCCCACTCAGCTGCTGGGGCTCCTGCTGCTCTGGCTCCCAGGT
GCCAGATGTGCATTCGAATTGACCCAGACTCCATCCTCCGTGTCTGAACCTGTGGGA
GGCACAGTCACCATCAAGTGCCAGGCCAGTCAGAGCATTTACAGCTACTTTTCCTGG
TATCAGCAGAAACCAGGGCAGCCTCCCAAGCGCCTGATTTACCAGGCATCCACTCTG
GCTTCTGGGGTCCCATCGCGGTTCAAAGGCAGTGGATCTGGGACAGATTTCACTCTC
ACCATCAGCGACCTGGAGTGTGCCGATGCTGCCACTTACTACTGTCAAAACAATTAT
GGTAGGGGTAGTGGTAGTTATTTTTTTGGTTTCGGCGGAGGGACCGAGGTGGTGGTC
AAAGGTGATCCAGTTGCACCTACTGTCCTCATCTTCCCACCAGCTGCTGATCAGGTG
GCAACTGGAACAGTCACCATCGTGTGTGTGGCGAATAAATACTTTCCCGATGTCACC
GTCACCTGGGAGGTGGATGGCACCACCCAAACAACTGGCATCGAGAACAGTAAAAC
ACCGCAGAATTCTGCAGATTGTACCTACAACCTCAGCAGCACTCTGACACTGACCAG
CACACAGTACAACAGCCACAAAGAGTACACCTGCAAGGTGACCCAGGGCACGACCT
CAGTCGTCCAGAGCTTCAATAGGGGTGACTGTTAG (SEQ ID NO: 92)
A11B1_3H9 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCAAAGCCTCTGGAATCGACTTCAGTAGCGGCTACGGCATGTGGTGGGTCCG
CCAGGCTCCAGGGAAGGGACTGGAGTATATCGGATACATTGATACTGGTAGTGGTA
GCACTTACTACGCGAACTGGGCGAAAGGCCGATTCACCATCTCCAAAACCTCGTCG
ACCATGGTGACTCTGCAAATGACCAGTCTGACAGTCGCGGACACGGCCACCTATTTC
TGTGCGAAAGGGGGCGCCATAGACCTCTGGGGCCCAGGGACCCTCGTCACCGTCTC
TTCAGGGCAACCTAAGGCTCCATCAGTCTTCCCACTGGCCCCCTGCTGCGGGGACAC
ACCCAGCTCCACGGTGACCCTGGGCTGCCTGGTCAAAGGCTACCTCCCGGAGCCAGT
GACCGTGACCTGGAACTCGGGCACCCTCACCAATGGGGTACGCACCTTCCCGTCCGT
CCGGCAGTCCTCAGGCCTCTACTCGCTGAGCAGCGTGGTGAGCGTGACCTCAAGCA
GCCAGCCCGTCACCTGCAACGTGGCCCACCCAGCCACCAACACCAAAGTGGACAAG
ACCGTTGCGCCCTCGACATGCAGCAAGCCCATGTGCCCACCCCCTGAACTCCCGGGG
GGACCGTCTGTCTTCATCTTCCCCCCAAAACCCAAGGACACCCTCATGATCTCACGC
ACCCCCGAGGTCACATGCGTGGTGGTGGACGTGAGCCAGGATGACCCCGAGGTGCA
GTTCACATGGTACATAAACAACGAGCAGGTGCGCACCGCCCGGCCGCCGCTACGGG
AGCAGCAGTTCAACAGCACGATCCGCGTGGTCAGCACCCTCCCCATCGCGCACCAG
GACTGGCTGAGGGGCAAGGAGTTCAAGTGCAAAGTCCACAACAAGGCACTCCCGGC
CCCCATCGAGAAAACCATCTCCAAAGCCAGAGGGCAGCCCCTGGAGCCGAAGGTCT
ACACCATGGGCCCTCCCCGGGAGGAGCTGAGCAGCAGGTCGGTCAGCCTGACCTGC
ATGATCAACGGCTTCTACCCTTCCGACATCTCGGTGGAGTGGGAGAAGAACGGGAA
GGCAGAGGACAACTACAAGACCACGCCGACCGTGCTGGACAGCGACGGCTCCTACT
TCCTCTACAGCAAGCTCTCAGTGCCCACGAGTGAGTGGCAGCGGGGCGACGTCTTCA
CCTGCTCCGTGATGCACGAGGCCTTGCACAACCACTACACGCAGAAGTCCATCTCCC
GCTCTCCGGGTAAATAG (SEQ ID NO: 93)
A11B1_3H9 Light Chain
ATGGACACGAGGGCCCCCACTCAGCTGCTGGGACTCCTACTGCTCTGGCTCCCAGGT
GCCAGATGTGCTGACATTGTGATGACCCAGACTCCAGCCTCCGTGGAGGCAGCTGTG
GGAGGCACAGTCACCATCAAGTGCCAGGCCAGTCAGAGCATTAGTAGTTACTTAGC
CTGGTATCAGCAGAAACCAGGGCAGCGTCCCAAGCTCCTGATCTACAGGGCATCCA
CTCTGGCATCTGGGGTCCCATCGCGGTTCAAAGGCAGTGGATCTGGGACAGACTACA
CTCTCACCATCAGCGACCTGGAGTGTGCCGATGCTGCCACTTATTATTGTCATACCTA
TTATCTTAGTAGTAGTATCAGTTATGGTAATACTTTCGGCGGAGGGACCGAGGTGGT
GGTCAAAGGTGATCCAGTTGCACCTACTGTCCTCATCTTCCCACCAGCTGCTGATCA
GGTGGCAACTGGAACAGTCACCATCGTGTGTGTGGCGAATAAATACTTTCCCGATGT
CACCGTCACCTGGGAGGTGGATGGCACCACCCAAACAACTGGCATCGAGAACAGTA
AAACACCGCAGAATTCTGCAGATTGTACCTACAACCTCAGCAGCACTCTGACACTGA
CCAGCACACAGTACAACAGCCACAAAGAGTACACCTGCAAGGTGACCCAGGGCACG
ACCTCAGTCGTCCAGAGCTTCAATAGGGGTGACTGTTAG (SEQ ID NO: 94)
A11B1_3G2 Heavy Chain
ATGGAGACTGGGCTGCGCTGGCTTCTCCTGGTCGCTGTGCTCAAAGGTGTCCAGTGT
CAGGAGCAGCTGGAGGAGTCCGGGGGAGACCTGGTCAAGCCTGAGGGATCCCTGAC
ACTCACCTGCAAAGCCTCTGGATTCTCCTTCAGTAGCATCTACTGGATTTGCTGGGTC
CGCCAGGCTCCAGGGAAGGGGCTGGAGTGGATCGCATGCACTACTGTTGTCAAAAG
TGGTAGAACTTACTACGCGAACTGGGCGAAAGGCCGATTCACCATCTCCAAAACCT
CGTCGACCACGGTGACTCTGCAAATGACCAGTCTGACAGCCGCGGACACGGCCACC
TATTTCTGTGCGAGAGAATTTGTTGATGGTGGTGGTAGTAGTGGTAGGGACTTGTGG
GGCCCAGGCACCCTGGTCACCGTCTCCTCAGGGCAACCTAAGGCTCCATCAGTCTTC
CCACTGGCCCCCTGCTGCGGGGACACACCCAGCTCCACGGTGACCCTGGGCTGCCTG
GTCAAAGGCTACCTCCCGGAGCCAGTGACCGTGACCTGGAACTCGGGCACCCTCAC
CAATGGGGTACGCACCTTCCCGTCCGTCCGGCAGTCCTCAGGCCTCTACTCGCTGAG
CAGCGTGGTGAGCGTGACCTCAAGCAGCCAGCCCGTCACCTGCAACGTGGCCCACC
CAGCCACCAACACCAAAGTGGACAAGACCGTTGCGCCCTCGACATGCAGCAAGCCC
ATGTGCCCACCCCCTGAACTCCCGGGGGGACCGTCTGTCTTCATCTTCCCCCCAAAA
CCCAAGGACACCCTCATGATCTCACGCACCCCCGAGGTCACATGCGTGGTGGTGGA
CGTGAGCCAGGATGACCCCGAGGTGCAGTTCACATGGTACATAAACAACGAGCAGG
TGCGCACCGCCCGGCCGCCGCTACGGGAGCAGCAGTTCAACAGCACGATCCGCGTG
GTCAGCACCCTCCCCATCGCGCACCAGGACTGGCTGAGGGGCAAGGAGTTCAAGTG
CAAAGTCCACAACAAGGCACTCCCGGCCCCCATCGAGAAAACCATCTCCAAAGCCA
GAGGGCAGCCCCTGGAGCCGAAGGTCTACACCATGGGCCCTCCCCGGGAGGAGCTG
AGCAGCAGGTCGGTCAGCCTGACCTGCATGATCAACGGCTTCTACCCTTCCGACATC
TCGGTGGAGTGGGAGAAGAACGGGAAGGCAGAGGACAACTACAAGACCACGCCGA
CCGTGCTGGACAGCGACGGCTCCTACTTCCTCTACAGCAAGCTCTCAGTGCCCACGA
GTGAGTGGCAGCGGGGCGACGTCTTCACCTGCTCCGTGATGCACGAGGCCTTGCAC
AACCACTACACGCAGAAGTCCATCTCCCGCTCTCCGGGTAAATAG (SEQ ID NO: 95)
A11B1_3G2 Light Chain
ATGGACACGAGGGCCCCCACTCAGCTGCTGGGGCTCCTGCTGCTCTGGCTCCCAGGT
GCCAGATGTGCCTATGATATGACCCAGACTCCAGCCTCCGTGGAGGCAGCTGTGGG
AGGCACAGTCACCATCAAGTGCCAGGCCAGTCAGAGCATTAGTAGGGACTTATCCT
GGTATCAGCAGAAACCTGGACAGCCTCCCAAGCGCCTAATCTACAAGGCATCCACT
CTGGCATCTGGGGTCCCATCGCGGTTCAAAGGCAGTGGATCTGGGACAGATTTCACT
CTCACCATCAGCGACCTGGAGTGTGCCGATGCTGCCACTTACTACTGTCAACAGGGT
TATAGTAGTATTGATGTTGATAATGATTTCGGCGGAGGGACCGAGGTGGTGGTCAAA
GGTGATCCAGTTGCACCTACTGTCCTCATCTTCCCACCAGCTGCTGATCAGGTGGCA
ACTGGAACAGTCACCATCGTGTGTGTGGCGAATAAATACTTTCCCGATGTCACCGTC
ACCTGGGAGGTGGATGGCACCACCCAAACAACTGGCATCGAGAACAGTAAAACACC
GCAGAATTCTGCAGATTGTACCTACAACCTCAGCAGCACTCTGACACTGACCAGCAC
ACAGTACAACAGCCACAAAGAGTACACCTGCAAGGTGACCCAGGGCACGACCTCAG
TCGTCCAGAGCTTCAATAGGGGTGACTGTTAG (SEQ ID NO: 96)
A11B1_3B1 Heavy Chain
ATGGAGACTGGGCTGCGCTGGCTTCTCCTGGTCGCTGTGCTCAAAGGTGTCCAGTGT
CAGGAGCAGCTGGAGGAGTCCGGGGGAGGCCTGGTCAAGCCTGAGGGATCCCTGAC
ACTCACCTGCAAAGCCTCTGGATTCGACCTCAGTAGCGGCTATGACATGTGCTGGGT
CCGCCAGGCTCCAGGGAAGGGGCTGGAGTGGATCGCATGCATTTATGCTGATTATA
GTGGTAGCACATACTACGCGAGCTGGGTGAATGGCCGATTCACCATCTCCAGCAGC
ACCAGCCTAAACACGGTGGATCTGAAAATGACCAGTCTGACAGCCGCGGACACGGC
CACCTATTTCTGTGCCAGAGGGGCTACTGGTAATGGTGGTTATGGATACTACTTTAA
CTTGTGGGGCCCAGGCACCCTGGTCACCGTCTCCTCAGGGCAACCTAAGGCTCCATC
AGTCTTCCCACTGGCCCCCTGCTGCGGGGACACACCCAGCTCCACGGTGACCCTGGG
CTGCCTGGTCAAAGGCTACCTCCCGGAGCCAGTGACCGTGACCTGGAACTCGGGCA
CCCTCACCAATGGGGTACGCACCTTCCCGTCCGTCCGGCAGTCCTCAGGCCTCTACT
CGCTGAGCAGCGTGGTGAGCGTGACCTCAAGCAGCCAGCCCGTCACCTGCAACGTG
GCCCACCCAGCCACCAACACCAAAGTGGACAAGACCGTTGCGCCCTCGACATGCAG
CAAGCCCATGTGCCCACCCCCTGAACTCCCGGGGGGACCGTCTGTCTTCATCTTCCC
CCCAAAACCCAAGGACACCCTCATGATCTCACGCACCCCCGAGGTCACATGCGTGG
TGGTGGACGTGAGCCAGGATGACCCCGAGGTGCAGTTCACATGGTACATAAACAAC
GAGCAGGTGCGCACCGCCCGGCCGCCGCTACGGGAGCAGCAGTTCAACAGCACGAT
CCGCGTGGTCAGCACCCTCCCCATCGCGCACCAGGACTGGCTGAGGGGCAAGGAGT
TCAAGTGCAAAGTCCACAACAAGGCACTCCCGGCCCCCATCGAGAAAACCATCTCC
AAAGCCAGAGGGCAGCCCCTGGAGCCGAAGGTCTACACCATGGGCCCTCCCCGGGA
GGAGCTGAGCAGCAGGTCGGTCAGCCTGACCTGCATGATCAACGGCTTCTACCCTTC
CGACATCTCGGTGGAGTGGGAGAAGAACGGGAAGGCAGAGGACAACTACAAGACC
ACGCCGACCGTGCTGGACAGCGACGGCTCCTACTTCCTCTACAGCAAGCTCTCAGTG
CCCACGAGTGAGTGGCAGCGGGGCGACGTCTTCACCTGCTCCGTGATGCACGAGGC
CTTGCACAACCACTACACGCAGAAGTCCATCTCCCGCTCTCCGGGTAAATAG (SEQ
ID NO: 97)
A11B1_3B1 Light Chain
ATGGACACGAGGGCCCCCACTCAGCTGCTGGGGCTCCTACTGCTCTGGCTCCCAGGT
GCCAGATGTGCTGACATTGTGATGACCCAGACTCCAGCCTCCGTGTCTGAACCTGTG
GGAGGCACAGTCACCATCAAGTGTCAGGCCAGTCAGAACATTAATAGCGGCTTAGC
CTGGTATCAGCAGAAACCAGGGCAGCCTCCCAAGCTCCTGATCTACAAGGCATCCA
CTCTGGCATCTGGGGTCTCATCGCGGTTCAAAGGCAGTGGATCTGGGACAGAATTCA
CTCTCACCATCAGCGACCTGGAGTGTGCCGATGCTGCCACTTACTACTGTCAAACCT
ATTATTATAGTAGTAGTAGTAGTGATAATGCTTTCGGCGGAGGGACCGAGGTGGTGG
TCAAAGGTGATCCAGTTGCACCTACTGTCCTCATCTTCCCACCAGCTGCTGATCAGG
TGGCAACTGGAACAGTCACCATCGTGTGTGTGGCGAATAAATACTTTCCCGATGTCA
CCGTCACCTGGGAGGTGGATGGCACCACCCAAACAACTGGCATCGAGAACAGTAAA
ACACCGCAGAATTCTGCAGATTGTACCTACAACCTCAGCAGCACTCTGACACTGACC
AGCACACAGTACAACAGCCACAAAGAGTACACCTGCAAGGTGACCCAGGGCACGA
CCTCAGTCGTCCAGAGCTTCAATAGGGGTGACTGTTAG (SEQ ID NO: 98)
A11B1_2D3 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCACAGCTTCTGGATTCTCCTTCAGTAGCAGTTATTGGATATGCTGGGTCCGC
CAGGCTCCAGGGAAGGGGCTGGAGTGGATCGCATGTATTTATGGTGGTAGTAGTGG
TAACATTGCCTACGCGAGCTGGGCGAAAGGCCGATTCACCATCTCCAAAACCTCGTC
GACCACGGTGACTCTACAAATGACCAGTCTGACAGCCGCGGACACGGCCACCTATT
TCTGTGCGAGAGATATTCCTAGTGATGCTTTCACCTTAGACTTGTGGGGCCCAGGCA
CCCTGGTCACCGTCTCCTCAGGGCAACCTAAGGCTCCATCAGTCTTCCCACTGGCCC
CCTGCTGCGGGGACACACCCAGCTCCACGGTGACCCTGGGCTGCCTGGTCAAAGGC
TACCTCCCGGAGCCAGTGACCGTGACCTGGAACTCGGGCACCCTCACCAATGGGGT
ACGCACCTTCCCGTCCGTCCGGCAGTCCTCAGGCCTCTACTCGCTGAGCAGCGTGGT
GAGCGTGACCTCAAGCAGCCAGCCCGTCACCTGCAACGTGGCCCACCCAGCCACCA
ACACCAAAGTGGACAAGACCGTTGCGCCCTCGACATGCAGCAAGCCCATGTGCCCA
CCCCCTGAACTCCCGGGGGGACCGTCTGTCTTCATCTTCCCCCCAAAACCCAAGGAC
ACCCTCATGATCTCACGCACCCCCGAGGTCACATGCGTGGTGGTGGACGTGAGCCA
GGATGACCCCGAGGTGCAGTTCACATGGTACATAAACAACGAGCAGGTGCGCACCG
CCCGGCCGCCGCTACGGGAGCAGCAGTTCAACAGCACGATCCGCGTGGTCAGCACC
CTCCCCATCGCGCACCAGGACTGGCTGAGGGGCAAGGAGTTCAAGTGCAAAGTCCA
CAACAAGGCACTCCCGGCCCCCATCGAGAAAACCATCTCCAAAGCCAGAGGGCAGC
CCCTGGAGCCGAAGGTCTACACCATGGGCCCTCCCCGGGAGGAGCTGAGCAGCAGG
TCGGTCAGCCTGACCTGCATGATCAACGGCTTCTACCCTTCCGACATCTCGGTGGAG
TGGGAGAAGAACGGGAAGGCAGAGGACAACTACAAGACCACGCCGACCGTGCTGG
ACAGCGACGGCTCCTACTTCCTCTACAGCAAGCTCTCAGTGCCCACGAGTGAGTGGC
AGCGGGGCGACGTCTTCACCTGCTCCGTGATGCACGAGGCCTTGCACAACCACTACA
CGCAGAAGTCCATCTCCCGCTCTCCGGGTAAATAG (SEQ ID NO: 99)
A11B1_2D3 Light Chain
ATGGACACGAGGGCCCCCACTCAGCTGCTGGGGCTCCTGCTGCTCTGGCTCCCAGGT
GCCACATTTGCCCAAGTGCTGACCCAGACTCCATCCTCCGTGTCTGCAGCTGTGGGA
AGCACAGTCACCATCAATTGCCAGGCCAGTCAGAGTGTTTATAAAGACAACAATTTA
GCCTGGTATCAGCAGAAACCAGGGCAGCCTCCCAAGCTCCTGATCTACAAGGCTTCC
ACTCTGGCATCTGGGGTCCCATCGCGGTTCAAAGGCAGTGGATCTGGGACACAGTTC
ACTCTCACCATCAGCGGCGTGCAGTGTGAAGATGCTGCCACTTACTACTGTCAAGGC
GAATTCAGTTGTGGTAGTGCTGATTGTATTGCTTTCGGCGGAGGGACCGAGGTGGTG
GTCAAAGGTGATCCAGTTGCACCTACTGTCCTCATCTTCCCACCAGCTGCTGATCAG
GTGGCAACTGGAACAGTCACCATCGTGTGTGTGGCGAATAAATACTTTCCCGATGTC
ACCGTCACCTGGGAGGTGGATGGCACCACCCAAACAACTGGCATCGAGAACAGTAA
AACACCGCAGAATTCTGCAGATTGTACCTACAACCTCAGCAGCACTCTGACACTGAC
CAGCACACAGTACAACAGCCACAAAGAGTACACCTGCAAGGTGACCCAGGGCACG
ACCTCAGTCGTCCAGAGCTTCAATAGGGGTGACTGTTAG (SEQ ID NO: 100)
A11B1_2A7 Heavy Chain
ATGGAGACTGGGCTGCGCTGGCTTCTCCTGGTCGCTGTGCTCAAAGGTGTCCAGTGT
CAGTCGTTGGAGGAGTCCGGGGGAGACCTGGTCAAGCCTGGGGCATCCCTGACACT
CACCTGCAAAGGCTCTGGAATCGACTTCAGTAGCGGCTACGGCATGTGGTGGGTCCG
CCAGGCTCCAGGGAAGGGACTGGAGTATATCGGATACATTGATACTGGTTATGGTA
GCACTTACTACGCGAGCTGGGCGAAAGGCCGATTCACCATCTCCAAGACCTCGTCG
ACCACGGTGACTCTGCAAATGACCAGTCTGACAGTCGCGGACACGGCCACCTATTTC
TGTGCGAAAGGGGGCGCCATAGACCTCTGGGGCCCAGGGACCCTCGTCACCGTCTC
TTCAGGGCAACCTAAGGCTCCATCAGTCTTCCCACTGGCCCCCTGCTGCGGGGACAC
ACCCAGCTCCACGGTGACCCTGGGCTGCCTGGTCAAAGGCTACCTCCCGGAGCCAGT
GACCGTGACCTGGAACTCGGGCACCCTCACCAATGGGGTACGCACCTTCCCGTCCGT
CCGGCAGTCCTCAGGCCTCTACTCGCTGAGCAGCGTGGTGAGCGTGACCTCAAGCA
GCCAGCCCGTCACCTGCAACGTGGCCCACCCAGCCACCAACACCAAAGTGGACAAG
ACCGTTGCGCCCTCGACATGCAGCAAGCCCATGTGCCCACCCCCTGAACTCCCGGGG
GGACCGTCTGTCTTCATCTTCCCCCCAAAACCCAAGGACACCCTCATGATCTCACGC
ACCCCCGAGGTCACATGCGTGGTGGTGGACGTGAGCCAGGATGACCCCGAGGTGCA
GTTCACATGGTACATAAACAACGAGCAGGTGCGCACCGCCCGGCCGCCGCTACGGG
AGCAGCAGTTCAACAGCACGATCCGCGTGGTCAGCACCCTCCCCATCGCGCACCAG
GACTGGCTGAGGGGCAAGGAGTTCAAGTGCAAAGTCCACAACAAGGCACTCCCGGC
CCCCATCGAGAAAACCATCTCCAAAGCCAGAGGGCAGCCCCTGGAGCCGAAGGTCT
ACACCATGGGCCCTCCCCGGGAGGAGCTGAGCAGCAGGTCGGTCAGCCTGACCTGC
ATGATCAACGGCTTCTACCCTTCCGACATCTCGGTGGAGTGGGAGAAGAACGGGAA
GGCAGAGGACAACTACAAGACCACGCCGACCGTGCTGGACAGCGACGGCTCCTACT
TCCTCTACAGCAAGCTCTCAGTGCCCACGAGTGAGTGGCAGCGGGGCGACGTCTTCA
CCTGCTCCGTGATGCACGAGGCCTTGCACAACCACTACACGCAGAAGTCCATCTCCC
GCTCTCCGGGTAAATAG (SEQ ID NO: 101)
A11B1_2A7 Light Chain
ATGGACACGAGGGCCCCCACTCAGCTGCTGGGGCTCCTGCTGCTCTGGCTCCCAGGT
GCCACATTTGCAGCCGTGCTGACCCAGACTCCGGCTTCCACGTCTGCAGCTGTGGGA
GGCACAGTCACCATCAATTGTCAGTCCAGTCAGAGCGTGTATCGTAGCAACTGGTTA
GCCTGGTATCAGCAGAAACCAGGGCAGCCTCCCAAGCTCCTGATCTATGATGTATTT
AATTTGGCATCTGGGGTCCCATCCCGGTTCAAGGGCAGTGGATCTGGGACACAGTTC
ACTCTCACCATCAGCGGCGTGCAGTGTGCCGATGCTGCCACTTACTACTGTCAAGGC
AGTTATTATAGTGGTAATTGGTACAGTGCTTTCGGCGGAGGGACCGAGGTGGTGGTC
AAAGGTGATCCAGTTGCACCTACTGTCCTCATCTTCCCACCAGCTGCTGATCAGGTG
GCAACTGGAACAGTCACCATCGTGTGTGTGGCGAATAAATACTTTCCCGATGTCACC
GTCACCTGGGAGGTGGATGGCACCACCCAAACAACTGGCATCGAGAACAGTAAAAC
ACCGCAGAATTCTGCAGATTGTACCTACAACCTCAGCAGCACTCTGACACTGACCAG
CACACAGTACAACAGCCACAAAGAGTACACCTGCAAGGTGACCCAGGGCACGACCT
CAGTCGTCCAGAGCTTCAATAGGGGTGACTGTTAG (SEQ ID NO: 102)
Protein sequences of anti-α11ß1 monoclonal antibodies
Rat mAb sequences
Signal peptide-FR1-CDR1-FR2-CDR2-FR3-CDR3-FR4
79E3E3 Heavy Chain Variable Region
MDWLWNLLFLMVVAQSAQAQIQLVQSGPEVKKPGESVKISCKASGYTFTDYAMNWVKQ
APGNGLKWMGWINTQTGKPTYADDFKQRFVFSLETSARTTYLQINNLNIEDTATYFCT
RLGTGNTKGFAYWGQGTLVTVSS (SEQ ID NO: 145)
79E3E3 Light Chain Variable Region
MESQTHVLISLLLCVSGTCGDILINQSPASLTVSAGERVTMSCKSSQSLLYSENNQDYLA
WYQQKPGQFPKLLIYGASNRHTGVPDRFTGSGSGTDFTLTISSVQAEDLADYYCEQTY
RYPFTFGSGTKLEIK (SEQ ID NO: 146)
24E4G6 Heavy Chain Variable Region
MELELSLIFIFSLLKDVQCEVQLVESGGSLVQPGGSLKLSCVASGYTFSNYWMDWVRQSP
GKSLEWIGEINTDGRRTNYAPSIKDRFTISRDNAKSTLYLQMSNVKSDDTAIYYCTILR
VYPHYFDYWGQGVMVTVSS (SEQ ID NO: 147)
24E4G6 Light Chain Variable Region
MMSPAQFLFLLMLWIQEARGDVVMTQTPPSLSVAIGQSVSISCKSSQSLVYSDGETYLH
WFLQSPGRSPKRLIYHVSNLGSGVPDRFSGTGSLTDFTLRISRVEAEDLGVYYCAQTTH
FPPTFGAGTKLELK (SEQ ID NO: 148)
8H8E9 Heavy Chain Variable Region
MAVLVLLLCLVTFPSCALSQVQLKESGPGLVQPSQTLSLTCTVSGFSLTSNSVSWVRQAPG
KGLEWMGAIWSGGSTDYNSALKSRLSISRDTSKSQVFLKMNSLQTEDTAIYFCTRSHW
EPFDYWGQGVMVTVSS (SEQ ID NO: 149)
8H8E9 Light Chain Variable Region
MESQTQALISLLLWVYGTCGDIVMTQSPFSLAVSEGEMVTINCKSSQGLLSSGNQKNYLA
WYQQRPGQSPKLLIYYASTRQSGVPDRFIGGGSGTDFTLTISDVQAEDLADYYCLQHYS
YPPTFGSGTKLEIK (SEQ ID NO: 150)
6E5C11 Heavy Chain Variable Region
MAVLVLLLCLVTFPSCALSQVQLRESGPGLVQPSQTLSLTCTVSGFSLTSNSVTWVRQPPG
KGLEWMGAIWSDGSTDYNSTLKSRLSISRDTSKSQVFLKMSSLQTEDTAIYFCTRSHW
EPFDYWGQGVMVTVSS (SEQ ID NO: 151)
6E5C11 Light Chain Variable Region
MESQTQALISLLLWVYGTCGDIVMTQSPLSLAVSEGETVTMNCKSSQSLFSSGNQKNYLA
WYQQKPGQSPKLLIYYASTRQSGVPDRFIGSGSGTDFTLTISDVQTEDLADYYCLQHYN
YPPTFGSGTKLEIK (SEQ ID NO: 152)
7D8B10 Heavy Chain Variable Region
MDLRLTYVFIVAILKGVLCEVKLEESGGGLVQPGMSVKLSCATSGFIFSDYWMEWVRQAP
GKGLEWVAEIRNKANNYATYYGKSMKGRFTISRDDSKSIVYLQVNSIRSEDTAIYYCA
PNFDYWGQGVMVTVSS (SEQ ID NO: 153)
7D8B10 Light Chain Variable Region
MSPVQSLFLLLLWILGTHGDVVLTQTPPTLSATIGQSVSISCRSSQSLLHSTGNTYLNWLL
QRPGQPPQLLIYLVSRLESGVPNRFSASGSGTDFTLKISGIEAEDLGVYYCVQSSHTPYT
FGTGTKLELK (SEQ ID NO: 154)
18E10F10 Heavy Chain Variable Region
MDIRLSLVFLVLFMKGVQCEVQLVESGGGLVQPGRSLKLSCAASRFTFSDYNMAWVRQA
PKKGLEWVATIYHDDSGSYYRDSVKGRFTISRNNAKSTLYLQMDSLRSEDMATYYCA
RHNNGFDYWGQGVMVTVAS (SEQ ID NO: 155)
18E10F10 Light Chain Variable Region
MKWPVRLLVLFFWIPASGGDVVMTQTPVSLPVRLGGQASISCRSSQSLVHSNGNTYLHW
YLQKPGQSPQLLINRVSNRFSGVPDRFSGSGSGTDFTLKINRVEPEDLGDYYCLQSTHFP
LTFGSGTKLETK (SEQ ID NO: 156)
40G10H11 Heavy Chain Variable Region
MDIRLSLGFLVLFIKGDQCAVQLVESGGGLVQPGRSLKLSCAASRITFTDYYMAWVRQA
PTKGLEWVATISSDGGDTFYRDSVKGRFTISRDNAKSTLYLQMVSLRSEDTATYYCST
DRGAQFGYWGQGTLVTVSS (SEQ ID NO: 157)
40G10H11 Light Chain Variable Region
MAPVqLLGLLLIWLPAMRCDIQMTQSPSFLSASVGDRVSINCKASQNVHENLNWYQQKL
GEAPKRLIYNTNNLQTGIPSRFSGSGSGADYTLTISSLQPEDFATYFCLQHNAFPYTFGP
GTKLELK (SEQ ID NO: 158)
Mouse mAb sequences
FR1-CDR1-FR2-CDR2-FR3-CDR3-FR4
9-G05 Heavy Chain Variable Region
EVQLQQSGPELVKPGASVKIPCKASGYTFPDYNMDWVKQSHGKSLEWIGYINPDNGGT
IYNQKFKGKATLTVDKSSSTAYMELRSLTSEDTAVYYCARLDSSGYGYYAMDYWGQG
TSVTVSS (SEQ ID NO: 103)
9-G05 Light Chain Variable Region
DIVLTQSPASLAVSLGQRATISCRASESVDNYGISFMHWYQQKPGQPPKLLIYRASNLD
SEIPARFSGSGSRTDFTLTIDPVETDDVATYYCQQSYKDPRTFGGGTKLEIK (SEQ ID
NO: 104)
8-P20 Heavy Chain Variable Region
KVMLVESGGALVKPGGSLKLSCVASGFTFSNYAMSWVRQTPEKRLEWVATISSGGYY
TYYPDSVKGRFTISRDNARNTLFLQMSSLRSEDTAMFYCAREDDYGRYSYTMDYWGQ
GTSVTVSS (SEQ ID NO: 105)
8-P20 Light Chain Variable Region
DVVMTQTPLSLPVSLGDQVSISCRCSQSLVHSNGNTYLHWYLQKPGQSPQLLIYKISNR
FSGVPDRFSGSGSGTDFTLKISRVEAEDLGVYFCSQSTHVPYTFGGGTELEIK (SEQ ID
NO: 106)
8-G15 Heavy Chain Variable Region
EVQLQQSGPELVKPGASVRISCKASGYTFTDYYIHWVKQKPGQGLECIGEIYPGTDNTY
YSKKFRGKATLTADKSSDTAYMQLSSLTSEDSAVYFCARGDYYRGYFDVWGAGTTVT
VSS (SEQ ID NO: 107)
8-G15 Light Chain Variable Region
DVVMTQTSLTLSVTIGQPASISCKSSQSLLHSNGKTYLNWLLQRPGQSPKFLIYLVSKL
ESGVPDRFSGSGSGTDFTLKISRVEAEDLGVYYCLQSTHFPWTFGGGTKLEIK (SEQ ID
NO: 108)
8-I14 Heavy Chain Variable Region
EVQLQQSGPELVKPGASVKKSCKASGYTFTDYYMHWVKQKPGQGLEWIGKIYPGSGN
THYNEKFKGKATLTADKSSSTAYMQLSSLTSEDSAVYFCATNYYGYRAMNYWGQGSS
VTVSS (SEQ ID NO: 109)
8-I14 Light Chain Variable Region
DIHLTQSPSSLSASLGERISLTCRASQDIYISLNWFQQKPDGTIKLLIYGTSSLDSGVPKRF
SGSRSGSDYSLTISSLESEDFADYYCLQYASSPYTFGGGTKLEIK (SEQ ID NO: 110)
9-E16 Heavy Chain Variable Region
EVQLQQSGPELVKPGASVKISCKASGYTFTDYYMHWVKQKPGQGLEWIGEIYPGSGNP
YYNEKFKGKATLTADKSSSSAYMQLSSLTSEDSAVYFCARTSYGRVGTGFAYWGQGT
LVTVSA (SEQ ID NO: 111)
9-E16 Light Chain Variable Region
NFVMTQTPLSLPVSLGDQASISCRSSQSLLHSNGNTYLHWYLQKPGQSPKLLIYKVSNR
FSGVPDRFSGSGSGTDFTLKINRVETEDLGIYFCSQSSHVPTFGAGTKLELK (SEQ ID
NO: 112)
8-J17 Heavy Chain Variable Region
QVQLQQSGAELAKPGASVKMSCKASGYTFTNYWMHWVKQRPGQGLEWIGYINPNNG
YTEYNQRFKDKATLTADRSSTTAYMQLSSLTSEDSAVYYCARSDIITTDYWGQGTTLT
VSS (SEQ ID NO: 113)
8-J17 Light Chain Variable Region
DVVMTQTPLSLPVSLGDQASISCRSSQSLVYSNGNTYLHWYLQKPGQSPKLLIYKVSNR
FSGVPDRFSGSGSGTDFTLKISRVEAEDLGVYFCSQSTHVPWTFGGGTKLEIK (SEQ ID
NO: 114)
9-B11 Heavy Chain Variable Region
QVQLQQPGAELVRPGTSVKLSCKASGYTFTSYWMHWVQQRPGQGLEWIGVIDPSDSY
TNYNQKFKGKATLTVDTSSSSAYMQLSSLTSEDSAVYYCARDDVAMDYWGQGTSVTV
SS (SEQ ID NO: 115)
9-B11 Light Chain Variable Region
DIVVTQSPASLAVSLGQRATISCRASESVDSYGNSFIHWYQQKPGQPPKLLIYRASNLKS
GIPARFSGSGSRTDFTLTINPVEADDVATYYCQQSNEDPYTFGGGTKLEIK (SEQ ID NO:
116)
For SEQ ID NO: 117-144, CDR3 is represented by bold and underlined text.
6-O12 Heavy Chain Variable Region
EVKLEESGGGLVQPGGSMKLSCAASGFTFSDAWMDWVRQSPEAGLEWVAEIRNKAHN
PATYYAESVKGRFTISRDDSKSSVYLQMNSLRAEDTGIYYCTLVAPDAMDYWGQGTS
VTVSS (SEQ ID NO: 117)
6-012 Light Chain Variable Region
DIVMSLSPSSLAVSVGEKVTMSCKSSQSLLYSRNQKNYLAWYQQKPGQSPKLLIYWAST
RASGVPDRFTGSGSGTDFTLTISSVKAEDLAVYYCQQYYSYPYTFGGGTKLEIK (SEQ
ID NO: 118)
10-L15 Heavy Chain Variable Region
QVQLQQSGPELVRPGASVKMSCKASGYTFTSYWMHWVKQRPGQGLEWIGMIDPSNSE
TWLNQKFKDKATLNVDKSSNTAYMQLSSLTSEDSAVYYCARYDGYYDYWGQGTTLT
VSS (SEQ ID NO: 119)
10-L15 Light Chain Variable Region
NIVLTQSPASLAVSLGQRATISCRASESVDSYGNSFMHWYQQKPGQPPKLLIYLASNVES
GVPARFSGSGSRTDFTLTIDPVEADDAATYYCQQNNEDPWTFGGGTKLEIK (SEQ ID
NO: 120)
7-H14 Heavy Chain Variable Region
QVQLQQPGAELVRPGASVKLSCKPSGYTFTSYWMNWVKQRPGQGLEWIGMIDPSDSET
HYNQMFKDKATLTVDKSSNTAYMQLSSLTSEDSAVYYCAQIYYAYDKAYWGQGTLV
TVSA (SEQ ID NO: 121)
7-H14 Light Chain Variable Region
DIVMSQSPSSLAVSVGEKVTMSCKSSQSLLYSSHQKNYLAWYQQKPGQSPKLLIYWAST
RESGVPDRFTGSGSGTDFSLTISSVKAEDLAVYYCQEYYSWTFGGGTKLEIK (SEQ ID
NO: 122)
6-B21 Heavy Chain Variable Region
EVQLQQSGPELVKPGASVKISCKASGYTFTDYYMNWVKQSHGKSLEWIGDINPHNGGT
SFIQKFKGKATLTVDKSSSTAYMELRSLTSEDSAVYYCAPLGRKEGFAYWGQGTLVTV
SA (SEQ ID NO: 123)
6-B21 Light Chain Variable Region
DTVLTQSPASLVVSLGQRATISCRASKSVSTSGYSYMHWYQQKPGQPPKLLIYLASNLES
GVPARFSGSGSGTAFTLNIHPVEEEDAATYYCQHSRELPYTFGGGTKLEIK (SEQ ID
NO: 124)
10-F23 Heavy Chain Variable Region
QVTLKESGPGILQPSQTLSLTCSFSGFSLSTFAMGVGWIRQPSGKGLEWLAHIWWDDDK
YYNPALKSRLTISKDTSKNHVFLKIANVDTADTATYYCARMPLTFYFDYWGQGTTLTV
SS (SEQ ID NO: 125)
10-F23 Light Chain Variable Region
DVLLTQTPLSLPVSLGDQASISCRSSQSIVHSNGHTYLEWYLQKPGQSPKLLIYKVSNRFS
GVPDRFSGSGSGTDFTLKISRVEAEDLGVYYCFQGSHVPFTFGGGTKLEIK (SEQ ID
NO: 126)
6-A12 Heavy Chain Variable Region
QVTLKESGPGILQPSQTLSLTCSFSGFSLRTFAMGVGWIRQPSGKGLEWLAHIWWDDDK
YYNPALKSRLTISKDTSKNQVFLKIANVDTADTATYYCARMPLTFYFDYWGQGTTLTV
SS (SEQ ID NO: 127)
6-A12 Light Chain Variable Region
DVLMTQTPLSLPVSLGDQASISCRSSQSIVHSNGNTYLEWYLQKPGQSPKLLIYKVSTRF
SGVPDRFSGSGSGTDFTLKISRVEAEDLGVYYCFQGSHVPFTFGGGTKLEIK (SEQ ID
NO: 128)
6-M8 Heavy Chain Variable Region
QVQLQQPGAELVMPGASVKLSCKASGYTFTNYWMHWVKQRPGQGLEWIGEIDPSDSY
TNYNQKFKGKATLTVDKSSSTAYMQLSSLTSEDSAVYYCTRQGSTYAWGQGTSVTVS
S (SEQ ID NO: 129)
6-M8 Light Chain Variable Region
DIVMTQAAFSNPVTLGTSASISCRSSKSLLHSNGITYLYWYLQKPGQSPQLLIYQMSNLA
SGVPDRFSSSGSGTDFTLRISRVEAEDVGVYYCAQNLELPPTFGGGTKLEIK (SEQ ID
NO: 130)
2-A3 Heavy Chain Variable Region
EVQLQQSGPELVKPGASVKMSCKASGYTFTDYYMMWVKQSHGKSLEWIGDINPYNGG
SSYNPKFKGRATLTVDKSSSTAYMQLNSLTSEDSAVYYCARGTYWGQGTLVTVSA
(SEQ ID NO: 131)
2-A3 Light Chain Variable Region
DVVMTQTPLTLSVTIGQPASISCKSSQSLLDSAGKTYLNWLLQRPGQSPKRLMYLVSKL
DSGVPDRFTGSGSGTDFTLKISRVEAEDLGVYYCWQGTHFPYTFGGGTKLEIK (SEQ
ID NO: 132)
6-017 Heavy Chain Variable Region
QVQLQQPGAELVKPGASVKLSCKASGYTFTSYWMHWIKQRPGQGLEWIGEINPSNGGS
NYNEKFKSKATLTVDKSSSTAYMQLSSLTSEDSAVYHCKSRGYWGQGTTLTVSS (SEQ
ID NO: 133)
6-017 Light Chain Variable Region
DVVMTQTPLTLSVTIGQPASISCKSSQSLLDSYGKTYLNWLLQRPGQSPKRLIYLVSKLD
SGVPDRFTGSGSGTDFTLKISRVEAEDLGIYYCWQGTHFPHTFGSGTKLEIK (SEQ ID
NO: 134)
3-G5 Heavy Chain Variable Region
QVQLQQSGAELARPGASVKLSCKASGYTFTSYGISWVKQRTGQGLEWIGEIFPRSSNTY
YNEKFKGKATLTADKSSSTVYMEFRSLTSEDSAVYFCAREGGLAWFAYWGQGTLVTV
SA (SEQ ID NO: 135)
3-G5 Light Chain Variable Region
DVVMTQTPLTLSVTIGQPASISCKSSQSLLYTNGNTYLNWLLQRPGQSPKRLIYLVSKLD
SGIPDRFSGSGSGTDFTLRISRVEAEDLGVYYCLQSTHFPFTFGSGTKLEIK (SEQ ID
NO: 136)
6-A15 Heavy Chain Variable Region
EVQLQQSGPELVKPGASVKMSCKASGYTITDYYMMWLKQSHGKSLEWIGDINPYTGGT
SYNQKFKGKATLTVDKSSSTAYLQLHSLTSEDSAVYYCARGAYWGQGTTLTVSS (SEQ
ID NO: 137)
6-A15 Light Chain Variable Region
DVVMTQTPLTLSVTIGQPASISCKSSQSLLDSDGKTYLNWLLQRPGQSPKRLIYLVSKLD
SGVPDRFTGSGSGTDFTLKISRVEAEDLGVYYCWQGTHFPYTFGGGTKLEIK (SEQ ID
NO: 138)
10-K10 Heavy Chain Variable Region
EVQLQQSGPELVKPGASVKMSCKASGYTITDYYMMWLKQSHGKSLEWIGDINPYTGGT
SYNQKFKGKATLTVDKSSSTAYMQLNSLTSEDSAVYYCARGAYWGQGTTLTVSS
(SEQ ID NO: 139)
10-K10 Light Chain Variable Region
DVVMTQTPLTLSVTIGQPASISCKSSQSLLDSDGKTYLNWLLQRPGQSPKRLIYLVSKLD
SGVPDRFTGSGSGTDFTLKISRVEAEDLGVYYCWQGTHFPYTFGGGTKLEIK (SEQ ID
NO: 140)
6-P20 Heavy Chain Variable Region
EVQLQQSGPELVKPGASVKISCKASGYTFTDYYMNWVKQSHGRSLELIGDINPNNGGSN
FNQKFRGKATLTVDKSSSTAYMELRSLTSEDSAIYYCARMGYWGQGTLVTVSA (SEQ
ID NO: 141)
6-P20 Light Chain Variable Region
DVVMTQTPLTLSVTIGQPASISCKSSQSLLHSDGKTYLNWMFQRPGQSPKRLIYLVSKLD
SGVPYRFTGGGSGTDFTLQISRVETEDLGVYYCWQGTHFPRTFGGGTKLEIK (SEQ ID
NO: 142)
7-08 Heavy Chain Variable Region
EVQLQQSGPELVKPGASVKMSCKASGYTFTDYYIHWVKQKPGQGLEYIGEIYPGSGNT
YYNGKFRGKATLTADKSSSTAYMQLSSLTSEDSAVYFCGSGYFDYWGQGTTLTVSS
(SEQ ID NO: 143)
7-08 Light Chain Variable Region
DVVMTQTPLTLSVTIGQPASISCKSSQSLLYSNGKTYLNWLLQSPGQSPKLLIYLVSKLES
GVPDRFSGSGSGTDFTLKLSRVEAEDLGVYYCVQGTHFPFTFGSGTKLEIK (SEQ ID
NO: 144)
Rabbit mAb sequences
A11B1_16G7 Heavy Chain
METGLRWLLLVAVFKGVQCQEQLVESGGDLVKPGASLTLTCTASGFSFNKNYWMCWV
RQAPGKGLEWIGCIYNGDGNTYYASWVNGRFTISKTSSTTVTLQMTSLTVADTAIYFCA
RLLNMWGPGTLVTVSSGQPKAPSVFPLAPCCGDTPSSTVTLGCLVKGYLPEPVTVTWNS
GTLTNGVRTFPSVRQSSGLYSLSSVVSVTSSSQPVTCNVAHPATNTKVDKTVAPSTCSKP
MCPPPELPGGPSVFIFPPKPKDTLMISRTPEVTCVVVDVSQDDPEVQFTWYINNEQVRTA
RPPLREQQFNSTIRVVSTLPIAHQDWLRGKEFKCKVHNKALPAPIEKTISKARGQPLEPK
VYTMGPPREELSSRSVSLTCMINGFYPSDISVEWEKNGKAEDNYKTTPTVLDSDGSYFL
YSKLSVPTSEWQRGDVFTCSVMHEALHNHYTQKSISRSPGK* (SEQ ID NO: 159)
A11B1_16G7 Light Chain
MDTRAPTQLLGLLLLWLPGARCADIVMTQTPASVEAAVGGTVTIKCQASESIGNALAW
YQQKPGQPPKLLIYTAATLASGVPSRFSGSGSGTEFTLTISGVQCDDAATYYCQSYYFTS
VSSYGNAFGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGTTSVVQSFNR
GDC* (SEQ ID NO: 160)
A11B1_16E10 Heavy Chain
METGLRWLLLVAVLKGVQCQSLEESGGDLVKPGASLTLTCRVSGFSFSSSYYMCWVRQ
APGKGLEWIACIGTTRGSTYYATWAKGRFTISKISSTTVTLQMTSLTDADTATYFCARDA
TGYRINTIGLYFNLWGPGTLVTVSSGQPKAPSVFPLAPCCGDTPSSTVTLGCLVKGYLPE
PVTVTWNSGTLTNGVRTFPSVRQSSGLYSLSSVVSVTSSSQPVTCNVAHPATNTKVDKT
VAPSTCSKPMCPPPELPGGPSVFIFPPKPKDTLMISRTPEVTCVVVDVSQDDPEVQFTWYI
NNEQVRTARPPLREQQFNSTIRVVSTLPIAHQDWLRGKEFKCKVHNKALPAPIEKTISKA
RGQPLEPKVYTMGPPREELSSRSVSLTCMINGFYPSDISVEWEKNGKAEDNYKTTPTVL
DSDGSYFLYSKLSVPTSEWQRGDVFTCSVMHEALHNHYTQKSISRSPGK* (SEQ ID NO:
161)
A11B1_16E10 Light Chain
MDTRAPTQLLGLLLLWLPGARCAFELTQTPSSVEAAVGGTPTIKCQASQTIYSYLSWYQ
QKPGQPPKLLIYEASKLASGVPSRFSGSGSGTDYTLTISDLECADAATYYCQSYHGTAST
EYNTFGGGTEVVVRGDPVAPTVLIFPPAADQVATGTVTIVCVANKYFPDVTVTWEVDG
TTQTTGIENSKTPQNSADCTYNLSSTLTLTSTQYNSHKEYTCKVTQGTTSVVQSFNRGDC
* (SEQ ID NO: 162)
A11B1_15G10 Heavy Chain
METGLRWLLLVAVLKGVQCQQQLVESGGGLVKPGAALTFTCTASGFSFSGNYWICWV
RQAPGKGLEWIACIGTITSRTYYASWAKGRFTISKTSSTTVTLQMTSLTAADTATYFCAR
GAVVSSGNAPYYFTLWGPGTLVTVSSGQPKAPSVFPLAPCCGDTPSSTVTLGCLVKGYL
PEPVTVTWNSGTLTNGVRTFPSVRQSSGLYSLSSVVSVTSSSQPVTCNVAHPATNTKVD
KTVAPSTCSKPMCPPPELPGGPSVFIFPPKPKDTLMISRTPEVTCVVVDVSQDDPEVQFT
WYINNEQVRTARPPLREQQFNSTIRVVSTLPIAHQDWLRGKEFKCKVHNKALPAPIEKTI
SKARGQPLEPKVYTMGPPREELSSRSVSLTCMINGFYPSDISVEWEKNGKAEDNYKTTP
TVLDSDGSYFLYSKLSVPTSEWQRGDVFTCSVMHEALHNHYTQKSISRSPGK* (SEQ ID
NO: 163)
A11B1_15G10 Light Chain
MDTRAPTQLLGLLLLWLPGARCAFELTQTPSSVEAAVGGTVTIKCQASQSISSYLSWYQ
QKPGQPPKLLIYRASTLESGVPSRFKGSGSGTEFTLTISDLECADAATYFCQSYYGVTFSG
FAFGGGTEVVVKGDPVAPTVLIFPPAADQVATGTVTIVCVANKYFPDVTVTWEVDGTT
QTTGIENSKTPQNSADCTYNLSSTLTLTSTQYNSHKEYTCKVTQGTTSVVQSFNRGDC*
(SEQ ID NO: 164)
A11B1_14H1 Heavy Chain
METGLRWLLLVAVLKGVQCQSLEESGGDLVKPGASLTLTCKASGIDENNYWITWVRQA
PGKGLEWIACIYVGITGRTWYANWAKGRFTISKASSTVDLKMTSLTAADTATYFCARN
GDGGIYALNLWGPGTLVTVSSGQPKAPSVFPLAPCCGDTPSSTVTLGCLVKGYLPEPVT
VTWNSGTLTNGVRTFPSVRQSSGLYSLSSVVSVTSSSQPVTCNVAHPATNTKVDKTVAP
STCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 165)
A11B1_14H1 Light Chain
MDTRAPTQLLGLLLLWLPGATFAQVLTQTASSVSAAVGGTVTISCQSSQSVYNNNWLA
WYQQKPGQPPKLLIYRASTLTSGVPSRFKGSGSGTQFTLTISDLECDDAATYYCAGGYS
GNIYVNDFGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGTTSVVQSFNR
GDC* (SEQ ID NO: 166)
A11B1_13G4 Heavy Chain
METGLRWLLLVAVLKGVQCQEQLEESGGDLVKPGGSLTLTCKASGFSFSNTYWACWV
RQAPGKGLEWIACMNPASSGSSYYASWAKGRFTISKTSSTTVTLHMPSLTAADTATYFC
AKWDTAFDVWGPGTLVTVSSGQPKAPSVFPLAPCCGDTPSSTVTLGCLVKGYLPEPVTV
TWNSGTLTNGVRTFPSVRQSSGLYSLSSVVSVTSSSQPVTCNVAHPATNTKVDKTVAPS
TCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 167)
A11B1_13G4 Light Chain
MDTRAPTQLLGLLLLWLPGARCADVVMTQTPSSVEAAVGGTVTIKCQASQSISSYLAW
YQQKPGQPPKLLIYGASNLESGVPSRFKGSGSGTEYTLTISGVQCDDAATYYCQNYYAI
DTYGHAFGGGTEVVVKGDPVAPTVLIFPPAADQVATGTVTIVCVANKYFPDVTVTWEV
DGTTQTTGIENSKTPQNSADCTYNLSSTLTLTSTQYNSHKEYTCKVTQGTTSVVQSFNRG
DC* (SEQ ID NO: 168)
A11B1_13C3 Heavy Chain
METGLRWLLLVAVLKGVQCQEQLEESGGDLVKPGASLTLTCTASGFSFSSNYHICWVR
QAPGKGLELIACIYVGDGSTYYASWAKGRFTISKSSSTTVALQMTSLTAADTATYFCGR
MFNLWGPGTLVTVSSGQPKAPSVFPLAPCCGDTPSSTVTLGCLVKGYLPEPVTVTWNSG
TLTNGVRTFPSVRQSSGLYSLSSVVSVTSSSQPVTCNVAHPATNTKVDKTVAPSTCSKPM
CPPPELPGGPSVFIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISKARGQPLEPKVY
TMGPPREELSSRSVSLTCMINGFYPSDISVEWEKNGKAEDNYKTTPTVLDSDGSYFLYSK
LSVPTSEWQRGDVFTCSVMHEALHNHYTQKSISRSPGK* (SEQ ID NO: 169)
A11B1_13C3 Light Chain
MDTRAPTQLLGLLLLWLPGAICDPVLTQTPSSVSAAVGVTVTINCQSSPSVYSNYLSWY
QQKPGQPPKLLIYLASTLASGVPSRFKGSGSGTQFTLTISDVQCDDAATYYCAGTYSGNI
WSFGGGTEVVVKGDPVAPTVLIFPPAADQVATGTVTIVCVANKYFPDVTVTWEVDGTT
QTTGIENSKTPQNSADCTYNLSSTLTLTSTQYNSHKEYTCKVTQGTTSVVQSFNRGDC*
(SEQ ID NO: 170)
A11B1_12F2 Heavy Chain
METGLRWLLLVAVLKGVQCQQQLVESGGGLVKPGASLTLTCTASGFSFSSGYHMCWV
RQAPGKGLEWIACFGVYTGTTTYASWAKGRFTISKTSSTTVTLQMTSLTVADTATYFCA
RISAENGGDLWGPGTLVTVSSGQPKAPSVFPLAPCCGDTPSSTVTLGCLVKGYLPEPVTV
TWNSGTLTNGVRTFPSVRQSSGLYSLSSVVSVTSSSQPVTCNVAHPATNTKVDKTVAPS
TCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 171)
A11B1_12F2 Light Chain
MDTRAPTQLLGLLLLWLPGARCDVVMTQTPASVEAAVGGTVTIKCQASQSISNYFSWY
QQKPGQPPKLLIYRASTLASGVPSRFSGSGSGTEFTLTISDLECADSATYYCQCTYGSSST
GFGFGGGTEVVVKGDPVAPTVPIFPPAADQVATGTVTIVCVANKYFPDVTVTWEVDGT
TQTTGIENSKTPQNSADCTYNLSSTLTLTSTQYNSHKEYTCKVTQGTTSVVQSFNRGDC*
(SEQ ID NO: 172)
A11B1_11D10 Heavy Chain
METGLRWLLLVAVLKGVQCQSLEESGGDLVKPGASLTLTCMASGIDFSSGYGMWWVR
QAPGKGLEYIGYIDTGDDNTYYANWAKGRFTISKTSSTTVTLQMTSLTVADTATYFCAK
GGAIDLWGPGTLVTVSSGQPKAPSVFPLAPCCGDTPSSTVTLGCLVKGYLPEPVTVTWN
SGTLTNGVRTFPSVRQSSGLYSLSSVVSVTSSSQPVTCNVAHPATNTKVDKTVAPSTCSK
PMCPPPELPGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYF
LYSKLSVPTSEWQRGDVFTCSVMHEALHNHYTQKSISRSPGK* (SEQ ID NO: 173)
A11B1_11D10 Light Chain
MDTRAPTQLLGLLLLWLPGARCADIVMTQTPASVEAAVGGTVTIKCQASQSISSYLAWY
QQKPGQRPKLLIYRASTLKSGVPSRFKGSGSGTEYTLTISDLECADAATYYCQAYYLSSS
ISYGNTFGGGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTSVVQSFNRGD
C* (SEQ ID NO: 174)
A11B1_10F9 Heavy Chain
METGLRWLLLVAVLKGVQCQSLEESGGDLVKPGASLTLTCTASGFSLSSGYGMCWVR
QAPGKGLEWIGYTDTATGTIHYASWAKGRFTISKTSSTTVTLQMTSLTAADTATYFCAK
GGAMDLWGPGTLVTVSSGQPKAPSVFPLAPCCGDTPSSTVTLGCLVKGYLPEPVTVTW
NSGTLTNGVRTFPSVRQSSGLYSLSSVVSVTSSSQPVTCNVAHPATNTKVDKTVAPSTCS
KPMCPPPELPGGPSVFIFPPKPKDTLMISRTPEVTCVVVDVSQDDPEVQFTWYINNEQVR
TARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYF
LYSKLSVPTSEWQRGDVFTCSVMHEALHNHYTQKSISRSPGK* (SEQ ID NO: 175)
A11B1_10F9 Light Chain
MDTRAPTQLLGLLLLWLPGARCADIVMTQTPASVEAAVGGTVTIKCQASQSISSYLAWY
QQKPGQPPKLLIYRTSTLASGVPSRFKGSGSGTEYTLTISDLECADAATYYCQSYAYSSSS
SYGNAFGGGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTSVVQSFNRGD
C* (SEQ ID NO: 176)
A11B1_7H12 Heavy Chain
METGLRWLLLVAVLKGVQCQSLEESGGDLVKPGASLTLTCTGSGIDFSSSYWICWVRQ
APGKGLEWIACIDGSDGNTYYASWARGRFTISKTSSTTVTLQMASLTAADTATYFCTRD
LRLWGPGTLVTVSSGQPKAPSVFPLAPCCGDTPSSTVTLGCLVKGYLPEPVTVTWNSGT
LTNGVRTFPSVRQSSGLYSLSSVVSVTSSSQPVTCNVAHPATNTKVDKTVAPSTCSKPM
CPPPELPGGPSVFIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISKARGQPLEPKVY
TMGPPREELSSRSVSLTCMINGFYPSDISVEWEKNGKAEDNYKTTPTVLDSDGSYFLYSK
LSVPTSEWQRGDVFTCSVMHEALHNHYTQKSISRSPGK* (SEQ ID NO: 177)
A11B1_7H12 Light Chain
MDTRAPTQLLGLLLLWLPGARCADIVLTQTPASVSAAVGGTVTINCQASQNVYSNNAL
AWHQQKPGQRPNLLIYKASTLASGVPSRFKGSGSGTQFTLTISDVQCDDAATYYCLGEF
SCSSGDCFVFGGGTEVVVKGDPVAPTVLIFPPAADQVATGTVTIVCVANKYFPDVTVTW
EVDGTTQTTGIENSKTPQNSADCTYNLSSTLTLTSTQYNSHKEYTCKVTQGTTSVVQSFN
RGDC* (SEQ ID NO: 178)
A11B1_7G12 Heavy Chain
METGLRWLLLVAVLKGVQCQSLEESGGDLVKPGASLTLTCMASGIDFSSGYGMWWVR
QAPGKGLEYIGYIDTGDDNTYYANWAKGRFTISKTSSTTVTLQMTSLTVADTATYFCAK
GGAIDLWGPGTLVTVSSGQPKAPSVFPLAPCCGDTPSSTVTLGCLVKGYLPEPVTVTWN
SGTLTNGVRTFPSVRQSSGLYSLSSVVSVTSSSQPVTCNVAHPATNTKVDKTVAPSTCSK
PMCPPPELPGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYF
LYSKLSVPTSEWQRGDVFTCSVMHEALHNHYTQKSISRSPGK* (SEQ ID NO: 179)
A11B1_7G12 Light Chain
MDTRAPTQLLGLLLLWLPGARCADIVMTQTPASVEAAVGGTVTIKCQASQSISSYLAWY
QQKPGQRPKLLIYRASTLKSGVPSRFKGSGSGTEYTLTISDLECADAATYYCQAYYLSSS
ISYGNTFGGGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTSVVQSFNRGD
C* (SEQ ID NO: 180)
A11B1_6G4 Heavy Chain
METGLRWLLLVAVLKGVQCQQQLEESGGGLVKPGGTLTLTCKASGVALNPYYYMCW
VRQAPGKGLEWIACVDADSSGSTYYASWAKGRFTISKTSSTTVTLKMTSLTAADTATYF
CARESVDYSSVGIGYVHGTDGLWGPGTLVTVSSGQPKAPSVFPLAPCCGDTPSSTVTLG
CLVKGYLPEPVTVTWNSGTLTNGVRTFPSVRQSSGLYSLSSVVSVTSSSQPVTCNVAHP
ATNTKVDKTVAPSTCSKPMCPPPELPGGPSVFIFPPKPKDTLMISRTPEVTCVVVDVSQD
DPEVQFTWYINNEQVRTARPPLREQQFNSTIRVVSTLPIAHQDWLRGKEFKCKVHNKAL
PAPIEKTISKARGQPLEPKVYTMGPPREELSSRSVSLTCMINGFYPSDISVEWEKNGKAED
NYKTTPTVLDSDGSYFLYSKLSVPTSEWQRGDVFTCSVMHEALHNHYTQKSISRSPGK*
(SEQ ID NO: 181)
A11B1_6G4 Light Chain
MDTRAPTQLLGLLLLWLPGARCADIVVTQTPSSVSAAVGGTVTIKCQASQSISNYFSWY
QQKPGQPPKLLIYRASTLASGVPSRFKGSGSGTEFTLTISDLECADAATYYCQCTYGRSN
SNFFYGFGGGTEVVVKGDPVAPTVLIFPPAADQVATGTVTIVCVANKYFPDVTVTWEV
DGTTQTTGIENSKTPQNSADCTYNLSSTLTLTSTQYNSHKEYTCKVTQGTTSVVQSFNRG
DC* (SEQ ID NO: 182)
A11B1_6F9 Heavy Chain
METGLRWLLLVAVLKGVQCQSLEESGGDLVKPGASLTLTCTASGSSFSSTYWNCWVRQ
APGKGLEWIACINAGSGTTYYASWAKGRFTVSKTSSTTVTLQMTSLTAADTATYFCTRD
SDGRFSSGYYFNLWGPGTLVTVSSGQPKAPSVFPLAPCCGDTPSSTVTLGCLVKGYLPEP
VTVTWNSGTLTNGVRTFPSVRQSSGLYSLSSVVSVTSSSQPVTCNVAHPATNTKVDKTV
APSTCSKPMCPPPELPGGPSVFIFPPKPKDTLMISRTPEVTCVVVDVSQDDPEVQFTWYIN
NEQVRTARPPLREQQFNSTIRVVSTLPIAHQDWLRGKEFKCKVHNKALPAPIEKTISKAR
GQPLEPKVYTMGPPREELSSRSVSLTCMINGFYPSDISVEWEKNGKAEDNYKTTPTVLDS
DGSYFLYSKLSVPTSEWQRGDVFTCSVMHEALHNHYTQKSISRSPGK* (SEQ ID NO:
183)
A11B1_6F9 Light Chain
MDTRAPTQLLGLLLLWLPGATFAQVLTQTASPVSAAVGGTVTINCQSSQSVYDNNWLA
WYQQKPGQPPKLLIDDASKLTSGVSSRFKGSGSGTQFTLTISGVQCDDAATYYCQGAYY
SSGWYWAFGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGTTSVVQSFNR
GDC* (SEQ ID NO: 184)
A11B1_6C7 Heavy Chain
METGLRWLLLVAVLKGVQCQQQLEESGGGLVKPGGTLTLTCKASGIDFSSYYYMCWV
RQAPGKGLELIVCIYTSSGGTWYASWVNGRLTISRSTSLNTVDLKMTSLTAADTATYFC
ARGVYSGSSDYPTRLDLWGQGTLVTVSLGQPKAPSVFPLAPCCGDTPSSTVTLGCLVKG
YLPEPVTVTWNSGTLTNGVRTFPSVRQSSGLYSLSSVVSVTSSSQPVTCNVAHPATNTK
VDKTVAPSTCSKPMCPPPELPGGPSVFIFPPKPKDTLMISRTPEVTCVVVDVSQDDPEVQF
TWYINNEQVRTARPPLREQQFNSTIRVVSTLPIAHQDWLRGKEFKCKVHNKALPAPIEKT
ISKARGQPLEPKVYTMGPPREELSSRSVSLTCMINGFYPSDISVEWEKNGKAEDNYKTTP
TVLDSDGSYFLYSKLSVPTSEWQRGDVFTCSVMHEALHNHYTQKSISRSPGK* (SEQ ID
NO: 185)
A11B1_6C7 Light Chain
MDTSTSTALLGLLLLWLTGARCAIEMTQSPPSLSASVGETVRIRCLASEDIYSGISWYQQ
KPEKPPTLLISGASNLESGVPPRFSGGGSGTDYTLTIGGVQAEDVATYYCLGGYSFSSTG
LTFGAGTKVEIKRDPVAPSVLLFPPSKEELTTGTATIVCVANKYFPDVTVTWEVDGTTQT
TGIENSKTPQNSADCTYNLSSTLTLTSTQYNSHKEYTCKVTQGTTSVVQSFNRGDC*
(SEQ ID NO: 186)
A11B1_6B6 Heavy Chain
METGLRWLLLVAVLKGVQCQQHLVESGGGLVKPGASLTLTCTASGFSFTTGYHMCWV
RQAPGKGLEWIACFGVYTSTTTYASWAKGRFTISKTSSTTVTLQMTSLTVADTATYFCA
RISAEDGGDLWGPGTLVTVSSGQPKAPSVFPLAPCCGDTPSSTVTLGCLVKGYLPEPVTV
TWNSGTLTNGVRTFPSVRQSSGLYSLSSVVSVTSSSQPVTCNVAHPATNTKVDKTVAPS
TCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 187)
A11B1_6B6 Light Chain
MDTRAPTQLLGLLLLWLPGARCDVVMTQTPASVEAAVGGTVTITCQASQSISNYFSWY
QQKPGQPPKLLIYRASTLASGVPSRFSGSGSGTQFTLTISDLECADSATYACQCTYGSSST
GFGFGGGTEVVVKGDPVAPTVLIFPPAADQVATGTVTIVCVANKYFPDVTVTWEVDGT
TQTTGIENSKTPQNSADCTYNLSSTLTLTSTQYNSHKEYTCKVTQGTTSVVQSFNRGDC*
(SEQ ID NO: 188)
A11B1_5F7 Heavy Chain
METGLRWLLLVAVLKGVQCQSLEESGGDLVKPGASLTLTCKASGFSFSSYFWICWVRQ
APGKGLEWSACIYGDSSGSSYYASWAKGRFTISKTSSTTVTLQMTSLTAADTATYFCAS
YGSSSYYYSNLWGPGTLVTVSSGQPKAPSVFPLAPCCGDTPSSTVTLGCLVKGYLPEPVT
VTWNSGTLTNGVRTFPSVRQSSGLYSLSSVVSVTSSSQPVTCNVAHPATNTKVDKTVAP
STCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 189)
A11B1_5F7 Light Chain
MDTRAPTQLLGLLLLWLPGAICDPVMTQTPSSTSAAVGGTVTISCQSSQSVYNNNYLAW
YQQKPGQPPKRLIYESSKLASGVPSRFRGSGSGAQFTLTISDLECDDAATYYCLGAYYTT
LDFGGGTEVVVRGDPVAPTVLIFPPAADQVATGTVTIVCVANKYFPDVTVTWEVDGTT
QTTGIENSKTPQNSADCTYNLSSTLTLTSTQYNSHKEYTCKVTQGTTSVVQSFNRGDC*
(SEQ ID NO: 190)
A11B1_5D7 Heavy Chain
METGLRWLLLVAVLKGVQCQEQLVESGGGLVQPEGSLTLTCKASGFDFSSNAMCWVR
QAPGKGLEWIACIYNGDGSTYYASWVNGRFTISKTSSTTVTLQMTSLTAADTATYFCAR
GLSNWNRDNLWGPGTLVTVSSGQPKAPSVFPLAPCCGDTPSSTVTLGCLVKGYLPEPVT
VTWNSGTLTNGVRTFPSVRQSSGLYSLSSVVSVTSSSQPVTCNVAHPATNTKVDKTVAP
STCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 191)
A11B1_5D7 Light Chain
MDTRAPTQLLGLLLLWLPGATFAQVLTQTPSSVSAAVGGTATINCQASQSLYSPKNLAW
YQQTPGQPPKLLIYSASKLASGVPSRFKGSGSGTQFTLTISGVQCDDAAIYYCQGEFSCTT
AACFAFGGGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTSVVQSFNRGD
C* (SEQ ID NO: 192)
A11B1_5A7 Heavy Chain
METGLRWLLLVAVLKGVQCQSLEESGGGLVQPEGSLTLACTASGFSFSSYYYICWVRQ
APGTGLEWIGCINTGSDDTHYASWLKGRFTFSKASSTTLTLQMTSLTAADTATYFCARS
SGSSDDAYDLWGPGTLVTVSSGQPKAPSVFPLAPCCGDTPSSTVTLGCLVKGYLPEPVT
VTWNSGTLTNGVRTFPSVRQSSGLYSLSSVVSVTSSSQPVTCNVAHPATNTKVDKTVAP
STCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 193)
A11B1_5A7 Light Chain
MDTRAPTQLLGLLLLWLPGARCDVVMTQTPASVSEPVGGAVTIKCQASQSIGSNLAWY
QHKPGQPPKLLIYFASSLASGVSSRFKGGRSGTQFTLTISDLECADAATYYCHCTYYPLS
YVTFGGGTEVVVKGDPVAPTVLIFPPAADQVATGTVTIVCVANKYFPDVTVTWEVDGT
TQTTGIENSKTPQNSADCTYNLSSTLTLTSTQYNSHKEYTCKVTQGTTSVVQSFNRGDC*
(SEQ ID NO: 194)
A11B1_4E1 Heavy Chain
METGLRWLLLVAVLKGVQCQSLEESGGDLVKPGTSLTLSCTASGFSFGSYYYMCWVRQ
APGKGLEWIACIDVGSSGDTYYASWVNGRFTISKTSSTTVTLQMTSLTAADTATYFCAR
DDTAAGGFGNLELWGPGTLVTVSSGQPKAPSVFPLAPCCGDTPSSTVTLGCLVKGYLPE
PVTVTWNSGTLTNGVRTFPSVRQSSGLYSLSSVVSVTSSSQPVTCNVAHPATNTKVDKT
VAPSTCSKPMCPPPELPGGPSVFIFPPKPKDTLMISRTPEVTCVVVDVSQDDPEVQFTWYI
NNEQVRTARPPLREQQFNSTIRVVSTLPIAHQDWLRGKEFKCKVHNKALPAPIEKTISKA
RGQPLEPKVYTMGPPREELSSRSVSLTCMINGFYPSDISVEWEKNGKAEDNYKTTPTVL
DSDGSYFLYSKLSVPTSEWQRGDVFTCSVMHEALHNHYTQKSISRSPGK* (SEQ ID NO:
195)
A11B1_4E1 Light Chain
MDTRAPTQLLGLLLLWLPGARCAFELTQTPSSVSEPVGGTVTIKCQASQSIYSYFSWYQ
QKPGQPPKRLIYQASTLASGVPSRFKGSGSGTDFTLTISDLECADAATYYCQNNYGRGS
GSYFFGFGGGTEVVVKGDPVAPTVLIFPPAADQVATGTVTIVCVANKYFPDVTVTWEV
DGTTQTTGIENSKTPQNSADCTYNLSSTLTLTSTQYNSHKEYTCKVTQGTTSVVQSFNRG
DC* (SEQ ID NO: 196)
A11B1_3H9 Heavy Chain
METGLRWLLLVAVLKGVQCQSLEESGGDLVKPGASLTLTCKASGIDFSSGYGMWWVR
QAPGKGLEYIGYIDTGSGSTYYANWAKGRFTISKTSSTMVTLQMTSLTVADTATYFCAK
GGAIDLWGPGTLVTVSSGQPKAPSVFPLAPCCGDTPSSTVTLGCLVKGYLPEPVTVTWN
SGTLTNGVRTFPSVRQSSGLYSLSSVVSVTSSSQPVTCNVAHPATNTKVDKTVAPSTCSK
PMCPPPELPGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYF
LYSKLSVPTSEWQRGDVFTCSVMHEALHNHYTQKSISRSPGK* (SEQ ID NO: 197)
A11B1_3H9 Light Chain
MDTRAPTQLLGLLLLWLPGARCADIVMTQTPASVEAAVGGTVTIKCQASQSISSYLAWY
QQKPGQRPKLLIYRASTLASGVPSRFKGSGSGTDYTLTISDLECADAATYYCHTYYLSSS
ISYGNTFGGGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTSVVQSFNRGD
C* (SEQ ID NO: 198)
A11B1_3G2 Heavy Chain
METGLRWLLLVAVLKGVQCQEQLEESGGDLVKPEGSLTLTCKASGFSFSSIYWICWVRQ
APGKGLEWIACTTVVKSGRTYYANWAKGRFTISKTSSTTVTLQMTSLTAADTATYFCA
REFVDGGGSSGRDLWGPGTLVTVSSGQPKAPSVFPLAPCCGDTPSSTVTLGCLVKGYLP
EPVTVTWNSGTLTNGVRTFPSVRQSSGLYSLSSVVSVTSSSQPVTCNVAHPATNTKVDK
TVAPSTCSKPMCPPPELPGGPSVFIFPPKPKDTLMISRTPEVTCVVVDVSQDDPEVQFTW
YINNEQVRTARPPLREQQFNSTIRVVSTLPIAHQDWLRGKEFKCKVHNKALPAPIEKTIS
KARGQPLEPKVYTMGPPREELSSRSVSLTCMINGFYPSDISVEWEKNGKAEDNYKTTPT
VLDSDGSYFLYSKLSVPTSEWQRGDVFTCSVMHEALHNHYTQKSISRSPGK* (SEQ ID
NO: 199)
A11B1_3G2 Light Chain
MDTRAPTQLLGLLLLWLPGARCAYDMTQTPASVEAAVGGTVTIKCQASQSISRDLSWY
QQKPGQPPKRLIYKASTLASGVPSRFKGSGSGTDFTLTISDLECADAATYYCQQGYSSID
VDNDFGGGTEVVVKGDPVAPTVLIFPPAADQVATGTVTIVCVANKYFPDVTVTWEVDG
TTQTTGIENSKTPQNSADCTYNLSSTLTLTSTQYNSHKEYTCKVTQGTTSVVQSFNRGDC
* (SEQ ID NO: 200)
A11B1_3B1 Heavy Chain
METGLRWLLLVAVLKGVQCQEQLEESGGGLVKPEGSLTLTCKASGFDLSSGYDMCWV
RQAPGKGLEWIACIYADYSGSTYYASWVNGRFTISSSTSLNTVDLKMTSLTAADTATYF
CARGATGNGGYGYYFNLWGPGTLVTVSSGQPKAPSVFPLAPCCGDTPSSTVTLGCLVK
GYLPEPVTVTWNSGTLTNGVRTFPSVRQSSGLYSLSSVVSVTSSSQPVTCNVAHPATNT
KVDKTVAPSTCSKPMCPPPELPGGPSVFIFPPKPKDTLMISRTPEVTCVVVDVSQDDPEV
QFTWYINNEQVRTARPPLREQQFNSTIRVVSTLPIAHQDWLRGKEFKCKVHNKALPAPIE
KTISKARGQPLEPKVYTMGPPREELSSRSVSLTCMINGFYPSDISVEWEKNGKAEDNYKT
TPTVLDSDGSYFLYSKLSVPTSEWQRGDVFTCSVMHEALHNHYTQKSISRSPGK* (SEQ
ID NO: 201)
A11B1_3B1 Light Chain
MDTRAPTQLLGLLLLWLPGARCADIVMTQTPASVSEPVGGTVTIKCQASQNINSGLAWY
QQKPGQPPKLLIYKASTLASGVSSRFKGSGSGTEFTLTISDLECADAATYYCQTYYYSSS
SSDNAFGGGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTSVVQSFNRGD
C* (SEQ ID NO: 202)
A11B1_2D3 Heavy Chain
METGLRWLLLVAVLKGVQCQSLEESGGDLVKPGASLTLTCTASGFSFSSSYWICWVRQ
APGKGLEWIACIYGGSSGNIAYASWAKGRFTISKTSSTTVTLQMTSLTAADTATYFCAR
DIPSDAFTLDLWGPGTLVTVSSGQPKAPSVFPLAPCCGDTPSSTVTLGCLVKGYLPEPVT
VTWNSGTLTNGVRTFPSVRQSSGLYSLSSVVSVTSSSQPVTCNVAHPATNTKVDKTVAP
STCSKPMCPPPELPGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPAPIEKTISKARGQ
PLEPKVYTMGPPREELSSRSVSLTCMINGFYPSDISVEWEKNGKAEDNYKTTPTVLDSDG
SYFLYSKLSVPTSEWQRGDVFTCSVMHEALHNHYTQKSISRSPGK* (SEQ ID NO: 203)
A11B1_2D3 Light Chain
MDTRAPTQLLGLLLLWLPGATFAQVLTQTPSSVSAAVGSTVTINCQASQSVYKDNNLA
WYQQKPGQPPKLLIYKASTLASGVPSRFKGSGSGTQFTLTISGVQCEDAATYYCQGEFS
CGSADCIAFGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGTTSVVQSFNR
GDC* (SEQ ID NO: 204)
A11B1_2A7 Heavy Chain
METGLRWLLLVAVLKGVQCQSLEESGGDLVKPGASLTLTCKGSGIDFSSGYGMWWVR
QAPGKGLEYIGYIDTGYGSTYYASWAKGRFTISKTSSTTVTLQMTSLTVADTATYFCAK
GGAIDLWGPGTLVTVSSGQPKAPSVFPLAPCCGDTPSSTVTLGCLVKGYLPEPVTVTWN
SGTLTNGVRTFPSVRQSSGLYSLSSVVSVTSSSQPVTCNVAHPATNTKVDKTVAPSTCSK
PMCPPPELPGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYF
LYSKLSVPTSEWQRGDVFTCSVMHEALHNHYTQKSISRSPGK* (SEQ ID NO: 205)
A11B1_2A7 Light Chain
MDTRAPTQLLGLLLLWLPGATFAAVLTQTPASTSAAVGGTVTINCQSSQSVYRSNWLA
WYQQKPGQPPKLLIYDVFNLASGVPSRFKGSGSGTQFTLTISGVQCADAATYYCQGSYY
SGNWYSAFGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGTTSVVQSFNR
GDC* (SEQ ID NO: 206)
Human mAb sequences
Heavy Chain and Light Chain Variable Region Sequences
2004_04_B03

| | |
|---|---|
| Heavy Chain FR1 | QVQLVESGGGVVQPGRSLRLSCAAS (SEQ ID NO: 208) |
| Heavy Chain CDR1 | GFTFSNYG (SEQ ID NO: 209) |
| Heavy Chain FR2 | MNWVRQAPGKGLEWVSY (SEQ ID NO: 210) |
| Heavy Chain CDR2 | ISSSGSTV (SEQ ID NO: 211) |
| Heavy Chain FR3 | YYADSVKGRFTISRDNAKNSLYLQMNSLRDEDTAVYYCAS |
| | (SEQ ID NO: 212) |
| Heavy Chain CDR3 | GOLDTSDAFDI (SEQ ID NO: 213) |
| Heavy Chain FR4 | WGQGTLVTVSS (SEQ ID NO: 214) |
| Heavy Chain V Gene | IGHV3-48 |
| Segment | |
| Light Chain FR1 | DIEMTQSPSSPSASVGDRVTITCRAS (SEQ ID NO: 215) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | AAS (SEQ ID NO: 218) |
| Light Chain FR3 | SLQSGVPSRFSGSGSGTDFTLTISSLQPEDFATYYC |
| | (SEQ ID NO: 219) |
| Light Chain CDR3 | QQSYSTPLT (SEQ ID NO: 220) |
| Light Chain FR4 | FGGGTKVEIK (SEQ ID NO: 221) |
| Light Chain V Gene | IGKV1-39; IGKV1D-39 |
| Segment | |
| Light Chain Locus | kappa |

2004_05_A06

| | |
|---|---|
| Heavy Chain FR1 | EVQLLESGGGVVQSGRSLRVSCAAS (SEQ ID NO: 222) |
| Heavy Chain CDR1 | GFSFSSYG (SEQ ID NO: 223) |
| Heavy Chain FR2 | MHWVRQAPGKGLEWVSY (SEQ ID NO: 224) |
| Heavy Chain CDR2 | ISSSGSTI (SEQ ID NO: 225) |
| Heavy Chain FR3 | YYADSVKGRFTISRDNAENSLYLQMNSLRAEDTAVYYCAR |
| | (SEQ ID NO: 226) |
| Heavy Chain CDR3 | DLGHFDSGSSYFDY (SEQ ID NO: 442) |
| Heavy Chain FR4 | WGQGTLVTVSS (SEQ ID NO: 214) |
| Heavy Chain V Gene | IGHV3-48 |
| Segment | |
| Light Chain FR1 | DIQMTQSPSSLSASVGDRVTITCRAS (SEQ ID NO: 227) |
| Light Chain CDR1 | QGISNY (SEQ ID NO: 228) |
| Light Chain FR2 | LAWYQQKPGKVPKLLIY (SEQ ID NO: 229) |
| Light Chain CDR2 | AAS (SEQ ID NO: 218) |
| Light Chain FR3 | TLQSGVPSRFSGSGSGTDFTLTISSLQPEDFATYYC |
| | (SEQ ID NO: 230) |
| Light Chain CDR3 | QQSYSTPLT (SEQ ID NO: 220) |
| Light Chain FR4 | FGGGTKVEVK (SEQ ID NO: 231) |
| Light Chain V Gene | IGKV1-27 |
| Segment | |
| Light Chain Locus | kappa |

2004_04_C12

| | |
|---|---|
| Heavy Chain FR1 | EVQLLESGGGVVQPGRSLRLSCAAS (SEQ ID NO: 232) |
| Heavy Chain CDR1 | GFTFSNYG (SEQ ID NO: 209) |
| Heavy Chain FR2 | MNWVRQAPGKGLEWVSY (SEQ ID NO: 210) |
| Heavy Chain CDR2 | ISSSSSTI (SEQ ID NO: 233) |
| Heavy Chain FR3 | YYADSVKGRFTISRDNAKNSLYLQMNSLRDEDTAVYYCAS |
| | (SEQ ID NO: 212) |
| Heavy Chain CDR3 | GQXDXSDAFDI (SEQ ID NO: 234) |
| Heavy Chain FR4 | WGQGTLVTVSS (SEQ ID NO: 214) |
| Heavy Chain V Gene | IGHV3OR16-8 |
| Segment | |
| Light Chain FR1 | DIEMTQSPSSPSASVGDRVTITCRAS (SEQ ID NO: 215) |
| Light Chain CDR1 | QSISSY (SEQ ID NO: 216) |
| Light Chain FR2 | LNXYQQKPGKAPKLLXY (SEQ ID NO: 235) |
| Light Chain CDR2 | XAS (SEQ ID NO: 236) |
| Light Chain FR3 | SLQSGVPSRFSGSGSGTDFTLTISSLQPEDXATYYC |
| | (SEQ ID NO: 237) |
| Light Chain CDR3 | QQSYSTPLT (SEQ ID NO: 220) |
| Light Chain FR4 | FGGGXKXEIK (SEQ ID NO: 238) |
| Light Chain V Gene | IGKV1-39; IGKV1D-39 |
| Segment | |
| Light Chain Locus | kappa |

2002_02_B07

| | |
|---|---|
| Heavy Chain FR1 | EVQLLESGGGVVQPGRSLRLSCAAS (SEQ ID NO: 232) |
| Heavy Chain CDR1 | GFTFSTYG (SEQ ID NO: 436) |
| Heavy Chain FR2 | MHWVRQAPGKGLEWVSY (SEQ ID NO: 224) |
| Heavy Chain CDR2 | ISSSGSTI (SEQ ID NO: 225) |
| Heavy Chain FR3 | YYADSVKGRFAISRDNAKNTLYLQMNSLRAEDTALYYCAK |
| | (SEQ ID NO: 239) |
| Heavy Chain CDR3 | ATRYDILTGYSDGVDYFDY (SEQ ID NO: 240) |
| Heavy Chain FR4 | WGQGTLVTVSS (SEQ ID NO: 214) |
| Heavy Chain V Gene | IGHV3-48 |
| Segment | |
| Light Chain FR1 | DIQMTQSPSSLSASVGDRVTITCRAS (SEQ ID NO: 227) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | AAS (SEQ ID NO: 218) |
| Light Chain FR3 | SLQSGVPSRFSGSGSGTDFTLTISSLQPEDFATYYC |
| | (SEQ ID NO: 219) |
| Light Chain CDR3 | HQSYSTPYT (SEQ ID NO: 241) |
| Light Chain FR4 | FGQGTKLEIK (SEQ ID NO: 242) |
| Light Chain V Gene | IGKV1-39; IGKV1D-39 |
| Segment | |
| Light Chain Locus | kappa |

2004_05_B04

| | |
|---|---|
| Heavy Chain FR1 | QVQLVESGGGLVQPGGSLRLSCAAS (SEQ ID NO: 243) |
| Heavy Chain CDR1 | GFTFSSYW (SEQ ID NO: 437) |
| Heavy Chain FR2 | MSWVRQAPGKGLEWVAN (SEQ ID NO: 244) |
| Heavy Chain CDR2 | IKQDGSEK (SEQ ID NO: 245) |
| Heavy Chain FR3 | YYVDSVKGRFTISRDNAKNSLYLQMNSLRAEDTAVYYCAR |
| | (SEQ ID NO: 246) |
| Heavy Chain CDR3 | VTSPHAFDI (SEQ ID NO: 247) |
| Heavy Chain FR4 | WGRGTLVTVSS (SEQ ID NO: 248) |
| Heavy Chain V Gene | IGHV3-7 |
| Segment | |
| Light Chain FR1 | DIQMTQSPSAMSASVGDRVTITCRAS (SEQ ID NO: 249) |
| Light Chain CDR1 | QGISNY (SEQ ID NO: 228) |
| Light Chain FR2 | LAWFQQKPGKVPKRLIY (SEQ ID NO: 250) |
| Light Chain CDR2 | AAS (SEQ ID NO: 218) |
| Light Chain FR3 | SLQSGVPSRFSGSGSGTDFTLTISSLQPEDFATYYC |
| | (SEQ ID NO: 219) |
| Light Chain CDR3 | QQSYSTPLT (SEQ ID NO: 220) |
| Light Chain FR4 | FGGGTKVEVK (SEQ ID NO: 231) |
| Light Chain V Gene | IGKV1-39; IGKV1D-17; IGKV1D-39 |
| Segment | |
| Light Chain Locus | kappa |

2003_03_E12

| | |
|---|---|
| Heavy Chain FR1 | QVQLVESGGGVVRPGGSLRLSCAAS (SEQ ID NO: 251) |
| Heavy Chain CDR1 | GFTFDDYG (SEQ ID NO: 438) |
| Heavy Chain FR2 | MSWVRQAPGKGLEWVSG (SEQ ID NO: 252) |
| Heavy Chain CDR2 | INWNGGST (SEQ ID NO: 253) |
| Heavy Chain FR3 | GYADSVKGRFTISRDNSKNTLYLQMNSLRGEDTAVYYCVT |
| | (SEQ ID NO: 254) |
| Heavy Chain CDR3 | QGSAFDI (SEQ ID NO: 255) |
| Heavy Chain FR4 | WGRGTLVTVSS (SEQ ID NO: 248) |
| Heavy Chain V Gene | IGHV3-20 |
| Segment | |
| Light Chain FR1 | SYELTQPPSLSVSPGQTARITCSGD (SEQ ID NO: 256) |
| Light Chain CDR1 | ALAKQY (SEQ ID NO: 257) |
| Light Chain FR2 | AYWYQQTPGQAPVLVIY (SEQ ID NO: 258) |
| Light Chain CDR2 | KDT (SEQ ID NO: 259) |
| Light Chain FR3 | ERPSGIPERFSGSSSGTTVTLTISGVQAEDEVDYYC |
| | (SEQ ID NO: 260) |
| Light Chain CDR3 | QSTDSSGTYQV (SEQ ID NO: 261) |
| Light Chain FR4 | FGGGTKLTVL (SEQ ID NO: 262) |
| Light Chain V Gene | IGLV3-25 |
| Segment | |
| Light Chain Locus | lambda |

1994_01_C07

| | |
|---|---|
| Heavy Chain FR1 | QVQLVQSGAEVKKPGASVKVSCKAS (SEQ ID NO: 263) |
| Heavy Chain CDR1 | GYTFTSYG (SEQ ID NO: 439) |
| Heavy Chain FR2 | ISWVRQAPGQGLEWMGW (SEQ ID NO: 264) |
| Heavy Chain CDR2 | ISAYNGNT (SEQ ID NO: 265) |
| Heavy Chain FR3 | NYAQKLQGRVTMTTDTSTSTAYMELRSLRSDDTAAYYCAR |
| | (SEQ ID NO: 266) |
| Heavy Chain CDR3 | VTGITGTTIDP (SEQ ID NO: 267) |
| Heavy Chain FR4 | WGQGTMVTVSS (SEQ ID NO: 268) |
| Heavy Chain V Gene | IGHV1-18 |
| Segment | |
| Light Chain FR1 | DIQMTQSPSSLSASVGDRVTITCRAS (SEQ ID NO: 227) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | DAS (SEQ ID NO: 269) |
| Light Chain FR3 | SLESGVPSRFSGSGSGTEFTLTISSLQPDDFAVYYC |
| | (SEQ ID NO: 270) |
| Light Chain CDR3 | QQYNNWPQT (SEQ ID NO: 271) |
| Light Chain FR4 | FGQGTKVEIK (SEQ ID NO: 272) |
| Light Chain V Gene | IGKV1-13; IGKV1D-13 |
| Segment | |
| Light Chain Locus | kappa |

1995_01_G07

| | |
|---|---|
| Heavy Chain FR1 | QVQLVESGGGLVKPGGSLRLSCAAS (SEQ ID NO: 273) |
| Heavy Chain CDR1 | GFTFSSYA (SEQ ID NO: 440) |
| Heavy Chain FR2 | MSWVRQAPGKGLEWVSA (SEQ ID NO: 274) |
| Heavy Chain CDR2 | ISGSGGST (SEQ ID NO: 275) |
| Heavy Chain FR3 | YYADSVKGRFTISRDNSKNTLYLQMNSLRAEDTAVYYCAR |
| | (SEQ ID NO: 276) |
| Heavy Chain CDR3 | DYGGYDGVYFDY (SEQ ID NO: 277) |
| Heavy Chain FR4 | WGRGTLVTVSS (SEQ ID NO: 248) |
| Heavy Chain V Gene | IGHV3-23 |
| Segment | |
| Light Chain FR1 | SYELTQDPAVSVALGQTVRITCQGD (SEQ ID NO: 278) |
| Light Chain CDR1 | SLRSYY (SEQ ID NO: 279) |
| Light Chain FR2 | ASWYQQKPGQAPVLVIY (SEQ ID NO: 280) |
| Light Chain CDR2 | GKN (SEQ ID NO: 281) |
| Light Chain FR3 | NRPSGIPDRFSGSSSGNTASLTITGAQAEDEADYYC |
| | (SEQ ID NO: 282) |
| Light Chain CDR3 | NSRDSSGNHVV (SEQ ID NO: 283) |
| Light Chain FR4 | FGGGTKVTVL (SEQ ID NO: 284) |
| Light Chain V Gene | IGLV3-19 |
| Segment | |
| Light Chain Locus | lamdba |

1995_01_G05

| | |
|---|---|
| Heavy Chain FR1 | EVQLLESGGGLVKPGGSLRLSCAAS (SEQ ID NO: 285) |
| Heavy Chain CDR1 | GFTFSSYA (SEQ ID NO: 440) |
| Heavy Chain FR2 | MHWVRQAPGKGLEWVAV (SEQ ID NO: 286) |
| Heavy Chain CDR2 | ISYDGSNK (SEQ ID NO: 287) |
| Heavy Chain FR3 | YYADSVKGRFAISRDNSKNTLYLQMNSLRAEDTAVYYCAR |
| | (SEQ ID NO: 288) |
| Heavy Chain CDR3 | DRDVGPTYYYYGMDV (SEQ ID NO: 289) |
| Heavy Chain FR4 | WGQGTMVTVSS (SEQ ID NO: 268) |
| Heavy Chain V Gene | IGHV3-30 |
| Segment | |
| Light Chain FR1 | SYELTQPPSLSVSPGQTARITCSGH (SEQ ID NO: 290) |
| Light Chain CDR1 | ALPKQY (SEQ ID NO: 291) |
| Light Chain FR2 | AYWYQQTPGQAPVLVIY (SEQ ID NO: 258) |
| Light Chain CDR2 | KDT (SEQ ID NO: 259) |
| Light Chain FR3 | ERPSGIPERFSGSSSGTTVTLTISGVQAEDEADYYC |
| | (SEQ ID NO: 292) |
| Light Chain CDR3 | QSADSSGPYQV (SEQ ID NO: 293) |
| Light Chain FR4 | FGGGTQLTVL (SEQ ID NO: 294) |
| Light Chain V Gene | IGLV3-25 |
| Segment | |
| Light Chain Locus | lamdba |

2004_03_G10

| | |
|---|---|
| Heavy Chain FR1 | EVQLLESGGGVVQPGRSLRLSCAAS (SEQ ID NO: 232) |
| Heavy Chain CDR1 | GFTFSSYA (SEQ ID NO: 440) |
| Heavy Chain FR2 | MSWVRQAPGKGLEWVSA (SEQ ID NO: 274) |
| Heavy Chain CDR2 | ISGSGGST (SEQ ID NO: 275) |
| Heavy Chain FR3 | YYADSVKGRFTISRDNSKNTLYLQMNSLRAEDTAVYYCAK |
| | (SEQ ID NO: 295) |
| Heavy Chain CDR3 | DREYIAVAADY (SEQ ID NO: 296) |
| Heavy Chain FR4 | WGQGTTVTVSS (SEQ ID NO: 297) |
| Heavy Chain V Gene | IGHV3-23 |
| Segment | |
| Light Chain FR1 | DIQMTQSPSSLSASVGDTISITCRAS (SEQ ID NO: 298) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | AAS (SEQ ID NO: 218) |
| Light Chain FR3 | SLQSGVPSRFSGSGSRTDFTLTISSVQPEDFATYYC |
| | (SEQ ID NO: 299) |
| Light Chain CDR3 | QQSYSTPFT (SEQ ID NO: 300) |
| Light Chain FR4 | FGPGTKVEIK (SEQ ID NO: 301) |
| Light Chain V Gene | IGKV1-39; IGKV1D-39 |
| Segment | |
| Light Chain Locus | kappa |

2002_02_B05

| | |
|---|---|
| Heavy Chain FR1 | EVQLLESGGGVVQPGRSLRLSCAAS (SEQ ID NO: 232) |
| Heavy Chain CDR1 | GFTFSTYG (SEQ ID NO: 436) |
| Heavy Chain FR2 | MHWVRQAPGKGLEWVSY (SEQ ID NO: 224) |
| Heavy Chain CDR2 | ISSSGSTI (SEQ ID NO: 225) |
| Heavy Chain FR3 | YYADSVKGRFAISRDNAKNTLYLQMNSLRAEDTALYYCAK |
| | (SEQ ID NO: 239) |
| Heavy Chain CDR3 | ATRYDILTGYSDGVDYFDY (SEQ ID NO: 240) |
| Heavy Chain FR4 | WGQGTLVTVSS (SEQ ID NO: 214) |
| Heavy Chain V Gene | IGHV3-48 |
| Segment | |
| Light Chain FR1 | DIQMTQSPSSLSASVGDRVTITCRAS (SEQ ID NO: 227) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | AAS (SEQ ID NO: 218) |
| Light Chain FR3 | SLQSGVPSRFSGSGSGTDFTLTISSLQPEDFATYYC |
| | (SEQ ID NO: 219) |
| Light Chain CDR3 | QQSYSTPFT (SEQ ID NO: 300) |
| Light Chain FR4 | FGPGTKVEIK (SEQ ID NO: 301) |
| Light Chain V Gene | IGKV1-39; IGKV1D-39 |
| Segment | |
| Light Chain Locus | kappa |

2003_03_F05

| | |
|---|---|
| Heavy Chain FR1 | EVQLVESGAEVKKPGASVKVSCKAS (SEQ ID NO: 302) |
| Heavy Chain CDR1 | GYTFTRYY (SEQ ID NO: 441) |
| Heavy Chain FR2 | MHWVRQAPGQGLEWMGI (SEQ ID NO: 303) |
| Heavy Chain CDR2 | INPSGGST (SEQ ID NO: 304) |
| Heavy Chain FR3 | IYAQKFQGRVTMTRDTSTSTVYMELSSLRSEDTAVYYCAR |
| | (SEQ ID NO: 305) |
| Heavy Chain CDR3 | SLRDGYNYIGSLGY (SEQ ID NO: 306) |
| Heavy Chain FR4 | WGQGTLVTVSS (SEQ ID NO: 214) |
| Heavy Chain V Gene | IGHV1-46 |
| Segment | |
| Light Chain FR1 | QSELTQPPSASGTPGQRVTISCSGS (SEQ ID NO: 307) |
| Light Chain CDR1 | SSNIGSNY (SEQ ID NO: 308) |
| Light Chain FR2 | VYWYQQLPGTAPKLLIY (SEQ ID NO: 309) |
| Light Chain CDR2 | RNN (SEQ ID NO: 310) |
| Light Chain FR3 | QRPSGVPDRFSGSKSGTSASLAIRGLQSEDEAGYYC |
| | (SEQ ID NO: 311) |
| Light Chain CDR3 | AAWDDSLNGLNWV (SEQ ID NO: 207) |
| Light Chain FR4 | FGGGTQLTVL (SEQ ID NO: 294) |
| Light Chain V Gene | IGLV1-44; IGLV1-47 |
| Segment | |
| Light Chain Locus | lambda |

1994_01_A07

| | |
|---|---|
| Heavy Chain FR1 | QVQLVESGGGLVQPGGSLRLSCAAS (SEQ ID NO: 243) |
| Heavy Chain CDR1 | GFTFDDYA (SEQ ID NO: 312) |
| Heavy Chain FR2 | MHWVRQAPGKGLEWVSG (SEQ ID NO: 313) |
| Heavy Chain CDR2 | ISWNSGST (SEQ ID NO: 314) |
| Heavy Chain FR3 | YYADSVKGRFTISRDNSKNTLYLQMNSLRAEDTAVYYCAG |
| | (SEQ ID NO: 315) |
| Heavy Chain CDR3 | GSRRYDSSGYYYESFDY (SEQ ID NO: 316) |
| Heavy Chain FR4 | WGQGTTVTVSS (SEQ ID NO: 297) |
| Light Chain FR1 | DIQMTQSPSSLSASVGDRVTITCRAS (SEQ ID NO: 227) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | DAS (SEQ ID NO: 269) |
| Light Chain FR3 | NLETGVPSRFSGSGSGTDFTLTISSLQPEDFATYYC (SEQ ID |
| | NO: 317) |
| Light Chain CDR3 | QQSYHTPYT (SEQ ID NO: 318) |
| Light Chain FR4 | FGQGTKVEIK (SEQ ID NO: 272) |
| Light Chain Locus | kappa |

1994_01_A09

| | |
|---|---|
| Heavy Chain FR1 | QMQLVQSGAEVKKPGSSVKVSCKAS (SEQ ID NO: 319) |
| Heavy Chain CDR1 | GGTFSSYA (SEQ ID NO: 320) |
| Heavy Chain FR2 | ISWVRQAPGQGLEWMGR (SEQ ID NO: 321) |
| Heavy Chain CDR2 | IIPILGIA (SEQ ID NO: 322) |
| Heavy Chain FR3 | NYAQKFQGRVTITADKSTSTAYMELSSLRSEDTAVYYCAR |
| | (SEQ ID NO: 323) |
| Heavy Chain CDR3 | DINRYNWNFRAFDI (SEQ ID NO: 324) |
| Heavy Chain FR4 | WGQGTLVTVSS (SEQ ID NO: 214) |
| Light Chain FR1 | DIVMTQSPDSLAVSLGERATINCKSS (SEQ ID NO: 435) |
| Light Chain CDR1 | QSVLYSSNNKNY (SEQ ID NO: 325) |
| Light Chain FR2 | LAWYQQKPRQPPKLLIY (SEQ ID NO: 326) |
| Light Chain CDR2 | WAS (SEQ ID NO: 327) |
| Light Chain FR3 | TRESGVPDRFSGNGSGTDFTLTISSLQAEDVAAYYC (SEQ ID |
| | NO: 328) |
| Light Chain CDR3 | QQHYSTPLT (SEQ ID NO: 329) |
| Light Chain FR4 | FGPGTKVEIK (SEQ ID NO: 301) |
| Light Chain Locus | kappa |

1994_01_D12

| | |
|---|---|
| Heavy Chain FR1 | QVQLVQSGAEVKKPGSSVKVSCKAS (SEQ ID NO: 330) |
| Heavy Chain CDR1 | GYTFTSYG (SEQ ID NO: 439) |
| Heavy Chain FR2 | ISWVRQAPGQGLEWMGW (SEQ ID NO: 264) |
| Heavy Chain CDR2 | ISAYNGNT (SEQ ID NO: 265) |
| Heavy Chain FR3 | NYAQKLQGRVTMTTNTSTSTAYMELRSLRSDDTAVYYCAR |
| | (SEQ ID NO: 331) |
| Heavy Chain CDR3 | VTGITGTTIDP (SEQ ID NO: 267) |
| Heavy Chain FR4 | WGQGTLVTVSS (SEQ ID NO: 214) |
| Light Chain FR1 | DIQMTQSPSSLSASVGDRVTITCRAS (SEQ ID NO: 227) |
| Light Chain CDR1 | QSISSY (SEQ ID NO: 216) |
| Light Chain FR2 | LNWYRQKPGKAPKLLIY (SEQ ID NO: 332) |
| Light Chain CDR2 | AAS (SEQ ID NO: 218) |
| Light Chain FR3 | SLQSGVPSRFSGSGSGTDFTLTISSLQPEDAATYYC (SEQ ID |
| | NO: 333) |
| Light Chain CDR3 | QQYDSQSGT (SEQ ID NO: 334) |
| Light Chain FR4 | FGQGTKLEIK (SEQ ID NO: 242) |
| Light Chain Locus | kappa |

1995_01_F05

| | |
|---|---|
| Heavy Chain FR1 | EVQLVESGGGVVQPGRSLRLSCAAS (SEQ ID NO: 335) |
| Heavy Chain CDR1 | GFTFSSYA (SEQ ID NO: 440) |
| Heavy Chain FR2 | MHWVRQAPGKGLEWVAV (SEQ ID NO: 286) |
| Heavy Chain CDR2 | ISYDGVKK (SEQ ID NO: 336) |
| Heavy Chain FR3 | YYADSVKGRFTISRDNSKSTLYLQMNSLRVDDTAVYYCAK |
| | (SEQ ID NO: 337) |
| Heavy Chain CDR3 | DLGWQNDY (SEQ ID NO: 338) |
| Heavy Chain FR4 | WGQGTLVTVSS (SEQ ID NO: 214) |
| Light Chain FR1 | QSVLTQPASVSGSPGQSITISCTGT DLGWQNDY (SEQ ID NO: |
| | 339) |
| Light Chain CDR1 | SSDVGGHNY (SEQ ID NO: 340) |
| Light Chain FR2 | VSWYQQHPGKAPKLMIY (SEQ ID NO: 341) |
| Light Chain CDR2 | DVS (SEQ ID NO: 342) |
| Light Chain FR3 | NRPSGVSNRFSGSKSGNTASLTISGLQAEDEADYYC (SEQ ID |
| | NO: 343) |
| Light Chain CDR3 | SSYTSSSPWV (SEQ ID NO: 344) |
| Light Chain FR4 | FGGGTKLTVLG (SEQ ID NO: 345) |
| Light Chain Locus | lambda |

1995_01_F09

| | |
|---|---|
| Heavy Chain FR1 | EVQLVESGGGLVQPGGSLRLSCAAS (SEQ ID NO: 346) |
| Heavy Chain CDR1 | GFTFSSYA (SEQ ID NO: 440) |
| Heavy Chain FR2 | MSWVRQAPGKGLEWVSA (SEQ ID NO: 274) |
| Heavy Chain CDR2 | ISGSGGST (SEQ ID NO: 275) |
| Heavy Chain FR3 | YYADSVKGRFTISRDNSKNALYLQMNSLRAEDTAVYYCRG |
| | (SEQ ID NO: 347) |
| Heavy Chain CDR3 | YCSSTSCYGRRGAFDI (SEQ ID NO: 348) |
| Heavy Chain FR4 | SGQGTLVTVSS (SEQ ID NO: 349) |
| Light Chain FR1 | QAVLTQPPSASGTPGQRVTISCSGR (SEQ ID NO: 350) |
| Light Chain CDR1 | NSNIGSNN (SEQ ID NO: 351) |
| Light Chain FR2 | VNWYQHLPGTAPKLLIY (SEQ ID NO: 352) |
| Light Chain CDR2 | SNN (SEQ ID NO: 353) |
| Light Chain FR3 | QRPSGVPDRFSASKSGTSASLAISGLQSEDEADYYC (SEQ ID |
| | NO: 354) |
| Light Chain CDR3 | AAWDDRMNGPV (SEQ ID NO: 355) |
| Light Chain FR4 | IGGGTKVTVLG (SEQ ID NO: 356) |
| Light Chain Locus | lambda |

1996_01_H07

| | |
|---|---|
| Heavy Chain FR1 | QVQLVESGGGLVQPGGSLRLSCAAS (SEQ ID NO: 243) |
| Heavy Chain CDR1 | GFTFSSYW (SEQ ID NO: 437) |
| Heavy Chain FR2 | MHWVRQAPAKGLVWVSR (SEQ ID NO: 357) |
| Heavy Chain CDR2 | INSDGSST (SEQ ID NO: 358) |
| Heavy Chain FR3 | SYADSVKGRFTISRDNSKNTLYLQMNSLRAEDTAVYYCAR |
| | (SEQ ID NO: 359) |
| Heavy Chain CDR3 | DFWSGRPYYYYMDV (SEQ ID NO: 360) |
| Heavy Chain FR4 | WGQGTTVTVSS (SEQ ID NO: 297) |
| Light Chain FR1 | DIQMTQSPSSLSASVGDRVTITCRAS (SEQ ID NO: 227) |
| Light Chain CDR1 | QDIGDD (SEQ ID NO: 361) |
| Light Chain FR2 | LAWFQQKPGKAPKRLIY (SEQ ID NO: 362) |
| Light Chain CDR2 | AAS (SEQ ID NO: 218) |
| Light Chain FR3 | TLQGGVPSRFSGSGSGTDFTLTISSLQPEDFATYYC (SEQ ID |
| | NO: 363) |
| Light Chain CDR3 | QQSYSTPRT (SEQ ID NO: 364) |
| Light Chain FR4 | FGPGTKVEIK (SEQ ID NO: 301) |
| Light Chain Locus | kappa |

1997_02_B01

| | |
|---|---|
| Heavy Chain FR1 | EVQLVESGGGVVQPGRSLRLSCAAS (SEQ ID NO: 335) |
| Heavy Chain CDR1 | GFTFSSYA (SEQ ID NO: 440) |
| Heavy Chain FR2 | MHWVRQAPGKGLEWVAV (SEQ ID NO: 286) |
| Heavy Chain CDR2 | ISYDGSNK (SEQ ID NO: 287) |
| Heavy Chain FR3 | YYADSVKGRFTISRDNSKNTLYLQMNSRRAEDTAVYYCAR |
| | (SEQ ID NO: 365) |
| Heavy Chain CDR3 | WGIVAARPNYYYGMDV (SEQ ID NO: 366) |
| Heavy Chain FR4 | WGQGTLVTVSS (SEQ ID NO: 214) |
| Light Chain FR1 | QSALTQPRSVSGSPGQSVTISCTGT (SEQ ID NO: 367) |
| Light Chain CDR1 | SSDVGGYNY (SEQ ID NO: 368) |
| Light Chain FR2 | VSWYQQHPGKAPKLMIY (SEQ ID NO: 341) |
| Light Chain CDR2 | DVS (SEQ ID NO: 342) |
| Light Chain FR3 | KRPSGVPDRFSGSKSGNTASLTISGLQAEDEADYHC (SEQ ID |
| | NO: 443) |
| Light Chain CDR3 | SSYANNSPWV (SEQ ID NO: 369) |
| Light Chain FR4 | FGGGTKVTVLG (SEQ ID NO: 370) |
| Light Chain Locus | lambda |

2002_02_E01

| | |
|---|---|
| Heavy Chain FR1 | QVQLVQSGAEVRKPGASVKVSCKAS (SEQ ID NO: 371) |
| Heavy Chain CDR1 | GYTFTSYG (SEQ ID NO: 439) |
| Heavy Chain FR2 | ISWVRQAPGQGLEWMGW (SEQ ID NO: 264) |
| Heavy Chain CDR2 | ISAYNGNT (SEQ ID NO: 265) |
| Heavy Chain FR3 | NYAQKLQGRVTMTTDTSTSTAYMELRSLRSDDTAVYYCAR |
| | (SEQ ID NO: 372) |
| Heavy Chain CDR3 | VTGITGTTIDP (SEQ ID NO: 267) |
| Heavy Chain FR4 | WGQGTLVTVSS (SEQ ID NO: 214) |
| Light Chain FR1 | DIQMTQSPSSLSASVGDRVTITCQAS (SEQ ID NO: 373) |
| Light Chain CDR1 | QDISNY (SEQ ID NO: 374) |
| Light Chain FR2 | LNWYQQKPGKAPKLLIY (SEQ ID NO: 217) |
| Light Chain CDR2 | DAS (SEQ ID NO: 269) |
| Light Chain FR3 | NLETGVPSRFSGSGSGTDFTFTISSLQPEDIATYYC (SEQ ID NO: |
| | 375) |
| Light Chain CDR3 | QQYANLPLT (SEQ ID NO: 376) |
| Light Chain FR4 | FGGGTKVEIK (SEQ ID NO: 221) |
| Light Chain Locus | kappa |

2002_02_G11

| | |
|---|---|
| Heavy Chain FR1 | EVQLVESGGGLVQPGGSLRLSCAAS (SEQ ID NO: 346) |
| Heavy Chain CDR1 | GFTVSSNY (SEQ ID NO: 377) |
| Heavy Chain FR2 | MSWVRQAPGKGLEWVSV (SEQ ID NO: 378) |
| Heavy Chain CDR2 | IYSGGST (SEQ ID NO: 379) |
| Heavy Chain FR3 | YYADSVKGRFTISRDNSKNTLYLQMNSLRAEDTAVYYCAR |
| | (SEQ ID NO: 276) |
| Heavy Chain CDR3 | GGLTGDDAFDI (SEQ ID NO: 380) |
| Heavy Chain FR4 | WGQGTLVTVSS (SEQ ID NO: 214) |
| Light Chain FR1 | DIQMTQSPSSLSASVGDRVTITCRAS (SEQ ID NO: 227) |
| Light Chain CDR1 | QSISSF (SEQ ID NO: 381) |
| Light Chain FR2 | LNWYQQKPGTAPKLLIY (SEQ ID NO: 382) |
| Light Chain CDR2 | TTS (SEQ ID NO: 383) |
| Light Chain FR3 | SLQSGVPSRFSGSGSGTDFTLTISSLQPEDFATYYC (SEQ ID |
| | NO: 219) |
| Light Chain CDR3 | QQGNSLPLT (SEQ ID NO: 384) |
| Light Chain FR4 | FGGGTKVEIK (SEQ ID NO: 221) |
| Light Chain Locus | kappa |

2003_03_A09

| | |
|---|---|
| Heavy Chain FR1 | QVQLVESGGGVVQPGRSLRLSCAAS (SEQ ID NO: 208) |
| Heavy Chain CDR1 | GFTFSSYA (SEQ ID NO: 440) |
| Heavy Chain FR2 | MHWVRQAPGKGLEWVAV (SEQ ID NO: 286) |
| Heavy Chain CDR2 | ISYDGSNK (SEQ ID NO: 287) |
| Heavy Chain FR3 | YYADSVKGRFTISRDNSKNTLYLQMNSLRAEDTAVYYCAR |
| | (SEQ ID NO: 276) |
| Heavy Chain CDR3 | DKELSY (SEQ ID NO: 385) |
| Heavy Chain FR4 | WGQGTLVTVSS (SEQ ID NO: 214) |
| Light Chain FR1 | QSGLTQPASVSGSPGQSITISCTGT (SEQ ID NO: 386) |
| Light Chain CDR1 | SSDVGGYNY (SEQ ID NO: 368) |
| Light Chain FR2 | VSWYQQHPGKAPKLMIY (SEQ ID NO: 341) |
| Light Chain CDR2 | EVS (SEQ ID NO: 387) |
| Light Chain FR3 | NRPSGVPDRFSGSKSGNTASLTISGLQAEDEADYYC (SEQ ID |
| | NO: 388) |
| Light Chain CDR3 | SSYTSSSPWV (SEQ ID NO: 344) |
| Light Chain FR4 | FGGGTKLTVLG (SEQ ID NO: 345) |
| Light Chain Locus | lambda |

2004_04_D03

| | |
|---|---|
| Heavy Chain FR1 | QVQLVESGGGVVQPGRSLRLSCAAS (SEQ ID NO: 208) |
| Heavy Chain CDR1 | GFTFSNYG (SEQ ID NO: 209) |
| Heavy Chain FR2 | MNWVRQAPGKGLEWVSY (SEQ ID NO: 210) |
| Heavy Chain CDR2 | ISSSSSTI (SEQ ID NO: 233) |
| Heavy Chain FR3 | YYADSVKGRFTISRDNAKNSLYLQMNSLRDEDTAVYYCAS |
| | (SEQ ID NO: 212) |
| Heavy Chain CDR3 | GQLDTSDAFDI (SEQ ID NO: 213) |
| Heavy Chain FR4 | WGQGTTVTVSS (SEQ ID NO: 297) |
| Light Chain FR1 | DIQMTQSPSSLSASVGDRVTITCRAS (SEQ ID NO: 227) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | KTS (SEQ ID NO: 389) |
| Light Chain FR3 | NLQSGVPSRFSGSGSGTDFTLTISSLQPEDFATYYC (SEQ ID |
| | NO: 390) |
| Light Chain CDR3 | QQSYSTPLT (SEQ ID NO: 220) |
| Light Chain FR4 | FGGGTKVEIK (SEQ ID NO: 221) |
| Light Chain Locus | kappa |

2004_04_F01

| | |
|---|---|
| Heavy Chain FR1 | EVQLVESGGGLVQPGGSLRLSCAAS (SEQ ID NO: 346) |
| Heavy Chain CDR1 | GFTFSSYA (SEQ ID NO: 440) |
| Heavy Chain FR2 | MSWVRQAPAKGLEWVSA (SEQ ID NO: 391) |
| Heavy Chain CDR2 | ISGSGGST (SEQ ID NO: 275) |
| Heavy Chain FR3 | YYADSVKGRFTISRDNSKNTLYLQMNSLRAEDTAVYYCAK |
| | (SEQ ID NO: 295) |
| Heavy Chain CDR3 | DRPYSYGKNDAFDI (SEQ ID NO: 392) |
| Heavy Chain FR4 | WGQGTTVTVSS (SEQ ID NO: 297) |
| Light Chain FR1 | DIQMTQSPSSLSASVGDRVTITCQAS (SEQ ID NO: 373) |
| Light Chain CDR1 | QDVSNY (SEQ ID NO: 393) |
| Light Chain FR2 | LNWYRQKPGKAPKLLIY (SEQ ID NO: 332) |
| Light Chain CDR2 | AAS (SEQ ID NO: 218) |
| Light Chain FR3 | SLQSGVPSRFSGSGSGTDFTLTISSLQPEDFATYYC (SEQ ID |
| | NO: 219) |
| Light Chain CDR3 | QQSYSTPLT (SEQ ID NO: 220) |
| Light Chain FR4 | FGGGTKLEIK (SEQ ID NO: 394) |
| Light Chain Locus | kappa |

2005_05_E05

| | |
|---|---|
| Heavy Chain FR1 | EVQLVQSGAEVKKPGASVKVSCKAS (SEQ ID NO: 395) |
| Heavy Chain CDR1 | GYTFTSYY (SEQ ID NO: 396) |
| Heavy Chain FR2 | MHWVRQAPGQGLEWMGI (SEQ ID NO: 303) |
| Heavy Chain CDR2 | INPSGGST (SEQ ID NO: 304) |
| Heavy Chain FR3 | SYAQKFQGRVTMTRDTSTSTVYMELSSLRSEDTAVYYCAR |
| | (SEQ ID NO: 397) |
| Heavy Chain CDR3 | SPWLITFGGVIAMGY (SEQ ID NO: 402) |
| Heavy Chain FR4 | WGQGTLVTVSS (SEQ ID NO: 214) |
| Light Chain FR1 | QSVLTQPPSASGTPGQRVTISCSGS (SEQ ID NO: 403) |
| Light Chain CDR1 | SSNIGSNY (SEQ ID NO: 308) |
| Light Chain FR2 | VYWYQQLPGTAPKLLIY (SEQ ID NO: 309) |
| Light Chain CDR2 | RNN (SEQ ID NO: 310) |
| Light Chain FR3 | QRPSGVPDRFSGSKSGTSASLAISGLRSEDEADYYC (SEQ ID |
| | NO: 404) |
| Light Chain CDR3 | AAWDDSLSGVV (SEQ ID NO: 405) |
| Light Chain FR4 | FGGGTQLTVLG (SEQ ID NO: 406) |
| Light Chain Locus | lambda |

1994_01_D04

| | |
|---|---|
| Heavy Chain FR1 | QVQLVQSGAEVRKPGASVKVSCKAS (SEQ ID NO: 371) |
| Heavy Chain CDR1 | GYTFTSYG (SEQ ID NO: 439) |
| Heavy Chain FR2 | ISWVRQAPGQGLEWMGW (SEQ ID NO: 264) |
| Heavy Chain CDR2 | ISAYNGNT (SEQ ID NO: 265) |
| Heavy Chain FR3 | NYAQKLQGRVTMTTDTSTSTAYMELRSLRSDDTAVYYCAR |
| | (SEQ ID NO: 372) |
| Heavy Chain CDR3 | VTGITGTTIDP (SEQ ID NO: 267) |
| Heavy Chain FR4 | WGQGTLVTVSS (SEQ ID NO: 214) |
| Light Chain FR1 | DIVMTQSPSSLSASVGDRVTITCRAS (SEQ ID NO: 407) |
| Light Chain CDR1 | QSISSY (SEQ ID NO: 216) |
| Light Chain FR2 | LNWYQQKPGKAPKLLIY (SEQ ID NO: 217) |
| Light Chain CDR2 | DAS (SEQ ID NO: 269) |
| Light Chain FR3 | NLETGVPSRFSGSGSGTDFTLTISSLQPEDFATYYC (SEQ ID |
| | NO: 317) |
| Light Chain CDR3 | QQFNNYPLT (SEQ ID NO: 408) |
| Light Chain FR4 | FGGGTKLEIK (SEQ ID NO: 394) |
| Light Chain Locus | kappa |

1997_02_B03

| | |
|---|---|
| Heavy Chain FR1 | QVQLVESGAEVKKPGASVKVSCKAS (SEQ ID NO: 409) |
| Heavy Chain CDR1 | GYTFTSYY (SEQ ID NO: 396) |
| Heavy Chain FR2 | MHWVRQAPGQGLEWMGI (SEQ ID NO: 303) |
| Heavy Chain CDR2 | INPSGGST (SEQ ID NO: 304) |
| Heavy Chain FR3 | SYAQKFQGRVTMTRDTSTSTVYMELSSLRSEDTAVYYCAR |
| | (SEQ ID NO: 397) |
| Heavy Chain CDR3 | AGGYYYYYMDV (SEQ ID NO: 398) |
| Heavy Chain FR4 | WGQGTLVTVSS (SEQ ID NO: 214) |
| Light Chain FR1 | QSGLTQPPSASGTPGQRVTISCSGS (SEQ ID NO: 399) |
| Light Chain CDR1 | GPNIGNNY (SEQ ID NO: 400) |
| Light Chain FR2 | VYWYQQLPGTAPKLLMY (SEQ ID NO: 401) |
| Light Chain CDR2 | RNN (SEQ ID NO: 310) |
| Light Chain FR3 | QRPSGVPDRFSGSKSGTSASLAISGLQSEDEADYYC (SEQ ID |
| | NO: 410) |
| Light Chain CDR3 | AAWDDSLNGYV (SEQ ID NO: 411) |
| Light Chain FR4 | FGTGTKLTVLG (SEQ ID NO: 412) |
| Light Chain Locus | lambda |

Humanized mAb sequences
Humanized 79E3E3 Heavy Chain Variable Region
QIQLVQSGAEVKKPGESLKISCKASGYTFTDYAIGWVRQMPGKGLEWMGIINTQTGKPK
YSPSFQGQFIFSLDTSINTTYLQWSSLKASDTAIYFCTRLGTGNTKGFAYWGQGTTVTVS
S (SEQ ID NO: 413)
Humanized 79E3E3 Light Chain Variable Region
DIQITQSPSSLSASLGDKVTITCRSSQSLLYSENNQDYLAWYQQKPGKAPKLLIYGASNL
QSGVPSRFSGRGSGTDFTLTISSLQPEDFATYYCEQTYRYPFTFGPGTKVDIKR (SEQ ID
NO: 414)
Humanized 9-G05 Heavy Chain VH_1
Leader sequence-VH-hIgG1CH-Stop codon*
MGWSCIILFLVATATGVHSQVQLVQSGAEVKKPGASVKVSCKASGYTFPDYNMDWVR
QAPGQRLEWMGYINPDNGGTIYNQKFKGRVTLTVDTSASTAYMELSSLRSEDTAVYYC
ARLDSSGYGYYAMDYWGQGTSVTVSSASTKGPSVFPLAPSSKSTSGGTAALGCLVKD
YFPEPVTVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRTPEVTCV
VVDVSHEDPEVKFNWYVDGVEVHNAKTKPREEQYASTYRVVSVLTVLHQDWLNG
KEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVM
HEALHNHYTQKSLSLSPGK* (SEQ ID NO: 415)
Humanized 9-G05 Heavy Chain VH_2
Leader sequence-VH-hIgG1CH-Stop codon*
MGWSCIILFLVATATGVHSQVQLVQSGAEVKKPGASVKVSCKASGYTFPDYNMDWVR
QAPGQRLEWIGYINPDNGGTIYNQKFKGRVTLTVDTSASTAYMELSSLRSEDTAVYYCA
RLDSSGYGYYAMDYWGQGTSVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVV
VDVSHEDPEVKFNWYVDGVEVHNAKTKPREEQYASTYRVVSVLTVLHQDWLNGK
EYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHE
ALHNHYTQKSLSLSPGK* (SEQ ID NO: 416)
Humanized 9-G05 Heavy Chain VH_3
Leader sequence-VH-hIgG1CH-Stop codon*
MGWSCIILFLVATATGVHSQVQLVQSGAEVKKPGASVKVSCKASGYTFPDYNMDWVR
QAPGQRLEWMGYINPDNGGTIYNQKFKGRATLTVDTSASTAYMELSSLRSEDTAVYYC
ARLDSSGYGYYAMDYWGQGTSVTVSSASTKGPSVFPLAPSSKSTSGGTAALGCLVKD
YFPEPVTVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRTPEVTCV
VVDVSHEDPEVKFNWYVDGVEVHNAKTKPREEQYASTYRVVSVLTVLHQDWLNG
KEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVM
HEALHNHYTQKSLSLSPGK* (SEQ ID NO: 417)
Humanized 9-G05 Heavy Chain VH_4
Leader sequence-VH-hIgG1CH-Stop codon*
MGWSCIILFLVATATGVHSQVQLVQSGAEVKKPGASVKVSCKASGYTFPDYNMDWVR
QAPGQSLEWIGYINPDNGGTIYNQKFKGRATLTVDTSASTAYMELSSLRSEDTAVYYCA
RLDSSGYGYYAMDYWGQGTSVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVV
VDVSHEDPEVKFNWYVDGVEVHNAKTKPREEQYASTYRVVSVLTVLHQDWLNGK
EYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHE
ALHNHYTQKSLSLSPGK* (SEQ ID NO: 418)
Humanized 9-G05 Light Chain VL_1
Leader sequence-VL-hIgKCL-Stop codon*
MGWSCIILFLVATATGVHSDIVMTQSPDSLAVSLGERATINCRASESVDNYGISFMHWY
QQKPGQPPKLLIYRASNLDSGVPDRFSGSGSGTDFTLTISSLQAEDVATYYCQQSYKDPR
TFGGGTKLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFN
RGEC* (SEQ ID NO: 419)
Humanized 9-G05 Light Chain VL_2
Leader sequence-VL-hIgKCL-Stop codon*
MGWSCIILFLVATATGVHSDIVLTQSPASLAVSPGQRATITCRASESVDNYGISFMHWYQ
QKPGQPPKLLIYRASNLDSEVPARFSGSGSRTDFTLTINPVEANDTATYYCQQSYKDPRT
FGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNR
GEC* (SEQ ID NO: 420)
16E10 Heavy Chain Variable Region
QSLEESGGDLVKPGASLTLTCRVSGFSFSSSYYMCWVRQAPGKGLEWIACIGTTRGSTY
YATWAKGRFTISKISSTTVTLQMTSLTDADTATYFCARDATGYRINTIGLYFNLWGPGTL
VTVSS (SEQ ID NO: 421)
Humanized 16E10 Heavy Chain Variable Region VH_1
QSLLESGGGLVKPGGSLRLSCAVSGFSFSSSYYMCWVRQAPGKGLEWVSCIGTTRGSTY
YADSAKGRFTISKISKNTVYLQMTSLRAEDTAVYFCARDATGYRINTIGLYFNLWGPGT
LVTVSS (SEQ ID NO: 422)
Humanized 16E10 Heavy Chain Variable Region VH 2
EVQLLESGGGLVKPGGSLRLSCAVSGFSFSSSYYMCWVRQAPGKGLEWVSCIGTTRGST
YYADSAKGRFTISKDNSKNTVYLQMTSLRAEDTAVYFCARDATGYRINTIGLYFNLWG
QGTLVTVSS (SEQ ID NO: 423)
Humanized 16E10 Heavy Chain Variable Region VH_3
QSLLESGGGLVKPGGSLRLSCAVSGFSFSSSYYMCWVRQAPGKGLEWVSCIGTTRGSTY
YADSAKGRFTISKESKNTVYLQMSSLRAEDTAVYFCARDATGYRINTIGLYFNLWGPGT
LVTVSS (SEQ ID NO: 424)
Humanized 16E10 Heavy Chain Variable Region VH 4
EVQLLESGGGLVKPGGSLRLSCAVSGFSFSSSYYMCWVRQAPGKGLEWVSCIGTTRGST
YYADSAKGRFTISKDNSKNTVYLQMSSLRAEDTAVYFCARDATGYRINTIGLYFNLWG
QGTLVTVSS (SEQ ID NO: 425)
Humanized 16E10 Heavy Chain Variable Region VH_5
QSLLESGGGLVKPGGSLRLSCAVSGFSFSSSYYMCWVRQAPGKGLEWVSCIGTTRGSTY
YADSAKGRFTISKESKNTVYLQMSSLRAEDTAVYFCARDATGYRIQTIGLYFNLWGPGT
LVTVSS (SEQ ID NO: 426)
Humanized 16E10 Heavy Chain Variable Region VH_6
EVQLLESGGGLVKPGGSLRLSCAVSGFSFSSSYYMCWVRQAPGKGLEWVSCIGTTRGST
YYADSAKGRFTISKDNSKNTVYLQMSSLRAEDTAVYFCARDATGYRIQTIGLYFNLWG
QGTLVTVSS (SEQ ID NO: 427)
16E10 Light Chain Variable Region
ELTQTPSSVEAAVGGTPTIKCQASQTIYSYLSWYQQKPGQPPKLLIYEASKLASGVPSRFS
GSGSGTDYTLTISDLECADAATYYCQSYHGTASTEYNTFGGGTEVVVK (SEQ ID NO:
428)
Humanized 16E10 Light Chain Variable Region VL_1
QLTQSPSSLSASVGDRVTITCQASQTIYSYLSWYQQKPGKPPKLLIYEASKLASGVPSRFS
GSGSGTDYTLTISSLQPEDFATYYCQSYHGTASTEYNTFGGGTKVEIK (SEQ ID NO:
429)
Humanized 16E10 Light Chain Variable Region VL_2
DIQLTQSPSSLSASVGDRVTITCQASQTIYSYLSWYQQKPGKPPKLLIYEASKLASGVPSR
FSGSGSGTDYTLTISSLQPEDFATYYCQSYHGTASTEYNTFGGGTKVEIK (SEQ ID NO:
430)
Humanized 16E10 Light Chain Variable Region VL_3
QLTQSPSSLSASVGDRVTITCQASQTIYSYLSWYQQKPGKPPKLLIYEASKLASGVPSRFS
GSGSGTDYTLTISSLQPEDTATYYCQSYHGTASTEYNTFGGGTKVEIK (SEQ ID NO:
431)
Humanized 16E10 Light Chain Variable Region VL_4
DIQLTQSPSSLSASVGDRVTITCQASQTIYSYLSWYQQKPGKPPKLLIYEASKLASGVPSR
FSGSGSGTDYTLTISSLQPEDTATYYCQSYHGTASTEYNTFGGGTKVEIK (SEQ ID NO:
432)
Humanized 16E10 Light Chain Variable Region VL_5
QLTQSPSSLSASVGDRVTITCQASQTIYSYLSWYQQKPGKPPKLLIYEASKLASGVPSRFS
GSGSGTDYTLTISSLQPEDTATYYCQSYHGTASTEYQTFGGGTKVEIK (SEQ ID NO:
433)
Humanized 16E10 Light Chain Variable Region VL_6
DIQLTQSPSSLSASVGDRVTITCQASQTIYSYLSWYQQKPGKPPKLLIYEASKLASGVPSR
FSGSGSGTDYTLTISSLQPEDTATYYCQSYHGTASTEYQTFGGGTKVEIK (SEQ ID NO:
434)

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An anti-integrin alpha 11 beta 1 (α11β1) antibody, or antigen-binding fragment thereof, comprising an amino acid sequence selected from a group consisting of SEQ ID NO: 103-443.

2. An anti-α11β1 antibody, or antigen-binding fragment thereof, comprising a CDR sequence encompassed within any one of SEQ ID NO: 103-207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443.

3. An anti-α11β1 antibody, or antigen-binding fragment thereof, comprising CDR1, CDR2, and CDR3 encompassed within any one of SEQ ID NO: 103-206, or 413-435.

4. The anti-α11β1 antibody, or antigen-binding fragment thereof, of claim 1, comprising an amino acid sequence selected from a group consisting of SEQ ID NO: 103-114, 207-311, and 312-443.

5. The anti-α11β1 antibody, or antigen-binding fragment thereof, of claim 2, comprising a CDR sequence encompassed within any one of SEQ ID NO: 103-114, 207, 209, 211, 213, 216, 218, 220, 223, 225, 228, 233, 234, 236, 240, 241, 245, 247, 253, 255, 257, 259, 261, 265, 267, 269, 271, 275, 277, 279, 281, 283, 287, 289, 291, 293, 296, 300, 304, 306, 308, 310, 312, 314, 316, 318, 320, 322, 324, 325, 327, 329, 334, 336, 338, 340, 342, 344, 348, 351, 353, 355, 358, 360, 361, 364, 366, 368, 369, 374, 376, 377, 379, 380, 381, 383, 384, 385, 387, 389, 392, 393, 396, 398, 400, 402, 405, 408, 411, or 413-443.

6. The anti-α11β1 antibody, or antigen-binding fragment thereof, of claim 2, comprising one or more CDR sequences encompassed within any one of SEQ ID NO: 103-114, or 413-434.

7. The anti-α11β1 antibody, or antigen-binding fragment thereof, of claim 3, comprising CDR1, CDR2, and CDR3 encompassed within any one of SEQ ID NO: 103-114 or 413-434.

8. The anti-α11β1 antibody, or antigen-binding fragment thereof, of claim 1, wherein the antibody, or antigen-binding fragment thereof, is a monoclonal antibody, or antigen-binding fragment thereof.

9. The anti-α11β1 antibody, or antigen-binding fragment thereof, of claim 1, wherein the antibody, or antigen-binding fragment thereof, is a humanized antibody, or antigen-binding fragment thereof.

10. The anti-α11β1 antibody, or antigen-binding fragment thereof, of claim 1, wherein the antibody, or antigen-binding fragment thereof, reduces interaction of α11β1 with collagen in human α11β1-expressing cells.

11. An anti-α11β1 antibody, or antigen-binding fragment thereof, that competes with the antibody, or antigen-binding fragment thereof, of claim 1.

12. A nucleic acid, comprising a nucleic acid sequence encoding an antibody, or antigen-binding fragment thereof, of claim 1.

13. The nucleic acid of claim 11, wherein the nucleic acid sequence comprises a sequence selected from a group consisting of SEQ ID NO: 1-102.

14. A vector comprising the nucleic acid of claim 12.

15. A host cell comprising the nucleic acid of claim 12.

16. A method of producing an antibody, or antigen-binding fragment thereof, comprising culturing the host cell of claim 14 under conditions suitable for expression of the antibody or antigen-binding fragment thereof.

17. A method of treating a subject having or at risk of chronic kidney disease, the method comprising administering to the subject a therapeutically effective amount of the antibody, or antigen-binding fragment thereof, of claim 1.

18. The method of claim 17, wherein the chronic kidney disease is or comprises Primary Glomerular Disease (including, but not limited to, IgA Nephropathy and focal segmental glomerular sclerosis), Secondary Glomerular Disease (including, but not limited to, lupus nephritis), Thrombotic Microangiopathy, Tubulointerstitial Diseases (including, but not limited to, Obstructive Uropathy), Diabetic Nephropathy, Hypertensive Nephropathy, Ischemic Nephropathy, Cardiorenal Syndromes in CKD, Inherited Disorders of the Glomerulus (including, but not limited to, Alport syndrome), Cystic Diseases of the Kidney (including, but not limited to, Polycystic Kidney Disease), or Inherited Disorders of the Renal Tubule.

19. The method of claim 17, wherein administering the therapeutically effective amount of the antibody, or antigen-binding fragment thereof results in a reduction in a measured marker, sign and/or symptom by at least about 10%, 15%, 20%, 25%, 30%, 35%, 40%, 50%, 55%, 60%, 65%, 70%, 75%, 80%, 85% or 90% relative to a control.

20. The method of claim 19, wherein the control comprises a level of measured marker, sign and/or symptom in the subject prior to administration of the antibody.

21. The method of claim 19, wherein the control comprises a level of measured marker, sign and/or symptom in a subject suffering from the kidney-related disorder.

22. The method of claim 19, wherein the control comprises an average level of measured marker, sign and/or symptom in a population of subjects suffering from the kidney-related disorder.

23. The method of claim 1, wherein the measured marker, sign and/or symptom is or comprises: COL1A1, Fibronectin, PAI-1, IL-11, CXCL1, MCP-1, IL-6, TIMP-1, Hyaluronic acid, TGFβ, CTGF, PDGF, MMP9, or a combination thereof.


SEQUENCE LISTING (443 sequences)

SEQ ID NO: 1 - DNA, 417 bp, Artificial Sequence
  source: /note=“Description of Artificial Sequence Synthetic polynucleotide”
atggattggttgtggaacttgctattcctgatggtagttgcccaaagtgctcaagcacagatccagttggtacagtctggacctgaagtaaagaagcctggagagtcagtgaagatctcctgcaaggcctctgggtataccttcacagactatgcaatgaactgggtgaaacaggctccaggaaatggcctgaagtggatgggctggatcaacacccaaactggaaagccaacatatgcggatgatttcaaacaacggtttgtcttctctttggaaacttctgccagaactacatatttgcagatcaacaacctcaatattgaagacacagctacatatttctgtacgagattgggtacaggtaatacgaaggggtttgcttactggggccaaggcactctggtcactgtctcttca

SEQ ID NO: 2 - DNA, 399 bp, Artificial Sequence
  source: /note=“Description of Artificial Sequence Synthetic polynucleotide”
atggaatcacagacgcatgtcctcatttcccttctgctctgtgtatcaggtacctgtggggacattttgataaaccagtctccagcctctctgactgtgtcagcaggagagagggtcactatgagctgcaagtccagtcagagtcttctatacagtgaaaacaaccaggactatttggcttggtaccagcagaaaccaggacagtttcctaaattgcttatctatggggcatccaaccggcacactggggtccctgatcgcttcacaggcagtggatctgggacagacttcactctgaccatcagcagtgtgcaggctgaagacctggctgattattattgtgagcagacctacagatatccattcacgttcggctcagggacgaagttggaaataaaa

SEQ ID NO: 3 - DNA, 414 bp, Artificial Sequence
  source: /note=“Description of Artificial Sequence Synthetic polynucleotide”
atggagttggaattgagcttaatttttattttttctcttttaaaagatgtccagtgtgaagtacagctggtggagtctggaggaagcttggttcaacctgggggttctctgaaactctcctgtgtagcctcaggatacactttcagtaactactggatggactgggttcggcagtctcctggaaagtccctggaatggattggagagattaacacggatggcagaaggaccaactatgcaccatccataaaggatcgattcacaatctccagagacaatgccaagagcaccctgtatctgcagatgagcaatgtgaaatcagatgacacagccatttattactgtaccatactacgggtatacccccactactttgattactggggccaaggagtcatggtcacagtctcctca

SEQ ID NO: 4 - DNA, 396 bp, Artificial Sequence
  source: /note=“Description of Artificial Sequence Synthetic polynucleotide”
atgatgagtcctgcccagttcctgtttctgctaatgctctggatccaggaagcccgcggagatgttgtgatgacccagacaccaccgtctttgtcggttgccattggacaatcagtctccatctcttgcaagtcaagtcagagcctcgtatatagtgatggagagacatatttgcattggtttttacagagtcctggcaggtctccgaagcgcctaatttatcacgtgtctaatctgggctctggagtccctgacaggttcagtggcactggatcactgacagattttacacttagaatcagcagagtggaggctgaggatttgggagtttattactgcgcgcaaactacacattttcctcccacgtttggagctgggaccaagctggaactgaaa

SEQ ID NO: 5 - DNA, 405 bp, Artificial Sequence
  source: /note=“Description of Artificial Sequence Synthetic polynucleotide”
atggctgtcctggtgctgttgctctgcctggtgacatttccaagctgtgccctgtcccaggtgcagttgaaggagtcaggacctggtctggtgcagccctcacagaccctgtccctcacctgcactgtctctgggttctcattaaccagcaatagtgttagctgggttcgccaggctccgggaaagggtctggagtggatgggagcaatatggagtggtggaagcacagattataattcagctctcaaatcccgactgagcatcagcagggacacctccaagagccaagttttcttaaaaatgaacagtctgcaaactgaagacacagccatttacttctgtaccagatctcactgggagccctttgattactggggccaaggagtcatggtcacagtctcctca

SEQ ID NO: 6 - DNA, 399 bp, Artificial Sequence
  source: /note=“Description of Artificial Sequence Synthetic polynucleotide”
atggaatcacaaactcaggccctcatatccctgctgctctgggtatatggtacctgtggggacattgtgatgacccagtctccattctccctggctgtgtcagaaggagagatggtcactataaactgcaagtccagtcagggtcttttatccagtggaaaccaaaagaactacttggcctggtaccagcagagaccagggcagtctcctaaactactgatctactatgcatccactaggcaatcaggggtccctgatcgcttcataggcggtggatctgggacagacttcactctgaccatcagcgatgtgcaggctgaagacctggcagattattactgcctgcagcattacagctatcctcccacgttcggttctgggaccaagctggagatcaaa

SEQ ID NO: 7 - DNA, 405 bp, Artificial Sequence
  source: /note=“Description of Artificial Sequence Synthetic polynucleotide”
atggctgtcctggtgctgttgctctgcctggtgacatttccaagctgtgccctgtcccaggtgcagctgagggagtcaggacctggtctggtgcagccctcacagaccctgtccctcacctgcactgtctctgggttctcattgaccagcaatagtgtgacctgggttcgccagcctccgggaaagggtctggagtggatgggagcgatatggagtgatggaagcacagattataattcaactctcaaatcccgactgagcatcagtagggacacctccaagagccaagttttcttaaaaatgagcagtctgcaaactgaagacacagccatttacttctgtaccagatcccactgggagccctttgattactggggccaaggagtcatggtcacagtctcctca

SEQ ID NO: 8 - DNA, 394 bp, Artificial Sequence
  source: /note=“Description of Artificial Sequence Synthetic polynucleotide”
atggaatcacaaactcaggccctcatatccctgctgctctgggtatatggtacctgtggggacattgtgatgacccagtctccactctccctggctgtgtcagaaggagagacggtcactatgaactgcaagtccagtcagagtcttttttccagtggaaatcaaaagaactacttggcctggtaccagcagaaaccagggcagtctcctaaactactgatctactatgcatccactaggcaatcaggggtccctgatcgcttcataggcagtggatctgggacagacttcactctgaccatcagcgatgtgcagactgaagacctggcagattattactgcctgcagcattacaactatcctcccacgttcggttctgggaccaagctggaga

SEQ ID NO: 9 - DNA, 402 bp, Artificial Sequence
  source: /note=“Description of Artificial Sequence Synthetic polynucleotide”
atggacttgcgactgacttatgtctttattgttgctattttaaaaggtgtcttgtgtgaggtgaaactggaggaatctgggggaggtttggtgcaacctggaatgtccgtgaaactctcttgtgcaacctctggattcattttcagtgactactggatggaatgggtccgccaggctccagggaaggggctagaatgggtagccgaaattagaaacaaagctaataattatgcaacatactatgggaagtctatgaaaggcagattcaccatctcaagagatgattccaaaagtatagtctacctacaagtgaacagcataagatctgaagatactgctatttattactgtgcaccgaattttgattactggggccaaggagtcatggtcacggtctcctca

SEQ ID NO: 10 - DNA, 393 bp, Artificial Sequence
  source: /note=“Description of Artificial Sequence Synthetic polynucleotide”
atgagtcctgtccagtccctgtttttgctattgctttggattctgggaacccatggtgatgttgtgctgacccagactccacccactttatcggctaccattggacaatcagtctctatctcttgcaggtcaagtcagagtctcttacatagtactggaaacacctatttaaattggttgctacagaggccaggccaacctccgcaacttctaatttatttggtttccagactggaatctggggtccccaacaggttcagtgccagtgggtcaggaactgatttcacactcaaaatcagtggaatagaggctgaggatttgggggtttattactgcgtgcaaagttcccatactccgtacacgtttgggactgggaccaagctggaactgaaa

SEQ ID NO: 11 - DNA, 405 bp, Artificial Sequence
  source: /note=“Description of Artificial Sequence Synthetic polynucleotide”
atggacatcaggctcagcttggttttccttgtcctttttatgaaaggtgtccagtgtgaggtgcagttggtggagtctgggggaggcttagtgcagcctggaaggtccctgaaactctcctgtgcagcctcacgattcacttttagtgactataacatggcctgggtccgccaggctccaaagaagggtctggagtgggtcgcaaccatttatcatgatgatagtggttcttactatcgagactccgtgaagggccgattcaccatctccagaaataatgcaaaaagcactctgtacctgcagatggacagtctgaggtctgaggacatggccacttattactgtgcaagacataacaatggctttgattactggggccaaggagtcatggtcacagtcgcctca

SEQ ID NO: 12 - DNA, 393 bp, Artificial Sequence
  source: /note=“Description of Artificial Sequence Synthetic polynucleotide”
atgaagtggcctgttaggctgttggtgctgttcttctggattcctgcttccgggggtgatgttgtgatgacacaaactccagtctccctgcctgtccgccttggaggtcaagcctctatctcttgccggtcaagtcagagcctggtacacagtaatggaaacacctacttgcattggtacctacagaagccaggccagtctccacagctcctcatcaatcgggtttccaacagattttctggggtgccagacaggttcagtggcagtgggtcagggacagatttcaccctcaagatcaacagagtagagcctgaggacttgggagattattactgcttacaaagtacacattttccactcacgttcggttctgggaccaagctggagaccaaa

SEQ ID NO: 13 - DNA, 408 bp, Artificial Sequence
  source: /note=“Description of Artificial Sequence Synthetic polynucleotide”
atggacatcaggctcagcttgggtttccttgtccttttcataaaaggtgaccagtgtgcggtgcaactggtggagtctgggggaggcttagtgcagcctggaaggtccctgaaactctcctgtgcagcctcaagaatcactttcactgactattacatggcctgggtccgccaggctccaacgaagggtctggagtgggtcgcaaccattagttctgatggtggtgacactttctatcgagactccgtgaagggccgatttactatctccagagacaatgcaaaaagcaccctatatttgcaaatggtcagtctgaggtctgaggacacggccacttattactgttcaacagatcggggagctcagtttggttactggggccaaggcactctggtcactgtctcttca

SEQ ID NO: 14 - DNA, 378 bp, Artificial Sequence
  source: /note=“Description of Artificial Sequence Synthetic polynucleotide”
atggctccagtccagctcttagggctgctgctgatttggctcccagccatgagatgtgacatccagatgacccagtctccttcattcctgtctgcatctgtgggagacagagtctctatcaactgcaaagcaagtcagaatgttcacgagaacctaaactggtatcagcaaaagcttggagaagctcccaaacgcctgatatataatacaaacaatttgcaaacaggcatcccatcaaggttcagtggcagtggatctggtgcagattacacactcaccatcagcagcctgcagcctgaagattttgccacatatttctgtttgcagcataatgcttttccgtacacgtttggacctgggacgaagctggaactgaaa

SEQ ID NO: 15 - DNA, 366 bp, Artificial Sequence
  source: /note=“Description of Artificial Sequence Synthetic polynucleotide”
gaggtccagctgcaacagtctggacctgagctggtgaagcctggggcttcagtgaagataccctgcaaggcttctggatacacattccctgactacaacatggactgggtgaagcagagccatggaaagagccttgagtggattggatatattaatcctgacaatggtggtactatctacaaccagaagttcaagggcaaggccacattgactgtagacaagtcctccagcacagcctacatggagctccgcagcctgacatctgaggacactgcagtctattactgtgcaagattagacagctcaggctacggttactatgctatggactactggggtcaaggaacctcagtcaccgtctcctca

SEQ ID NO: 16 - DNA, 333 bp, Artificial Sequence
  source: /note=“Description of Artificial Sequence Synthetic polynucleotide”
gacattgtgctgacccaatctccagcttctttggctgtgtctctagggcagagggccaccatctcctgcagagccagcgaaagtgttgataattatggcattagttttatgcactggtaccagcagaaaccaggacagccacccaaactcctcatctatcgtgcatccaacctagactctgagatccctgccaggttcagtggcagtgggtctaggacagacttcaccctcaccattgatcctgtggagactgatgatgttgcaacctattactgtcagcaaagttataaggatcctcggacgttcggtggaggcaccaagctggaaatcaaa

SEQ ID NO: 17 - DNA, 366 bp, Artificial Sequence
  source: /note=“Description of Artificial Sequence Synthetic polynucleotide”
aaagtgatgctggtggagtctgggggagccttagtgaagcctggagggtccctgaaactctcctgtgtagcctctggattcactttcagtaactatgccatgtcttgggttcgccagactccagagaagaggctggagtgggtcgcaaccattagtagtggtggttattacacttactatccagacagtgtgaagggtcgattcaccatctccagagacaatgccaggaacaccctgttcctgcaaatgagcagtctgaggtctgaggacacggccatgttttactgtgcaagagaggatgattacggaagatattcctatactatggactactggggtcaaggaacctcagtcaccgtctcctca

SEQ ID NO: 18 - DNA, 336 bp, Artificial Sequence
  source: /note=“Description of Artificial Sequence Synthetic polynucleotide”
gatgttgtgatgacccaaactccactctccctgcctgtcagtcttggagatcaagtctccatctcttgcagatgtagtcagagccttgtacacagtaatggaaacacctatttacattggtacctgcagaagccaggccagtctccacagctcctgatctacaaaatttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccgagctggaaataaaa

SEQ ID NO: 19 - DNA, 357 bp, Artificial Sequence
  source: /note=“Description of Artificial Sequence Synthetic polynucleotide”
gaggtccagctgcagcagtctggacctgagctggtaaagcctggggcttcagtgaggatatcctgcaaggcttctggatacacattcactgactactacatacactgggtgaagcagaagcctgggcagggccttgaatgcattggagagatttatcctggaactgataatacttactacagtaaaaaattcaggggcaaggccacactgactgcagacaaatcctccgacacagcctacatgcagctcagcagcctgacatctgaggactctgcagtctatttctgtgcaagaggagactactatagggggtacttcgatgtctggggcgcagggaccacggtcaccgtctcctca

SEQ ID NO: 20 - DNA, 336 bp, Artificial Sequence
  source: /note=“Description of Artificial Sequence Synthetic polynucleotide”
gatgttgtgatgactcagacctcactcactttgtcggttaccattggacaaccagcctccatctcttgcaagtcaagtcagagcctcttacatagtaatggaaagacatatttgaattggttattacagaggccaggccagtctccaaagttcctaatctatctggtgtctaaactggaatctggagtccctgacaggttcagtggcagtggatcagggacagatttcacactgaaaatcagcagagtggaggctgaggatttgggggtttattactgcttgcaatctacacattttccttggacgttcggtggaggcaccaagctggaaatcaaa

SEQ ID NO: 21 - DNA, 357 bp, Artificial Sequence
  source: /note=“Description of Artificial Sequence Synthetic polynucleotide”
gaggtccagctgcagcagtctggacctgagctggtaaagcctggggcttcagtgaagaaatcctgcaaggcttctggatacacattcactgactactacatgcactgggtgaagcagaagcctgggcagggccttgagtggattggaaagatttatcctggaagtggtaatactcactacaatgagaagttcaagggcaaggccacactgactgcagacaaatcctccagcacagcctacatgcagctcagcagcctgacatctgaggactctgcagtctatttctgtgcaaccaattactacggctacagggcaatgaactattggggtcaaggatcctcagtcaccgtctcctca

SEQ ID NO: 22 - DNA, 321 bp, Artificial Sequence
  source: /note=“Description of Artificial Sequence Synthetic polynucleotide”
gacatccatttgacccagtctccatcctccttatctgcctctctgggagaaagaatcagtctcacttgccgggcaagtcaggacatttatattagcttaaactggtttcagcagaaaccagatggaactattaaactcctgatctacggcacatccagtttagattctggtgtccccaaaaggttcagtggcagtaggtctgggtcagattattctctcaccatcagcagccttgagtctgaagattttgcagactattactgtctacaatatgctagttctccgtacacgttcggaggggggaccaagctggaaataaaa

SEQ ID NO: 23 - DNA, 363 bp, Artificial Sequence
  source: /note=“Description of Artificial Sequence Synthetic polynucleotide”
gaggtccagctgcagcagtctggacctgagctggtaaagcctggggcttcagtgaagatatcctgcaaggcttctggatacacattcactgactactacatgcactgggtgaagcagaagcctgggcagggccttgagtggattggagagatttatcctggaagtggtaatccttactacaatgagaagttcaagggcaaggccacactgactgcagacaaatcatccagctcagcctacatgcagctcagcagcctgacatctgaggactctgcagtctatttctgtgcaagaacctcctacggtagagtagggacagggtttgcttactggggccaagggactctggtcactgtctctgca

SEQ ID NO: 24 - DNA, 333 bp, Artificial Sequence
  source: /note=“Description of Artificial Sequence Synthetic polynucleotide”
aattttgtgatgacccaaactccactctccctgcctgtcagtcttggagatcaagcctccatctcttgcagatctagtcagagccttctacacagtaacggaaacacctatttacattggtacctgcagaagccaggccagtctccaaagctcctgatctacaaagtttccaaccgattttctggggtcccagacaggttcagtggcagtggatcagggacagatttcacactcaagatcaacagagtggagactgaggatctgggaatttatttctgctctcaaagttcacatgttcccacgttcggtgctgggaccaagctggagctgaaa

SEQ ID NO: 25 - DNA, 351 bp, Artificial Sequence
  source: /note=“Description of Artificial Sequence Synthetic polynucleotide”
caggtccagctgcagcagtctggggctgaactggcaaaacctggggcctcagtgaagatgtcctgcaaggcttctggctacacctttactaactactggatgcactgggtaaaacagaggcctggacagggtctggaatggattggatacattaatcctaacaatggttatactgagtacaatcagcgattcaaggacaaggccacattgactgcagacagatcctccaccacagcctacatgcaactaagcagcctgacatctgaggactctgcagtctattactgtgcaagatccgatatcattacgacagactactggggccaaggcaccactctcacagtctcctca

SEQ ID NO: 26 - DNA, 336 bp, Artificial Sequence
  source: /note=“Description of Artificial Sequence Synthetic polynucleotide”
gatgttgtgatgacccaaactccactctccctgcctgtcagtcttggagatcaagcctccatctcttgcagatctagtcagagccttgtatatagtaatggaaatacctatttacattggtacctgcagaagccaggccagtctccaaagctcctgatctacaaagtttccaaccgattttctggggtcccagacaggttcagtggcagtggatcagggacagatttcacactcaagataagcagagtggaggctgaggatctgggagtttatttctgctctcaaagtacacatgttccgtggacgttcggtggaggcaccaagctggaaatcaaa

SEQ ID NO: 27 - DNA, 357 bp, Artificial Sequence
  source: /note=“Description of Artificial Sequence Synthetic polynucleotide”
gaagtgaagcttgaggagtctggaggaggcttggtgcaacctggaggatccatgaaactctcttgtgctgcctctggattcacttttagtgacgcctggatggactgggtccgccagtctccagaggcggggcttgagtgggttgctgaaattagaaacaaagctcataatcctgcaacatactatgctgagtctgtgaaagggagattcaccatctcaagagatgattccaaaagtagtgtctacctgcaaatgaacagcttaagagctgaagacactggcatttattactgtaccttagtagcccctgatgctatggactactggggtcaaggaacctcagtcaccgtctcctca

SEQ ID NO: 28 - DNA, 339 bp, Artificial Sequence
  source: /note=“Description of Artificial Sequence Synthetic polynucleotide”
gacattgtgatgtcactgtctccatcctccctagctgtgtcagttggagagaaggttactatgagctgcaagtccagtcagagccttttatatagtcgcaatcaaaagaactacttggcctggtaccagcagaaaccagggcagtctcctaaactgctgatttactgggcatccactagggcatctggagtccctgatcgcttcacaggcagtggatctgggacagatttcactctcaccatcagcagtgtgaaggctgaagacctggcagtttattactgtcagcaatattatagctatccgtacacgttcggaggggggaccaagctggaaataaaa

SEQ ID NO: 29 - DNA, 348 bp, Artificial Sequence
  source: /note=“Description of Artificial Sequence Synthetic polynucleotide”
caggtccaactgcagcagtctgggcctgagctggtgaggcctggggcttcagtgaagatgtcctgcaaggcttcaggctataccttcaccagctactggatgcactgggtgaaacagaggcctggacaaggccttgagtggattggcatgattgatccttccaatagtgaaacttggttaaatcagaagttcaaggacaaggccacattgaatgtagacaaatcctccaacacagcctacatgcagctcagcagcctgacatctgaggactctgcagtctattactgtgcaagatatgatggttactacgactactggggccaaggcaccactctcacagtctcctca

SEQ ID NO: 30 - DNA, 333 bp, Artificial Sequence
  source: /note=“Description of Artificial Sequence Synthetic polynucleotide”
aacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacgtagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 31 - DNA, 354 bp, Artificial Sequence
  source: /note=“Description of Artificial Sequence Synthetic polynucleotide”
caggtccaactgcagcagcctggggctgagctggtgaggcctggggcttcagtgaagctgtcctgcaagccttctggctacaccttcaccagctactggatgaactgggtgaagcagaggcctggacaaggccttgaatggattggtatgattgatccttcagacagtgaaactcactacaatcaaatgttcaaggacaaggccacattgactgttgacaaatcctccaacacagcctacatgcagctcagcagcctgacatctgaggactctgcggtctattactgtgcgcagatctactatgcttacgacaaggcttactggggccaagggactctggtcactgtctctgca

SEQ ID NO: 32 - DNA, 333 bp, Artificial Sequence
  source: /note=“Description of Artificial Sequence Synthetic polynucleotide”
gacattgtgatgtcacagtctccatcctccctagctgtgtcagttggagagaaggttactatgagctgcaagtccagtcagagccttttatatagtagccatcaaaagaactacttggcctggtaccagcagaaaccagggcagtctcctaaactgctgatttactgggcatccactagggaatctggggtccctgatcgcttcacaggcagtggatctgggacagatttcagtctcaccatcagcagtgtgaaggctgaagacctggcagtttattactgtcaggaatattatagctggacgttcggtggaggcaccaagctggaaatcaaa

SEQ ID NO: 33 - DNA, 354 bp, Artificial Sequence
  source: /note=“Description of Artificial Sequence Synthetic polynucleotide”
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctcacaatggtggtactagcttcatccagaagttcaagggcaaggccacattgactgtagacaagtcctccagcacagcctacatggagctccgcagcctgacatctgaggactctgcagtctattattgtgcccctctgggacgaaaggaggggtttgcttactggggccaagggactctggtcactgtctctgca

SEQ ID NO: 34 - DNA, 333 bp, Artificial Sequence
  source: /note=“Description of Artificial Sequence Synthetic polynucleotide”
gacactgtgctgacacagtctcctgcttccttagttgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagccttcaccctcaacatccatcctgtggaggaggaggatgctgcaacctattactgtcagcacagtagggagcttccgtacacgttcggaggggggaccaagctggaaataaaa

SEQ ID NO: 35 - DNA, 357 bp, Artificial Sequence
  source: /note=“Description of Artificial Sequence Synthetic polynucleotide”
caggttactctgaaagagtctggccctgggatattgcagccctcccagaccctcagtctgacttgttctttctctgggttttcactgagcacttttgctatgggtgtaggctggattcgtcagccttcagggaagggtctggagtggctggcacacatttggtgggatgatgataagtactataacccagccctgaagagccggctcacaatctccaaggatacctccaaaaaccatgtattcctcaagatcgccaatgtggacactgcagatactgccacatactactgtgctcgaatgccgctaactttctactttgactactggggccaaggcaccactctcacagtctcctca

SEQ ID NO: 36 - DNA, 336 bp, Artificial Sequence
  source: /note=“Description of Artificial Sequence Synthetic polynucleotide”
gatgttttgctgacccaaactccactctccctgcctgtcagtcttggagatcaagcctccatctcttgcagatctagtcagagcattgtacatagtaatggacacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgttcacgttcggaggggggaccaagctggaaataaaa

SEQ ID NO: 37 - DNA, 357 bp, Artificial Sequence
  source: /note=“Description of Artificial Sequence Synthetic polynucleotide”
caggttactctgaaagagtctggccctgggatattgcagccctcccagaccctcagtctgacttgttctttctctgggttttcactgagaacttttgctatgggtgtaggctggattcgtcagccttcagggaagggtctggagtggctggcacacatttggtgggatgatgataagtactataacccagccctgaagagccggctcacaatctccaaggatacctccaaaaaccaggtattcctcaagatcgccaatgtggacactgcagatactgccacatactactgtgctcgaatgccgctaactttctactttgactactggggccaaggcaccactctcacagtctcctca

SEQ ID NO: 38 - DNA, 336 bp, Artificial Sequence
  source: /note=“Description of Artificial Sequence Synthetic polynucleotide”
gatgttttgatgacccaaactccactctccctgcctgtcagtcttggagatcaagcctccatctcttgtagatctagtcagagcattgtacatagtaatggaaacacctatttagaatggtacctgcagaaaccaggccagtctccaaagctcctgatctacaaagtttccacccgattttctggggtcccagacaggttcagtggcagtggatcagggacagatttcacactcaagatcagcagagtggaggctgaggatctgggagtttattactgctttcaaggttcacatgttccgttcacgttcggaggggggaccaagctggaaataaaa

SEQ ID NO: 39 - DNA, 345 bp, Artificial Sequence
  source: /note=“Description of Artificial Sequence Synthetic polynucleotide”
caggtccaactgcagcagcctggggctgaacttgtgatgcctggggcttcagtgaagctgtcctgcaaggcttctggctacaccttcaccaactactggatgcactgggtgaaacagaggcctggacaaggccttgagtggatcggagagattgatccttctgatagttatactaactacaatcaaaagttcaagggcaaggccacattgactgtagacaaatcctccagcacagcctacatgcagctcagcagcctgacatctgaggactctgcggtctattactgtacaagacagggtagtacctacgcgtggggtcaaggaacctcagtcaccgtctcctca

SEQ ID NO: 40 - DNA, 336 bp, Artificial Sequence
  source: /note=“Description of Artificial Sequence Synthetic polynucleotide”
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctccgacgttcggtggaggcaccaagctggaaatcaaa

SEQ ID NO: 41 - DNA, 336 bp, Artificial Sequence
  source: /note=“Description of Artificial Sequence Synthetic polynucleotide”
gaggtccagctgcaacaatctggacctgagctggtgaagcctggggcttcagtgaagatgtcctgtaaggcttctggatacacattcactgactactacatgatgtgggtgaagcagagtcatggaaagagccttgagtggattggagatattaatccttacaatggtggttctagctacaacccgaagttcaagggcagggccacattgactgtagacaaatcctccagcacagcctacatgcagctcaacagcctgacatctgaggactctgcagtctattactgtgcaagagggacttactggggccaagggactctggtcactgtctctgca

SEQ ID NO: 42 - DNA, 336 bp, Artificial Sequence
  source: /note=“Description of Artificial Sequence Synthetic polynucleotide”
gatgttgtgatgacccagactccactcactttgtcggttaccattggacaaccagcctccatctcttgcaagtcaagtcagagcctcttagatagtgctggaaagacatatttgaattggttgttacagaggccaggccagtctccaaagcgcctaatgtatctggtgtctaaactggactctggagtccctgacaggttcactggcagcggatcagggacagatttcacactgaaaatcagcagagtggaggctgaggatttgggagtttattattgctggcaaggtacacattttccgtacacgttcggaggggggaccaagctggaaataaaa

SEQ ID NO: 43 - DNA, 336 bp, Artificial Sequence
  source: /note=“Description of Artificial Sequence Synthetic polynucleotide”
caggtccaactgcagcagcctggggctgagcttgtgaagcctggggcttcagtgaagttgtcctgcaaggcttctggctacaccttcaccagctactggatgcactggataaagcagagacctggacaaggccttgagtggattggagagattaaccctagcaatggtggttctaactacaatgagaagttcaagagcaaggccacactgactgtagacaaatcctccagcacagcctacatgcaactcagcagcctgacatctgaggactctgcggtctatcactgtaaaagcagaggctactggggccaaggcaccactctcacagtctcctca

SEQ ID NO: 44 - DNA, 336 bp, Artificial Sequence
  source: /note=“Description of Artificial Sequence Synthetic polynucleotide”
gatgttgtgatgacccagactccactcactttgtcggttaccattggacaaccagcctccatctcttgcaagtcaagtcagagcctcttagatagttatggaaagacatatttgaattggttgttacagcggccaggccagtctccaaagcgcctaatctatctggtgtctaaattggactctggagtccctgacaggttcactggcagtggatcagggacagatttcacactgaaaatcagcagagtggaggctgaggatttgggaatttattattgctggcaaggtacacattttcctcacacgttcggctcggggacaaagttggaaataaaa

SEQ ID NO: 45 - DNA, 354 bp, Artificial Sequence
  source: /note=“Description of Artificial Sequence Synthetic polynucleotide”
caggttcagctgcagcagtctggagctgagctggcgaggcctggggcttcagtgaaactgtcctgcaaggcttctggctacaccttcacaagctatggtataagctgggtgaaacagagaactggacagggccttgagtggattggagagatttttcctagaagtagtaatacttactataatgagaagttcaagggcaaggccacactgactgcagacaagtcctccagcacagtgtacatggagttccgcagcctgacatctgaggactctgcggtctatttctgtgcaagagaggggggcctggcctggtttgcttactggggccaagggactctggtcactgtctctgca

SEQ ID NO: 46 - DNA, 336 bp, Artificial Sequence
  source: /note=“Description of Artificial Sequence Synthetic polynucleotide”
gatgttgtgatgacccagactccactcactttgtcggttaccattggacaaccagcttccatctcttgcaagtcaagtcagagcctcttatatactaatggaaacacctatttgaattggttattacagaggccaggccagtctccaaaacgcctaatctatctggtgtctaaattggactctggaatccctgacaggttcagtggcagtggatcagggacagatttcacactgagaatcagcagagtggaggctgaggatttgggagtttattactgcttgcagagtacacattttccattcacgttcggctcggggacaaagttggaaataaaa

SEQ ID NO: 47 - DNA, 336 bp, Artificial Sequence
  source: /note=“Description of Artificial Sequence Synthetic polynucleotide”
gaggtccagctgcaacaatctggacctgagctggtgaagcctggggcttcagtgaagatgtcctgtaaggcttctggatacacaatcactgactactacatgatgtggttgaagcagagtcatggaaagagccttgaatggattggagatattaatccttacactggtggtactagctacaaccagaagttcaagggcaaggccacattgactgtagacaaatcctccagcacagcctacctgcagctccacagcctgacatctgaggactctgcagtctattactgtgcaagaggggcctactggggccaaggcaccactctcacagtctcctca

SEQ ID NO: 48 - DNA, 336 bp, Artificial Sequence
  source: /note=“Description of Artificial Sequence Synthetic polynucleotide”
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaagctggaaataaaa

SEQ ID NO: 49 - DNA, 336 bp, Artificial Sequence
  source: /note=“Description of Artificial Sequence Synthetic polynucleotide”
gaggtccagctgcaacaatctggacctgagctggtgaagcctggggcttcagtgaagatgtcctgtaaggcttctggatacacaatcactgactactacatgatgtggttgaagcagagtcatggaaagagccttgaatggattggagatattaatccttacactggtggtactagctacaaccagaagttcaagggcaaggccacattgactgtagacaaatcctccagcacagcctacatgcagctcaacagcctgacatctgaggactctgcagtctattactgtgcaagaggggcctactggggccaaggcaccactctcacagtctcctca

SEQ ID NO: 50 - DNA, 336 bp, Artificial Sequence; identical to SEQ ID NO: 48
  source: /note=“Description of Artificial Sequence Synthetic polynucleotide”

SEQ ID NO: 51 - DNA, 336 bp, Artificial Sequence
  source: /note=“Description of Artificial Sequence Synthetic polynucleotide”
gaggtccagctgcaacaatctggacctgagctggtgaagcctggggcttcagtgaagatatcctgtaaggcttctggatacacgttcactgactactacatgaactgggtgaagcagagccatggcaggagccttgagttgattggagatattaatcctaacaatggtggttctaacttcaaccagaagttcaggggcaaggccacattgactgtagacaagtcctccagcacagcctatatggagctccgcagcctgacatctgaggactctgcaatctattactgtgcaagaatgggttactggggccaagggactctggtcactgtctctgca

SEQ ID NO: 52 - DNA, 336 bp, Artificial Sequence
  source: /note=“Description of Artificial Sequence Synthetic polynucleotide”
gatgttgtgatgacccagactccactcactttgtcggttaccattggacaaccagcctccatctcttgcaagtcaagtcagagcctcttacatagtgatggaaagacatatttgaattggatgttccagaggccaggccagtctccaaagcgcctaatctatctggtgtctaaactggactctggagtcccttacaggttcactggcggtggatcagggacagatttcacactgcaaatcagcagagtggagactgaggatttgggagtttattattgctggcaaggtacacattttcctcggacgttcggtggaggcaccaagctggaaatcaaa

SEQ ID NO: 53 - DNA, 342 bp, Artificial Sequence
  source: /note=“Description of Artificial Sequence Synthetic polynucleotide”
gaggtccagctgcagcagtctggacctgaactggtcaagcctggggcttcagtgaagatgtcctgcaaggcttctggatacacattcactgactactacatacactgggtgaagcagaagcctgggcagggccttgagtacattggagagatttatcctggaagtggtaatacttactacaatgggaagttcaggggcaaggccacactgactgcagacaagtcctccagcacagcctacatgcagctcagcagcctgacatctgaggactctgcagtctatttctgtggtagtggctactttgactactggggccaaggcaccactctcacagtctcctca

SEQ ID NO: 54 - DNA, 336 bp, Artificial Sequence
  source: /note=“Description of Artificial Sequence Synthetic polynucleotide”
gatgttgtgatgacccagactccactcactttgtctgttaccattggacagccagcttccatttcttgcaagtcaagtcagagcctcttatatagtaatggaaaaacctatttgaattggttattacagagtccaggccagtctccaaagctcctaatctatctggtgtctaaactggaatctggagtccctgacagattcagtggcagtggatcagggacagattttacactgaaactcagcagagtggaggctgaggatttgggagtatattactgcgtgcaaggtacacatttcccattcacgttcggctcggggacaaagttggaaataaaa

SEQ ID NO: 55 - DNA, 1368 bp, Artificial Sequence
  source: /note=“Description of Artificial Sequence Synthetic polynucleotide”
atggagactgggctgcgctggcttctcctggtcgctgtgttcaaaggtgtccagtgtcaggagcaactggtggagtccgggggagacctggtcaagcctggggcatccctgacactcacctgcacagcctctggattctccttcaataagaattattggatgtgctgggtccgccaggctccagggaaggggctggagtggatcggatgcatttataatggtgatggcaacacatactacgcgagctgggtgaatggccgattcaccatctccaaaacctcgtcgaccacggtgactctgcaaatgaccagtctgacagtcgcggacacggccatctatttctgtgcgagactacttaatatgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 56 - DNA, 720 bp, Artificial Sequence
  source: /note=“Description of Artificial Sequence Synthetic polynucleotide”
atggacacgagggcccccactcagctgctggggctcctactgctctggctcccaggtgccagatgtgctgacattgtgatgacccagactccagcctccgtggaggcagctgtgggaggcacagtcaccatcaagtgccaggccagtgagagcattggcaatgcattagcctggtatcagcagaaaccagggcagcctcccaagctcctgatctatactgcagccactctggcatctggggtcccatcgcggttcagcggcagtggatctgggacagagttcactctcaccatcagtggcgtgcagtgtgacgatgctgccacttactactgtcaaagctattattttactagtgttagtagttatggcaatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 57 - DNA, 1401 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcagagtctctggattctccttcagtagcagttattatatgtgttgggtccgccaggctccagggaaggggctggaatggatcgcatgtattggtactactcgtggtagcacttactacgcgacctgggcgaaaggccgattcaccatttctaaaatctcgtcgaccacggtgactctacaaatgaccagtctgacagacgcggacacggccacctatttctgtgcgagagatgctactggttataggattaacacgattggcctctattttaatttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 58 - DNA, 714 bp, Artificial Sequence
  source: /note=“Description of Artificial Sequence Synthetic polynucleotide”
atggacacgagggcccccactcagctgctggggctcctgctgctctggctcccaggtgccagatgtgcgttcgaattgacccagactccatcctccgtggaggctgctgtgggaggcacgcccaccatcaagtgccaggccagtcagaccatttacagttacttatcctggtatcagcagaaaccagggcagcctcccaagctcctgatctatgaagcgtccaaactggcctctggggtcccatcgcggttcagcggcagtggatctgggacagactacactctcaccatcagcgacctggagtgtgccgatgctgccacttactactgtcaaagctatcatggtactgctagtactgaatataatactttcggcggggggaccgaggtggtggtcagaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 59 - DNA, 1401 bp, Artificial Sequence
  source: /note=“Description of Artificial Sequence Synthetic polynucleotide”
atggagactgggctgcgctggcttctcctggtcgctgtgctcaaaggtgtccagtgtcagcagcagctggtggagtccgggggaggcctggtcaagcctggggcagccctgacattcacctgcacagcctctggattctccttcagtggcaattattggatatgctgggtccgccaggctccagggaaggggttggagtggatcgcgtgcattggtactattactagtaggacatactacgcgagctgggcgaaaggccgattcaccatttccaaaacctcgtcgaccacggtgactctgcaaatgaccagtctgacagccgcggacacggccacgtatttctgtgcgagaggtgcggttgttagtagtggtaatgctccctactactttaccttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 60 - DNA, 711 bp, Artificial Sequence
  source: /note=“Description of Artificial Sequence Synthetic polynucleotide”
atggacacgagggcccccactcagctgctggggctcctgctgctctggctcccaggtgccagatgtgcattcgaattgacgcagactccatcctccgtggaggcagctgtgggaggcacagtcaccatcaagtgccaggccagtcagagcattagtagttacttatcctggtatcagcagaaaccagggcagcctcccaagctcctgatctacagggcatccactctggaatctggggtcccatcgcggtttaaaggcagtggatctgggacagagttcactctcaccatcagcgacctggagtgtgccgatgctgccacttacttctgtcaaagctattatggtgttacttttagtggttttgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 61 - DNA, 1383 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcaaagcctctggaatcgacttcaataactattggataacctgggtccgccaggctccagggaaggggctggagtggatcgcatgcatttatgttggaattaccggccgcacatggtacgcgaactgggcgaaaggccgattcaccatctccaaggcctcgagcacggtggatctgaaaatgaccagtctgacagccgcggacacggccacctatttctgtgcgaggaatggtgatggtggtatttatgctcttaacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 62 - DNA, 717 bp, Artificial Sequence
  source: /note=“Description of Artificial Sequence Synthetic polynucleotide”
atggacacgagggcccccactcagctgctggggctcctgctgctctggctcccaggtgccacatttgcacaagtgctgacccagactgcatcgtccgtgtctgcagctgtgggaggcacagtcaccatcagttgccagtccagtcagagtgtttataataataattggttagcctggtatcagcagaaaccagggcagcctcccaagctcctgatctacagggcatccactctgacatctggggtcccatcgcggttcaaaggcagtggatctgggacacagttcactctcaccatcagcgacctggagtgtgacgatgctgccacttactactgtgcaggcggttatagtggtaatatttacgtaaatgatttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 63 - DNA, 1380 bp, Artificial Sequence
  source: /note=“Description of Artificial Sequence Synthetic polynucleotide”
atggagactgggctgcgctggcttctcctggtcgctgtgctcaaaggtgtccagtgtcaggagcagctggaggagtccgggggagacctggtcaagcctgggggatccctgacactcacctgcaaagcctctggattctccttcagtaatacctactgggcatgctgggtccgccaggctccagggaaggggctggagtggatcgcatgcatgaatcctgctagtagtggtagctcttactacgcgagctgggcgaaaggccgattcaccatctccaaaacctcgtcgaccacggtgactctgcacatgcccagtctgacagccgcggacacggccacctatttctgtgcgaaatgggatactgctttcgatgtgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 64 - DNA, 714 bp, Artificial Sequence
  source: /note=“Description of Artificial Sequence Synthetic polynucleotide”
atggacacgagggcccccactcagctgctggggctcctgctgctctggctcccaggtgccagatgtgccgatgttgtgatgacccagactccatcctccgtggaggcagctgtgggaggcacagtcaccatcaagtgccaggccagtcagagcattagtagctacttagcctggtatcagcagaaaccagggcagcctcccaagctcctgatctatggtgcatccaatctggagtctggggtcccatcgcggttcaaaggcagtggatctgggacagagtacactctcaccatcagcggcgtgcagtgtgacgatgctgccacttactactgtcaaaactattatgctattgatacttatggtcatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 65 - DNA, 1368 bp, Artificial Sequence
  source: /note=“Description of Artificial Sequence Synthetic polynucleotide”
atggagactgggctgcgctggcttctcctggtcgctgtgctcaaaggtgtccagtgtcaggagcagctggaggagtccgggggagacctggtcaagcctggggcatccctgacactcacctgcacagcctctggattctcctttagtagcaactatcacatctgctgggtccgccaggctccaggaaaggggctggagttgatcgcatgcatttatgttggtgatggcagcacatactacgcgagctgggcgaaaggccgattcaccatctccaaatcctcgtcgaccacggtagctctgcaaatgaccagtctgacagccgcggacacggccacctatttctgtgggagaatgtttaacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 66 - DNA, 708 bp, Artificial Sequence
  source: /note=“Description of Artificial Sequence Synthetic polynucleotide”
atggacacgagggcccccactcagctgctggggctcctgctgctctggctcccaggtgccatatgtgaccctgtgctgacccagactccatcctccgtgtctgcggctgtgggagtcacagtcaccatcaactgccagtccagtccgagtgtttatagtaactacttatcctggtatcagcagaaaccagggcagcctcccaagctcctcatctatctggcatctactctggcatctggggtcccatcgcggttcaaaggcagtggatctgggacacagttcactctcaccatcagcgacgtgcagtgtgacgatgctgccacttactactgtgcaggcacttatagtggtaatatttggtctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 67 - DNA, 1383 bp, Artificial Sequence
  source: /note=“Description of Artificial Sequence Synthetic polynucleotide”
atggagactgggctgcgctggcttctcctggtcgctgtgctcaaaggtgtccagtgtcagcagcagctggtggagtccgggggaggcctggtcaagcctggggcatccctgacactcacctgcacagcctctggattctccttcagtagcggctatcacatgtgctgggtccgccaggctccagggaaggggctggagtggatcgcatgctttggtgtttatactggtaccactacctacgcgagctgggcgaaaggtcgattcaccatctccaaaacctcgtcgaccacggtgactctacaaatgaccagtctaacagtcgcggacacggccacctatttctgtgcgagaatcagtgctgaaaatggtggggacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 68 - DNA, 711 bp, Artificial Sequence
  source: /note=“Description of Artificial Sequence Synthetic polynucleotide”
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtcagagcattagcaactacttttcttggtatcagcagaaaccagggcagcctcccaagctcctgatctacagggcgtccactctggcatctggggtcccatcgcggttcagcggcagtggatctgggacagagttcactctcaccatcagcgacctggagtgtgccgattctgccacttactactgtcagtgcacttatggtagtagtagtactggttttggtttcggcggagggaccgaggtggtggtcaaaggtgatccagttgcacctactgtccccatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 69 - DNA, 1371 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcatggcctctggaatcgacttcagtagcggctacggcatgtggtgggtccgccaggctccagggaagggactggagtatatcggatacattgatactggtgatgataacacatactacgcgaactgggcgaaaggccgattcaccatctccaaaacctcgtcgaccacggtgactctgcaaatgaccagtctgacagtcgcggacacggccacctatttctgtgcgaaagggggcgccatagacctctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 70 - DNA, 720 bp, Artificial Sequence
  source: /note=“Description of Artificial Sequence Synthetic polynucleotide”
atggacacgagggcccccactcagctgctgggactcctactgctctggctcccaggtgccagatgtgctgacattgtgatgacccagactccagcctccgtggaggcagctgtgggaggcacagtcaccatcaagtgccaggccagtcagagcattagtagttacttagcctggtatcagcagaaaccagggcagcgtcccaagctcctgatctacagggcatccactctaaaatctggggtcccatcgcggttcaaaggcagtggatctgggacagagtacactctcaccatcagcgacctggagtgtgccgatgctgccacttattattgtcaagcgtattatcttagtagtagtatcagttatggtaatactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 71 - DNA, 1371 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcgtccctgacactcacctgcacagcctctggattctccctcagtagcgggtatggcatgtgctgggtccgccaggctccagggaagggactggagtggatcggatacactgatactgctactggtaccattcactacgcgagctgggcgaaaggccgattcaccatctccaaaacctcgtcgaccacggtgactctgcaaatgaccagtctgacagccgcggacacggccacctatttctgtgcgaaagggggcgccatggacctctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 72 - DNA, 720 bp, Artificial Sequence
  source: /note=“Description of Artificial Sequence Synthetic polynucleotide”
atggacacgagggcccccactcagctgctggggctcctactgctctggctcccaggtgccagatgtgctgacattgtgatgacccagactccagcctccgtggaggcagctgtgggaggcacagtcaccatcaagtgccaggccagtcagagcattagtagctacttagcctggtatcagcagaaaccagggcagcctcccaagctcctgatctacaggacatccactctggcatctggggtcccatcgcggttcaaaggcagtggatctgggacagagtacactctcaccatcagcgacctggagtgtgccgatgctgccacttactattgtcaaagctatgcttatagtagtagtagcagttatggtaatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 73 - DNA, 1365 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcacaggctctggaatcgacttcagtagcagctactggatatgctgggtccgccaggctccagggaaggggctggagtggatcgcatgcatcgatggtagtgatggtaacacttactacgcgagctgggcgagaggccgattcaccatctccaaaacctcgtcgaccacggtgactctgcaaatggccagtctgacagccgcggacacggccacctatttctgtacgagagatctcaggttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 74 - DNA, 723 bp, Artificial Sequence
  source: /note=“Description of Artificial Sequence Synthetic polynucleotide”
atggacacgagggcccccactcagctgctggggctcctgctgctctggctcccaggtgccagatgtgctgacattgtgctgacccagactccagcctcggtgtctgcagctgtgggaggcacagtcaccatcaactgccaggccagtcagaatgtttatagtaacaatgccttagcctggcatcagcagaaaccagggcagcgtcccaacctcctgatctacaaggcttccactctggcatctggggtcccatcgcggttcaaaggcagtggatctgggacacagtttactctcaccatcagcgacgtgcagtgtgacgatgctgccacttactactgtctaggcgaatttagttgtagtagtggtgattgttttgttttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 75 - DNA, 1371 bp, Artificial Sequence; identical to SEQ ID NO: 69
  source: /note=“Description of Artificial Sequence Synthetic polynucleotide”

SEQ ID NO: 76 - DNA, 720 bp, Artificial Sequence; identical to SEQ ID NO: 70
  source: /note=“Description of Artificial Sequence Synthetic polynucleotide”

SEQ ID NO: 77 - DNA, 1416 bp, Artificial Sequence
  source: /note=“Description of Artificial Sequence Synthetic polynucleotide”
atggagactgggctgcgctggcttctcctggtcgctgtgctcaaaggtgtccagtgtcagcagcagctggaggagtccgggggaggcctggtcaagcctggaggaaccctgacactcacctgcaaagcctctggagtcgccctcaatccctactactatatgtgctgggtccgccaggctccagggaaggggctggagtggatcgcatgcgtggatgctgatagtagtggtagcacttactacgcgagctgggcgaaaggccgattcaccatctccaaaacctcgtcgaccacggtgactctgaaaatgaccagtctgacagccgcggacacggccacctatttctgtgcgagagaatcggttgactatagttctgttggtattggctatgtacatggtacggatggcttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 78 - DNA, 720 bp, Artificial Sequence
  source: /note=“Description of Artificial Sequence Synthetic polynucleotide”
atggacacgagggcccccactcagctgctggggctcctgctgctctggctcccaggtgccagatgtgccgacatcgtggtgacccagactccatcctccgtgtctgcagctgtgggaggcacagtcaccatcaagtgccaggccagtcagagcattagcaactacttttcttggtatcagcagaaaccagggcagcctcccaagctcctgatctacagggcgtccactctggcatctggggtcccatcgcggttcaaaggcagtggatctgggacagagttcactctcaccatcagcgacctggagtgtgccgatgctgccacttactactgtcaatgcacttacggtagaagtaatagtaattttttttatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 79 - DNA, 1395 bp, Artificial Sequence
  source: /note=“Description of Artificial Sequence Synthetic polynucleotide”
atggagactgggctgcgctggcttctcctggtcgccgtgctcaaaggtgtccagtgtcagtcgttggaggagtccgggggagacctggtcaagcctggggcctccctgacactcacctgcacagcctctggatcctccttcagtagtacctactggaactgctgggtccgccaggctccagggaaggggctggagtggatcgcatgcattaatgctggtagtggtaccacttactacgcgagctgggcgaaaggccgattcaccgtctccaaaacctcgtcgaccacggtgactctgcaaatgaccagtctgacagccgcggacacggccacctatttctgtacgagagatagtgatggtcgttttagtagtggctactattttaacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 80 - DNA, 717 bp, Artificial Sequence
  source: /note=“Description of Artificial Sequence Synthetic polynucleotide”
atggacacgagggcccccactcagctgctggggctcctgctgctctggctcccaggtgccacatttgcccaagtgctgacccagactgcatcccctgtgtctgcagctgtgggaggcacagtcaccatcaattgtcagtccagtcagagtgtttatgataacaactggttagcctggtatcagcaaaaaccagggcagcctcccaaactcttgatcgacgatgcatccaaattgacatctggggtctcatcgcggttcaaaggcagtggatctgggacgcagttcactctcaccatcagcggcgtgcagtgtgacgatgctgccacttactactgtcaaggcgcttattatagtagtggttggtactgggctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 81 - DNA, 1404 bp, Artificial Sequence
  source: /note=“Description of Artificial Sequence Synthetic polynucleotide”
atggagactgggctgcgctggcttctcctggtcgctgtgctcaaaggtgtccagtgtcagcagcagctggaggagtccgggggaggcctggtcaagcctggaggaaccctgacactcacctgcaaagcctctggaatcgacttcagtagctactactacatgtgttgggtccgccaggctccagggaaggggctggagttgatcgtatgtatttatactagtagtggtggcacatggtacgcgagctgggtgaatggccgactcaccatctccagaagcaccagcctaaacacggtggatctgaaaatgaccagtctgacagccgcggacacggccacctatttctgtgccagaggggtttattctggtagtagtgattatccaactcggttggatctctggggccagggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 82 - DNA, 711 bp, Artificial Sequence
  source: /note=“Description of Artificial Sequence Synthetic polynucleotide”
atggacacgagcacctccactgcgctcctggggctcctgctgctctggctcacaggtgccagatgtgccatcgagatgacccagtctccaccctccctgtctgcatctgtgggagaaactgtcaggattaggtgcctggccagtgaggacatttacagtggtatatcctggtaccaacagaagccagagaaacctcctacactcctgatctctggtgcatccaatttagaatctggggtcccaccacggttcagtggcggtggatccgggacagattacaccctcaccatcggcggcgtgcaggctgaagatgttgccacctactactgtctaggcggttatagtttcagtagtaccggtttgacttttggagctggcaccaaggtggaaatcaaacgtgatccagttgcgccttctgtcctcctcttcccaccatctaaggaggagctgacaactggaacagccaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 83 - DNA, 1383 bp, Artificial Sequence
  source: /note=“Description of Artificial Sequence Synthetic polynucleotide”
atggagactgggctgcgctggcttctcctggtcgctgtgctcaaaggtgtccagtgtcagcaacatctggtggagtccgggggaggcctggtcaagcctggggcatccctgacactcacctgcacagcctctggattctccttcactaccggctatcacatgtgctgggtccgccaggctccagggaaggggctggagtggatcgcatgttttggtgtttatactagtaccactacctacgcgagctgggcgaaaggtcgattcaccatctccaaaacctcgtcgaccacggtgactctacaaatgaccagtctaacagtcgcggacacggccacctatttctgtgcgagaatcagtgctgaagatggtggggacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 84 - DNA, 711 bp, Artificial Sequence
  source: /note=“Description of Artificial Sequence Synthetic polynucleotide”
atggacacgagggcccccactcagctgctggggctcctgctgctctggctcccaggtgccagatgtgatgttgtgatgacccagactccagcctccgtggaggcagctgtgggaggcacagtcaccatcacgtgccaggccagtcagagcattagcaactacttttcttggtatcagcagaaaccagggcagcctcccaagctcctgatctacagggcgtccactctggcatctggggtcccatcgcggttcagcggcagtggatctgggacacagttcactctcaccatcagcgacctggagtgtgccgattctgccacttacgcctgtcagtgcacttatggtagtagtagtactggttttggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 85 - DNA, 1389 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcaaagcctctggattctccttcagtagttacttctggatatgctgggtccgccaggctccagggaaggggctggagtggagcgcatgcatctatggtgatagtagtggtagtagttactacgcgagctgggcgaaaggccgattcaccatctccaaaacctcgtcgaccacggtgactctgcaaatgaccagtctgacagccgcggacacggccacctatttctgtgcgagttatggtagtagtagttattactactctaatttatggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 86 - DNA, 708 bp, Artificial Sequence
  source: /note=“Description of Artificial Sequence Synthetic polynucleotide”
atggacacgagggcccccactcagctgctggggctcctgctgctctggctcccaggtgccatatgtgaccctgtgatgacccagactccatcttccacgtctgcggctgtgggaggcacagtcaccatcagttgccagtccagtcagagtgtttataataacaactacttagcctggtatcagcagaaaccagggcagcctcccaaacgcctgatctacgaatcatccaaactggcatctggggtcccatcgcggttcagaggcagtggatctggggcacagttcactctcaccatcagcgacctggagtgtgacgatgctgccacttactactgtctaggcgcatattatactactcttgatttcggcggagggaccgaggtggtggtcagaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 87 - DNA, 1383 bp, Artificial Sequence
  source: /note=“Description of Artificial Sequence Synthetic polynucleotide”
atggagactgggctgcgctggcttctcctggtcgctgtgctcaaaggtgtccagtgtcaggagcagttggtggagtccgggggaggcctggtccagcctgagggatccctgacactcacctgcaaagcctctggattcgacttcagtagcaatgcaatgtgctgggtccgccaggctccagggaaggggctggagtggatcgcatgcatttataatggtgatggcagcacatactacgcgagctgggtgaatggccgattcaccatctccaagacctcgtcgaccacggtgactctgcaaatgaccagtctgacagccgcggacacggccacctatttctgtgcgagaggtctctctaattggaatagggataacttatggggccctggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 88 - DNA, 720 bp, Artificial Sequence
  source: /note=“Description of Artificial Sequence Synthetic polynucleotide”
atggacacgagggcccccactcagctgctggggctcctgctgctctggctcccaggtgccacatttgcccaagtgctgacccagactccatcctccgtgtctgcagctgtgggaggcacagccaccatcaactgccaggccagtcagagtctttatagtcccaagaatttagcctggtatcagcagacaccagggcagcctcccaagctcctgatctattctgcatcgaaactggcatctggggtcccatcgcggttcaaaggcagtggatctgggacacagttcactctcaccatcagcggcgtgcagtgtgacgatgctgcaatttactactgtcaaggcgaatttagttgtactactgctgcttgttttgcttttggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 89 - DNA, 1386 bp, Artificial Sequence
  source: /note=“Description of Artificial Sequence Synthetic polynucleotide”
atggagactgggctgcgctggcttctcctggtcgctgtgctcaaaggtgtccagtgtcagtctttggaggagtccgggggaggcctggtccagcctgagggatccctgacactcgcctgcacagcttcgggattctccttcagtagctactactacatctgctgggtccgccaggctccagggacggggctggagtggatcggatgcattaatactggtagtgatgacactcactacgcgagctggttgaaaggccgattcaccttctccaaggcctcgtcgaccacgttgactctgcaaatgaccagtctgacagccgcggacacggccacctatttctgtgcgagatcatctggtagtagtgatgatgcttatgatctctggggcccaggcaccctggtcactgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 90 - DNA, 708 bp, Artificial Sequence
  source: /note=“Description of Artificial Sequence Synthetic polynucleotide”
atggacacgagggcccccactcagctgctggggctcctgctgctctggctcccaggtgccagatgtgatgttgtgatgacccagactccagcctccgtgtctgaacctgtgggaggcgcagtcaccatcaagtgccaggccagtcagagcattggtagtaatttagcctggtatcagcacaaaccagggcagcctcccaagctcctgatctattttgcatccagcctggcatctggggtctcgtcgcggttcaagggcggtagatctgggacacagttcactctcaccatcagcgacctggagtgtgccgatgctgccacttactactgtcactgtacttattatcctcttagttatgttactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 91 - DNA, 1395 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctgggacatccctgacactctcctgcacagcctctggattctccttcggtagctattattatatgtgctgggtccgccaggctccagggaaggggctggagtggatcgcatgcattgatgttggtagtagtggtgacacatactacgcgagctgggtgaatggccgattcaccatctccaaaacctcgtcgaccacggtgactctgcaaatgaccagtctgacagccgcggacacggccacctatttctgtgcgagagatgatactgctgctggtggttttggtaatttggaattgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 92 - DNA, 717 bp, Artificial Sequence
  source: /note=“Description of Artificial Sequence Synthetic polynucleotide”
atggacacgagggcccccactcagctgctggggctcctgctgctctggctcccaggtgccagatgtgcattcgaattgacccagactccatcctccgtgtctgaacctgtgggaggcacagtcaccatcaagtgccaggccagtcagagcatttacagctacttttcctggtatcagcagaaaccagggcagcctcccaagcgcctgatttaccaggcatccactctggcttctggggtcccatcgcggttcaaaggcagtggatctgggacagatttcactctcaccatcagcgacctggagtgtgccgatgctgccacttactactgtcaaaacaattatggtaggggtagtggtagttatttttttggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 93 - DNA, 1371 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcaaagcctctggaatcgacttcagtagcggctacggcatgtggtgggtccgccaggctccagggaagggactggagtatatcggatacattgatactggtagtggtagcacttactacgcgaactgggcgaaaggccgattcaccatctccaaaacctcgtcgaccatggtgactctgcaaatgaccagtctgacagtcgcggacacggccacctatttctgtgcgaaagggggcgccatagacctctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 94 - DNA, 720 bp, Artificial Sequence
  source: /note=“Description of Artificial Sequence Synthetic polynucleotide”
atggacacgagggcccccactcagctgctgggactcctactgctctggctcccaggtgccagatgtgctgacattgtgatgacccagactccagcctccgtggaggcagctgtgggaggcacagtcaccatcaagtgccaggccagtcagagcattagtagttacttagcctggtatcagcagaaaccagggcagcgtcccaagctcctgatctacagggcatccactctggcatctggggtcccatcgcggttcaaaggcagtggatctgggacagactacactctcaccatcagcgacctggagtgtgccgatgctgccacttattattgtcatacctattatcttagtagtagtatcagttatggtaatactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 95 - DNA, 1398 bp, Artificial Sequence
  source: /note=“Description of Artificial Sequence Synthetic polynucleotide”
atggagactgggctgcgctggcttctcctggtcgctgtgctcaaaggtgtccagtgtcaggagcagctggaggagtccgggggagacctggtcaagcctgagggatccctgacactcacctgcaaagcctctggattctccttcagtagcatctactggatttgctgggtccgccaggctccagggaaggggctggagtggatcgcatgcactactgttgtcaaaagtggtagaacttactacgcgaactgggcgaaaggccgattcaccatctccaaaacctcgtcgaccacggtgactctgcaaatgaccagtctgacagccgcggacacggccacctatttctgtgcgagagaatttgttgatggtggtggtagtagtggtagggacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 96 - DNA, 711 bp, Artificial Sequence
  source: /note=“Description of Artificial Sequence Synthetic polynucleotide”
atggacacgagggcccccactcagctgctggggctcctgctgctctggctcccaggtgccagatgtgcctatgatatgacccagactccagcctccgtggaggcagctgtgggaggcacagtcaccatcaagtgccaggccagtcagagcattagtagggacttatcctggtatcagcagaaacctggacagcctcccaagcgcctaatctacaaggcatccactctggcatctggggtcccatcgcggttcaaaggcagtggatctgggacagatttcactctcaccatcagcgacctggagtgtgccgatgctgccacttactactgtcaacagggttatagtagtattgatgttgataatgatttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 97 - DNA, 1404 bp, Artificial Sequence
  source: /note=“Description of Artificial Sequence Synthetic polynucleotide”
atggagactgggctgcgctggcttctcctggtcgctgtgctcaaaggtgtccagtgtcaggagcagctggaggagtccgggggaggcctggtcaagcctgagggatccctgacactcacctgcaaagcctctggattcgacctcagtagcggctatgacatgtgctgggtccgccaggctccagggaaggggctggagtggatcgcatgcatttatgctgattatagtggtagcacatactacgcgagctgggtgaatggccgattcaccatctccagcagcaccagcctaaacacggtggatctgaaaatgaccagtctgacagccgcggacacggccacctatttctgtgccagaggggctactggtaatggtggttatggatactactttaacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 98 - DNA, 717 bp, Artificial Sequence
  source: /note=“Description of Artificial Sequence Synthetic polynucleotide”
atggacacgagggcccccactcagctgctggggctcctactgctctggctcccaggtgccagatgtgctgacattgtgatgacccagactccagcctccgtgtctgaacctgtgggaggcacagtcaccatcaagtgtcaggccagtcagaacattaatagcggcttagcctggtatcagcagaaaccagggcagcctcccaagctcctgatctacaaggcatccactctggcatctggggtctcatcgcggttcaaaggcagtggatctgggacagaattcactctcaccatcagcgacctggagtgtgccgatgctgccacttactactgtcaaacctattattatagtagtagtagtagtgataatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 99 - DNA, 1389 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcacagcttctggattctccttcagtagcagttattggatatgctgggtccgccaggctccagggaaggggctggagtggatcgcatgtatttatggtggtagtagtggtaacattgcctacgcgagctgggcgaaaggccgattcaccatctccaaaacctcgtcgaccacggtgactctacaaatgaccagtctgacagccgcggacacggccacctatttctgtgcgagagatattcctagtgatgctttcaccttagacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 100 - DNA, 720 bp, Artificial Sequence
  source: /note=“Description of Artificial Sequence Synthetic polynucleotide”
atggacacgagggcccccactcagctgctggggctcctgctgctctggctcccaggtgccacatttgcccaagtgctgacccagactccatcctccgtgtctgcagctgtgggaagcacagtcaccatcaattgccaggccagtcagagtgtttataaagacaacaatttagcctggtatcagcagaaaccagggcagcctcccaagctcctgatctacaaggcttccactctggcatctggggtcccatcgcggttcaaaggcagtggatctgggacacagttcactctcaccatcagcggcgtgcagtgtgaagatgctgccacttactactgtcaaggcgaattcagttgtggtagtgctgattgtattgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 101 - DNA, 1371 bp, Artificial Sequence
  source: /note=“Description of Artificial Sequence Synthetic polynucleotide”
atggagactgggctgcgctggcttctcctggtcgctgtgctcaaaggtgtccagtgtcagtcgttggaggagtccgggggagacctggtcaagcctggggcatccctgacactcacctgcaaaggctctggaatcgacttcagtagcggctacggcatgtggtgggtccgccaggctccagggaagggactggagtatatcggatacattgatactggttatggtagcacttactacgcgagctgggcgaaaggccgattcaccatctccaagacctcgtcgaccacggtgactctgcaaatgaccagtctgacagtcgcggacacggccacctatttctgtgcgaaagggggcgccatagacctctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 102 - DNA, 717 bp, Artificial Sequence
  source: /note=“Description of Artificial Sequence Synthetic polynucleotide”
atggacacgagggcccccactcagctgctggggctcctgctgctctggctcccaggtgccacatttgcagccgtgctgacccagactccggcttccacgtctgcagctgtgggaggcacagtcaccatcaattgtcagtccagtcagagcgtgtatcgtagcaactggttagcctggtatcagcagaaaccagggcagcctcccaagctcctgatctatgatgtatttaatttggcatctggggtcccatcccggttcaagggcagtggatctgggacacagttcactctcaccatcagcggcgtgcagtgtgccgatgctgccacttactactgtcaaggcagttattatagtggtaattggtacagtgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 103 - protein, 122 aa, Artificial Sequence
  source: /note=“Description of Artificial Sequence Synthetic polypeptide”
EVQLQQSGPELVKPGASVKIPCKASGYTFPDYNMDWVKQSHGKSLEWIGYINPDNGGTIYNQKFKGKATLTVDKSSSTAYMELRSLTSEDTAVYYCARLDSSGYGYYAMDYWGQGTSVTVSS

SEQ ID NO: 104 - protein, 111 aa, Artificial Sequence
  source: /note=“Description of Artificial Sequence Synthetic polypeptide”
DIVLTQSPASLAVSLGQRATISCRASESVDNYGISFMHWYQQKPGQPPKLLIYRASNLDSEIPARFSGSGSRTDFTLTIDPVETDDVATYYCQQSYKDPRTFGGGTKLEIK

SEQ ID NO: 105 - protein, 122 aa, Artificial Sequence
  source: /note=“Description of Artificial Sequence Synthetic polypeptide”
KVMLVESGGALVKPGGSLKLSCVASGFTFSNYAMSWVRQTPEKRLEWVATISSGGYYTYYPDSVKGRFTISRDNARNTLFLQMSSLRSEDTAMFYCAREDDYGRYSYTMDYWGQGTSVTVSS

SEQ ID NO: 106 - protein, 112 aa, Artificial Sequence
  source: /note=“Description of Artificial Sequence Synthetic polypeptide”
DVVMTQTPLSLPVSLGDQVSISCRCSQSLVHSNGNTYLHWYLQKPGQSPQLLIYKISNRFSGVPDRFSGSGSGTDFTLKISRVEAEDLGVYFCSQSTHVPYTFGGGTELEIK

SEQ ID NO: 107 - protein, 119 aa, Artificial Sequence
  source: /note=“Description of Artificial Sequence Synthetic polypeptide”
EVQLQQSGPELVKPGASVRISCKASGYTFTDYYIHWVKQKPGQGLECIGEIYPGTDNTYYSKKFRGKATLTADKSSDTAYMQLSSLTSEDSAVYFCARGDYYRGYFDVWGAGTTVTVSS

SEQ ID NO: 108 - protein, 112 aa, Artificial Sequence
  source: /note=“Description of Artificial Sequence Synthetic polypeptide”
DVVMTQTSLTLSVTIGQPASISCKSSQSLLHSNGKTYLNWLLQRPGQSPKFLIYLVSKLESGVPDRFSGSGSGTDFTLKISRVEAEDLGVYYCLQSTHFPWTFGGGTKLEIK

SEQ ID NO: 109 - protein, 119 aa, Artificial Sequence
  source: /note=“Description of Artificial Sequence Synthetic polypeptide”
EVQLQQSGPELVKPGASVKKSCKASGYTFTDYYMHWVKQKPGQGLEWIGKIYPGSGNTHYNEKFKGKATLTADKSSSTAYMQLSSLTSEDSAVYFCATNYYGYRAMNYWGQGSSVTVSS

SEQ ID NO: 110 - protein, 107 aa, Artificial Sequence
  source: /note=“Description of Artificial Sequence Synthetic polypeptide”
DIHLTQSPSSLSASLGERISLTCRASQDIYISLNWFQQKPDGTIKLLIYGTSSLDSGVPKRFSGSRSGSDYSLTISSLESEDFADYYCLQYASSPYTFGGGTKLEIK

SEQ ID NO: 111 - protein, 121 aa, Artificial Sequence
  source: /note=“Description of Artificial Sequence Synthetic polypeptide”
EVQLQQSGPELVKPGASVKISCKASGYTFTDYYMHWVKQKPGQGLEWIGEIYPGSGNPYYNEKFKGKATLTADKSSSSAYMQLSSLTSEDSAVYFCARTSYGRVGTGFAYWGQGTLVTVSA

SEQ ID NO: 112 - protein, 111 aa, Artificial Sequence
  source: /note=“Description of Artificial Sequence Synthetic polypeptide”
NFVMTQTPLSLPVSLGDQASISCRSSQSLLHSNGNTYLHWYLQKPGQSPKLLIYKVSNRFSGVPDRFSGSGSGTDFTLKINRVETEDLGIYFCSQSSHVPTFGAGTKLELK

SEQ ID NO: 113 - protein, 117 aa, Artificial Sequence
  source: /note=“Description of Artificial Sequence Synthetic polypeptide”
QVQLQQSGAELAKPGASVKMSCKASGYTFTNYWMHWVKQRPGQGLEWIGYINPNNGYTEYNQRFKDKATLTADRSSTTAYMQLSSLTSEDSAVYYCARSDIITTDYWGQGTTLTVSS

SEQ ID NO: 114 - protein, 112 aa, Artificial Sequence
  source: /note=“Description of Artificial Sequence Synthetic polypeptide”
DVVMTQTPLSLPVSLGDQASISCRSSQSLVYSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 115 - protein, 116 aa, Artificial Sequence
  source: /note=“Description of Artificial Sequence Synthetic polypeptide”
QVQLQQPGAELVRPGTSVKLSCKASGYTFTSYWMHWVQQRPGQGLEWIGVIDPSDSYTNYNQKFKGKATLTVDTSSSSAYMQLSSLTSEDSAVYYCARDDVAMDYWGQGTSVTVSS

SEQ ID NO: 116 - protein, 111 aa, Artificial Sequence
  source: /note=“Description of Artificial Sequence Synthetic polypeptide”
DIVVTQSPASLAVSLGQRATISCRASESVDSYGNSFIHWYQQKPGQPPKLLIYRASNLKSGIPARFSGSGSRTDFTLTINPVEADDVATYYCQQSNEDPYTFGGGTKLEIK

SEQ ID NO: 117 - protein, 119 aa, Artificial Sequence
  source: /note=“Description of Artificial Sequence Synthetic polypeptide”
EVKLEESGGGLVQPGGSMKLSCAASGFTFSDAWMDWVRQSPEAGLEWVAEIRNKAHNPATYYAESVKGRFTISRDDSKSSVYLQMNSLRAEDTGIYYCTLVAPDAMDYWGQGTSVTVSS

SEQ ID NO: 118 - protein, 113 aa, Artificial Sequence
  source: /note=“Description of Artificial Sequence Synthetic polypeptide”
DIVMSLSPSSLAVSVGEKVTMSCKSSQSLLYSRNQKNYLAWYQQKPGQSPKLLIYWASTRASGVPDRFTGSGSGTDFTLTISSVKAEDLAVYYCQQYYSYPYTFGGGTKLEIK

SEQ ID NO: 119 - protein, 116 aa, Artificial Sequence
  source: /note=“Description of Artificial Sequence Synthetic polypeptide”
QVQLQQSGPELVRPGASVKMSCKASGYTFTSYWMHWVKQRPGQGLEWIGMIDPSNSETWLNQKFKDKATLNVDKSSNTAYMQLSSLTSEDSAVYYCARYDGYYDYWGQGTTLTVSS

SEQ ID NO: 120 - protein, 111 aa, Artificial Sequence
  source: /note=“Description of Artificial Sequence Synthetic polypeptide”
NIVLTQSPASLAVSLGQRATISCRASESVDSYGNSFMHWYQQKPGQPPKLLIYLASNVESGVPARFSGSGSRTDFTLTIDPVEADDAATYYCQQNNEDPWTFGGGTKLEIK

SEQ ID NO: 121 - protein, 118 aa, Artificial Sequence
  source: /note=“Description of Artificial Sequence Synthetic polypeptide”
QVQLQQPGAELVRPGASVKLSCKPSGYTFTSYWMNWVKQRPGQGLEWIGMIDPSDSETHYNQMFKDKATLTVDKSSNTAYMQLSSLTSEDSAVYYCAQIYYAYDKAYWGQGTLVTVSA

SEQ ID NO: 122 - protein, 111 aa, Artificial Sequence
  source: /note=“Description of Artificial Sequence Synthetic polypeptide”
DIVMSQSPSSLAVSVGEKVTMSCKSSQSLLYSSHQKNYLAWYQQKPGQSPKLLIYWASTRESGVPDRFTGSGSGTDFSLTISSVKAEDLAVYYCQEYYSWTFGGGTKLEIK

SEQ ID NO: 123 - protein, 118 aa, Artificial Sequence
  source: /note=“Description of Artificial Sequence Synthetic polypeptide”
EVQLQQSGPELVKPGASVKISCKASGYTFTDYYMNWVKQSHGKSLEWIGDINPHNGGTSFIQKFKGKATLTVDKSSSTAYMELRSLTSEDSAVYYCAPLGRKEGFAYWGQGTLVTVSA

SEQ ID NO: 124 - protein, 111 aa, Artificial Sequence
  source: /note=“Description of Artificial Sequence Synthetic polypeptide”
DTVLTQSPASLVVSLGQRATISCRASKSVSTSGYSYMHWYQQKPGQPPKLLIYLASNLESGVPARFSGSGSGTAFTLNIHPVEEEDAATYYCQHSRELPYTFGGGTKLEIK

SEQ ID NO: 125 - protein, 119 aa, Artificial Sequence
  source: /note=“Description of Artificial Sequence Synthetic polypeptide”
QVTLKESGPGILQPSQTLSLTCSFSGFSLSTFAMGVGWIRQPSGKGLEWLAHIWWDDDKYYNPALKSRLTISKDTSKNHVFLKIANVDTADTATYYCARMPLTFYFDYWGQGTTLTVSS

SEQ ID NO: 126 - protein, 112 aa, Artificial Sequence
  source: /note=“Description of Artificial Sequence Synthetic polypeptide”
DVLLTQTPLSLPVSLGDQASISCRSSQSIVHSNGHTYLEWYLQKPGQSPKLLIYKVSNRFSGVPDRFSGSGSGTDFTLKISRVEAEDLGVYYCFQGSHVPFTFGGGTKLEIK

SEQ ID NO: 127 - protein, 119 aa, Artificial Sequence
  source: /note=“Description of Artificial Sequence Synthetic polypeptide”
QVTLKESGPGILQPSQTLSLTCSFSGFSLRTFAMGVGWIRQPSGKGLEWLAHIWWDDDKYYNPALKSRLTISKDTSKNQVFLKIANVDTADTATYYCARMPLTFYFDYWGQGTTLTVSS

SEQ ID NO: 128 - protein, 112 aa, Artificial Sequence
  source: /note=“Description of Artificial Sequence Synthetic polypeptide”
DVLMTQTPLSLPVSLGDQASISCRSSQSIVHSNGNTYLEWYLQKPGQSPKLLIYKVSTRFSGVPDRFSGSGSGTDFTLKISRVEAEDLGVYYCFQGSHVPFTFGGGTKLEIK

SEQ ID NO: 129 - protein, 115 aa, Artificial Sequence
  source: /note=“Description of Artificial Sequence Synthetic polypeptide”
QVQLQQPGAELVMPGASVKLSCKASGYTFTNYWMHWVKQRPGQGLEWIGEIDPSDSYTNYNQKFKGKATLTVDKSSSTAYMQLSSLTSEDSAVYYCTRQGSTYAWGQGTSVTVSS

SEQ ID NO: 130 - protein, 112 aa, Artificial Sequence
  source: /note=“Description of Artificial Sequence Synthetic polypeptide”
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPPTFGGGTKLEIK

SEQ ID NO: 131 - protein, 112 aa, Artificial Sequence
  source: /note=“Description of Artificial Sequence Synthetic polypeptide”
EVQLQQSGPELVKPGASVKMSCKASGYTFTDYYMMWVKQSHGKSLEWIGDINPYNGGSSYNPKFKGRATLTVDKSSSTAYMQLNSLTSEDSAVYYCARGTYWGQGTLVTVSA

SEQ ID NO: 132 - protein, 112 aa, Artificial Sequence
  source: /note=“Description of Artificial Sequence Synthetic polypeptide”
DVVMTQTPLTLSVTIGQPASISCKSSQSLLDSAGKTYLNWLLQRPGQSPKRLMYLVSKLDSGVPDRFTGSGSGTDFTLKISRVEAEDLGVYYCWQGTHFPYTFGGGTKLEIK

SEQ ID NO: 133 - protein, 112 aa, Artificial Sequence
  source: /note=“Description of Artificial Sequence Synthetic polypeptide”
QVQLQQPGAELVKPGASVKLSCKASGYTFTSYWMHWIKQRPGQGLEWIGEINPSNGGSNYNEKFKSKATLTVDKSSSTAYMQLSSLTSEDSAVYHCKSRGYWGQGTTLTVSS

SEQ ID NO: 134 - protein, 112 aa, Artificial Sequence
  source: /note=“Description of Artificial Sequence Synthetic polypeptide”
DVVMTQTPLTLSVTIGQPASISCKSSQSLLDSYGKTYLNWLLQRPGQSPKRLIYLVSKLDSGVPDRFTGSGSGTDFTLKISRVEAEDLGIYYCWQGTHFPHTFGSGTKLEIK

SEQ ID NO: 135 - protein, 118 aa, Artificial Sequence
  source: /note=“Description of Artificial Sequence Synthetic polypeptide”
QVQLQQSGAELARPGASVKLSCKASGYTFTSYGISWVKQRTGQGLEWIGEIFPRSSNTYYNEKFKGKATLTADKSSSTVYMEFRSLTSEDSAVYFCAREGGLAWFAYWGQGTLVTVSA

SEQ ID NO: 136 - protein, 112 aa, Artificial Sequence
  source: /note=“Description of Artificial Sequence Synthetic polypeptide”
DVVMTQTPLTLSVTIGQPASISCKSSQSLLYTNGNTYLNWLLQRPGQSPKRLIYLVSKLDSGIPDRFSGSGSGTDFTLRISRVEAEDLGVYYCLQSTHFPFTFGSGTKLEIK

SEQ ID NO: 137 - protein, 112 aa, Artificial Sequence
  source: /note=“Description of Artificial Sequence Synthetic polypeptide”
EVQLQQSGPELVKPGASVKMSCKASGYTITDYYMMWLKQSHGKSLEWIGDINPYTGGTSYNQKFKGKATLTVDKSSSTAYLQLHSLTSEDSAVYYCARGAYWGQGTTLTVSS

SEQ ID NO: 138 - protein, 112 aa, Artificial Sequence
  source: /note=“Description of Artificial Sequence Synthetic polypeptide”
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIK

SEQ ID NO: 139 - protein, 112 aa, Artificial Sequence
  source: /note=“Description of Artificial Sequence Synthetic polypeptide”
EVQLQQSGPELVKPGASVKMSCKASGYTITDYYMMWLKQSHGKSLEWIGDINPYTGGTSYNQKFKGKATLTVDKSSSTAYMQLNSLTSEDSAVYYCARGAYWGQGTTLTVSS

SEQ ID NO: 140 - protein, 112 aa, Artificial Sequence; identical to SEQ ID NO: 138
  source: /note=“Description of Artificial Sequence Synthetic polypeptide”

SEQ ID NO: 141 - protein, 112 aa, Artificial Sequence
  source: /note=“Description of Artificial Sequence Synthetic polypeptide”
EVQLQQSGPELVKPGASVKISCKASGYTFTDYYMNWVKQSHGRSLELIGDINPNNGGSNFNQKFRGKATLTVDKSSSTAYMELRSLTSEDSAIYYCARMGYWGQGTLVTVSA

SEQ ID NO: 142 - protein, 112 aa, Artificial Sequence
  source: /note=“Description of Artificial Sequence Synthetic polypeptide”
DVVMTQTPLTLSVTIGQPASISCKSSQSLLHSDGKTYLNWMFQRPGQSPKRLIYLVSKLDSGVPYRFTGGGSGTDFTLQISRVETEDLGVYYCWQGTHFPRTFGGGTKLEIK

SEQ ID NO: 143 - protein, 114 aa, Artificial Sequence
  source: /note=“Description of Artificial Sequence Synthetic polypeptide”
EVQLQQSGPELVKPGASVKMSCKASGYTFTDYYIHWVKQKPGQGLEYIGEIYPGSGNTYYNGKFRGKATLTADKSSSTAYMQLSSLTSEDSAVYFCGSGYFDYWGQGTTLTVSS

SEQ ID NO: 144 - protein, 112 aa, Artificial Sequence
  source: /note=“Description of Artificial Sequence Synthetic polypeptide”
DVVMTQTPLTLSVTIGQPASISCKSSQSLLYSNGKTYLNWLLQSPGQSPKLLIYLVSKLESGVPDRFSGSGSGTDFTLKLSRVEAEDLGVYYCVQGTHFPFTFGSGTKLEIK

SEQ ID NO: 145 - protein, 139 aa, Artificial Sequence
  source: /note=“Description of Artificial Sequence Synthetic polypeptide”
MDWLWNLLFLMVVAQSAQAQIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSS

SEQ ID NO: 146 - protein, 133 aa, Artificial Sequence
  source: /note=“Description of Artificial Sequence Synthetic polypeptide”
MESQTHVLISLLLCVSGTCGDILINQSPASLTVSAGERVTMSCKSSQSLLYSENNQDYLAWYQQKPGQFPKLLIYGASNRHTGVPDRFTGSGSGTDFTLTISSVQAEDLADYYCEQTYRYPFTFGSGTKLEIK

SEQ ID NO: 147 - protein, 138 aa, Artificial Sequence
  source: /note=“Description of Artificial Sequence Synthetic polypeptide”
MELELSLIFIFSLLKDVQCEVQLVESGGSLVQPGGSLKLSCVASGYTFSNYWMDWVRQSPGKSLEWIGEINTDGRRTNYAPSIKDRFTISRDNAKSTLYLQMSNVKSDDTAIYYCTILRVYPHYFDYWGQGVMVTVSS

SEQ ID NO: 148 - protein, 132 aa, Artificial Sequence
  source: /note=“Description of Artificial Sequence Synthetic polypeptide”
MMSPAQFLFLLMLWIQEARGDVVMTQTPPSLSVAIGQSVSISCKSSQSLVYSDGETYLHWFLQSPGRSPKRLIYHVSNLGSGVPDRFSGTGSLTDFTLRISRVEAEDLGVYYCAQTTHFPPTFGAGTKLELK

SEQ ID NO: 149 - protein, 135 aa, Artificial Sequence
  source: /note=“Description of Artificial Sequence Synthetic polypeptide”
MAVLVLLLCLVTFPSCALSQVQLKESGPGLVQPSQTLSLTCTVSGFSLTSNSVSWVRQAPGKGLEWMGAIWSGGSTDYNSALKSRLSISRDTSKSQVFLKMNSLQTEDTAIYFCTRSHWEPFDYWGQGVMVTVSS

SEQ ID NO: 150 - protein, 133 aa, Artificial Sequence
  source: /note=“Description of Artificial Sequence Synthetic polypeptide”
MESQTQALISLLLWVYGTCGDIVMTQSPFSLAVSEGEMVTINCKSSQGLLSSGNQKNYLAWYQQRPGQSPKLLIYYASTRQSGVPDRFIGGGSGTDFTLTISDVQAEDLADYYCLQHYSYPPTFGSGTKLEIK

SEQ ID NO: 151 - protein, 135 aa, Artificial Sequence
  source: /note=“Description of Artificial Sequence Synthetic polypeptide”
MAVLVLLLCLVTFPSCALSQVQLRESGPGLVQPSQTLSLTCTVSGFSLTSNSVTWVRQPPGKGLEWMGAIWSDGSTDYNSTLKSRLSISRDTSKSQVFLKMSSLQTEDTAIYFCTRSHWEPFDYWGQGVMVTVSS

SEQ ID NO: 152 - protein, 133 aa, Artificial Sequence
  source: /note=“Description of Artificial Sequence Synthetic polypeptide”
MESQTQALISLLLWVYGTCGDIVMTQSPLSLAVSEGETVTMNCKSSQSLFSSGNQKNYLAWYQQKPGQSPKLLIYYASTRQSGVPDRFIGSGSGTDFTLTISDVQTEDLADYYCLQHYNYPPTFGSGTKLEIK

SEQ ID NO: 153 - protein, 134 aa, Artificial Sequence
  source: /note=“Description of Artificial Sequence Synthetic polypeptide”
MDLRLTYVFIVAILKGVLCEVKLEESGGGLVQPGMSVKLSCATSGFIFSDYWMEWVRQAPGKGLEWVAEIRNKANNYATYYGKSMKGRFTISRDDSKSIVYLQVNSIRSEDTAIYYCAPNFDYWGQGVMVTVSS

SEQ ID NO: 154 - protein, 131 aa, Artificial Sequence
  source: /note=“Description of Artificial Sequence Synthetic polypeptide”
MSPVQSLFLLLLWILGTHGDVVLTQTPPTLSATIGQSVSISCRSSQSLLHSTGNTYLNWLLQRPGQPPQLLIYLVSRLESGVPNRFSASGSGTDFTLKISGIEAEDLGVYYCVQSSHTPYTFGTGTKLELK

SEQ ID NO: 155 - protein, 135 aa, Artificial Sequence
  source: /note=“Description of Artificial Sequence Synthetic polypeptide”
MDIRLSLVFLVLFMKGVQCEVQLVESGGGLVQPGRSLKLSCAASRFTFSDYNMAWVRQAPKKGLEWVATIYHDDSGSYYRDSVKGRFTISRNNAKSTLYLQMDSLRSEDMATYYCARHNNGFDYWGQGVMVTVAS

SEQ ID NO: 156 - protein, 131 aa, Artificial Sequence
  source: /note=“Description of Artificial Sequence Synthetic polypeptide”
MKWPVRLLVLFFWIPASGGDVVMTQTPVSLPVRLGGQASISCRSSQSLVHSNGNTYLHWYLQKPGQSPQLLINRVSNRFSGVPDRFSGSGSGTDFTLKINRVEPEDLGDYYCLQSTHFPLTFGSGTKLETK

SEQ ID NO: 157 - protein, 136 aa, Artificial Sequence
  source: /note=“Description of Artificial Sequence Synthetic polypeptide”
MDIRLSLGFLVLFIKGDQCAVQLVESGGGLVQPGRSLKLSCAASRITFTDYYMAWVRQAPTKGLEWVATISSDGGDTFYRDSVKGRFTISRDNAKSTLYLQMVSLRSEDTATYYCSTDRGAQFGYWGQGTLVTVSS

SEQ ID NO: 158 - protein, 126 aa, Artificial Sequence
  source: /note=“Description of Artificial Sequence Synthetic polypeptide”
MAPVQLLGLLLIWLPAMRCDIQMTQSPSFLSASVGDRVSINCKASQNVHENLNWYQQKLGEAPKRLIYNTNNLQTGIPSRFSGSGSGADYTLTISSLQPEDFATYFCLQHNAFPYTFGPGTKLELK

SEQ ID NO: 159 - protein, 455 aa, Artificial Sequence
  source: /note=“Description of Artificial Sequence Synthetic polypeptide”
METGLRWLLLVAVFKGVQCQEQLVESGGDLVKPGASLTLTCTASGFSFNKNYWMCWVRQAPGKGLEWIGCIYNGDGNTYYASWVNGRFTISKTSSTTVTLQMTSLTVADTAIYFCARLLNMWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 160 - protein, 239 aa, Artificial Sequence
  source: /note=“Description of Artificial Sequence Synthetic polypeptide”
MDTRAPTQLLGLLLLWLPGARCADIVMTQTPASVEAAVGGTVTIKCQASESIGNALAWYQQKPGQPPKLLIYTAATLASGVPSRFSGSGSGTEFTLTISGVQCDDAATYYCQSYYFTSVSSYGNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 161 - protein, 466 aa, Artificial Sequence
  source: /note=“Description of Artificial Sequence Synthetic polypeptide”
METGLRWLLLVAVLKGVQCQSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 162 - protein, 237 aa, Artificial Sequence
  source: /note=“Description of Artificial Sequence Synthetic polypeptide”
MDTRAPTQLLGLLLLWLPGARCAFELTQTPSSVEAAVGGTPTIKCQASQTIYSYLSWYQQKPGQPPKLLIYEASKLASGVPSRFSGSGSGTDYTLTISDLECADAATYYCQSYHGTASTEYNTFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 163 - protein, 466 aa, Artificial Sequence
  source: /note=“Description of Artificial Sequence Synthetic polypeptide”
METGLRWLLLVAVLKGVQCQQQLVESGGGLVKPGAALTFTCTASGFSFSGNYWICWVRQAPGKGLEWIACIGTITSRTYYASWAKGRFTISKTSSTTVTLQMTSLTAADTATYFCARGAVVSSGNAPYYFTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 164 - protein, 236 aa, Artificial Sequence
  source: /note=“Description of Artificial Sequence Synthetic polypeptide”
MDTRAPTQLLGLLLLWLPGARCAFELTQTPSSVEAAVGGTVTIKCQASQSISSYLSWYQQKPGQPPKLLIYRASTLESGVPSRFKGSGSGTEFTLTISDLECADAATYFCQSYYGVTFSGFAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 165 - protein, 460 aa, Artificial Sequence
  source: /note=“Description of Artificial Sequence Synthetic polypeptide”
METGLRWLLLVAVLKGVQCQSLEESGGDLVKPGASLTLTCKASGIDFNNYWITWVRQAPGKGLEWIACIYVGITGRTWYANWAKGRFTISKASSTVDLKMTSLTAADTATYFCARNGDGGIYALNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 166 - protein, 238 aa, Artificial Sequence
  source: /note=“Description of Artificial Sequence Synthetic polypeptide”
MDTRAPTQLLGLLLLWLPGATFAQVLTQTASSVSAAVGGTVTISCQSSQSVYNNNWLAWYQQKPGQPPKLLIYRASTLTSGVPSRFKGSGSGTQFTLTISDLECDDAATYYCAGGYSGNIYVNDFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 167 - protein, 459 aa, Artificial Sequence
  source: /note=“Description of Artificial Sequence Synthetic polypeptide”
METGLRWLLLVAVLKGVQCQEQLEESGGDLVKPGGSLTLTCKASGFSFSNTYWACWVRQAPGKGLEWIACMNPASSGSSYYASWAKGRFTISKTSSTTVTLHMPSLTAADTATYFCAKWDTAFDVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 168 - protein, 237 aa, Artificial Sequence
  source: /note=“Description of Artificial Sequence Synthetic polypeptide”
MDTRAPTQLLGLLLLWLPGARCADVVMTQTPSSVEAAVGGTVTIKCQASQSISSYLAWYQQKPGQPPKLLIYGASNLESGVPSRFKGSGSGTEYTLTISGVQCDDAATYYCQNYYAIDTYGHAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 169 - protein, 455 aa, Artificial Sequence
  source: /note=“Description of Artificial Sequence Synthetic polypeptide”
METGLRWLLLVAVLKGVQCQEQLEESGGDLVKPGASLTLTCTASGFSFSSNYHICWVRQAPGKGLELIACIYVGDGSTYYASWAKGRFTISKSSSTTVALQMTSLTAADTATYFCGRMFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 170 - protein, 235 aa, Artificial Sequence
  source: /note=“Description of Artificial Sequence Synthetic polypeptide”
MDTRAPTQLLGLLLLWLPGAICDPVLTQTPSSVSAAVGVTVTINCQSSPSVYSNYLSWYQQKPGQPPKLLIYLASTLASGVPSRFKGSGSGTQFTLTISDVQCDDAATYYCAGTYSGNIWSFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 171 - protein, 460 aa, Artificial Sequence
  source: /note=“Description of Artificial Sequence Synthetic polypeptide”
METGLRWLLLVAVLKGVQCQQQLVESGGGLVKPGASLTLTCTASGFSFSSGYHMCWVRQAPGKGLEWIACFGVYTGTTTYASWAKGRFTISKTSSTTVTLQMTSLTVADTATYFCARISAENGGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 172 - protein, 236 aa, Artificial Sequence
  source: /note=“Description of Artificial Sequence Synthetic polypeptide”
MDTRAPTQLLGLLLLWLPGARCDVVMTQTPASVEAAVGGTVTIKCQASQSISNYFSWYQQKPGQPPKLLIYRASTLASGVPSRFSGSGSGTEFTLTISDLECADSATYYCQCTYGSSSTGFGFGGGTEVVVKGDPVAPTVPIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 173 - protein, 456 aa, Artificial Sequence
  source: /note=“Description of Artificial Sequence Synthetic polypeptide”
METGLRWLLLVAVLKGVQCQSLEESGGDLVKPGASLTLTCMASGIDFSSGYGMWWVRQAPGKGLEYIGYIDTGDDNTYYANWAKGRFTISKTSSTTVTLQMTSLTVADTATYFCAKGGAIDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 174 - protein, 239 aa, Artificial Sequence
  source: /note=“Description of Artificial Sequence Synthetic polypeptide”
MDTRAPTQLLGLLLLWLPGARCADIVMTQTPASVEAAVGGTVTIKCQASQSISSYLAWYQQKPGQRPKLLIYRASTLKSGVPSRFKGSGSGTEYTLTISDLECADAATYYCQAYYLSSSISYGNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 175 - protein, 456 aa, Artificial Sequence
  source: /note=“Description of Artificial Sequence Synthetic polypeptide”
METGLRWLLLVAVLKGVQCQSLEESGGDLVKPGASLTLTCTASGFSLSSGYGMCWVRQAPGKGLEWIGYTDTATGTIHYASWAKGRFTISKTSSTTVTLQMTSLTAADTATYFCAKGGAMDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 176 - protein, 239 aa, Artificial Sequence
  source: /note=“Description of Artificial Sequence Synthetic polypeptide”
MDTRAPTQLLGLLLLWLPGARCADIVMTQTPASVEAAVGGTVTIKCQASQSISSYLAWYQQKPGQPPKLLIYRTSTLASGVPSRFKGSGSGTEYTLTISDLECADAATYYCQSYAYSSSSSYGNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 177 - protein, 454 aa, Artificial Sequence
  source: /note=“Description of Artificial Sequence Synthetic polypeptide”
METGLRWLLLVAVLKGVQCQSLEESGGDLVKPGASLTLTCTGSGIDFSSSYWICWVRQAPGKGLEWIACIDGSDGNTYYASWARGRFTISKTSSTTVTLQMASLTAADTATYFCTRDLRLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 178 - protein, 240 aa, Artificial Sequence
  source: /note=“Description of Artificial Sequence Synthetic polypeptide”
MDTRAPTQLLGLLLLWLPGARCADIVLTQTPASVSAAVGGTVTINCQASQNVYSNNALAWHQQKPGQRPNLLIYKASTLASGVPSRFKGSGSGTQFTLTISDVQCDDAATYYCLGEFSCSSGDCFVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 179 - protein, 456 aa, Artificial Sequence; identical to SEQ ID NO: 173
  source: /note=“Description of Artificial Sequence Synthetic polypeptide”

SEQ ID NO: 180 - protein, 239 aa, Artificial Sequence; identical to SEQ ID NO: 174
  source: /note=“Description of Artificial Sequence Synthetic polypeptide”

SEQ ID NO: 181 - protein, 471 aa, Artificial Sequence
  source: /note=“Description of Artificial Sequence Synthetic polypeptide”
METGLRWLLLVAVLKGVQCQQQLEESGGGLVKPGGTLTLTCKASGVALNPYYYMCWVRQAPGKGLEWIACVDADSSGSTYYASWAKGRFTISKTSSTTVTLKMTSLTAADTATYFCARESVDYSSVGIGYVHGTDGLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 182 - protein, 239 aa, Artificial Sequence
  source: /note=“Description of Artificial Sequence Synthetic polypeptide”
MDTRAPTQLLGLLLLWLPGARCADIVVTQTPSSVSAAVGGTVTIKCQASQSISNYFSWYQQKPGQPPKLLIYRASTLASGVPSRFKGSGSGTEFTLTISDLECADAATYYCQCTYGRSNSNFFYGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 183 - protein, 464 aa, Artificial Sequence
  source: /note=“Description of Artificial Sequence Synthetic polypeptide”
METGLRWLLLVAVLKGVQCQSLEESGGDLVKPGASLTLTCTASGSSFSSTYWNCWVRQAPGKGLEWIACINAGSGTTYYASWAKGRFTVSKTSSTTVTLQMTSLTAADTATYFCTRDSDGRFSSGYYFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 184 - protein, 238 aa, Artificial Sequence
  source: /note=“Description of Artificial Sequence Synthetic polypeptide”
MDTRAPTQLLGLLLLWLPGATFAQVLTQTASPVSAAVGGTVTINCQSSQSVYDNNWLAWYQQKPGQPPKLLIDDASKLTSGVSSRFKGSGSGTQFTLTISGVQCDDAATYYCQGAYYSSGWYWAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 185 - protein, 467 aa, Artificial Sequence
  source: /note=“Description of Artificial Sequence Synthetic polypeptide”
METGLRWLLLVAVLKGVQCQQQLEESGGGLVKPGGTLTLTCKASGIDFSSYYYMCWVRQAPGKGLELIVCIYTSSGGTWYASWVNGRLTISRSTSLNTVDLKMTSLTAADTATYFCARGVYSGSSDYPTRLDLWGQGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 186 - protein, 236 aa, Artificial Sequence
  source: /note=“Description of Artificial Sequence Synthetic polypeptide”
MDTSTSTALLGLLLLWLTGARCAIEMTQSPPSLSASVGETVRIRCLASEDIYSGISWYQQKPEKPPTLLISGASNLESGVPPRFSGGGSGTDYTLTIGGVQAEDVATYYCLGGYSFSSTGLTFGAGTKVEIKRDPVAPSVLLFPPSKEELTTGTATIVCVANKYFPDVTVTWEVDGTTQTTGIENSKTPQNSADCTYNLSSTLTLTSTQYNSHKEYTCKVTQGTTSVVQSFNRGDC

SEQ ID NO: 187 - protein, 460 aa, Artificial Sequence
  source: /note=“Description of Artificial Sequence Synthetic polypeptide”
METGLRWLLLVAVLKGVQCQQHLVESGGGLVKPGASLTLTCTASGFSFTTGYHMCWVRQAPGKGLEWIACFGVYTSTTTYASWAKGRFTISKTSSTTVTLQMTSLTVADTATYFCARISAEDGGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 188 - protein, 236 aa, Artificial Sequence
  source: /note=“Description of Artificial Sequence Synthetic polypeptide”
MDTRAPTQLLGLLLLWLPGARCDVVMTQTPASVEAAVGGTVTITCQASQSISNYFSWYQQKPGQPPKLLIYRASTLASGVPSRFSGSGSGTQFTLTISDLECADSATYACQCTYGSSSTGFGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 189 - protein, 462 aa, Artificial Sequence
  source: /note=“Description of Artificial Sequence Synthetic polypeptide”
METGLRWLLLVAVLKGVQCQSLEESGGDLVKPGASLTLTCKASGFSFSSYFWICWVRQAPGKGLEWSACIYGDSSGSSYYASWAKGRFTISKTSSTTVTLQMTSLTAADTATYFCASYGSSSYYYSNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 190 - protein, 235 aa, Artificial Sequence
  source: /note=“Description of Artificial Sequence Synthetic polypeptide”
MDTRAPTQLLGLLLLWLPGAICDPVMTQTPSSTSAAVGGTVTISCQSSQSVYNNNYLAWYQQKPGQPPKRLIYESSKLASGVPSRFRGSGSGAQFTLTISDLECDDAATYYCLGAYYTTLDFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 191 - protein, 460 aa, Artificial Sequence
  source: /note=“Description of Artificial Sequence Synthetic polypeptide”
METGLRWLLLVAVLKGVQCQEQLVESGGGLVQPEGSLTLTCKASGFDFSSNAMCWVRQAPGKGLEWIACIYNGDGSTYYASWVNGRFTISKTSSTTVTLQMTSLTAADTATYFCARGLSNWNRDNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 192 - protein, 239 aa, Artificial Sequence
  source: /note=“Description of Artificial Sequence Synthetic polypeptide”
MDTRAPTQLLGLLLLWLPGATFAQVLTQTPSSVSAAVGGTATINCQASQSLYSPKNLAWYQQTPGQPPKLLIYSASKLASGVPSRFKGSGSGTQFTLTISGVQCDDAAIYYCQGEFSCTTAACFAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 193 - protein, 461 aa, Artificial Sequence
  source: /note=“Description of Artificial Sequence Synthetic polypeptide”
METGLRWLLLVAVLKGVQCQSLEESGGGLVQPEGSLTLACTASGFSFSSYYYICWVRQAPGTGLEWIGCINTGSDDTHYASWLKGRFTFSKASSTTLTLQMTSLTAADTATYFCARSSGSSDDAYDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 194 - protein, 235 aa, Artificial Sequence
  source: /note=“Description of Artificial Sequence Synthetic polypeptide”
MDTRAPTQLLGLLLLWLPGARCDVVMTQTPASVSEPVGGAVTIKCQASQSIGSNLAWYQHKPGQPPKLLIYFASSLASGVSSRFKGGRSGTQFTLTISDLECADAATYYCHCTYYPLSYVTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 195 - protein, 464 aa, Artificial Sequence
  source: /note=“Description of Artificial Sequence Synthetic polypeptide”
METGLRWLLLVAVLKGVQCQSLEESGGDLVKPGTSLTLSCTASGFSFGSYYYMCWVRQAPGKGLEWIACIDVGSSGDTYYASWVNGRFTISKTSSTTVTLQMTSLTAADTATYFCARDDTAAGGFGNLELWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 196 - protein, 238 aa, Artificial Sequence
  source: /note=“Description of Artificial Sequence Synthetic polypeptide”
MDTRAPTQLLGLLLLWLPGARCAFELTQTPSSVSEPVGGTVTIKCQASQSIYSYFSWYQQKPGQPPKRLIYQASTLASGVPSRFKGSGSGTDFTLTISDLECADAATYYCQNNYGRGSGSYFFGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 197 - protein, 456 aa, Artificial Sequence
  source: /note=“Description of Artificial Sequence Synthetic polypeptide”
METGLRWLLLVAVLKGVQCQSLEESGGDLVKPGASLTLTCKASGIDFSSGYGMWWVRQAPGKGLEYIGYIDTGSGSTYYANWAKGRFTISKTSSTMVTLQMTSLTVADTATYFCAKGGAIDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 198 - protein, 239 aa, Artificial Sequence
  source: /note=“Description of Artificial Sequence Synthetic polypeptide”
MDTRAPTQLLGLLLLWLPGARCADIVMTQTPASVEAAVGGTVTIKCQASQSISSYLAWYQQKPGQRPKLLIYRASTLASGVPSRFKGSGSGTDYTLTISDLECADAATYYCHTYYLSSSISYGNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 199 - protein, 465 aa, Artificial Sequence
  source: /note=“Description of Artificial Sequence Synthetic polypeptide”
METGLRWLLLVAVLKGVQCQEQLEESGGDLVKPEGSLTLTCKASGFSFSSIYWICWVRQAPGKGLEWIACTTVVKSGRTYYANWAKGRFTISKTSSTTVTLQMTSLTAADTATYFCAREFVDGGGSSGRDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 200 - protein, 236 aa, Artificial Sequence
  source: /note=“Description of Artificial Sequence Synthetic polypeptide”
MDTRAPTQLLGLLLLWLPGARCAYDMTQTPASVEAAVGGTVTIKCQASQSISRDLSWYQQKPGQPPKRLIYKASTLASGVPSRFKGSGSGTDFTLTISDLECADAATYYCQQGYSSIDVDNDFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 201 - protein, 467 aa, Artificial Sequence
  source: /note=“Description of Artificial Sequence Synthetic polypeptide”
METGLRWLLLVAVLKGVQCQEQLEESGGGLVKPEGSLTLTCKASGFDLSSGYDMCWVRQAPGKGLEWIACIYADYSGSTYYASWVNGRFTISSSTSLNTVDLKMTSLTAADTATYFCARGATGNGGYGYYFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 202 - protein, 238 aa, Artificial Sequence
  source: /note=“Description of Artificial Sequence Synthetic polypeptide”
MDTRAPTQLLGLLLLWLPGARCADIVMTQTPASVSEPVGGTVTIKCQASQNINSGLAWYQQKPGQPPKLLIYKASTLASGVSSRFKGSGSGTEFTLTISDLECADAATYYCQTYYYSSSSSDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 203 - protein, 462 aa, Artificial Sequence
  source: /note=“Description of Artificial Sequence Synthetic polypeptide”
METGLRWLLLVAVLKGVQCQSLEESGGDLVKPGASLTLTCTASGFSFSSSYWICWVRQAPGKGLEWIACIYGGSSGNIAYASWAKGRFTISKTSSTTVTLQMTSLTAADTATYFCARDIPSDAFTLDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 204 - protein, 239 aa, Artificial Sequence
  source: /note=“Description of Artificial Sequence Synthetic polypeptide”
MDTRAPTQLLGLLLLWLPGATFAQVLTQTPSSVSAAVGSTVTINCQASQSVYKDNNLAWYQQKPGQPPKLLIYKASTLASGVPSRFKGSGSGTQFTLTISGVQCEDAATYYCQGEFSCGSADCIAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 205 - protein, 456 aa, Artificial Sequence
  source: /note=“Description of Artificial Sequence Synthetic polypeptide”
METGLRWLLLVAVLKGVQCQSLEESGGDLVKPGASLTLTCKGSGIDFSSGYGMWWVRQAPGKGLEYIGYIDTGYGSTYYASWAKGRFTISKTSSTTVTLQMTSLTVADTATYFCAKGGAIDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 206 - protein, 238 aa, Artificial Sequence
  source: /note=“Description of Artificial Sequence Synthetic polypeptide”
MDTRAPTQLLGLLLLWLPGATFAAVLTQTPASTSAAVGGTVTINCQSSQSVYRSNWLAWYQQKPGQPPKLLIYDVFNLASGVPSRFKGSGSGTQFTLTISGVQCADAATYYCQGSYYSGNWYSAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 207 - protein, 13 aa, Artificial Sequence
  source: /note=“Description of Artificial Sequence Synthetic peptide”
AAWDDSLNGLNWV

SEQ ID NO: 208 - protein, 25 aa, Artificial Sequence
  source: /note=“Description of Artificial Sequence Synthetic peptide”
QVQLVESGGGVVQPGRSLRLSCAAS

SEQ ID NO: 209 - protein, 8 aa, Artificial Sequence
  source: /note=“Description of Artificial Sequence Synthetic peptide”
GFTFSNYG

SEQ ID NO: 210 - protein, 17 aa, Artificial Sequence
  source: /note=“Description of Artificial Sequence Synthetic peptide”
MNWVRQAPGKGLEWVSY

SEQ ID NO: 211 - protein, 8 aa, Artificial Sequence
  source: /note=“Description of Artificial Sequence Synthetic peptide”
ISSSGSTV

SEQ ID NO: 212 - protein, 40 aa, Artificial Sequence
  source: /note=“Description of Artificial Sequence Synthetic polypeptide”
YYADSVKGRFTISRDNAKNSLYLQMNSLRDEDTAVYYCAS

SEQ ID NO: 213 - protein, 11 aa, Artificial Sequence
  source: /note=“Description of Artificial Sequence Synthetic peptide”
GQLDTSDAFDI

SEQ ID NO: 214 - protein, 11 aa, Artificial Sequence
  source: /note=“Description of Artificial Sequence Synthetic peptide”
WGQGTLVTVSS

SEQ ID NO: 215 - protein, 26 aa, Artificial Sequence
  source: /note=“Description of Artificial Sequence Synthetic peptide”
DIEMTQSPSSPSASVGDRVTITCRAS

SEQ ID NO: 216 - protein, 6 aa, Artificial Sequence
  source: /note=“Description of Artificial Sequence Synthetic peptide”
QSISSY

SEQ ID NO: 217 - protein, 17 aa, Artificial Sequence
  source: /note=“Description of Artificial Sequence Synthetic peptide”
LNWYQQKPGKAPKLLIY

SEQ ID NO: 218 - protein, 3 aa, Artificial Sequence
  source: /note=“Description of Artificial Sequence Synthetic peptide”
AAS

SEQ ID NO: 219 - protein, 36 aa, Artificial Sequence
  source: /note=“Description of Artificial Sequence Synthetic polypeptide”
SLQSGVPSRFSGSGSGTDFTLTISSLQPEDFATYYC

SEQ ID NO: 220 - protein, 9 aa, Artificial Sequence
  source: /note=“Description of Artificial Sequence Synthetic peptide”
QQSYSTPLT

SEQ ID NO: 221 - protein, 10 aa, Artificial Sequence
  source: /note=“Description of Artificial Sequence Synthetic peptide”
FGGGTKVEIK

SEQ ID NO: 222 - protein, 25 aa, Artificial Sequence
  source: /note=“Description of Artificial Sequence Synthetic peptide”
EVQLLESGGGVVQSGRSLRVSCAAS

SEQ ID NO: 223 - protein, 8 aa, Artificial Sequence
  source: /note=“Description of Artificial Sequence Synthetic peptide”
GFSFSSYG

SEQ ID NO: 224 - protein, 17 aa, Artificial Sequence
  source: /note=“Description of Artificial Sequence Synthetic peptide”
MHWVRQAPGKGLEWVSY

SEQ ID NO: 225 - protein, 8 aa, Artificial Sequence
  source: /note=“Description of Artificial Sequence Synthetic peptide”
ISSSGSTI

SEQ ID NO: 226 - protein, 40 aa, Artificial Sequence
  source: /note=“Description of Artificial Sequence Synthetic polypeptide”
YYADSVKGRFTISRDNAENSLYLQMNSLRAEDTAVYYCAR

SEQ ID NO: 227 - protein, 26 aa, Artificial Sequence [sequence omitted]

SEQ ID NO: 228 - protein, 6 aa, Artificial Sequence
  source: /note=“Description of Artificial Sequence Synthetic peptide”
QGISNY

SEQ ID NO: 229 - protein, 17 aa, Artificial Sequence
  source: /note=“Description of Artificial Sequence Synthetic peptide”
LAWYQQKPGKVPKLLIY

SEQ ID NO: 230 - protein, 36 aa, Artificial Sequence
  source: /note=“Description of Artificial Sequence Synthetic polypeptide”
TLQSGVPSRFSGSGSGTDFTLTISSLQPEDFATYYC

SEQ ID NO: 231 - protein, 10 aa, Artificial Sequence
  source: /note=“Description of Artificial Sequence Synthetic peptide”
FGGGTKVEVK

SEQ ID NO: 232 - protein, 25 aa, Artificial Sequence
  source: /note=“Description of Artificial Sequence Synthetic peptide”
EVQLLESGGGVVQPGRSLRLSCAAS

SEQ ID NO: 233 - protein, 8 aa, Artificial Sequence
  source: /note=“Description of Artificial Sequence Synthetic peptide”
ISSSSSTI

SEQ ID NO: 234 - protein, 11 aa, Artificial Sequence
  source: /note=“Description of Artificial Sequence Synthetic peptide”
  MOD_RES (3)..(3): Any amino acid
  MOD_RES (5)..(5): Any amino acid
GQXDXSDAFDI

SEQ ID NO: 235 - protein, 17 aa, Artificial Sequence
  source: /note=“Description of Artificial Sequence Synthetic peptide”
  MOD_RES (3)..(3): Any amino acid
  MOD_RES (16)..(16): Any amino acid
LNXYQQKPGKAPKLLXY

SEQ ID NO: 236 - protein, 3 aa, Artificial Sequence
  source: /note=“Description of Artificial Sequence Synthetic peptide”
  MOD_RES (1)..(1): Any amino acid
XAS

SEQ ID NO: 237 - protein, 36 aa, Artificial Sequence
  source: /note=“Description of Artificial Sequence Synthetic polypeptide”
  MOD_RES (31)..(31): Any amino acid
SLQSGVPSRFSGSGSGTDFTLTISSLQPEDXATYYC

SEQ ID NO: 238 - protein, 10 aa, Artificial Sequence
  source: /note=“Description of Artificial Sequence Synthetic peptide”
  MOD_RES (5)..(5): Any amino acid
  MOD_RES (7)..(7): Any amino acid
FGGGXKXEIK

SEQ ID NO: 239 - protein, 40 aa, Artificial Sequence
  source: /note=“Description of Artificial Sequence Synthetic polypeptide”
YYADSVKGRFAISRDNAKNTLYLQMNSLRAEDTALYYCAK

SEQ ID NO: 240 - protein, 19 aa, Artificial Sequence
  source: /note=“Description of Artificial Sequence Synthetic peptide”
ATRYDILTGYSDGVDYFDY

SEQ ID NO: 241 - protein, 9 aa, Artificial Sequence
  source: /note=“Description of Artificial Sequence Synthetic peptide”
HQSYSTPYT

SEQ ID NO: 242 - protein, 10 aa, Artificial Sequence
  source: /note=“Description of Artificial Sequence Synthetic peptide”
FGQGTKLEIK

SEQ ID NO: 243 - protein, 25 aa, Artificial Sequence
  source: /note=“Description of Artificial Sequence Synthetic peptide”
QVQLVESGGGLVQPGGSLRLSCAAS

SEQ ID NO: 244 - protein, 17 aa, Artificial Sequence
  source: /note=“Description of Artificial Sequence Synthetic peptide”
MSWVRQAPGKGLEWVAN

SEQ ID NO: 245 - protein, 8 aa, Artificial Sequence
  source: /note=“Description of Artificial Sequence Synthetic peptide”
IKQDGSEK

SEQ ID NO: 246 - protein, 40 aa, Artificial Sequence
  source: /note=“Description of Artificial Sequence Synthetic polypeptide”
YYVDSVKGRFTISRDNAKNSLYLQMNSLRAEDTAVYYCAR

SEQ ID NO: 247 - protein, 9 aa, Artificial Sequence
  source: /note=“Description of Artificial Sequence Synthetic peptide”
VTSPHAFDI

SEQ ID NO: 248 - protein, 11 aa, Artificial Sequence
  source: /note=“Description of Artificial Sequence Synthetic peptide”
WGRGTLVTVSS

SEQ ID NO: 249 - protein, 26 aa, Artificial Sequence
  source: /note=“Description of Artificial Sequence Synthetic peptide”
DIQMTQSPSAMSASVGDRVTITCRAS

SEQ ID NO: 250 - protein, 17 aa, Artificial Sequence
  source: /note=“Description of Artificial Sequence Synthetic peptide”
LAWFQQKPGKVPKRLIY

SEQ ID NO: 251 - protein, 25 aa, Artificial Sequence
  source: /note=“Description of Artificial Sequence Synthetic peptide”
QVQLVESGGGVVRPGGSLRLSCAAS

SEQ ID NO: 252 - protein, 17 aa, Artificial Sequence
  source: /note=“Description of Artificial Sequence Synthetic peptide”
MSWVRQAPGKGLEWVSG

SEQ ID NO: 253 - protein, 8 aa, Artificial Sequence
  source: /note=“Description of Artificial Sequence Synthetic peptide”
INWNGGST

SEQ ID NO: 254 - protein, 40 aa, Artificial Sequence
  source: /note=“Description of Artificial Sequence Synthetic polypeptide”
GYADSVKGRFTISRDNSKNTLYLQMNSLRGEDTAVYYCVT

SEQ ID NO: 255 - protein, 7 aa, Artificial Sequence
  source: /note=“Description of Artificial Sequence Synthetic peptide”
QGSAFDI

SEQ ID NO: 256 - protein, 25 aa, Artificial Sequence
  source: /note=“Description of Artificial Sequence Synthetic peptide”
SYELTQPPSLSVSPGQTARITCSGD

SEQ ID NO: 257 - protein, 6 aa, Artificial Sequence
  source: /note=“Description of Artificial Sequence Synthetic peptide”
ALAKQY

SEQ ID NO: 258 - protein, 17 aa, Artificial Sequence
  source: /note=“Description of Artificial Sequence Synthetic peptide”
AYWYQQTPGQAPVLVIY

SEQ ID NO: 259 - protein, 3 aa, Artificial Sequence
  source: /note=“Description of Artificial Sequence Synthetic peptide”
KDT

SEQ ID NO: 260 - protein, 36 aa, Artificial Sequence
  source: /note=“Description of Artificial Sequence Synthetic polypeptide”
ERPSGIPERFSGSSSGTTVTLTISGVQAEDEVDYYC

SEQ ID NO: 261 - protein, 11 aa, Artificial Sequence
  source: /note=“Description of Artificial Sequence Synthetic peptide”
QSTDSSGTYQV

SEQ ID NO: 262 - protein, 10 aa, Artificial Sequence
  source: /note=“Description of Artificial Sequence Synthetic peptide”
FGGGTKLTVL

SEQ ID NO: 263 - protein, 25 aa, Artificial Sequence [sequence omitted]

SEQ ID NO: 264 - protein, 17 aa, Artificial Sequence
  source: /note=“Description of Artificial Sequence Synthetic peptide”
ISWVRQAPGQGLEWMGW

SEQ ID NO: 265 - protein, 8 aa, Artificial Sequence
  source: /note=“Description of Artificial Sequence Synthetic peptide”
ISAYNGNT

SEQ ID NO: 266 - protein, 40 aa, Artificial Sequence
  source: /note=“Description of Artificial Sequence Synthetic polypeptide”
NYAQKLQGRVTMTTDTSTSTAYMELRSLRSDDTAAYYCAR

SEQ ID NO: 267 - protein, 11 aa, Artificial Sequence
  source: /note=“Description of Artificial Sequence Synthetic peptide”
VTGITGTTIDP

SEQ ID NO: 268 - protein, 11 aa, Artificial Sequence
  source: /note=“Description of Artificial Sequence Synthetic peptide”
WGQGTMVTVSS

SEQ ID NO: 269 - protein, 3 aa, Artificial Sequence
  source: /note=“Description of Artificial Sequence Synthetic peptide”
DAS

SEQ ID NO: 270 - protein, 36 aa, Artificial Sequence
  source: /note=“Description of Artificial Sequence Synthetic polypeptide”
SLESGVPSRFSGSGSGTEFTLTISSLQPDDFAVYYC

SEQ ID NO: 271 - protein, 9 aa, Artificial Sequence
  source: /note=“Description of Artificial Sequence Synthetic peptide”
QQYNNWPQT

SEQ ID NO: 272 - protein, 10 aa, Artificial Sequence
  source: /note=“Description of Artificial Sequence Synthetic peptide”
FGQGTKVEIK

SEQ ID NO: 273 - protein, 25 aa, Artificial Sequence
  source: /note=“Description of Artificial Sequence Synthetic peptide”
QVQLVESGGGLVKPGGSLRLSCAAS

SEQ ID NO: 274 - protein, 17 aa, Artificial Sequence
  source: /note=“Description of Artificial Sequence Synthetic peptide”
MSWVRQAPGKGLEWVSA

SEQ ID NO: 275 - protein, 8 aa, Artificial Sequence
  source: /note=“Description of Artificial Sequence Synthetic peptide”
ISGSGGST

SEQ ID NO: 276 - protein, 40 aa, Artificial Sequence
  source: /note=“Description of Artificial Sequence Synthetic polypeptide”
YYADSVKGRFTISRDNSKNTLYLQMNSLRAEDTAVYYCAR

SEQ ID NO: 277 - protein, 12 aa, Artificial Sequence
  source: /note=“Description of Artificial Sequence Synthetic peptide”
DYGGYDGVYFDY

SEQ ID NO: 278 - protein, 25 aa, Artificial Sequence
  source: /note=“Description of Artificial Sequence Synthetic peptide”
SYELTQDPAVSVALGQTVRITCQGD

SEQ ID NO: 279 - protein, 6 aa, Artificial Sequence
  source: /note=“Description of Artificial Sequence Synthetic peptide”
SLRSYY

SEQ ID NO: 280 - protein, 17 aa, Artificial Sequence
  source: /note=“Description of Artificial Sequence Synthetic peptide”
ASWYQQKPGQAPVLVIY

SEQ ID NO: 281 - protein, 3 aa, Artificial Sequence
  source: /note=“Description of Artificial Sequence Synthetic peptide”
GKN

SEQ ID NO: 282 - protein, 36 aa, Artificial Sequence
  source: /note=“Description of Artificial Sequence Synthetic polypeptide”
NRPSGIPDRFSGSSSGNTASLTITGAQAEDEADYYC

SEQ ID NO: 283 - protein, 11 aa, Artificial Sequence
  source: /note=“Description of Artificial Sequence Synthetic peptide”
NSRDSSGNHVV

SEQ ID NO: 284 - protein, 10 aa, Artificial Sequence
  source: /note=“Description of Artificial Sequence Synthetic peptide”
FGGGTKVTVL

SEQ ID NO: 285 - protein, 25 aa, Artificial Sequence
  source: /note=“Description of Artificial Sequence Synthetic peptide”
EVQLLESGGGLVKPGGSLRLSCAAS

SEQ ID NO: 286 - protein, 17 aa, Artificial Sequence
  source: /note=“Description of Artificial Sequence Synthetic peptide”
MHWVRQAPGKGLEWVAV

SEQ ID NO: 287 - protein, 8 aa, Artificial Sequence
  source: /note=“Description of Artificial Sequence Synthetic peptide”
ISYDGSNK

SEQ ID NO: 288 - protein, 40 aa, Artificial Sequence
  source: /note=“Description of Artificial Sequence Synthetic polypeptide”
YYADSVKGRFAISRDNSKNTLYLQMNSLRAEDTAVYYCAR

SEQ ID NO: 289 - protein, 15 aa, Artificial Sequence
  source: /note=“Description of Artificial Sequence Synthetic peptide”
DRDVGPTYYYYGMDV

SEQ ID NO: 290 - protein, 25 aa, Artificial Sequence
  source: /note=“Description of Artificial Sequence Synthetic peptide”
SYELTQPPSLSVSPGQTARITCSGH

SEQ ID NO: 291 - protein, 6 aa, Artificial Sequence
  source: /note=“Description of Artificial Sequence Synthetic peptide”
ALPKQY

SEQ ID NO: 292 - protein, 36 aa, Artificial Sequence
  source: /note=“Description of Artificial Sequence Synthetic polypeptide”
ERPSGIPERFSGSSSGTTVTLTISGVQAEDEADYYC

SEQ ID NO: 293 - protein, 11 aa, Artificial Sequence
  source: /note=“Description of Artificial Sequence Synthetic peptide”
QSADSSGPYQV

SEQ ID NO: 294 - protein, 10 aa, Artificial Sequence
  source: /note=“Description of Artificial Sequence Synthetic peptide”
FGGGTQLTVL

SEQ ID NO: 295 - protein, 40 aa, Artificial Sequence
  source: /note=“Description of Artificial Sequence Synthetic polypeptide”
YYADSVKGRFTISRDNSKNTLYLQMNSLRAEDTAVYYCAK

SEQ ID NO: 296 - protein, 11 aa, Artificial Sequence
  source: /note=“Description of Artificial Sequence Synthetic peptide”
DREYIAVAADY

SEQ ID NO: 297 - protein, 11 aa, Artificial Sequence
  source: /note=“Description of Artificial Sequence Synthetic peptide”
WGQGTTVTVSS

SEQ ID NO: 298 - protein, 26 aa, Artificial Sequence
  source: /note=“Description of Artificial Sequence Synthetic peptide”
DIQMTQSPSSLSASVGDTISITCRAS

SEQ ID NO: 299 - protein, 36 aa, Artificial Sequence
  source: /note=“Description of Artificial Sequence Synthetic polypeptide”
SLQSGVPSRFSGSGSRTDFTLTISSVQPEDFATYYC

SEQ ID NO: 300 - protein, 9 aa, Artificial Sequence
  source: /note=“Description of Artificial Sequence Synthetic peptide”
QQSYSTPFT

SEQ ID NO: 301 - protein, 10 aa, Artificial Sequence
  source: /note=“Description of Artificial Sequence Synthetic peptide”
FGPGTKVEIK

SEQ ID NO: 302 - protein, 25 aa, Artificial Sequence
  source: /note=“Description of Artificial Sequence Synthetic peptide”
EVQLVESGAEVKKPGASVKVSCKAS

SEQ ID NO: 303 - protein, 17 aa, Artificial Sequence
  source: /note=“Description of Artificial Sequence Synthetic peptide”
MHWVRQAPGQGLEWMGI

SEQ ID NO: 304 - protein, 8 aa, Artificial Sequence
  source: /note=“Description of Artificial Sequence Synthetic peptide”
INPSGGST

SEQ ID NO: 305 - protein, 40 aa, Artificial Sequence
  source: /note=“Description of Artificial Sequence Synthetic polypeptide”
IYAQKFQGRVTMTRDTSTSTVYMELSSLRSEDTAVYYCAR

SEQ ID NO: 306 - protein, 14 aa, Artificial Sequence
  source: /note=“Description of Artificial Sequence Synthetic peptide”
SLRDGYNYIGSLGY

SEQ ID NO: 307 - protein, 25 aa, Artificial Sequence
  source: /note=“Description of Artificial Sequence Synthetic peptide”
QSELTQPPSASGTPGQRVTISCSGS

SEQ ID NO: 308 - protein, 8 aa, Artificial Sequence
  source: /note=“Description of Artificial Sequence Synthetic peptide”
SSNIGSNY

SEQ ID NO: 309 - protein, 17 aa, Artificial Sequence
  source: /note=“Description of Artificial Sequence Synthetic peptide”
VYWYQQLPGTAPKLLIY

SEQ ID NO: 310 - protein, 3 aa, Artificial Sequence
  source: /note=“Description of Artificial Sequence Synthetic peptide”
RNN

SEQ ID NO: 311 - protein, 36 aa, Artificial Sequence
  source: /note=“Description of Artificial Sequence Synthetic polypeptide”
QRPSGVPDRFSGSKSGTSASLAIRGLQSEDEAGYYC

SEQ ID NO: 312 - protein, 8 aa, Artificial Sequence
  source: /note=“Description of Artificial Sequence Synthetic peptide”
GFTFDDYA

SEQ ID NO: 313 - protein, 17 aa, Artificial Sequence
  source: /note=“Description of Artificial Sequence Synthetic peptide”
MHWVRQAPGKGLEWVSG

SEQ ID NO: 314 - protein, 8 aa, Artificial Sequence
  source: /note=“Description of Artificial Sequence Synthetic peptide”
ISWNSGST

SEQ ID NO: 315 - protein, 40 aa, Artificial Sequence
  source: /note=“Description of Artificial Sequence Synthetic polypeptide”
YYADSVKGRFTISRDNSKNTLYLQMNSLRAEDTAVYYCAG

SEQ ID NO: 316 - protein, 17 aa, Artificial Sequence
  source: /note=“Description of Artificial Sequence Synthetic peptide”
GSRRYDSSGYYYESFDY

SEQ ID NO: 317 - protein, 36 aa, Artificial Sequence
  source: /note=“Description of Artificial Sequence Synthetic polypeptide”
NLETGVPSRFSGSGSGTDFTLTISSLQPEDFATYYC

SEQ ID NO: 318 - protein, 9 aa, Artificial Sequence
  source: /note=“Description of Artificial Sequence Synthetic peptide”
QQSYHTPYT

SEQ ID NO: 319 - protein, 25 aa, Artificial Sequence
  source: /note=“Description of Artificial Sequence Synthetic peptide”
QMQLVQSGAEVKKPGSSVKVSCKAS

SEQ ID NO: 320 - protein, 8 aa, Artificial Sequence
  source: /note=“Description of Artificial Sequence Synthetic peptide”
GGTFSSYA

SEQ ID NO: 321 - protein, 17 aa, Artificial Sequence
  source: /note=“Description of Artificial Sequence Synthetic peptide”
ISWVRQAPGQGLEWMGR

SEQ ID NO: 322 - protein, 8 aa, Artificial Sequence
  source: /note=“Description of Artificial Sequence Synthetic peptide”
IIPILGIA

SEQ ID NO: 323 - protein, 40 aa, Artificial Sequence
  source: /note=“Description of Artificial Sequence Synthetic polypeptide”
NYAQKFQGRVTITADKSTSTAYMELSSLRSEDTAVYYCAR

SEQ ID NO: 324 - protein, 14 aa, Artificial Sequence
  source: /note=“Description of Artificial Sequence Synthetic peptide”
DINRYNWNFRAFDI

SEQ ID NO: 325 - protein, 12 aa, Artificial Sequence
  source: /note=“Description of Artificial Sequence Synthetic peptide”
QSVLYSSNNKNY

SEQ ID NO: 326 - protein, 17 aa, Artificial Sequence
  source: /note=“Description of Artificial Sequence Synthetic peptide”
LAWYQQKPRQPPKLLIY

SEQ ID NO: 327 - protein, 3 aa, Artificial Sequence
  source: /note=“Description of Artificial Sequence Synthetic peptide”
WAS

SEQ ID NO: 328 - protein, 36 aa, Artificial Sequence
  source: /note=“Description of Artificial Sequence Synthetic polypeptide”
TRESGVPDRFSGNGSGTDFTLTISSLQAEDVAAYYC

SEQ ID NO: 329 - protein, 9 aa, Artificial Sequence
  source: /note=“Description of Artificial Sequence Synthetic peptide”
QQHYSTPLT

SEQ ID NO: 330 - protein, 25 aa, Artificial Sequence [sequence omitted]

SEQ ID NO: 331 - protein, 40 aa, Artificial Sequence
  source: /note=“Description of Artificial Sequence Synthetic polypeptide”
NYAQKLQGRVTMTTNTSTSTAYMELRSLRSDDTAVYYCAR

SEQ ID NO: 332 - protein, 17 aa, Artificial Sequence
  source: /note=“Description of Artificial Sequence Synthetic peptide”
LNWYRQKPGKAPKLLIY

SEQ ID NO: 333 - protein, 36 aa, Artificial Sequence
  source: /note=“Description of Artificial Sequence Synthetic polypeptide”
SLQSGVPSRFSGSGSGTDFTLTISSLQPEDAATYYC

SEQ ID NO: 334 - protein, 9 aa, Artificial Sequence
  source: /note=“Description of Artificial Sequence Synthetic peptide”
QQYDSQSGT

SEQ ID NO: 335 - protein, 25 aa, Artificial Sequence
  source: /note=“Description of Artificial Sequence Synthetic peptide”
EVQLVESGGGVVQPGRSLRLSCAAS

SEQ ID NO: 336 - protein, 8 aa, Artificial Sequence
  source: /note=“Description of Artificial Sequence Synthetic peptide”
ISYDGVKK

SEQ ID NO: 337 - protein, 40 aa, Artificial Sequence
  source: /note=“Description of Artificial Sequence Synthetic polypeptide”
YYADSVKGRFTISRDNSKSTLYLQMNSLRVDDTAVYYCAK

SEQ ID NO: 338 - protein, 8 aa, Artificial Sequence
  source: /note=“Description of Artificial Sequence Synthetic peptide”
DLGWQNDY

SEQ ID NO: 339 - protein, 33 aa, Artificial Sequence
  source: /note=“Description of Artificial Sequence Synthetic polypeptide”
QSVLTQPASVSGSPGQSITISCTGTDLGWQNDY

SEQ ID NO: 340 - protein, 9 aa, Artificial Sequence
  source: /note=“Description of Artificial Sequence Synthetic peptide”
SSDVGGHNY

SEQ ID NO: 341 - protein, 17 aa, Artificial Sequence
  source: /note=“Description of Artificial Sequence Synthetic peptide”
VSWYQQHPGKAPKLMIY

SEQ ID NO: 342 - protein, 3 aa, Artificial Sequence
  source: /note=“Description of Artificial Sequence Synthetic peptide”
DVS

SEQ ID NO: 343 - protein, 36 aa, Artificial Sequence
  source: /note=“Description of Artificial Sequence Synthetic polypeptide”
NRPSGVSNRFSGSKSGNTASLTISGLQAEDEADYYC

SEQ ID NO: 344 - protein, 10 aa, Artificial Sequence
  source: /note=“Description of Artificial Sequence Synthetic peptide”
SSYTSSSPWV

SEQ ID NO: 345 - protein, 11 aa, Artificial Sequence
  source: /note=“Description of Artificial Sequence Synthetic peptide”
FGGGTKLTVLG

SEQ ID NO: 346 - protein, 25 aa, Artificial Sequence [sequence omitted]

SEQ ID NO: 347 - protein, 40 aa, Artificial Sequence
  source: /note=“Description of Artificial Sequence Synthetic polypeptide”
YYADSVKGRFTISRDNSKNALYLQMNSLRAEDTAVYYCRG

SEQ ID NO: 348 - protein, 16 aa, Artificial Sequence
  source: /note=“Description of Artificial Sequence Synthetic peptide”
YCSSTSCYGRRGAFDI

SEQ ID NO: 349 - protein, 11 aa, Artificial Sequence
  source: /note=“Description of Artificial Sequence Synthetic peptide”
SGQGTLVTVSS

SEQ ID NO: 350 - protein, 25 aa, Artificial Sequence
  source: /note=“Description of Artificial Sequence Synthetic peptide”
QAVLTQPPSASGTPGQRVTISCSGR

SEQ ID NO: 351 - protein, 8 aa, Artificial Sequence
  source: /note=“Description of Artificial Sequence Synthetic peptide”
NSNIGSNN

SEQ ID NO: 352 - protein, 17 aa, Artificial Sequence
  source: /note=“Description of Artificial Sequence Synthetic peptide”
VNWYQHLPGTAPKLLIY

SEQ ID NO: 353 - protein, 3 aa, Artificial Sequence
  source: /note=“Description of Artificial Sequence Synthetic peptide”
SNN

SEQ ID NO: 354 - protein, 36 aa, Artificial Sequence
  source: /note=“Description of Artificial Sequence Synthetic polypeptide”
QRPSGVPDRFSASKSGTSASLAISGLQSEDEADYYC

SEQ ID NO: 355 - protein, 11 aa, Artificial Sequence
  source: /note=“Description of Artificial Sequence Synthetic peptide”
AAWDDRMNGPV

SEQ ID NO: 356 - protein, 11 aa, Artificial Sequence
  source: /note=“Description of Artificial Sequence Synthetic peptide”
IGGGTKVTVLG

SEQ ID NO: 357 - protein, 17 aa, Artificial Sequence
  source: /note=“Description of Artificial Sequence Synthetic peptide”
MHWVRQAPAKGLVWVSR

SEQ ID NO: 358 - protein, 8 aa, Artificial Sequence
  source: /note=“Description of Artificial Sequence Synthetic peptide”
INSDGSST

SEQ ID NO: 359 - protein, 40 aa, Artificial Sequence
  source: /note=“Description of Artificial Sequence Synthetic polypeptide”
SYADSVKGRFTISRDNSKNTLYLQMNSLRAEDTAVYYCAR

SEQ ID NO: 360 - protein, 14 aa, Artificial Sequence
  source: /note=“Description of Artificial Sequence Synthetic peptide”
DFWSGRPYYYYMDV

SEQ ID NO: 361 - protein, 6 aa, Artificial Sequence
  source: /note=“Description of Artificial Sequence Synthetic peptide”
QDIGDD

SEQ ID NO: 362 - protein, 17 aa, Artificial Sequence
  source: /note=“Description of Artificial Sequence Synthetic peptide”
LAWFQQKPGKAPKRLIY

SEQ ID NO: 363 - protein, 36 aa, Artificial Sequence
  source: /note=“Description of Artificial Sequence Synthetic polypeptide”
TLQGGVPSRFSGSGSGTDFTLTISSLQPEDFATYYC

SEQ ID NO: 364 - protein, 9 aa, Artificial Sequence
  source: /note=“Description of Artificial Sequence Synthetic peptide”
QQSYSTPRT

SEQ ID NO: 365 - protein, 40 aa, Artificial Sequence
  source: /note=“Description of Artificial Sequence Synthetic polypeptide”
YYADSVKGRFTISRDNSKNTLYLQMNSRRAEDTAVYYCAR

SEQ ID NO: 366 - protein, 16 aa, Artificial Sequence
  source: /note=“Description of Artificial Sequence Synthetic peptide”
WGIVAARPNYYYGMDV

SEQ ID NO: 367 - protein, 25 aa, Artificial Sequence
  source: /note=“Description of Artificial Sequence Synthetic peptide”
QSALTQPRSVSGSPGQSVTISCTGT

SEQ ID NO: 368 - protein, 9 aa, Artificial Sequence
  source: /note=“Description of Artificial Sequence Synthetic peptide”
SSDVGGYNY

SEQ ID NO: 369 - protein, 10 aa, Artificial Sequence
  source: /note=“Description of Artificial Sequence Synthetic peptide”
SSYANNSPWV

SEQ ID NO: 370 - protein, 11 aa, Artificial Sequence
  source: /note=“Description of Artificial Sequence Synthetic peptide”
FGGGTKVTVLG

SEQ ID NO: 371 - protein, 25 aa, Artificial Sequence
  source: /note=“Description of Artificial Sequence Synthetic peptide”
QVQLVQSGAEVRKPGASVKVSCKAS

SEQ ID NO: 372 - protein, 40 aa, Artificial Sequence
  source: /note=“Description of Artificial Sequence Synthetic polypeptide”
NYAQKLQGRVTMTTDTSTSTAYMELRSLRSDDTAVYYCAR

SEQ ID NO: 373 - protein, 26 aa, Artificial Sequence
  source: /note=“Description of Artificial Sequence Synthetic peptide”
DIQMTQSPSSLSASVGDRVTITCQAS

SEQ ID NO: 374 - protein, 6 aa, Artificial Sequence
  source: /note=“Description of Artificial Sequence Synthetic peptide”
QDISNY

SEQ ID NO: 375 - protein, 36 aa, Artificial Sequence
  source: /note=“Description of Artificial Sequence Synthetic polypeptide”
NLETGVPSRFSGSGSGTDFTFTISSLQPEDIATYYC

SEQ ID NO: 376 - protein, 9 aa, Artificial Sequence
  source: /note=“Description of Artificial Sequence Synthetic peptide”
QQYANLPLT

SEQ ID NO: 377 - protein, 8 aa, Artificial Sequence
  source: /note=“Description of Artificial Sequence Synthetic peptide”
GFTVSSNY

SEQ ID NO: 378 - protein, 17 aa, Artificial Sequence
  source: /note=“Description of Artificial Sequence Synthetic peptide”
MSWVRQAPGKGLEWVSV

SEQ ID NO: 379 - protein, 7 aa, Artificial Sequence
  source: /note=“Description of Artificial Sequence Synthetic peptide”
IYSGGST

SEQ ID NO: 380 - protein, 11 aa, Artificial Sequence
  source: /note=“Description of Artificial Sequence Synthetic peptide”
GGLTGDDAFDI

SEQ ID NO: 381 - protein, 6 aa, Artificial Sequence
  source: /note=“Description of Artificial Sequence Synthetic peptide”
QSISSF

SEQ ID NO: 382 - protein, 17 aa, Artificial Sequence
  source: /note=“Description of Artificial Sequence Synthetic peptide”
LNWYQQKPGTAPKLLIY

SEQ ID NO: 383 - protein, 3 aa, Artificial Sequence
  source: /note=“Description of Artificial Sequence Synthetic peptide”
TTS

SEQ ID NO: 384 - protein, 9 aa, Artificial Sequence
  source: /note=“Description of Artificial Sequence Synthetic peptide”
QQGNSLPLT

SEQ ID NO: 385 - protein, 6 aa, Artificial Sequence
  source: /note=“Description of Artificial Sequence Synthetic peptide”
DKELSY

SEQ ID NO: 386 - protein, 25 aa, Artificial Sequence
  source: /note=“Description of Artificial Sequence Synthetic peptide”
QSGLTQPASVSGSPGQSITISCTGT

SEQ ID NO: 387 - protein, 3 aa, Artificial Sequence
  source: /note=“Description of Artificial Sequence Synthetic peptide”
EVS

SEQ ID NO: 388 - protein, 36 aa, Artificial Sequence
  source: /note=“Description of Artificial Sequence Synthetic polypeptide”
NRPSGVPDRFSGSKSGNTASLTISGLQAEDEADYYC

SEQ ID NO: 389 - protein, 3 aa, Artificial Sequence
  source: /note=“Description of Artificial Sequence Synthetic peptide”
KTS

SEQ ID NO: 390 - protein, 36 aa, Artificial Sequence
  source: /note=“Description of Artificial Sequence Synthetic polypeptide”
NLQSGVPSRFSGSGSGTDFTLTISSLQPEDFATYYC

SEQ ID NO: 391 - protein, 17 aa, Artificial Sequence
  source: /note=“Description of Artificial Sequence Synthetic peptide”
MSWVRQAPAKGLEWVSA

SEQ ID NO: 392 - protein, 14 aa, Artificial Sequence
  source: /note=“Description of Artificial Sequence Synthetic peptide”
DRPYSYGKNDAFDI

SEQ ID NO: 393 - protein, 6 aa, Artificial Sequence
  source: /note=“Description of Artificial Sequence Synthetic peptide”
QDVSNY

SEQ ID NO: 394 - protein, 10 aa, Artificial Sequence
  source: /note=“Description of Artificial Sequence Synthetic peptide”
FGGGTKLEIK

SEQ ID NO: 395 - protein, 25 aa, Artificial Sequence
  source: /note=“Description of Artificial Sequence Synthetic peptide”
EVQLVQSGAEVKKPGASVKVSCKAS

SEQ ID NO: 396 - protein, 8 aa, Artificial Sequence
  source: /note=“Description of Artificial Sequence Synthetic peptide”
GYTFTSYY

SEQ ID NO: 397 - protein, 40 aa, Artificial Sequence
  source: /note=“Description of Artificial Sequence Synthetic polypeptide”
SYAQKFQGRVTMTRDTSTSTVYMELSSLRSEDTAVYYCAR

SEQ ID NO: 398 - protein, 11 aa, Artificial Sequence
  source: /note=“Description of Artificial Sequence Synthetic peptide”
AGGYYYYYMDV

SEQ ID NO: 399 - protein, 25 aa, Artificial Sequence
  source: /note=“Description of Artificial Sequence Synthetic peptide”
QSGLTQPPSASGTPGQRVTISCSGS

SEQ ID NO: 400 - protein, 8 aa, Artificial Sequence
  source: /note=“Description of Artificial Sequence Synthetic peptide”
GPNIGNNY

SEQ ID NO: 401 - protein, 17 aa, Artificial Sequence
  source: /note=“Description of Artificial Sequence Synthetic peptide”
VYWYQQLPGTAPKLLMY

SEQ ID NO: 402 - protein, 15 aa, Artificial Sequence
  source: /note=“Description of Artificial Sequence Synthetic peptide”
SPWLITFGGVIAMGY

SEQ ID NO: 403 - protein, 25 aa, Artificial Sequence
  source: /note=“Description of Artificial Sequence Synthetic peptide”
QSVLTQPPSASGTPGQRVTISCSGS

SEQ ID NO: 404 - protein, 36 aa, Artificial Sequence
  source: /note=“Description of Artificial Sequence Synthetic polypeptide”
QRPSGVPDRFSGSKSGTSASLAISGLRSEDEADYYC

SEQ ID NO: 405 - protein, 11 aa, Artificial Sequence
  source: /note=“Description of Artificial Sequence Synthetic peptide”
AAWDDSLSGVV

SEQ ID NO: 406 - protein, 11 aa, Artificial Sequence
  source: /note=“Description of Artificial Sequence Synthetic peptide”
FGGGTQLTVLG

SEQ ID NO: 407 - protein, 26 aa, Artificial Sequence
  source: /note=“Description of Artificial Sequence Synthetic peptide”
DIVMTQSPSSLSASVGDRVTITCRAS

SEQ ID NO: 408 - protein, 9 aa, Artificial Sequence
  source: /note=“Description of Artificial Sequence Synthetic peptide”
QQFNNYPLT

SEQ ID NO: 409 - protein, 25 aa, Artificial Sequence
  source: /note=“Description of Artificial Sequence Synthetic peptide”
QVQLVESGAEVKKPGASVKVSCKAS

SEQ ID NO: 410 - protein, 36 aa, Artificial Sequence
  source: /note=“Description of Artificial Sequence Synthetic polypeptide”
QRPSGVPDRFSGSKSGTSASLAISGLQSEDEADYYC

SEQ ID NO: 411 - protein, 11 aa, Artificial Sequence
  source: /note=“Description of Artificial Sequence Synthetic peptide”
AAWDDSLNGYV

SEQ ID NO: 412 - protein, 11 aa, Artificial Sequence
  source: /note=“Description of Artificial Sequence Synthetic peptide”
FGTGTKLTVLG

SEQ ID NO: 413 - protein, 120 aa, Artificial Sequence
  source: /note=“Description of Artificial Sequence Synthetic polypeptide”
QIQLVQSGAEVKKPGESLKISCKASGYTFTDYAIGWVRQMPGKGLEWMGIINTQTGKPKYSPSFQGQFIFSLDTSINTTYLQWSSLKASDTAIYFCTRLGTGNTKGFAYWGQGTTVTVSS

SEQ ID NO: 414 - protein, 114 aa, Artificial Sequence
  source: /note=“Description of Artificial Sequence Synthetic polypeptide”
DIQITQSPSSLSASLGDKVTITCRSSQSLLYSENNQDYLAWYQQKPGKAPKLLIYGASNLQSGVPSRFSGRGSGTDFTLTISSLQPEDFATYYCEQTYRYPFTFGPGTKVDIKR

SEQ ID NO: 415 - protein, 471 aa, Artificial Sequence
  source: /note=“Description of Artificial Sequence Synthetic polypeptide”
MGWSCIILFLVATATGVHSQVQLVQSGAEVKKPGASVKVSCKASGYTFPDYNMDWVRQAPGQRLEWMGYINPDNGGTIYNQKFKGRVTLTVDTSASTAYMELSSLRSEDTAVYYCARLDSSGYGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 416 - protein, 471 aa, Artificial Sequence
  source: /note=“Description of Artificial Sequence Synthetic polypeptide”
MGWSCIILFLVATATGVHSQVQLVQSGAEVKKPGASVKVSCKASGYTFPDYNMDWVRQAPGQRLEWIGYINPDNGGTIYNQKFKGRVTLTVDTSASTAYMELSSLRSEDTAVYYCARLDSSGYGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 417 - protein, 471 aa, Artificial Sequence
  source: /note=“Description of Artificial Sequence Synthetic polypeptide”
MGWSCIILFLVATATGVHSQVQLVQSGAEVKKPGASVKVSCKASGYTFPDYNMDWVRQAPGQRLEWMGYINPDNGGTIYNQKFKGRATLTVDTSASTAYMELSSLRSEDTAVYYCARLDSSGYGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 471 aa, Artificial Sequence
  source: /note=“Description of Artificial Sequence Synthetic polypeptide”
MGWSCIILFLVATATGVHSQVQLVQSGAEVKKPGASVKVSCKASGYTFPDYNMDWVRQAPGQSLEWIGYINPDNGGTIYNQKFKGRATLTVDTSASTAYMELSSLRSEDTAVYYCARLDSSGYGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 419 - protein, 237 aa, Artificial Sequence
  source: /note=“Description of Artificial Sequence Synthetic polypeptide”
MGWSCIILFLVATATGVHSDIVMTQSPDSLAVSLGERATINCRASESVDNYGISFMHWYQQKPGQPPKLLIYRASNLDSGVPDRFSGSGSGTDFTLTISSLQAEDVATYYCQQSYKDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 420 - protein, 237 aa, Artificial Sequence
  source: /note=“Description of Artificial Sequence Synthetic polypeptide”
MGWSCIILFLVATATGVHSDIVLTQSPASLAVSPGQRATITCRASESVDNYGISFMHWYQQKPGQPPKLLIYRASNLDSEVPARFSGSGSRTDFTLTINPVEANDTATYYCQQSYKDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 124 aa, Artificial Sequence
  source: /note=“Description of Artificial Sequence Synthetic polypeptide”
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSS

SEQ ID NO: 422 - protein, 124 aa, Artificial Sequence
  source: /note=“Description of Artificial Sequence Synthetic polypeptide”
QSLLESGGGLVKPGGSLRLSCAVSGFSFSSSYYMCWVRQAPGKGLEWVSCIGTTRGSTYYADSAKGRFTISKISKNTVYLQMTSLRAEDTAVYFCARDATGYRINTIGLYFNLWGPGTLVTVSS

SEQ ID NO: 423 - protein, 126 aa, Artificial Sequence
  source: /note=“Description of Artificial Sequence Synthetic polypeptide”
EVQLLESGGGLVKPGGSLRLSCAVSGFSFSSSYYMCWVRQAPGKGLEWVSCIGTTRGSTYYADSAKGRFTISKDNSKNTVYLQMTSLRAEDTAVYFCARDATGYRINTIGLYFNLWGQGTLVTVSS

SEQ ID NO: 424 - protein, 124 aa, Artificial Sequence
  source: /note=“Description of Artificial Sequence Synthetic polypeptide”
QSLLESGGGLVKPGGSLRLSCAVSGFSFSSSYYMCWVRQAPGKGLEWVSCIGTTRGSTYYADSAKGRFTISKESKNTVYLQMSSLRAEDTAVYFCARDATGYRINTIGLYFNLWGPGTLVTVSS

SEQ ID NO: 425 - protein, 126 aa, Artificial Sequence
  source: /note=“Description of Artificial Sequence Synthetic polypeptide”
EVQLLESGGGLVKPGGSLRLSCAVSGFSFSSSYYMCWVRQAPGKGLEWVSCIGTTRGSTYYADSAKGRFTISKDNSKNTVYLQMSSLRAEDTAVYFCARDATGYRINTIGLYFNLWGQGTLVTVSS

SEQ ID NO: 426 - protein, 124 aa, Artificial Sequence
  source: /note=“Description of Artificial Sequence Synthetic polypeptide”
QSLLESGGGLVKPGGSLRLSCAVSGFSFSSSYYMCWVRQAPGKGLEWVSCIGTTRGSTYYADSAKGRFTISKESKNTVYLQMSSLRAEDTAVYFCARDATGYRIQTIGLYFNLWGPGTLVTVSS

SEQ ID NO: 427 - protein, 126 aa, Artificial Sequence
  source: /note=“Description of Artificial Sequence Synthetic polypeptide”
EVQLLESGGGLVKPGGSLRLSCAVSGFSFSSSYYMCWVRQAPGKGLEWVSCIGTTRGSTYYADSAKGRFTISKDNSKNTVYLQMSSLRAEDTAVYFCARDATGYRIQTIGLYFNLWGQGTLVTVSS

SEQ ID NO: 428 - protein, 109 aa, Artificial Sequence
  source: /note=“Description of Artificial Sequence Synthetic polypeptide”
ELTQTPSSVEAAVGGTPTIKCQASQTIYSYLSWYQQKPGQPPKLLIYEASKLASGVPSRFSGSGSGTDYTLTISDLECADAATYYCQSYHGTASTEYNTFGGGTEVVVK

SEQ ID NO: 429 - protein, 109 aa, Artificial Sequence
  source: /note=“Description of Artificial Sequence Synthetic polypeptide”
QLTQSPSSLSASVGDRVTITCQASQTIYSYLSWYQQKPGKPPKLLIYEASKLASGVPSRFSGSGSGTDYTLTISSLQPEDFATYYCQSYHGTASTEYNTFGGGTKVEIK

SEQ ID NO: 430 - protein, 111 aa, Artificial Sequence
  source: /note=“Description of Artificial Sequence Synthetic polypeptide”
DIQLTQSPSSLSASVGDRVTITCQASQTIYSYLSWYQQKPGKPPKLLIYEASKLASGVPSRFSGSGSGTDYTLTISSLQPEDFATYYCQSYHGTASTEYNTFGGGTKVEIK

SEQ ID NO: 431 - protein, 109 aa, Artificial Sequence
  source: /note=“Description of Artificial Sequence Synthetic polypeptide”
QLTQSPSSLSASVGDRVTITCQASQTIYSYLSWYQQKPGKPPKLLIYEASKLASGVPSRFSGSGSGTDYTLTISSLQPEDTATYYCQSYHGTASTEYNTFGGGTKVEIK

SEQ ID NO: 432 - protein, 111 aa, Artificial Sequence
  source: /note=“Description of Artificial Sequence Synthetic polypeptide”
DIQLTQSPSSLSASVGDRVTITCQASQTIYSYLSWYQQKPGKPPKLLIYEASKLASGVPSRFSGSGSGTDYTLTISSLQPEDTATYYCQSYHGTASTEYNTFGGGTKVEIK

SEQ ID NO: 433 - protein, 109 aa, Artificial Sequence
  source: /note=“Description of Artificial Sequence Synthetic polypeptide”
QLTQSPSSLSASVGDRVTITCQASQTIYSYLSWYQQKPGKPPKLLIYEASKLASGVPSRFSGSGSGTDYTLTISSLQPEDTATYYCQSYHGTASTEYQTFGGGTKVEIK

SEQ ID NO: 434 - protein, 111 aa, Artificial Sequence
  source: /note=“Description of Artificial Sequence Synthetic polypeptide”
DIQLTQSPSSLSASVGDRVTITCQASQTIYSYLSWYQQKPGKPPKLLIYEASKLASGVPSRFSGSGSGTDYTLTISSLQPEDTATYYCQSYHGTASTEYQTFGGGTKVEIK

SEQ ID NO: 435 - protein, 26 aa, Artificial Sequence
  source: /note=“Description of Artificial Sequence Synthetic peptide”
DIVMTQSPDSLAVSLGERATINCKSS

SEQ ID NO: 436 - protein, 8 aa, Artificial Sequence
  source: /note=“Description of Artificial Sequence Synthetic peptide”
GFTFSTYG

SEQ ID NO: 437 - protein, 8 aa, Artificial Sequence
  source: /note=“Description of Artificial Sequence Synthetic peptide”
GFTFSSYW

SEQ ID NO: 438 - protein, 8 aa, Artificial Sequence
  source: /note=“Description of Artificial Sequence Synthetic peptide”
GFTFDDYG

SEQ ID NO: 439 - protein, 8 aa, Artificial Sequence
  source: /note=“Description of Artificial Sequence Synthetic peptide”
GYTFTSYG

SEQ ID NO: 440 - protein, 8 aa, Artificial Sequence
  source: /note=“Description of Artificial Sequence Synthetic peptide”
GFTFSSYA

SEQ ID NO: 441 - protein, 8 aa, Artificial Sequence
  source: /note=“Description of Artificial Sequence Synthetic peptide”
GYTFTRYY

SEQ ID NO: 442 - protein, 14 aa, Artificial Sequence
  source: /note=“Description of Artificial Sequence Synthetic peptide”
DLGHFDSGSSYFDY

SEQ ID NO: 443 - protein, 36 aa, Artificial Sequence
  source: /note=“Description of Artificial Sequence Synthetic polypeptide”
KRPSGVPDRFSGSKSGNTASLTISGLQAEDEADYHC